(19) United States
(12) Patent Application Publication
(10) Pub. No.: US 2024/0261426 A1
(43) Pub. Date: Aug. 8, 2024

(54) CONJUGATES COMPRISING PHOSPHOANTIGENS AND THEIR USE IN THERAPY

(71) Applicant: BYONDIS B.V., Nijmegen (NL)

(72) Inventors: Ronald Christiaan ELGERSMA, Nijmegen (NL); Dennis Christian Johannes WAALBOER, Nijmegen (NL)

(21) Appl. No.: 18/573,284

(22) PCT Filed: Jun. 28, 2022
(86) PCT No.: PCT/EP2022/067693

§ 371 Date: Dec. 21, 2023

(30) Foreign Application Priority Data
Jun. 28, 2021 (EP) 21182160.8

(51) Int. Cl. A61K 47/68; A61P 35/00

(52) U.S. Cl. CPC A61K 47/6851; A61K 47/6803; A61K 47/6889; A61P 35/00

(57) ABSTRACT

The present invention relates to novel conjugates comprising a targeting moiety linked to a phosphoantigen moiety, and the use thereof in the treatment of diseases, such as cancer, infectious diseases and autoimmune diseases, optionally in combination with other therapeutic agents. A targeting moiety may be an antibody, or binding fragment thereof. The phosphoantigen may be a prodrug. The invention further relates to linker-drug compounds comprising a phosphoantigen moiety, for use in the manufacture of conjugates, and pharmaceutical compositions comprising said immunoconjugates.

### FIELD OF THE INVENTION

The present invention relates to novel conjugates comprising a targeting moiety, for example an antibody or a binding fragment thereof, linked to a phosphoantigen moiety, and the use thereof in the treatment of diseases, such as cancer, infectious diseases and autoimmune diseases, optionally in combination with other therapeutic agents. The invention further relates to linker-drug compounds comprising a phosphoantigen moiety for use in the manufacture of conjugates and pharmaceutical compositions comprising said immunoconjugates.

### BACKGROUND OF THE PRESENT INVENTION

Conventional methods to treat cancer involve surgery, chemotherapy with cytotoxic agents and radiation therapy, or a combination of these treatments. Due to their toxic and non-specific nature, treatment with cytotoxic agents or radiation often lead to severe side effects. Since it was discovered that the immune system plays an important role in eradicating neoplastic cells, more recent cancer therapies aim to use components of the immune system as a tool to treat cancer.

One approach used in cancer immunotherapy is to target “immune checkpoints”, such as T-lymphocyte associated protein 4 (CTLA-4) or programmed cell death protein 1 (PD-1), aiming to activate anti-tumor immune responses in patients with cancer. Both CTLA-4 and PD-1 are proteins involved in negative feedback systems, which function to restrain immune cell activation. Tumor cells can escape from the immune system by “abusing” this suppression mechanism by overexpressing immune-checkpoint ligands on their surface, to protect themselves from an attack by cells of the immune system. Activation of immune checkpoints, by interaction with their ligands, leads to T-cell inactivation and exhaustion. Immune checkpoint inhibitors, such as antibodies directed against immune checkpoints or their ligands, are a new class of anti-cancer drugs that block the immune checkpoints overexpressed on cancer cells. Examples of approved immune checkpoint inhibitors are ipilimumab (blocking CTLA-4; brand name Yervoy®, produced by BMS), approved in 2011 for treatment of melanoma, PD-1 antibody nivolumab (sold under the brand name Opdivo® and developed by BMS) and pembrolizumab (brand name Keytruda®, another PD-1 inhibitor, produced by Merck). While checkpoint inhibitors can reinvigorate an anti-tumor response, activated immune cells can also attack normal tissue, leading to immunological adverse side-effects.

Another approach to cancer therapy involves the use of Antibody-Drug Conjugates (ADCs). ADCs combine the specificity of a monoclonal antibody for a tumor specific antigen with the cell killing activity of a chemical cytotoxic agent. The antibody of an ADC acts as a targeting agent and carrier for the cytotoxic payload. The binding of the antibody to its target effectuates efficient uptake of the ADC, with its cytotoxic payload, into the target tumor cells. The cytotoxic payload may be an inactive precursor (prodrug) of a cytotoxic agent, grafted onto the antibody via a linker which is stable in circulation, and is cleaved after being internalized into the tumor cell, for example by intracellular proteases. The cleavage of the linker may trigger the release of the active, cytotoxic form of the payload in the tumor cell. ADCs have the advantage that toxic, and non-specific side-effects on healthy tissue, can be greatly reduced. ADCs that have been clinically approved include, gemtuzumab (anti-CD33) ozogamicin (Mylotarg®; Wyeth Pharmaceuticals, a subsidiary of Pfizer), brentuximab (anti-CD30) vedotin (Adcetris®; Seattle Genetics/Millennium Pharmaceuticals), (ado-)trastuzumab (anti-HER2) emtansine (Kadcyla®; Genentech/Roche), inotuzumab (anti-CD22) ozogamicin (Besponsa®; Wyeth Pharmaceuticals, a subsidiary of Pfizer), enfortumab (anti-nectin-4) vedotin (Padcev™; Astellas Pharma/Seattle Genetics), fam-trastuzumab deruxtecan (Enhertu®; Daiichi Sankyo/AstraZeneca), polatuzumab (anti-CD79b) vedotin (Polivy™; Genentech/Roche) and sacituzumab (anti-TROP-2) govitecan (Trodelvy™; Immunomedics). Many more are in clinical development.

Yet another approach to cancer therapy is immunotherapy using therapeutic compounds that activate the immune system, in particular T-cells, to attack and destroy tumor cells. Such therapeutic compounds may be agonists of immune cell receptors and can be large molecules or relatively small chemical structures. An example of such compounds are ligands activating Toll Like Receptors (TLRs). Several TLR ligands have been approved for cancer therapy. The first approved TLR ligand (TLR agonist) form part of an attenuated strain of Mycobacterium bovis called Bacillus Calmette-Guerin (BCG). First developed as a tuberculosis vaccine, BCG contains active TLR2/4 ligand and has been used as a treatment for bladder cancer. Other approved TLR ligands are the TLR4 ligand monophosphoryl lipid A (MPLA) and the small molecule TLR7 agonist imiquimod, an imidazoquinoline.

TLR ligands have also been used in immunoconjugates. Such immunoconjugates comprise an antibody specific for a tumor antigen as targeting vehicle for a TLR ligand, with the aim to induce localized activation of cells of the immune system in the tumor microenvironment. Immunoconjugates, for the treatment of breast cancer, wherein TLR agonist were coupled to anti-HER antibodies are described in WO2017/072662 (Novartis A.G.). A further anti-HER conjugates with a TLR8 agonist payload were developed by Silverback Therapeutics (ImmunoTAC™ SBT6050). Bolt Therapeutics (WO2020/047187) and Ackerman et al., 2021, Nature Cancer, Vol. 2(8), 18-33, also describe TLR immunoconjugates, comprising a tumor-targeting monoclonal antibody, conjugated to a TLR 7/8 agonist (T785) via a non-cleavable linker; The tumor targeting antibody bound to a tumor antigen activates antigen presenting cells present in the tumor microenvironment (TME) via Fc effector functions, while the TLR agonist bound thereto directly stimulates APCs through their TLR receptors, which in turn promotes anti-tumor immunity.

A specific subset of T-cells known to display cytotoxicity against cancer cells are gammadelta T-cells. (T-cells with T-cells receptors (TCRs) composed of gamma and delta chains). Gamma delta T-cells are considered a unique subset of T-lymphocytes due to their ability to effectuate a rapid, innate-like immune response to infection and to tumor cells. Tumor-infiltrating gammadelta T-cells (γδ T-cells) were found in many different malignancies (Gentles et al., Nature Medicine, 2015, 21(8), 938-945). Gammadelta T-cells, or more specifically; Vgamma9Vdelta2 T-cells (Vγ9Vδ2 T-cells), which form a major subset of gammadelta T cells, can be activated by a specific set of antigens known as “phosphoantigens”. Naturally occurring phosphoantigens are low molecular alkyl pyrophosphates, such as 4-hydroxy-3-methyl-but-2-enyl-pyrophosphate (HMBPP) and isopentenyl pyrophosphate (IPP). These natural phosphoantigens are produced by pathogenic cells where HMBPP is the immediate precursor of IPP (HMBPP is a pathogenic phosphate antigen that does not occur in humans). Bacteria and parasites can produce isoprenoid precursors using a mevalonate-independent pathway (MEP) pathway or 2-C-methyl-D-erythritol 4-phosphate/1-deoxy-D-xylulose 5-phosphate (MEP/DOXP) pathway), resulting in the biosynthesis of the isoprenoid precursor IPP. In humans pAg production is driven by the mevalonate pathway.

In contrast to TLR agonists, phosphoantigens do not work directly on receptors displayed on myeloid cells or T-cells. It is believed that intracellular (e.g. within a cancer cell) binding of a phosphoantigen to an intracellular domain of a cell surface molecule, butyrophilin 3A1 (BTN3A1) causes conformational changes in relation to the extracellular portion of the BTN3A1 complex, which also includes a role for BTN2A1 (Sandstrom A, et al., 2014, Immunity, 40(4), 490-500, doi: 10.1016/j.immuni.2014.03.003).

The conformational changes in the extracellular BTN3A1/BTN2A1 complex result in binding to the gammadelta TCR, which results in cytokine production and killing of the tumor/pathogenic cell by the activated gammadelta T-cell (Rigau et al., Science, 2020, 367, 642). The activation of gammadelta T-cells using phosphoantigens as therapeutic agents is thus indirect; The phosphoantigen acts from within a cell (e.g. a tumor cell or infected cell), to effectuate a conformational change in the extracellular BTN3A1/BTN2A1 complex on the surface of said cell, which in turn provides an activating signal to gammadelta TCRs on gammadelta T-cells. The gammadelta T-cells will in turn exert their cell killing effect on the tumor cells or infected cells.

Because pyrophosphate HMBPP has poor pharmacokinetic properties (it is rapidly hydrolyzed in plasma), (nitrogenous) bisphosphonate analogs have been developed, as well as (monophosphate) prodrug forms that are converted to active phosphoantigens after they are administered to a subject. In phosphoantigen-prodrugs, the negatively charged non-binding oxygen atoms of the phosphonate group(s) are protected with neutral groups to increase, for example, diffusion over the cell membrane. The protecting groups are removed once inside the cell to release the active phosphoantigen. Another approach to improve the half-life in circulation of phosphoantigens (in particular of bisphosphonate phosphoantigens) is described in WO2012/042024. Phosphoantigens were complexed to nanoparticles with inorganic and lipid nano vectors, serving as delivery vehicles for the phosphoantigens. It was mentioned that the resulting nanoparticles can be coated with targeting ligands on their surface, to target specific cells. Examples mentioned include molecules that induce targeting to cancer cells, such as antibodies. The use of human transferrin was exemplified.

Phosphoantigens have been tested for use in cancer therapy, with the aim to promote the cytotoxic effect of gammadelta T-cells on tumor cells, either in vivo or by expanding gammadelta T-cells in vitro together with antigen presenting cells, for administration to a subject. Synthetic phosphoantigens, such as BrHPP (Phosphostim, manufactured by Innate Pharma) and Zoledronate (Novartis) have been the subject of clinical testing in patients with cancer. Phosphoantigens that were the subject of clinical testing showed an acceptable safety profile. However, their efficacy was general not sufficient. (Sebestyen et al., Nature Reviews Drug Discovery, 2020, 19(3), 169-184).

Finding an acceptable therapeutic window for such treatment may be greatly improved by more robust, selective, as well as effective, ways to deliver phosphoantigens to cells (over) expressing butyrophilin (BTN3A1/BTN2A1) complexes, such as tumor- or pathogenic cells.

### BRIEF DESCRIPTION OF THE PRESENT INVENTION

The present invention provides more effective and selective ways of using phosphoantigens in treatment of, for example, cancer. The present invention relates to conjugates, comprising a targeting moiety covalently linked to an immunomodulating moiety, wherein the immunomodulating moiety is a phosphoantigen moiety (pAg). Preferably the targeting moiety is a tumor-targeting antibody or antigen binding fragment thereof. Such conjugates can be used to activate gammadelta T-cells, for example, in the treatment of diseases such as cancer, infection, or autoimmune disease. Conjugates according to the present invention can be used either alone, or in combination with other therapeutic agents.

Preferably conjugates according to the invention are immunoconjugates comprising a tumor-targeting antibody or an antigen binding fragment thereof as targeting moiety. Such conjugates according to the invention, comprising tumor-targeting antibodies as targeting moiety, can be used to specifically deliver phosphoantigens to localized tumor cells, where they may be internalized into the tumor cell after binding, of the antibody or antigen binding fragment thereof, to its tumor specific or tumor associated antigen (TAA). The invention also provides linker-drug compounds for use in the manufacture of conjugates according to the invention, wherein the “drug” is a phosphoantigen moiety. The invention further provides pharmaceutical compositions comprising a conjugate according to the invention and one or more pharmaceutical excipients. Conjugates according to the invention may be used as a medicament, for example for the treatment of cancer.

### BRIEF DESCRIPTION OF THE DRAWINGS

FIG. 1: FCC/Gating strategy to discriminate indicated various immune cell populations. First, a time gate was applied to assure a constant flow (A). Subsequently, lymphocyte doublets were excluded on the FSC-A versus FSC-H (B) and SSC-A versus SSC-H plots (C). Viable cells were then selected (D), followed by selection of lymphocytes (E). CD3-negative, CD56-positive cells were then identified as NK cells (F). CD3-positive cells were further divided into Vd2 and Vd1 positive cells (G). CD3-positive Vd2-negative Vd1-negative lymphocytes were also subdivided into CD8-positive cytotoxic T-cells (H).

FIG. 2: CD107a (FIG. 2A) or IFNγ (FIG. 2C) production by Vδ2 γδ T-cells. Levels of activation are indicated by the proportion of Vδ2 γδ T-cells that are IFNγ+ or CD107a+. Measurements are performed using N=4 healthy donors, which are all depicted for the Vδ2 γδ T cell population. (Some graphs in FIG. 2 and FIG. 4 reflect “IFNγ” as “IFNy”.) For one representative donor CD107a+ production (FIG. 2B) and IFNγ production (FIG. 2D) were shown on NK cells, Vδ1 γδ T-cells or CD8+ T-cells gated from total PBMCs co-cultured with a concentration range of prodrug/HMBPP (M) pre-treated Raji cells.

FIG. 3: CD107a or IFNγ production of PHA activated Vδ2 γδ T-cells (A,I), NK cells (B,J), Vδ1 γδ T-cells (C, K) and CD8⁺ T-cells (D,L) or of unstimulated Vδ2 γδ T-cells (E,M), NK cells (F,N), Vδ1 γδ T-cells (G,O) and CD8⁺ T-cells (H,P). FACS plots show CD107a (A-H) or IFNγ (I-P) profiles of a representative healthy donor.

FIG. 4: CD107a (A-D) and IFNγ (E-H) production by gated Vδ2 γδ T-cells (A,E), NK cells (B,F), Vδ1 γδ T-cells (C,G) and CD8⁺ T-cells (D,H) after co-culture of PBMCs with a concentration range of ADC or rituximab pre-treated Raji cells. Levels of activation are indicated by the proportion of immune cell subsets that are IFNγ⁺ or CD107a⁺. Measurements were performed using N=3 (ADC-XD13-r, ADC-XD13-i) or N=4 (ADC-XC4-r, ADC-XC4-i, ADC-XD4-r, ADC-XD4-i, rituximab) different healthy donors. Results are shown for one representative donor.

FIG. 5: EC50 values (A-B), maximum proportions of Vδ2 γδ T-cells positive (C-D) and median fluorescent intensity (MFI, E-F) of CD107a (A, C, E) or IFNγ (B, D, F) production of Vδ2 γδ T-cells after co-culture with ADC-or rituximab-treated Raji cells. The MFI was determined on CD107a⁺ or IFNγ⁺ Vδ2 γδ T-cells. Measurements were performed using N=3 (ADC-XD13-r) or N=4 (ADC-XC4-r, ADC-XD4-r, rituximab) different healthy donors and each symbol represents an individual donor.

FIG. 6: Representative IFNγ/CD107a profile of electronically gated Vδ2 γδ T-cells co-cultured with Raji cells pretreated with ADC-XC4-r (A), ADC-XD4-r (B), ADC-XD13-r (C), rituximab (D).

FIG. 7: Direct effect of rituximab, zoledronate, HMBPP, pAg conjugates or duocarmycin as positive control, on Raji cell survival measured after 6 days incubation. Duocarmycin was used as positive control. (A) A concentration range of indicated compounds was incubated with Raji cells and cell death was determined. The controls and free drugs are displayed in one graph, and for readability, the different pAg conjugates were split over three graphs. (B) Overview of cell survival (%) at the highest pAg conjugate or rituximab concentration (10 μg/mL).

FIG. 8: CD107a production by gated Vδ2 γδ T-cells after co-culture of PBMCs with a concentration range of pAg conjugates or rituximab pretreated Raji cells. Levels of activation are indicated by the proportion of immune cell subsets that are CD107a+. Measurements were performed using different healthy donors. Each plots represents a different experiment and each letter indicates the donor that was used. When the same donor was tested in two different experiments this was indicated with ‘-1’ or ‘-2’.

FIG. 9: CD107a (A, D, G), IFNγ (B, E, H) and TNFα (C, F, I) production by gated Vδ2 γδ T-cells after coculture of PBMCs with a concentration range of pAg conjugates or rituximab pretreated Raji cells. Boxplots show EC₅₀ values (A, B, C), % activated cells (D, E, F) or median fluorescence intensity (MFI, G, H, I). The dots depicted in the box plots (D-I) represent the mean values. The data are a graphical representation of Table 5-10. Of note, TNFα production was not assessed in every experiment.

FIG. 10: Correlations between CD107a and IFNγ production (EC₅₀ values, % activity and MFI) by γδ T-cells cocultured with pAg conjugate pretreated Raji cells. The activity of pAg conjugates was tested in different experiments with different donors, and geomean EC₅₀ values (A), or mean % activity (B) and mean ‘median fluorescence intensities’ (MFIs, C) were calculated for each pAg conjugate. The correlation between the CD107a and IFNγ EC₅₀, % activity and MFI was plotted. A dotted line was indicated for rituximab.

FIG. 11: CD107a (A, D), IFNγ (B, E) and TNFα(C, F) EC₅₀ values (A-C) and maximum proportions of positive Vδ2 γδ T cells (D-F) after coculture with Raji cells pretreated with ADC-XD65-r, ADCs-XD65-r or rituximab. Measurements were performed using N=2 different healthy donors as indicated.

FIG. 12: Killing of pAg conjugate pretreated Raji cells by Vδ2 γδ T cells. Raji cells were pretreated with no compound (effector+target; E+T; grey squares), HMBPP (‘+’ symbol), or a concentration range of ADC-XD18-r (black closed circles), ADC-XD18-i (black open circles, dotted line) or rituximab (grey diamonds) for 16 hours and subsequently cocultured with expanded Vδ2 γδ T cells for 1 hour. The killing of Raji cells was then assessed using flow cytometry. (A) Dose-dependent killing of Raji cells by expanded Vδ2 γδ T-cells from indicated donors. (B) Correlation between the rituximab efficacy and the CD16 expression on expanded Vδ2 γδ T-cells. (C,D) Summarizing graphs show EC₅₀ values of the % dead Raji cells (C) and the % efficacy (D) of all tested donors.

FIG. 13: Activity of ADC-XD18-i, ADC-XD18-r, rituximab and HMBPP after pretreatment with multiple CD20 positive cell lines on Vδ2 γδ T-cells. (A) Proportions of CD107a-positive Vδ2 γδ T-cells after coculture with HMBPP pretreated cell lines. (B-F) Proportions of CD107a-positive Vδ2 γδ T-cells after coculture with a concentration range of ADC-XD18-i, ADC-XD18-r or rituximab pretreated cell lines. All experiments have been performed with N=2 different healthy donors that are depicted in the graphs.

FIG. 14: Proportion of Vδ2 γδ T-cells producing CD107a (A-D), IFNγ (E-H) or TNFα (I-L) after coculture with pretreated BT-474 (A, E, I), SK-BR-3 (B, F, J), SK-OV-3 (C, G, K) or HCT-116 cells (D, H, L), with a concentration range of ADC-XD18-t, ADC-XD18-i or trastuzumab. Results are shown for one representative donor from N=2 (BT-474, SK-OV-3 and HCT-116) and N=4 (SK-BR-3).

FIG. 15: EC₅₀ values (A-C) and % efficacy (D-F) of CD107a (A, D), IFNγ (B, E) or TNFα (C, F) production of Vδ2 γδ T-cells after 6 hours coculture with pAg conjugate- or trastuzumab-treated cell lines. Each symbol represents a healthy donor and geomean (A-C) or mean (D-F) values are indicated.

FIG. 16: Proportions of CD107a-(A), IFNγ-(B) or TNFα-positive (C) Vδ2 γδ T-cells after 6 hours coculture with HMBPP pretreated cell lines. Each symbol represents an individual healthy donor and mean values are indicated.

FIG. 17: Proportion of NK-cells producing CD107a after coculture with pretreated BT-474, SK-BR-3, SK-OV-3 or HCT-116 cells with a concentration range of ADC-XD18-t, ADC-XD18-i or trastuzumab. Results are shown for one representative donor from N=2 (BT-474, SK-OV-3 and HCT-116) and N=4 (SK-BR-3).

FIG. 18: CD107a production by gated Vδ2 γδ T-cells after co-culture of PBMCs with a concentration range of pAg conjugates or control compound pretreated Raji or MOLM-13 cells. (A) % activity (CD107a) of Vδ2 γδ T-cells after coculture with HMBPP pretreated Raji or MOLM-13 cells. (B-E) Levels of activation of Vδ2 γδ T-cells in PBMCs from different healthy donors after coculture with pretreated Raji (top) or MOLM-13 cells (bottom). Each plots represents a different experiment and each letter indicates the donor that was used. (F-H) Summarizing graphs show EC₅₀ values (F), % efficacy (G), and median fluorescence intensity (MFI, H) of all tested donors.

### DETAILED DESCRIPTION OF THE PRESENT INVENTION

With the present invention conjugates are provided, comprising a targeting moiety covalently linked to an immunomodulating moiety, wherein the immunomodulating moiety is a phosphoantigen (pAg) moiety.

### Conjugates

The present invention provides a conjugate, comprising a targeting moiety covalently linked to an immunomodulating moiety, wherein the immunomodulating moiety is a phosphoantigen (pAg) moiety.

Conjugates according to the invention comprise a targeting moiety that specifically binds to a target cell. Preferably the targeting moiety is a tumor-targeting antibody or antigen binding fragment thereof. The targeting moiety serves as a delivery vehicle; it delivers, to a target cell, the pAg moiety covalently linked to the targeting moiety. The pAg moiety may be coupled directly to, for example, an amino acid side chain in a (polypeptide) targeting moiety. Preferably, however, the pAg is conjugated to a targeting moiety, via a linking moiety.

Preferred conjugates according to the present invention can be represented by the general formula I

Tm-(L-(pAg)ₓ)_{y} (I),

wherein Tm represents a targeting moiety, preferably an antibody or an antigen binding fragment thereof, L represents a linking moiety, pAg represents a phosphoantigen moiety, x represents the number of phosphoantigen moieties per linking moiety, and has a value ranging from 1-5 and y represents the average number of L-(pAg)ₓ, per Tm (linker moieties per targeting moiety) and is an integer ranging from 1-10, preferably 1-8. The number of pAg moieties per conjugate (pAg to Tm ratio) in formula I is x multiplied by y. The average pAg-to-Tm ratio can be in the range from 1 to 16, or even 20, or higher. The ratio of pAg units per targeting moiety can be varied, for example, based on structural or functional characteristics of either the phosphoantigen moiety or the targeting moiety. In practice, the number of pAg per targeting moiety in the range of 2-8 or 2-6, or even as low as 2 may provide a sufficient therapeutic effect. Preferably, a linking moiety carries 1 or 2 pAg moieties. In most instances it may suffice for each linking moiety to carry 1 pAg. Preferably the target pAg to Tm ratio is 2 (x is 1 and y is 2).

Linker moieties preferably are cleavable linker moieties. In conjugates according to the invention straight or branched linker moieties may be used. When multiple phosphoantigen moieties are linked to one targeting moiety, each phosphoantigen moiety may be covalently coupled to the targeting moiety by a separate linking moiety. In practice, when the targeting moiety is an antibody, and coupling occurs to reduced interchain disulfides, there may be as many as 8 separate linking moieties attached to one targeting moiety, resulting in 8 phosphoantigen moieties per target moiety when each phosphoantigen moiety is carried by its own linking moiety. In the alternative branched linker moieties may carry 1-5 phosphoantigen moieties per linking moiety (x is 1, 2, 3, 4 or 5). Especially when a higher pAg to Tm ratio is desired, or when only a limited number of binding places are available on a targeting moiety, branched linkers are preferred. For example, branched linkers carrying 2 pAgs (x is 2) can be used to increase the number of phosphoantigen moieties per targeting moiety to a higher value. By using such linking moieties, e.g. 16 phosphoantigen moieties can be bound to a targeting moiety using only 8 linking moieties. Antibodies can be modified to introduce additional cysteines, in addition to the number of cysteines, in the antibody amino acid sequence, that form disulfide bonds and can be reduced and conjugated to a linker-drug molecule. For example, additional cysteines can be introduced at positions such as the 41C position, as disclosed in WO2015177360. For antibodies containing as many as 10 cysteines available for conjugation to which linking moieties can be bound, a DAR of 20 (x is 2, y is 10) or higher can even be reached, when branched linkers carrying two pAg moieties per linker (x is 2) are used. Under optimal conditions, all binding sites in a targeting moiety will be occupied by a linking moiety. In practice, a conjugate mixture may be produced wherein the exact number of phosphoantigen moieties per target moiety may vary somewhat, depending on the reaction conditions, and y values are average numbers.

Conjugates according to the invention, comprising a phosphoantigen moiety, may be used in combination with other pharmaceutically active compounds that may be simultaneously or sequentially administered to a subject in need thereof. Additionally, a targeting moiety may carry a combination of a phosphoantigen and a different payload. The advantage of such a “multiple payload” approach is that both actives will be targeted by the same targeting moiety. The ratio between the payloads of course has to be appropriately set by the (conjugation) reaction conditions and binding sites. Separate linker-drug compounds for each payload may, for example, be conjugated to different binding sites (e.g. different types of amino acids) on the targeting moiety and/or be conjugated by different conjugation methods and/or different linker chemistry to control the binding, distribution and drug to antibody ratio (DAR) of both payloads. Antibody drug conjugates (ADCs) carrying multiple cytotoxic payloads are known in the art. Conjugates according to the invention may combine a phosphoantigen moiety, for example, with a cytotoxic payload or with another immunomodulatory payload designed to enhance the overall desired therapeutic effect. Any non-specific binding to- and/or effects on non-target tissue of a phosphoantigen at non-target sites, is thus diminished.

As is well-known in the art, the drug load distribution in an ADC can be determined, for example, by using hydrophobic interaction chromatography (HIC) or reversed phase high-performance liquid chromatography (RP-HPLC). HIC is particularly suitable for determining the average DAR (pAg to Tm ratio in a conjugate according to the invention).

### Targeting Moiety

A targeting moiety specifically or preferably binds to a target cell and can be a targeting antibody, or an antigen binding fragment thereof, or another targeting moiety such as, for example, nucleic acids (aptamers) or (poly)peptides, which may be enzyme inhibitors, enzyme substrates, receptor ligands, and/or fusion proteins. Also small-molecule inhibitors can be used as targeting moieties (resulting in small molecule drug conjugates (SMDC's). The binding specificity (and affinity) of the targeting moiety for its target determine where, in the body, a conjugate according to the invention will exert its therapeutic effect.

Thus, by selecting an appropriate targeting moiety, it is ensured that a phosphoantigen moiety is delivered at the site where it has to exert its therapeutic effect.

Preferably the targeting moiety in a conjugate according to the invention is an antibody, or an antigen binding fragment thereof. In case the targeting moiety is an antibody, or an antigen binding fragment thereof, conjugates are commonly referred to as immunoconjugates, or antibody drug conjugates (ADC). Targeting antibodies are antibodies that recognize an antigen expressed by a target cell, such as a tumor associated antigen, with high specificity.

The specificity of the antibody or fragment for its antigen allows for the specific delivery of an effector molecule (or “payload”) to the target cell, leaving healthy tissue largely unaffected. An effector molecule is covalently coupled to the antibody via a linker that ensures that the effector molecule stays connected to the antibody, at least until the antibody reaches the target cell, e.g. a cancer cell. Effector molecules exert their effect on or in (when the conjugate is internalized) the target cell when the antibody binds to its target. Effector molecules can be cytotoxic agents, radioisotopes, or immunomodulating moieties. In conjugates according to the invention the effector molecule is a phosphoantigen moiety.

### Antibody

The term “antibody” as used herein preferably refers to an antibody comprising two heavy chains and two light chains. Generally, the antibody or any antigen-binding fragment thereof, is one that has a therapeutic activity, but such independent efficacy is not necessarily required, as is known in the art of ADCs. The antibodies to be used in accordance with the invention may be of any isotype such as IgA, IgE, IgG, or IgM antibodies. Preferably, the antibody is an IgG antibody, more preferably an IgG₁ or IgG₂ antibody. The antibodies may be chimeric, humanized or human. Preferably, the antibodies are humanized or human. Even more preferably, the antibody is a humanized or human IgG antibody, more preferably a humanized or human IgG₁ monoclonal antibody. The antibody may have κ (kappa) or λ (lambda) light chains, preferably κ (kappa) light chains, i.e., a humanized or human IgG₁-κ antibody.

The term “antigen-binding fragment” as used herein includes a Fab, Fab′, F(ab′)₂, Fv, scFv or reduced IgG (rIgG) fragment, a single chain (sc) antibody, a single domain (sd) antibody, a diabody, or a minibody.

“Humanized” forms of non-human (e.g., rodent) antibodies are antibodies (e.g., non-human-human chimeric antibodies) that contain minimal sequences derived from the non-human antibody. Various methods for humanizing non-human antibodies are known in the art. For example, the antigen-binding complementarity determining regions (CDRs) in the variable regions (VRs) of the heavy chain (HC) and light chain (LC) are derived from antibodies from a non-human species, commonly mouse, rat or rabbit. These non-human CDRs may be combined with human framework regions (FRs, i.e., FR1, FR2, FR3 and FR4) of the variable regions of the HC and LC, in such a way that the functional properties of the antibodies, such as binding affinity and specificity, are at least partially retained. Selected amino acids in the human FRs may be exchanged for the corresponding original non-human species amino acids to further refine antibody performance, such as to improve binding affinity, while retaining low immunogenicity. The thus humanized variable regions are typically combined with human constant regions. Exemplary methods for humanization of non-human antibodies are the method of Winter and co-workers (Jones et al, 1986, Nature, 321, 522-525; Riechmann et al, 1988, Nature, 332, 323-327; Verhoeyen et al, 1988, Science 239, 1534-1536). Alternatively, non-human antibodies can be humanized by modifying their amino acid sequence to increase similarity to antibody variants produced naturally in humans. For example, selected amino acids of the original non-human species FRs are exchanged for their corresponding human amino acids to reduce immunogenicity, while retaining the antibody's binding affinity. For further details, see Jones et al, vide supra; Riechmann et al., vide supra and Presta, 1992, Curr. Op. Struct. Biol. 2, 593-596. See also the following review articles and references cited therein: Vaswani and Hamilton, 1998, Ann. Allergy, Asthma and Immunol., 1, 105-115; Harris, 1995, Biochem. Soc. Transactions, 23, 1035-1038; and Hurle and Gross, 1994, Curr. Op. Biotech., 5, 428-433.

The CDRs may be determined using the approach of Kabat (in Kabat, E. A. et al, (1991), Sequences of Proteins of Immunological Interest, 5th Ed. Public Health Service, National Institutes of Health, Bethesda, MD, NIH publication no. 91-3242, pp. 662, 680, 689), Chothia (Chothia et al, 1989, Nature, 342, 877-883) or IMGT (Lefranc, 1999, The Immunologist, 7, 132-136).

Typically, the antibody is a monospecific (i.e., specific for one antigen; such antigen may be common between species or have similar amino acid sequences between species) or bispecific (i.e., specific for two different antigens of a species) antibody comprising at least one HC and LC variable region binding to an antigen target, preferably a membrane bound antigen target which may be internalizing or not internalizing. Preferably, the antibody is internalized by the target cell after binding to the (antigen) target, after which an active effector molecule, which in a conjugate according to the invention is a phosphoantigen, is released intracellularly.

Targeting antibodies, that may be used in conjugates according to the invention for use in cancer therapy, may be a tumor targeting antibody, selectively binding to a tumor-specific or tumor-associated antigen. Tumor-specific antigens only occur on tumor cells, while tumor associated antigens are antigens that are expressed at higher levels (e.g. overexpressed) in cancer cells, when compared to normal (healthy) cells.

The antigen target to which the antibody or antigen binding fragment of a conjugate according to the invention binds may, for example, be selected from the group consisting of: annexin A1, B7H3, B7H4, BCMA, CA6, CA9, CA15-3, CA19-9, CA27-29, CA125, CA242 (cancer antigen 242), CAIX, CCR2, CCR5, CD2, CD19, CD20, CD22, CD24, CD30 (tumor necrosis factor 8), CD33, CD37, CD38 (cyclic ADP ribose hydrolase), CD40, CD44, CD47 (integrin associated protein), CD56 (neural cell adhesion molecule), CD70, CD71, CD73, CD74, CD79, CD115 (colony stimulating factor 1 receptor), CD123 (interleukin-3 receptor), CD138 (Syndecan 1), CD203c (ENPP3), CD303, CD333, CDCP1, CEA, CEACAM, Claudin 4, Claudin 7, CLCA-1 (C-type lectin-like molecule-1), CLL 1, c-MET (hepatocyte growth factor receptor), Cripto, DLL3, EGFL, EGFR, EPCAM, EphA2, EPhB3, ETBR (endothelin type B receptor), FAP, FcRL5 (Fc receptor-like protein 5, CD307), FGFR3, FOLR1 (folate receptor alpha), FRbeta, GCC (guanylyl cyclase C), GD2, GITR, GLOBO H, GPA33, GPC3, GPNMB, HER2, p95HER2, HER3, HMW-MAA (high molecular weight melanoma-associated antigen), integrin α (e.g., αvβ3 and αvβ5), IGF1R, TM4SF1 (L6), Lewis A like carbohydrate, Lewis X, Lewis Y (CD174), LGR5, LIV1, mesothelin (MSLN), MN (CA9), MUC1, MUC16, NaPi2b, Nectin-4, Notch3, PD-L1, PSMA, PTK7, SLC44A4, STEAP-1, 5T4 (or TPBG, trophoblast glycoprotein), TF (tissue factor, thromboplastin, CD142), TF-Ag, Tag72, TNFalpha, TNFR, TROP2 (tumor-associated calcium signal transducer 2), uPAR, VEGFR and VLA.

Examples of suitable antibodies known in the art include blinatumomab (CD19), rituximab (CD20), or other anti-CD20 antibodies such as ofatumumab, ublituximab or ocrelizumab, epratuzumab (CD22), iratumumab and brentuximab (CD30), gemtuzumab, vadastuximab (CD33), tetulumab (CD37), darartumumab, isatuximab (CD38), bivatuzumab (CD44), alemtuzumab (CD52), lorvotuzumab (CD56), vorsetuzumab (CD70), milatuzumab (CD74), polatuzumab (CD79), rovalpituzumab (DLL3), futuximab (EGFR), oportuzumab (EPCAM), farletuzumab (FOLR1), glembatumumab (GPNMB), trastuzumab, pertuzumab and margetuximab (HER2), etaracizumab (integrin), anetumab (mesothelin), pankomab (MUC1), enfortumab (Nectin-4), H8, A1, and A3 (5T4), and antibodies to TROP2 such as sacituzumab, datopotamab and PF-06664178. Conjugates according to the invention, wherein the targeting moiety is a tumor targeting antibody against CD20 (e.g. rituximab), HER2 (e.g. trastuzumab) or an anti-CD123 antibody are exemplified in the Examples.

Because pAg activity of the pAg moiety should be displayed in the cell, internalizing antibodies are preferred.

The antibody or antigen-binding fragment thereof, if applicable, may comprise (1) a constant region that is engineered, i.e., one or more mutations may have been introduced to e.g., increase half-life, provide a site of attachment for the linker-drug and/or increase or decrease effector function; or (2) a variable region that is engineered, i.e., one or more mutations may have been introduced to e.g., provide a site of attachment for the linker-drug. Antibodies or antigen-binding fragments thereof may be produced recombinantly, synthetically, or by other known suitable methods. Mutations that may decrease Fc mediated effector function of antibodies are, for example, mutations such as those described in Leabman et al., 2013, MAbs, 5(6):896-903 and Bruhns P, et al., 2015, Immunol Rev., 268(1):25-51. doi: 10.1111/imr.12350. PMID: 26497511.

Conjugates according to the present invention may be wild-type or site-specific (meaning a specific conjugation site, such as a cysteine or non-natural amino acid, has been engineered into the antibody protein sequence) or a combination thereof, and can be produced by any method known in the art.

Immunoconjugates according to the invention contain, as an immunomodulating moiety, a phosphoantigen moiety (pAg). It was found that immunoconjugates according to the invention deliver their pAg payload, to antigen-presenting cells such as cancer cells, very efficiently, resulting in an active phosphoantigen within the antigen-presenting cells. Antigen-presenting cells can be tumor cells, expressing or overexpressing certain tumor antigens on their surface. Such cells may also express or overexpress TCR activating molecules involved in the indirect activation of gammadelta T-cells by pAgs, such as BTN3A1/BTN2A1 receptor complex molecules.

Phosphoantigen Moiety (pAg)

A phosphoantigen moiety comprises a non-peptidic antigen with a relatively small mass, that can stimulate gammadelta T-cells (more specifically Vγ9Vδ2 cells) in the presence of antigen-presenting cells. The term “phosphoantigen moiety” or “pAg” as used throughout the present specification refers to any naturally occurring phosphoantigens, as well as non-naturally occurring (synthetic) pAgs, including modified pAgs, such as analogs of naturally occurring pAgs, or prodrugs thereof pAgs suitable for use in the present invention may be pyrophosphates (diphosphates), pyrophosphonates, bisphosphonates (or diphosphonates), monophosphates or monophosphonates, or prodrugs thereof. Preferred pAgs for use in conjugates and linker drugs of the present invention are (mono)phosphonates. Preferred phosphoantigens for use in conjugates and linker-drug compounds of the invention comprise an allylalcohol group, for example an allylalcohol group present in natural phosphoantigens like HMBPP. Preferably the phosphoantigen is a monophosphonate comprising an allylalcohol group.

A “phosphoantigen moiety” as part of a conjugate or linker-drug compound according to the invention, does not necessarily contain the phosphoantigen in its active form. The phosphoantigen moiety in the conjugate or linker-drug compound may comprise an inactive precursor form of an active phosphoantigen and/or may release an active phosphoantigen only after the conjugate binds to its target and has been processed. The phosphoantigen moiety, in its bound state, as part of a conjugate or linker-drug compound, may therefore be structurally different from the active phosphoantigen released therefrom. For example; disconnection from—or cleavage of—a linking moiety may initiate a structural rearrangement and/or a chemical or enzymatic reaction that leads to the formation of a functionally active phosphoantigen. Also the removal- or rearrangement of prodrug moieties, for example in response to changes in the environment or as a result of enzymatic activity at the target site, may release a functionally active phosphoantigen.

Compounds with cellular pAg activity are believed to be able to display their activity directly, through binding to a pAg receptor in a target cell (“direct pAgs”). This receptor is believed to be the intracellular domain of a cell surface molecule, butyrophilin 3A1 (BTN3A1). An example of a natural direct pAg is HMBPP. HMBPP is produced by pathogenic bacteria. It was found that the allylic alcohol in natural pAgs such as HMBPP, is important for BTN3A1 binding and maximal pAg activity. Direct pAgs, such as HMBPP bind directly to BTN3A1 in its intracellular B30.2 domain. Analogs of HMBPP, for example halohydrins such as BrHPP, IHPP and ClHPP are also known in the art (Wiemer et al., 2020, Chem. Med. Chem., 15, 1030-1039).

Other compound show indirect pAg activity, through accumulation of IPP. Such compounds can be referred to as “indirect pAgs”. Indirect pAgs act on pathways that increase cellular levels of (endogenous) direct pAgs, such as IPP and concomitant activation of Vγ9Vδ2 T cells. In contrast to direct pAgs, indirect pAgs do not interact directly with the butyrophilin receptors in target cells, nor are they pAg precursors (compounds that are converted, enzymatically or chemically, to direct pAgs). Indirect pAgs can be compounds that, for example, inhibit downstream enzymes, such as farnesyl pyrophosphate synthase (FPPS). Inhibition of FPPS blocks use of IPP, and leads to accumulation of IPP in a cell. Known FPPS inhibitors are aminobisphosphonates (N-BPs), such as zoledronate. (Wiemer et al., 2020, Chem. Med. Chem., 15, 1030-1039; Park et al., 2021, Frontiers in Chemistry, Vol. 8, Article 612728).

Aminobisphosphonates (N-BPs), such as zoledronate, pamidronate and alendronate, are also known as “bone targeting agents”, because of their ability to specifically bind to hydroxyapatite (HA) (Farrell et al., 2018, Bone Reports, 9, 47-60). Alendronate was also conjugated to trastuzumab, with the aim to target trastuzumab to bone metastasis, using alendronate as the bone targeting agent (Tian et al., 2021, Sci.Adv., 7, 2-11). Due to its negative charge, alendronate has a high affinity for HA, resulting in preferential binding to the bone. Tian et al. thus proposed the use of negatively charged aminobisphosphonates like alendronate as targeting agent for an antibody for treatment of bone-related diseases.

In conjugates according to the invention, the specific binding of, e.g., an antibody (targeting moiety), to its specific binding partner (e.g. a tumor specific antigen) will direct a pAg moiety to its target site, not the other way around (the pAg moiety is not the targeting moiety). In a conjugate according to the invention, it is the binding specificity and affinity of the targeting moiety (e.g. the antibody) which ensures that a phosphoantigen moiety is delivered at the site where it has to exert its therapeutic effect.

Preferred pAg moieties for use in the present invention, comprise an allylic alcohol, or prodrugs thereof (e.g. pAg moieties wherein the allylic alcohol is generated after a prodrug group is removed or after a linker moiety, conjugated through or to the isoprene unit, is cleaved). Such compounds are believed to be examples of pAg moieties comprising direct pAg activity (pAgs that serve as a BTN3A1 ligand). In the alternative precursors, e.g. compounds which are metabolized into compounds having (direct) pAg activity, or prodrugs of direct pAgs or precursors, can be used as pAg moiety in a conjugate according to the invention.

The activity of a phosphoantigen on Vγ9Vδ2 T cells can be measured in a cellular assay, as is exemplified in the Examples. In the cell based assay used, in a first step, target cells, e.g. tumor cells such as, for example, cells from the CD20-positive Burkitt's Lymphoma human tumor cell line Raji, are incubated (overnight) with a phosphoantigen, or a phosphoantigen bearing conjugate according to the invention.

In this first step a phosphoantigen or a conjugate according to the invention will be internalized into the target (tumor) cells. It is assumed that after internalization (and cleavage of the linker in case of a conjugate) the phosphoantigen will bind to the intracellular domain of the BTN3A1 receptor, which will lead to activation of the BTN3A1/BTN2A1 dimer.

In a second step the pre-treated, washed, tumor cells from the first step can be cocultured with gammadelta T-cells. When Vγ9Vδ2 T cells become activated, they produce cytokines and release cytotoxic granules (degranulation), leading to immune activation and target cell killing, respectively.

To assess activity of a phosphoantigen on gammadelta T-cells, monensin and/or brefeldin A are added during co-culture of gammadelta T-cells and targets. This will trap produced cytokines (e.g. interferon gamma (IFNγ) and tumor necrosis factor alpha (TNFα)) in activated cells. Staining with fluorescently-labeled antibodies in the presence of saponin, allowing anti-cytokine antibodies to enter the cell, will identify cytokine-producing cells. Fluorescently-labeled antibodies against CD107a can also be added during co-culture and will stain cells that have undergone degranulation. Degranulation correlates with tumor cell killing (Aktas et al., 2009, Cell Immunol., 254(2), 149-154).

Thus, by combining fluorescently-labeled immune-cell specific markers and CD107a- and cytokine-markers, it is possible to determine the activation status of the gammadelta T-cells and/or other immune cell subsets after co-culture with pretreated target cells.

The ability of gammadelta T-cells to kill pretreated tumor cells can be examined by determining proportions of dead tumor cells after coculture. Tumor cells can be easily identified with a fluorescent tag and their cell dead can already be determined as early as 1 hour after coculture with gammadelta T-cells.

### Phosphoantigen Analogs

(Chemical) analogs are compounds that differ from natural phosphoantigens in their structural characteristics, but resemble natural phosphoantigens in their functional bio-activity. (i.e. they display an (indirect) immune-stimulating activity, in particular, on gammadelta T-cells). Analogs may be designed to improve one or more characteristics of natural occurring pAgs, such as improved characteristics as to stability, potency, bio-availability, or linkage to a linking moiety in the context of their use in immunoconjugates and linker-drug compounds according to the present invention. Natural phosphoantigens include pyrophosphates (diphosphates) such as HMBPP and IPP. Known analogs of natural phosphoantigens include bromohydrin pyrophosphate (BrHPP) and pyrophosphonates such as C-HMBPP, which is the pyrophosphonate equivalent of the naturally occurring HMBPP. Phosphonates, with phosphoantigen activity, known in the art further include bisphosphonates differing in the substituents on the central carbon between the two phosphate groups. Examples include etidronate, clodronate, tiludronate, and a class of bisphosphonates with a nitrogen or amino-group in one of the substituents on the central carbon atom, believed to increase the potency of the bisphosphonate (Drake et al., Mayo Clin. Proc., 2008, 83(9), 1032-1045). These nitrogen containing bisphosphonate phosphoantigens include zoledronate (zoledronic acid), alendronate, risedronate, ibandronate, pamidronate, neridronate and olpadronate.

Another class of phosphoantigen analogs with alleged increased potency, phosphoramidite esters, are described in WO2005/05258 (Innate Pharma), for example N-HDMAPP, wherein the isoprene unit present in natural HMBPP is linked to the pyrophosphate through an NH group.

### Phosphoantigen Prodrugs

With prodrugs, inactive precursors of phosphoantigen moieties are meant, that are converted into an active phosphoantigen, after the removal or conversion of protective groups (e.g. neutral protecting groups on the negatively charged non-binding oxygen atoms of the phosphonate group(s)). After a conjugate, comprising a phosphoantigen moiety in the form of a prodrug, according to the invention, is administered to the body, protective groups may be metabolically removed at the target site. A prodrug may also be formed because of binding of the linking moiety to the phosphoantigen moiety. In this case an active phosphoantigen may be formed because the linker in the conjugate, used to bind the phosphoantigen prodrug moiety to the targeting moiety, is cleaved, resulting in the release of an active phosphoantigen, and/or because protective groups are removed from the phosphoantigen moiety. Preferably such conversions, releasing an active phosphoantigen, take place only after a conjugate according to the invention reaches the site where it has to exert its therapeutic effect, for example, after it is internalized by a tumor cell, or at least in the tumor microenvironment, to prevent unwanted and non-specific side effects of a phosphoantigen moiety in healthy and/or non-target tissue.

For example, certain bisphosphonates have a high affinity for bone mineral and are used as “bone targeting agents”. Such bisphosphonate acts as a targeting molecule for a different drug, conjugated to the bisphosphonate, and target the drug to the bone where the drug exert a therapeutic effect, for example, on bone localized cells (Farrell et al., 2018, Bone Reports, 9, 47-60).

In contrast, in a conjugate according to the invention, a phosphoantigen is conjugated to a targeting moiety (e.g. a tumor specific antibody). In a conjugate according to the invention, it is the binding specificity of the targeting moiety which ensures that a phosphoantigen moiety is delivered at the site where it has to exert its therapeutic effect.

In the context of the present invention, any unwanted reactivity of the phosphoantigen moiety (e.g., binding to non-target tissue by the phosphoantigen as such) can further be prevented by including a prodrug rather than an active phosphoantigen in the conjugate.

The negatively charged phosphonate groups of, for example, a bisphosphonate may be masked by prodrug moieties. The prodrug is delivered to the target site by the targeting moiety of the conjugate, where it is converted into an active phosphoantigen.

Prodrug forms include protecting groups known in the art such as arylesters, aryl amides or pivaloyloxymethyl (POM) prodrug forms. C-HMBP (monophosphonate) phosphoantigen analog/prodrugs are described in WO2019/182904. With the aim to synthesize phosphoantigen prodrugs that are as potent as the natural phosphoantigens such as HMBPP, aryloxy triester phosphoamidite prodrugs of (monophosphonate) phosphoantigens were synthesized, as described in Davey et al., 2018, J. Med. Chem., 61, 2111-2117. In these prodrugs the monophosphonate groups are masked by an aryl motif and an amino acid ester moiety. These compounds (“HMBP ProPagens”) still had rather low serum stability due to the cleavage of the —P—O— bond between the phosphate moiety and the isoprenoid moiety in the molecule. Similar “ProPagens” compounds, wherein the oxygen in the —P—O— bond was replaced by a carbon are described in WO2020/008189. Proposed structure activity relationship (SAR) of phosphoantigen (prodrug)s is described by Wiemer et al., 2020, Chem.Med.Chem., 15, 1030-1039.

A cleavable linking moiety may conveniently be coupled through the alcohol group of the allylalcohol moiety to the phosphoantigen. In this case the allylalcohol may be (re-) formed within the cell when the cleavable linking moiety is cleaved.

Prodrug moieties in a phosphoantigen prodrug as part of a conjugate according to the invention may be the same or different. For example, all prodrug moieties may be POM groups or the phosphoantigen moiety may comprise a combination of, for example, “proTide” groups such as an aryloxy- and an amino acid ester radical, for example such as those described for phosphoantigen prodrugs in WO2020/008189 or WO2019/182904.

Suitable phosphonate prodrug technologies and synthesis of phosphonate prodrugs are known in the art. Such prodrug technologies are further reviewed in, for example, Pradere et al., 2014, Chem. Rev., 114, 9154-9218, and include the use of carbonyloxymethyl prodrug moieties such as pivaloyloxymethyl (POM) and isopropyloxycarbonyloxymethyl (POC) derivatives, S-Acyl-2-thioethyl (SATE) and S-[(2-hydroxyethyl)sulfidyl]-2-thioethyl (DTE) based prodrugs, cyclosaligenyl (cycloSal) phosphate and phosphonate based prodrugs and alkoxyalkyl monoester (hexadecyloxypropyl-(HDP), octadecyloxyethyl-(ODE)) based prodrugs, phosphoramidite and phosphonamidite based prodrugs (including the aryloxy amino acid amidate (ProTide) prodrugs), and phosphordiamidates and phosphonodiamidates.

### Linker-Drug Compounds

The present invention also provides linker-drug compounds comprising at least one phosphoantigen moiety covalently bound to a linking moiety. Such linker-drug compounds may be used as intermediates in the synthesis of conjugates according to the invention. For example, when the targeting moiety is an antibody or antigen binding fragment thereof, one or more linker-drug compounds according to the invention can be conjugated to the targeting antibody, thus creating a conjugate according to the invention.

Linker-drug compounds according to the invention comprise at least one phosphoantigen moiety (pAg or “drug”), and a linking moiety (L or “linker”). Such linker-drug molecules can be used in the manufacture of conjugates according to the invention.

Preferred linker-drug compounds according to the invention may be represented by general formula II:

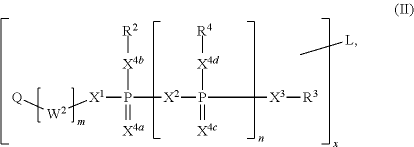

wherein
Q represents a structure reflected in formula IIa or IIb:

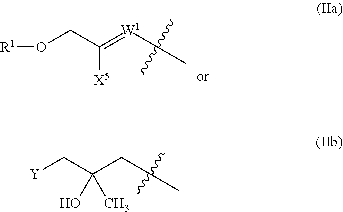

Y is a halogen,
W¹ is N, CH or CF, preferably CH;
W² is CH₂, CHF, CF₂ or O;
X¹ is O, S, NH, CH₂, CHF or CF₂;
X² is O, CH₂, CHF or CF₂;
X³ is absent or O or NH;
each of X^{4a-d} is independently selected from O and S;
X⁵ is CH₃, CH₂F, CHF₂ or CF₃ or CCl₃;
x is an integer ranging from 1-5;
m is 1, 2 or 3;
n is 0, 1 or 2;
R¹ is H or a connection to the linking moiety (L) or a prodrug moiety;
R² is H or a connection to the linking moiety (L) or Cat+ or a prodrug moiety;
R³ is H or a connection to the linking moiety (L) or Cat+ or a prodrug moiety;
R⁴ is H or a connection to the linking moiety (L) or Cat+ or a prodrug moiety;
or, when n is O, R³ and R² are connected by a C₁₋₆ (hetero)alkyl group or;
when n is 1 or 2, R³ and R⁴ are connected by a C₁₋₆ (hetero)alkyl group.

Linker drug compounds wherein Q has formula IIb, may have phosphoantigen moieties resembling (E)-4-Hydroxy-3-methyl-but-2-enyl pyrophosphate (HMBPP) analogs such as BrHPP (Phosphostim), IHPP or ClHPP.

In a preferred embodiment of the invention, linker-drug compounds encompass compounds according to formula II, wherein Q represents a structure reflected in formula IIa Such compounds are represented by general formula III:

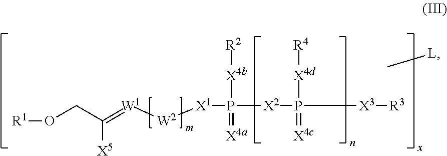

wherein
W¹ is N, CH or CF, preferably CH;
W² is CH₂, CHF, CF₂ or O;
X¹ is O, S, NH, CH₂, CHF or CF₂;
X² is O, CH₂, CHF or CF₂;
X³ is absent or O or NH;
each of X^{4a-d} is independently selected from O and S;
X⁵ is CH₃, CH₂F, CHF₂ or CF₃ or CCl₃;
x is an integer from 1-5;
m is 1, 2 or 3;
n is 0, 1 or 2;
R¹ is H or a connection to the linking moiety (L) or a prodrug moiety;
R² is H or a connection to the linking moiety (L) or Cat+ or a prodrug moiety;
R³ is H or a connection to the linking moiety (L) or Cat+ or a prodrug moiety;
R⁴ is H or a connection to the linking moiety (L) or Cat+ or a prodrug moiety;
or, when n is O, R³ and R² are connected by a C₁₋₆ (hetero)alkyl group or;
when n is 1 or 2, R³ and R⁴ are connected by a C₁₋₆ (hetero)alkyl group.

The formula between the outer brackets represents a pAg moiety, while L represents a linker moiety. There is only one connection to a linking moiety per linker drug molecule. (But when x is larger than 1, there are multiple pAg moieties connected to one (branched) linker moiety).

Preferably n is 0 or 1, most preferably 0. When n is 1 or 2, X² preferably is O. Linker-drug compounds with phosphoantigen moieties wherein n is 1 and X² is CH₂ or where n is 1 and X² is O are likewise part of the present invention. In this case each of X^{4a-d} preferably is O. In such linker drug compounds R³ or R¹ may represent a connection to the linking moiety, preferably R³ represent a connection to the linking moiety.

When n is 2, X² will appear twice in formula II, and can be referred to as X^{2a} and X^{2b} which can be independently selected from O, CH₂, CHF and CF₂. When n is 2, R⁴ will also appear twice, and can be referred to as R^{4a} and R^{4b}, which can be independently selected from H, a connection to the linking moiety (L), Cat+ and a prodrug moiety. The same goes for X^{4c} and X^{4d}, when n is 2. Both appear twice (X^{4c}, X^{4ci}, X^{4d} and X^{4di}), and may be independently selected from O and S.

When m is 2 or 3, W² will appear multiple times in formula II and each W² can independently be selected from CH₂, CHF, CF₂ or O. Preferably W² is CH₂. In a preferred embodiment m is 1, and most preferably, when m is 1, W² is CH₂.

Cat+ represents an (organic or mineral) cation, including a proton.

X¹ preferably is CH₂, O or S, most preferably CH₂.

Each of X^{4a-d} (when present) preferably are O. Part of the present invention are compounds wherein n is 1 or 0 and wherein X^{4a-b} and X^{4c-d} (when present) are O and wherein R² and R⁴ (when present) preferably are H. Preferably n is O, and X^{4a} as well as X^{4b} are O and R² preferably is H.

In linker drug compounds wherein Q represents a structure reflected in formula IIa, preferably W¹ is CH or CF, most preferably CH.

When Q represents a structure reflected in formula IIa, X⁵ preferably is CH₃.

In linker drug compounds wherein Q represents a structure reflected in formula IIa, R¹ preferably is H or a connection to the linking moiety (L), most preferably H.

In linker drug compounds wherein Q represents a structure reflected in formula IIa, preferably, W¹ is CH, X⁵ is CH₃, X¹ is CH₂ and R₁ is H, resulting in a linker drug molecule carrying a pAg moiety with an allylic alcohol group. Preferably, when W¹ is CH, X⁵ is CH₃, X¹ is CH₂ and R₁ is H, W₂ is CH₂ and m is 1. When W¹ is CH, W² is CH₂, m is 1, X⁵ is CH₃, X¹ is O and R₁ is H, the phosphoantigen moiety comprises the allylalcohol chain present in natural phosphoantigens such as HMBPP.

Preferred linker drug compounds are those wherein Q represents a structure reflected in formula IIa, X³ is O, R³ is a connection to a cleavable linking moiety, W¹ is CH, X⁵ is CH₃ and R¹ is H, W² is CH₂ and m is 1, and X¹ is CH₂.

In such compounds R², R³ and/or R⁴ can be a prodrug moiety, either alone or in combination with X^{4b}, X^{4d}, and/or X³ (when X³ is present) respectively (the prodrug moiety being —X^{4b}—R², —X^{4d}—R⁴ and/or —X³—R³).

Preferably, in linker-drug compounds wherein Q represents a structure reflected in formula IIa, W¹ is CH, W² is CH₂, X^{4a-d} are O, R² and R⁴ are H, X⁵ is CH₃ and m is 1.

In a preferred embodiment, Q represents a structure reflected in formula IIa, W¹ is CH, W² is CH₂, n is O, X^{4a-d} are O, X⁵ is CH₃ and m is 1.

In formula II, x represents the number of phosphoantigen moieties (pAg) per linking moiety (L), wherein the structure between the brackets thus is a structural representation of phosphoantigen moieties preferably used in linker-drug compounds according to the invention. X can be an integer in the range from 1-5 (each linking moiety carries one to 5 pAgs). Preferably, a linking moiety carries 1 or 2 pAg moieties. In most instances it may suffice for each linking moiety to carry 1 pAg.

The connection to the linking moiety can be (part of) R¹, or, in the alternative, the linking moiety may be (connected to) R², R³ or R⁴. Preferably either R¹ or R³ is a connection to the linking moiety, more preferably R³. When the linking moiety is connected at R³, X³, preferably, is O. When R³ is a connection to the linker moiety, preferably X³ is O and R¹ is preferably H.

When the linking moiety is attached at the R² or R⁴ position, X^{4b} or X^{4d} respectively, preferably is O.

Preferred linker drug compounds are those wherein Q represents a structure reflected in formula IIa, X³ is O and R³ is a connection to a cleavable linking moiety, wherein preferably W¹ is CH, X⁵ is CH₃ and R¹ is H, W² is CH₂ and m is 1, and X¹ is CH₂. In such compounds n is preferably 0.

With “a connection to the linking moiety” the location in the molecule where the linker is connected to the phosphoantigen moiety is meant. “Connection” doesn't necessarily mean that R¹, R², R³ or R⁴ (depending on where the linker is connected) represent actual (remaining) structural elements of the linker-drug compound between the linker and the remainder of the phosphoantigen moiety. For example, depending on the linker chemistry used, when R represents a connection to the linking moiety, this also includes the situation where the linker is directly connected to the oxygen atom of the phosphoantigen moiety in the linker-drug molecule. In the alternative R¹ is a connection to the linking moiety (L). In such instances where R¹ is a connection to the linking moiety (L), preferably W¹ is CH, W² is CH₂, m is 1, X⁵ is CH₃ and X¹ is CH₂. When such a linker-drug molecule is incorporated into a conjugate according to the invention, cleavage of the linker after administration may result in the (re-)formation of an allyl alcohol group (R¹ is H, in the actual functionally active phosphoantigen moiety released from the conjugate). R¹ can also be a prodrug moiety. Suitable alcohol prodrug moieties are known in the art. For example, an alcohol can be masked by an ester based prodrug group. Creation of the active alcohol relies on the hydrolysis of the ester bond by (cellular) esterases, resulting in the metabolic regeneration of an alcohol (drug) and a carboxylic acid (leaving group).

R², R³, and R⁴ can each independently be H or a connection to the linking moiety (L) or Cat+ or a prodrug moiety. In a preferred embodiment, compounds according to the invention are monophosphonates (n is 0) and R⁴ is thus absent.

Cat+ represents an (organic or mineral) cation, including a proton (and may be exchanged in a formulation buffer or plasma). When R², R³ and/or R⁴ are Cat+, Cat+ may be identical or different. Preferably, when R², R³ and/or R⁴ are Cat+, X^{4b} and X^{4d} (when present, i.e., n is not 0), and/or X³ are O, resulting in O⁻Cat⁺.

In another embodiment of the invention, where n is O, R³ and R² are connected by a C₁₋₆ (hetero)alkyl group. In this case R³ and R² together form a substituted or non-substituted 5-8 membered ring. In such an embodiment the linking moiety is preferably connected at the R¹ position. In an alternative embodiment where n is not 0, R³ and R⁴ may be connected in a similar way by a C₁₋₆ (hetero)alkyl group.

R², R³, and/or R⁴ can also be a prodrug moiety, either alone or in combination with X^{4b}, X^{4d}, and/or X³ (when X³ is present) respectively (the prodrug moiety being —X^{4b}—R², —X^{4d}—R⁴ and/or —X³—R³).

A “prodrug moiety” can be a group that can either be non-enzymatically or enzymatically cleaved (releasing the active compound). A “prodrug moiety” may induce release of a second prodrug moiety on another position in the molecule, after a conjugate according to the invention is administered to a subject. Preferably a phosphoantigen moiety in the form of a prodrug is converted to a functionally active phosphoantigen inside the target cell (e.g., a tumor cell), for example by enzymatic removal of prodrug moieties.

Examples of prodrug technologies known in the art include the use of pivaloyloxymethyl (POM) or isopropyloxycarbonyloxymethyl (POC) groups. In a preferred embodiment, at least R² is and R³ are independently selected from a POM- or POC-group (for example, when n is 0). When n is 1 or 2, R⁴ may be a POM or POC group as well.

Phosphoantigen prodrugs of this kind are described, for example, in WO2019/182904. Such phosphoantigen prodrugs can be used as the basis for the pAg moiety in linker-drug compounds and conjugates according to the invention.

In the alternative a combination of leaving groups can be used; An example of such prodrug technology is the “ProTide” technology, developed for intracellular delivery of monophosphates and monophosphonates. The hydroxyls of the monophosphate or monophosphonate groups in a ProTide prodrug are masked (or replaced) by an aromatic group and an amino acid ester moiety, which are enzymatically cleaved-off inside cells to release the free monophosphate and monophosphonate (Mehellou et al. 2018, Journal of Medicinal Chemistry, 61(6), 2211-2226).

Linker-drug compounds and conjugates according to the invention, wherein the phosphoantigen moiety is a monophosphate or monophosphonate, and wherein R² and R³ are a combination of “ProTide” leaving groups are therefore also part of the present invention. In such cases wherein the phosphoantigen moiety is a ProTide prodrug of a phosphoantigen, either R² is an aromatic moiety and R³ is an amino acid ester moiety or vice versa. In preferred embodiment of the invention, when n is O, either R² or R³ may be a substituted or non-substituted (hetero)aryl group, while the other (either R³ or R²) may be selected from a structure according to formula IV and V

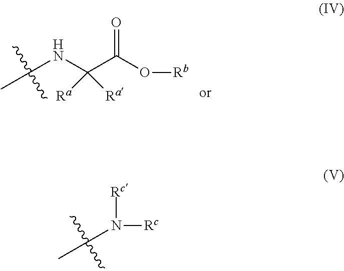

wherein;
R^{a} and R^{a′} are independently selected from H, an optionally substituted amino acid side chain and a non-polar side chain comprising an optionally substituted C₁₋₁₄ alkyl chain,
R^{b} is H, benzyl or a substituted or non-substituted (C₁₋₈)-alkyl,
R^{c} and R^{c′} are independently selected from H, or an optionally substituted (C¹⁻⁶)-alkyl, (C₃₋₆)cycloalkyl, aryl or heteroaryl. R^{c} and R^{c′} may also, together with the nitrogen they are bound to, form an, optionally substituted ring, such as an aziridino-, azetidino-, morpholino-, piperazino-, pyrrolidino- or piperidino-ring.

Optional substituents on R^{c} and/or R^{c′} are a carboxylic acid bioisostere, amino, tetrazole, sulfonate, hydroxyl, halo or alkyl.

When R^{b} is a substituted alkyl, substituents may be one or more groups independently selected from the group consisting of hydroxy, amino, halo, nitro, cyano, carboxy, NRₓR_{y}, (C₁₋₆)alkoxy, (C₁₋₆)alkanoyl, (C₁₋₆)alkoxycarbonyl, (C₁₋₆)alkylthio, and (C₂₋₆)alkanoyloxy, wherein each Rₓ and R_{y} is independently selected from the group consisting of H, (C₁-C₆)alkyl, (C₃₋₆)cycloalkyl, and (C₃₋₆)cycloalkyl(C₁₋₆)alkyl. In the alternative Rₓ and R_{y} together with the nitrogen to which they are attached form a aziridino, azetidino, morpholino, piperazino, pyrrolidino or piperidino group.

### Linking Moiety

A linking moiety (or “linker”) for use in a conjugate or linker-drug compound according to the invention preferably is a synthetic linker. The structure of a linker is such that the linker can be easily chemically attached to a small effector molecule (the phosphoantigen moiety), and so that the resulting linker-drug compound can be easily conjugated to a further substance such as for example a polypeptide (e.g. an antibody). The choice of linker can influence the stability of such eventual conjugates when in circulation, and it can influence in what manner the small molecule effector compound (a phosphoantigen) is released, if it is released. Suitable linkers are for example described in Ducry et a.l, 2010, Bioconjugate Chem., 21, 5-13, King and Wagner, 2014, Bioconjugate Chem., 25, 825-839; Gordon et al., 2015, Bioconjugate Chem., 26, 2198-2215; Tsuchikama and An, 2018, Protein & Cell, 9, 33-46 DOI: 10.1007/s13238-016-0323-0; Polakis, 2016, Pharmacological Reviews, 68 (1), 3-19, DOI: 10.1124/pr.114.009373; Bargh et al., 2019, Chem. Soc. Rev., 48, 4361-4374, DOI: 10.1039/c8cs00676h; WO 02/083180, WO2004/043493, WO2010/062171, WO2011/133039, WO2015/177360, and in WO2018/069375. Linkers may be cleavable or non-cleavable as described in e.g., van Delft, F and Lambert, J. M., 2021, Chemical Linkers in Antibody-Drug Conjugates (ADCs), 1st Ed. Royal Society of Chemistry, ISBN-10: 1839162635. Another way of coupling linker-drugs to antibodies is by making use transpeptidases such as bacterial sortases or plant asparaginyl endopeptidases, enabling the site-specific installation of chemical moieties attached to an appropriate synthetic peptide. Sortase A (Sort-A) recognizes a C-terminal peptide sequence (LPXTG) and creates a bond between the threonine within this sequence and a glycine provided on the N terminus of the conjugation partner, e.g. a glycine tagged payload for an ADC (Combs et al., 2015, the AAPS Journal, Vol. 17, No. 2, 339-351, DOI: 10.1208/s12248-014-9710-8). Antibody drug conjugation can also be achieved through site-specific glycoengineering, for example by using endo-β-N-acetylglucosaminidase (ENGases) and monosaccharyl transferase mutants (Manabe et al., 2021, Chem Rec, (11), 3005-3014, doi: 10.1002/tcr.202100054; Wang et al., 2019, Annu Rev Biochem, 20; 88, 433-459, doi: 10.1146/annurev-biochem-062917-012911).

The use of cleavable linkers in conjugates according to the invention is preferred. Cleavable linkers comprise moieties that can be cleaved, e.g., when exposed to lysosomal proteases or to an environment having an acidic pH or a higher reducing potential. Suitable cleavable linkers are known in the art and comprise e.g., a mono-, di-, tri- or tetrapeptide, i.e., a single-, two, three or four amino acid residues. Additionally, the cleavable linker may comprise a selfimmolative moiety such as an ω-amino aminocarbonyl cyclization spacer, see Saari et al, 1990, J. Med. Chem., 33, 97-101, or a —NH—CH₂—O— moiety. Cleavage of the linker makes the immunomodulating effector moiety (phosphoantigen or “pAg” moiety) in a conjugate according to the invention available to the surrounding environment. Non-cleavable linkers can still effectively release (an active derivative of) the phosphoantigen moiety from the immunoconjugate according to the invention, for example when a conjugated polypeptide (antibody) is degraded in the lysosome. Non-cleavable linkers include e.g., succinimidyl-4-(N-maleimidomethyl(cyclohexane)-1-carboxylate and maleimidocaproic acid and analogs thereof.

To be able to conjugate a linking moiety or linker-drug compound to a polypeptide, such as an antibody, the side of the linking moiety that will be (covalently) bonded to the antibody, typically contains a functional group that can react with an amino acid residue of the antibody, under relatively mild conditions. This functional group is referred to herein as a reactive moiety (RM). Examples of reactive moieties include, but are not limited to, carbamoyl halide, acyl halide, active ester, anhydride, alpha-halo acetyl, alpha-halo acetamide, maleimide, isocyanate, isothiocyanate, disulfide, thiol, hydrazine, hydrazide, sulfonyl chloride, aldehyde, methyl ketone, vinyl sulfone, halo methyl, methyl sulfonate, cyclooctyn and trans-cyclooctene (TCO). Such amino acid residue with which the functional group reacts may be a natural or non-natural amino acid residue, or a (non-)natural glycan (Manabe et al., Wang et al., vide supra). The term “non-natural amino acid” as used herein is intended to represent a (synthetically) modified amino acid or the D-stereoisomer of a naturally occurring amino acid. Preferably, the amino acid residue with which the functional group reacts is a natural amino acid.

Linking moieties (L) for use in conjugates or linker-drug compounds according to the present invention may comprises a structure according to formula VI or VII

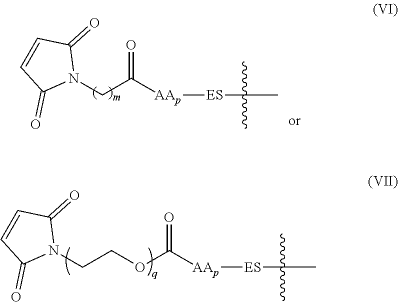

wherein m is an integer ranging from 1 to 10, preferably 5; A is an amino acid, preferably a natural amino acid and p is 0, 1, 2, 3, or 4. When p is more than 1, the aminoacids may be the same or different.

Suitable amino-acid combinations are known in the art and include amino acids selected from the group consisting of alanine, glycine, lysine, phenylalanine, valine, and citrulline. Preferably p is 2. When p is 2, AA₂ may be, for example, phenylalanyllysine, valylalanine, valylcitrulline or valyllysine. When p is 2, AA₂ preferably is valylalanine or valylcitrulline. When p is 3, AA₃ may be, for example, alanylphenylalanyllysine, when p is 4, AA₄ may be, for example, glycylglycylphenylalanylglycine.

“q” is an integer ranging from 1 to 12, preferably 2; ES is either absent or an elongation spacer selected from

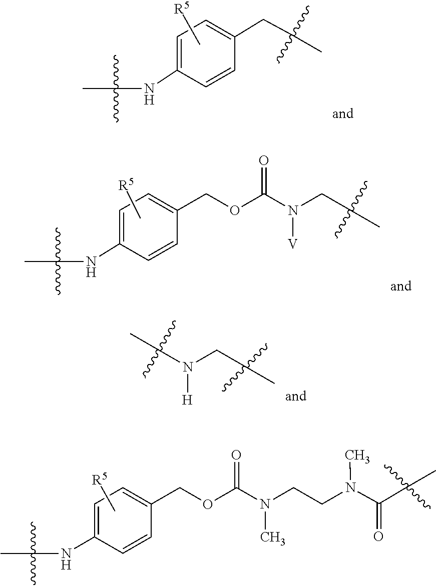

wherein R⁵ is H, halogen, CF₃, C1-4 alkyl, C2-4 alkenyl, C2-4 alkynyl, C1-4 alkoxyl, or C1-4 alkylthio, preferably H, F, CH₃ or CF₃, more preferably H or F; and V is H, ethyl, —(CH₂CH₂O)ₚ—OMe, CH₂CH₂SO₂Me or CH₂CH₂N(Me)₂, wherein p is an integer ranging from 1 to 12.

Linking moieties can also be branched, which results in one linking moiety being able to carry multiple phosphoantigen moieties. Examples of branched linking moieties are:

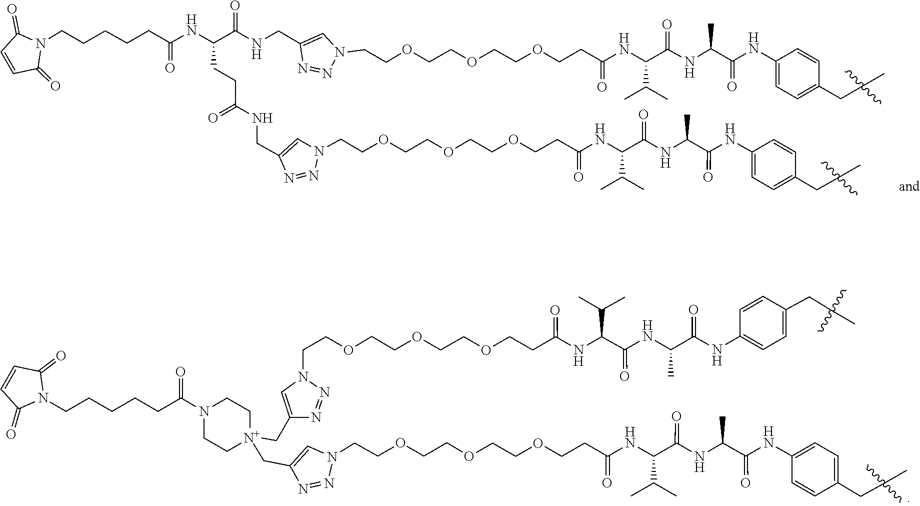 [SMILES: *Cc1ccc(NC(=O)[C@H](C)NC(=O)[C@@H](NC(=O)CCOCCOCCOCCn2cc(CNC(=O)CC[C@H](NC(=O)CCCCCN3C(=O)C=CC3=O)C(=O)NCc3cn(CCOCCOCCOCCC(=O)N[C@H](C(=O)N[C@@H](C)C(=O)Nc4ccc(C*)cc4)C(C)C)nn3)nn2)C(C)C)cc1 ; *Cc1ccc(NC(=O)[C@H](C)NC(=O)[C@@H](NC(=O)CCOCCOCCOCCn2cc(C[N+]3(Cc4cn(CCOCCOCCOCCC(=O)N[C@H](C(=O)N[C@@H](C)C(=O)Nc5ccc(C*)cc5)C(C)C)nn4)CCN(C(=O)CCCCCN4C(=O)C=CC4=O)CC3)nn2)C(C)C)cc1]

These branched linker moieties can be used to create conjugates with a relatively high pAg to targeting moiety ratio (“DAR”). Using such branched linkers, conjugates with a DAR of 16 and even 20 or higher can be synthesized. Antibody based conjugates according to the invention may only need a DAR of about 2. However, for antibodies to tumor specific targets that are known to be expressed at a relatively low level on target tumor cells, conjugates with a high pAg to targeting moiety ratio may be preferred. Linker-drug compounds for use in a linker-drug compound according to the invention can, for example, contain any linking moiety selected from:

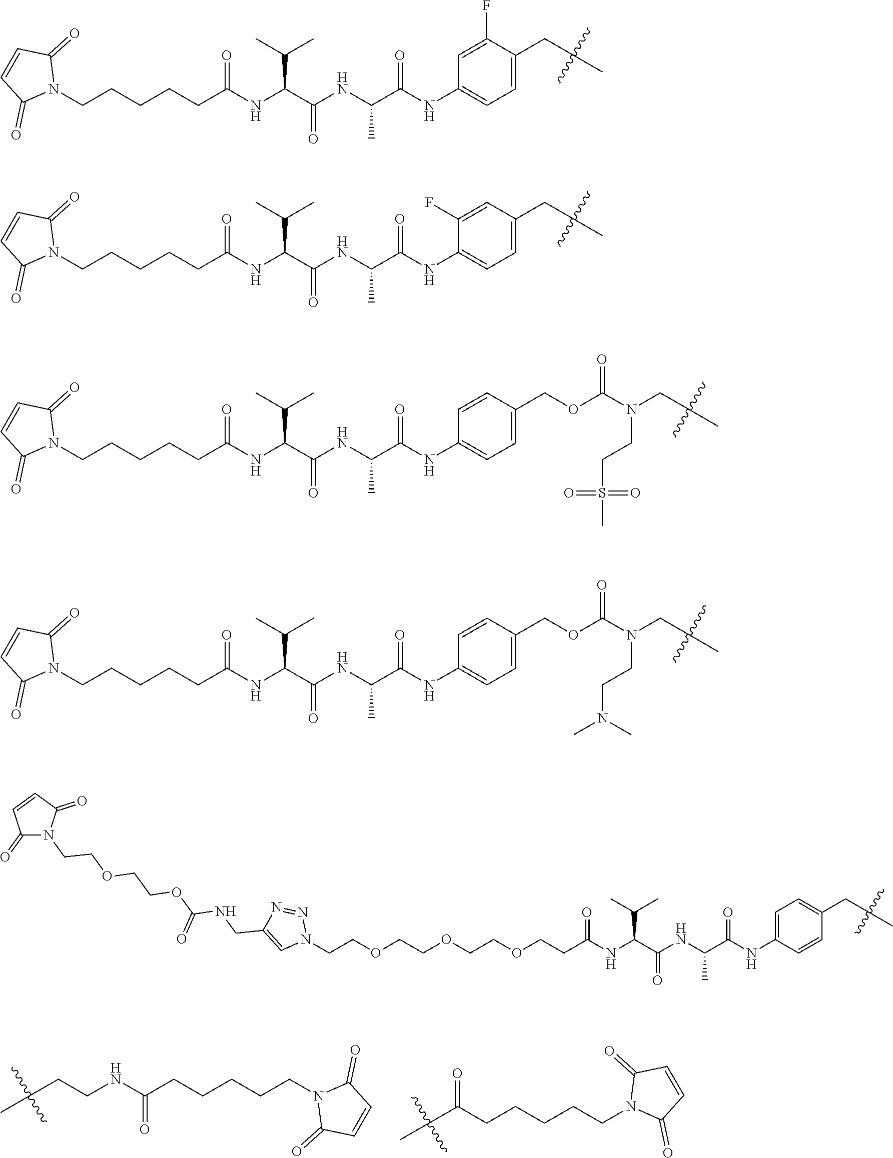

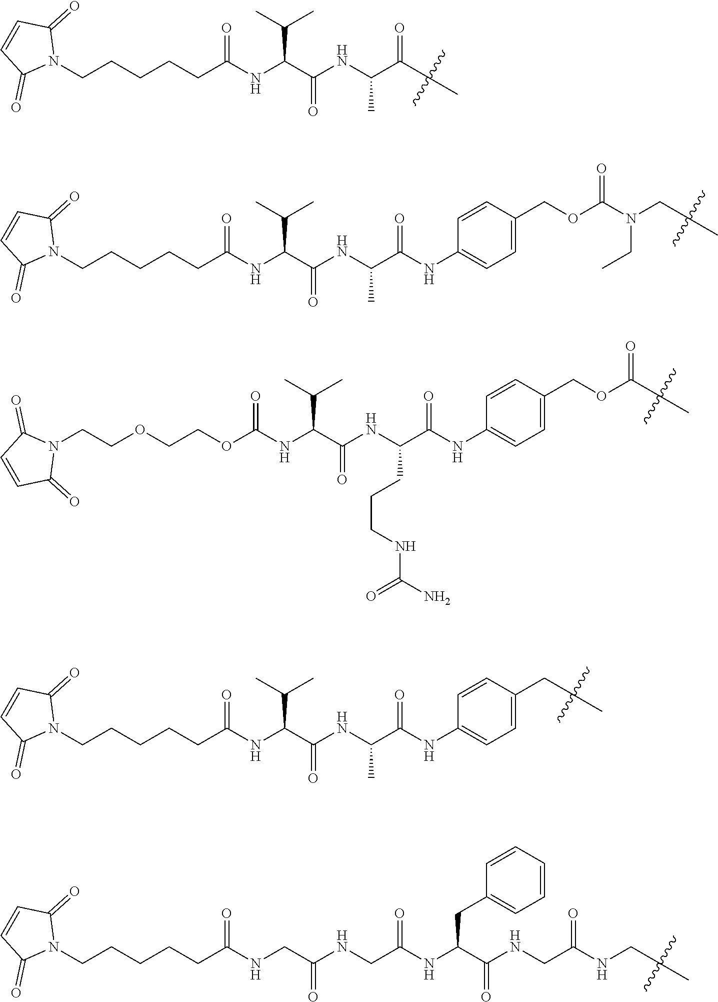 [SMILES: *C(=O)[C@H](C)NC(=O)[C@@H](NC(=O)CCCCCN1C(=O)C=CC1=O)C(C)C ; *CN(CC)C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)OCCOCCN2C(=O)C=CC2=O)C(C)C)cc1 ; *Cc1ccc(NC(=O)[C@H](C)NC(=O)[C@@H](NC(=O)CCCCCN2C(=O)C=CC2=O)C(C)C)cc1 ; *CNC(=O)CNC(=O)[C@H](Cc1ccccc1)NC(=O)CNC(=O)CNC(=O)CCCCCN1C(=O)C=CC1=O]

Linker moieties (L) may be conjugated to pAg moieties resulting in linker drug compounds according to the invention with the general formula depicted in formula II.

Linker-drug compounds according to the invention can be conjugated to a targeting moiety, to create a conjugate according to the invention. Preferred conjugates according to the invention comprise a tumor targeting antibody, or antigen binding fragment thereof, conjugated to a linker drug compound according to the invention.

In a specific embodiment of the invention, the phosphoantigen moiety, as part of a conjugate according to the invention, is a monophosphonate prodrug, wherein the negatively charged non-binding oxygen atoms of the phosphonate group are protected by prodrug moieties such as a combination of ProTide moieties (a (hetero)aryl group and an amino ester radical) or one or more POM or POC, while a cleavable linking moiety may be attached to an isoprene unit of the phosphoantigen molecule, which will be converted to an allylic alcohol, found in phosphoantigens such as HMBPP, once the linker is cleaved.

It is to be understood that a linker-drug compound comprising at least one phosphoantigen moiety covalently bound to a linking moiety according to the invention, when comprised in a conjugate according to the invention, may lack or gain certain atoms or groups of atoms, for example, it may lack a hydrogen atom as compared to the same linker-drug compound according to the invention when not comprised in a conjugate. This can be for example because the linker-drug compound according to the invention is conjugated to a polypeptide via, for example, esterification to a hydroxyl moiety.

The synthesis of examples of linker-drug molecules according to the invention is further exemplified in the Examples. An example of a preferred linker drug compound according to the invention is XD18, having the following structural formula:

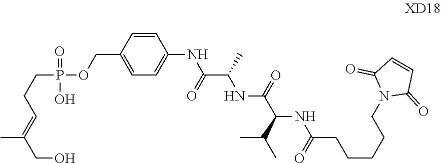 [SMILES: C/C(=C\CCP(=O)(O)OCc1ccc(NC(=O)[C@H](C)NC(=O)[C@@H](NC(=O)CCCCCN2C(=O)C=CC2=O)C(C)C)cc1)CO]

A conjugate according to the invention, based on linker-drug XD18, may comprise 1-20, preferably 1-8, more preferably 2 linker drug molecules per antibody (e.g. rituximab, as exemplified in the Examples).

### Synthesis of Conjugates According to the Invention.

To synthesize a conjugate according to the invention, one or more linker-drug compound(s) according to the invention may be conjugated to a suitable target moiety. When the target moiety is a polypeptide (antibody, or a binding fragment thereof) the linker-drug compound may be conjugated via a reactive native amino acid residue present in the suitable polypeptide, e.g., a lysine or a cysteine, or via an N-terminus or C-terminus. Alternatively, a reactive amino acid residue, natural or non-natural, may be genetically engineered into the suitable polypeptide, or a reactive group may be introduced via post-translational modification.

Conjugates according to the invention may be produced by conjugating a linker-drug compound according to the invention to an antibody or antigen-binding fragment thereof through e.g., the lysine 8-amino groups of the antibody, preferably using an intermediate comprising an amine-reactive group such as an activated ester. Such methods are known for producing conventional Antibody-drug Conjugates (ADCs).

Alternatively, immunoconjugates can be produced by conjugating the linker through the free thiols of the side chains of cysteines generated through reduction of interchain disulfide bonds, using methods and conditions known in the art, see e.g., Doronina et al, 2006, Bioconjugate Chem., 17, 114-124. The manufacturing process involves partial reduction of the solvent-exposed interchain disulfides followed by modification of the resulting thiols with Michael acceptor-containing linkers such as maleimide-containing linkers, alfa-haloacetic amides or esters. The cysteine attachment strategy results in maximally two linker containing linker-drugs per reduced disulfide.

Preferred antibodies used as targeting moieties in conjugates according to the invention are of the human IgG type. Most human IgG molecules have four solvent-exposed disulfide bonds, which equates to a range of integers of from zero to eight linked linking moieties per antibody. The exact number of linked phosphoantigen moieties per target moiety is determined by the number of phosphoantigen moieties per linking moiety, the extent of disulfide reduction and the number of molar equivalents of linker containing linker-drugs in the ensuing conjugation reaction. Full reduction of all four disulfide bonds gives a homogeneous construct with eight linker moieties per antibody, while a partial reduction typically results in a heterogeneous mixture with zero, two, four, six, or eight linking moieties per antibody.

In a preferred embodiment, the present invention relates to a conjugate, wherein the linker-drug compound according to the invention is conjugated to an antibody or antigen-binding fragment thereof through a cysteine residue of the antibody or the antigen-binding fragment.

### Site Specific Conjugation to Antibodies or Antigen Binding Fragments Thereof

Because antibodies contain many lysine residues and cysteine disulfide bonds, conventional conjugation typically produces heterogeneous mixtures that present challenges with respect to analytical characterization and manufacturing. Furthermore, the individual constituents of these mixtures exhibit different physicochemical properties and pharmacology with respect to their pharmacokinetic, efficacy, and safety profiles, hindering a rational approach to optimizing this modality.

To improve conjugate homogeneity, antibodies used in (immuno)conjugates according to the invention may be modified to allow for site-specific conjugation of the linker. Methods for site-specific drug conjugation to antibodies are comprehensively reviewed by C. R. Behrens and B. Liu, 2014, mAbs, 6 (1), 1-8, and can be found in WO2015/177360, WO2005/084390, and WO2006/034488.

Site-specific immunoconjugates are preferably produced by conjugating the linker-drug compound to the antibody or antigen-binding fragment thereof through the side chains of engineered cysteine residues in suitable positions of the mutated antibody or antigen-binding fragment thereof. Engineered cysteines are usually capped by other thiols, such as cysteine or glutathione, to form disulfides. These capped residues need to be uncapped before linker-drug attachment can occur. Linker-drug attachment to the engineered residues is either achieved (1) by reducing both the native interchain and mutant disulfides, then re-oxidizing the native interchain cysteines using a mild oxidant such as CuSO₄ or dehydroascorbic acid, followed by standard conjugation of the uncapped engineered cysteine with a linker-drug, or (2) by using mild reducing agents which reduce mutant disulfides at a higher rate than the interchain disulfide bonds, followed by standard conjugation of the uncapped engineered cysteine with a linker-drug. Suitable methods for site-specifically conjugating linker-drugs can for example be found in WO 2015/177360 which describes the process of reduction and re-oxidation, WO 2017/137628 which describes a method using mild reducing agents and WO 2018/215427 which describes a method for conjugating both the reduced interchain cysteines and the uncapped engineered cysteines.

### Pharmaceutical Compositions

In a further aspect, the invention provides a composition comprising a conjugate according to the invention, preferably wherein the composition is a pharmaceutical composition, more preferably further comprising one or more a pharmaceutically acceptable excipient(s). Such composition is referred to hereinafter as a composition according to the invention. The composition may for example be a liquid formulation, a lyophilized formulation, or in the form of e.g., capsules or tablets.

Typically, pharmaceutical compositions comprising immunoconjugates according to the invention take the form of lyophilized cakes (lyophilized powders), which require (aqueous) dissolution (i.e., reconstitution) before intravenous infusion, or frozen (aqueous) solutions, which require thawing before use. Accordingly, in preferred embodiments, the invention provides a lyophilized composition comprising an immunoconjugate according to the invention, preferably wherein the composition is a pharmaceutical composition, more preferably further comprising one or more pharmaceutically acceptable excipient(s). In further preferred embodiments, the invention provides a frozen composition comprising water and an immunoconjugate according to the invention, preferably wherein the composition is a pharmaceutical composition, more preferably further comprising one or more pharmaceutically acceptable excipient(s). In this context, the frozen solution is preferably at atmospheric pressure, and the frozen solution was preferably obtained by freezing a liquid composition according to the invention at temperatures below 0° C. Suitable pharmaceutically acceptable excipients for inclusion into the pharmaceutical composition (before freeze-drying) in accordance with the present invention include buffer solutions (e.g., citrate, amino acids such as histidine, or succinate containing salts in water), lyoprotectants (e.g., sucrose, trehalose), tonicity modifiers (e.g., chloride salts, such as sodium chloride), surfactants (e.g., polysorbate), and bulking agents (e.g., mannitol, glycine). Excipients used for freeze-dried protein formulations are selected for their ability to prevent protein denaturation during the freeze-drying process as well as during storage.

### Medical Uses

In a further aspect, the invention provides a conjugate according to the invention, or a composition according to the invention, for use as a medicament, preferably for the treatment of cancer, autoimmune or infectious diseases. Conjugates according to the invention can be used to induce a cytotoxic effect of gammadelta T-cells on, for example, tumor- and/or infected cells.

Conjugates and compositions are collectively referred to hereinafter as products for use according to the invention.

In one embodiment, the products for use according to the invention are for use in the treatment of a solid tumor or hematological malignancy. In a second embodiment, the products for use according to the invention are for use in the treatment of an autoimmune disease. In a third embodiment, the products for use according to the invention are for use in the treatment of an infectious disease, such as a bacterial, viral, fungal, parasitic or other infection.

A cancer in the context of the present invention, preferably is a tumor expressing the antigen to which the products for use according to the invention are directed. Such tumor may be a solid tumor or hematological malignancy. Examples of tumors or hematological malignancies that may be treated with products for use according to the invention as defined above may include, but are not limited to, breast cancer; brain cancer (e.g., glioblastoma); head and neck cancer; thyroid cancer; parotic gland cancer, adrenal cancer (e.g., neuroblastoma, paraganglioma, or pheochromocytoma); bone cancer (e.g., osteosarcoma); soft tissue sarcoma (STS); ocular cancer (e.g., uveal melanoma); esophageal cancer; gastric cancer; small intestine cancer; colorectal cancer; urothelial cell cancer (e.g., bladder, penile, ureter, or renal cancer); ovarian cancer; uterine cancer; vaginal, vulvar and cervical cancer; lung cancer (especially non-small cell lung cancer (NSCLC) and small-cell lung cancer (SCLC)); melanoma; mesothelioma (especially malignant pleural and abdominal mesothelioma); liver cancer (e.g., hepatocellular carcinoma); pancreatic cancer; skin cancer (e.g., basalioma, squamous cell carcinoma, or dermatofibrosarcoma protuberans); testicular cancer; prostate cancer; acute myeloid leukemia (AML); chronic myeloid leukemia (CML); chronic lymphatic leukemia (CLL); acute lymphoblastic leukemia (ALL); myelodysplastic syndrome (MDS); blastic plasmacytoid dendritic cell neoplasia (BPDCN); Hodgkin's lymphoma; non-Hodgkin's lymphoma (NHL) (including follicular lymphoma (FL), CNS lymphoma, and diffuse large B-cell lymphoma (DLBCL)); light chain amyloidosis; plasma cell leukemia; and multiple myeloma (MM).

An autoimmune disease in the context of the present invention, preferably is an autoimmune disease associated with the antigen to which the products for use according to the invention are directed. An autoimmune disease represents a condition arising from an abnormal immune response to normal body cells and tissues. There is a wide variety of at least 80 types of autoimmune diseases. Some diseases are organ specific and are restricted to affecting certain tissues, while others resemble systemic inflammatory diseases that impact many tissues throughout the body. The appearance and severity of these signs and symptoms depend on the location and type of inflammatory response that occurs and may fluctuate over time. Examples of autoimmune diseases that may be treated with products for use according to the invention as defined above may include, but are not limited to, rheumatoid arthritis; juvenile dermatomyositis; psoriasis; psoriatic arthritis; lupus; sarcoidosis; Crohn's disease; eczema; nephritis; uveitis; polymyositis; neuritis including Guillain-Barre syndrome; encephalitis; arachnoiditis; systemic sclerosis; autoimmune mediated musculoskeletal and connective tissue diseases; neuromuscular degenerative diseases including Alzheimer's disease, multiple sclerosis (MS), amyotrophic lateral sclerosis (ALS), neuromyelitis optica, and large, middle size, small vessel Kawasaki and Henoch Schonlein vasculitis; cold and warm agglutinin disease; autoimmune hemolytic anemia (AIHA); immune thrombocytopenic purpura ITP), type 1 diabetes mellitus; Hashimoto's thyroiditis; Graves' disease; Graves' ophthalmopathy; adrenalitis; hypophysitis; pemphigus vulgaris; Addison's disease; ankyloses spondylitis; Behcet's syndrome; celiac disease; Goodpasture's syndrome; myasthenia gravis; sarcoidosis; scleroderma; primary sclerosing cholangitis, epidermolysis bullosa acquisita, and bullous pemphigoid.

An infectious disease in the context of the present invention, preferably is an infectious disease associated with the antigen to which the products for use according to the invention are directed. Such infectious disease may be a bacterial, viral, fungal, parasitic or other infection. Examples of infectious diseases that may be treated with products for use according to the invention as defined above may include, but are not limited to, malaria; toxoplasmosis; pneumocystis jirovecii melioidosis; shigellosis; listeria; diseases caused by Cyclospora or mycobacterium leprae; tuberculosis; and infectious prophylaxis in immune compromised individuals, such as in HIV-positive individuals, individuals on immunosuppressive treatment, or individuals with inborn errors such as cystic fibrosis or benign proliferative diseases (e.g., mola hydatidosa or endometriosis).

Products for use according to the invention as described herein can be for the use in the manufacture of a medicament as described herein. Products for use according to the invention as described herein are preferably for methods of treatment, wherein the products for use are administered to a subject, preferably to a subject in need thereof, in a therapeutically effective amount. Thus, alternatively, or in combination with any of the other embodiments, in an embodiment, the present invention relates to a use of products for use according to the invention for the manufacture of a medicament for the treatment of cancer, autoimmune or infectious diseases, in particular for the treatment of cancer. For illustrative, non-limitative, cancers or other diseases to be treated according to the invention: see hereinabove.

Alternatively, or in combination with any of the other embodiments, in an embodiment, the present invention relates to a method for treating cancer, autoimmune or infectious diseases, in particular cancer, which method comprises administering to a subject in need of said treatment a therapeutically effective amount of a product for use according to the invention. For illustrative, non-limitative, cancers or other diseases to be treated according to the invention: see hereinabove.

Products for use according to the invention are for administration to a subject. Products for use according to the invention can be used in the methods of treatment described hereinabove by administration of an effective amount of the composition to a subject in need thereof. The term “subject” as used herein refers to all animals classified as mammals and includes, but is not restricted to, primates and humans. The subject is preferably a human. The expression “therapeutically effective amount” means an amount sufficient to effect a desired response, or to ameliorate a symptom or sign. A therapeutically effective amount for a particular subject may vary depending on factors such as the condition being treated, the overall health of the subject, the method, route, and dose of administration and the severity of side effects.

### Combined Use

In further embodiments, the invention provides the product for use according to the invention, wherein the use is combined with one or more other therapeutic agents. Products for use according to the invention may be used concomitantly or sequentially with the one or more other therapeutic agents.

Suitable chemotherapeutic agents include alkylating agents, such as nitrogen mustards, hydroxyurea, nitrosoureas, tetrazines (e.g., temozolomide) and aziridines (e.g., mitomycin); drugs interfering with the DNA damage response, such as PARP inhibitors, ATR and ATM inhibitors, CHK1 and CHK2 inhibitors, DNA-PK inhibitors, and WEE1 inhibitors; anti-metabolites, such as antifolates (e.g., pemetrexed), fluoropyrimidines (e.g, gemcitabine), deoxynucleoside analogues and thiopurines; anti-microtubule agents, such as vinca alkaloids and taxanes; topoisomerase I and II inhibitors; cytotoxic antibiotics, such as anthracyclines and bleomycins; hypomethylating agents such as decitabine and azacitidine; histone deacetylase inhibitors; all-trans retinoic acid; and arsenic trioxide. Suitable radiation therapeutics include radio-isotopes, such as ¹³¹I-metaiodobenzylguanidine (MIBG), ³²P as sodium phosphate, ²²³Ra chloride, ⁸⁹Sr chloride and ¹⁵³Sm diamine tetramethylene phosphonate (EDTMP). Suitable agents to be used as hormonal therapeutics include inhibitors of hormone synthesis, such as aromatase inhibitors and GnRH analogues; hormone receptor antagonists, such as selective estrogen receptor modulators (e.g., tamoxifen and fulvestrant) and antiandrogens, such as bicalutamide, enzalutamide and flutamide; CYP17A1 inhibitors, such as abiraterone; and somatostatin analogs.

Targeted therapeutics are therapeutics that interfere with specific proteins involved in tumorigenesis and proliferation and may be small-molecule drugs; proteins, such as therapeutic antibodies; peptides and peptide derivatives; or protein-small molecule hybrids, such as ADCs. Examples of targeted small molecule drugs include TLR ligands, mTor inhibitors, such as everolimus, temsirolimus and rapamycin; kinase inhibitors, such as imatinib, dasatinib and nilotinib; VEGF inhibitors, such as sorafenib and regorafenib; EGFR/HER2 inhibitors, such as gefitinib, lapatinib, and erlotinib; and CDK4/6 inhibitors, such as palbociclib, ribociclib and abemaciclib. Examples of peptide or peptide derivative targeted therapeutics include proteasome inhibitors, such as bortezomib and carfilzomib.

Suitable anti-inflammatory drugs include D-penicillamine, azathioprine and 6-mercaptopurine, cyclosporine, anti-TNF biologicals (e.g., infliximab, etanercept, adalimumab, golimumab, certolizumab, or certolizumab pegol), lenflunomide, abatacept, tocilizumab, anakinra, ustekinumab, rituximab, daratumumab, ofatumumab, obinutuzumab, secukinumab, apremilast, acetretin, and JAK inhibitors (e.g., tofacitinib, baricitinib, or upadacitinib).

Immunotherapeutic agents include agents that induce, enhance or suppress an immune response, such as cytokines (IL-2 and IFN-α); immuno modulatory imide drugs, e.g., thalidomide, lenalidomide, pomalidomide, or imiquimod; therapeutic cancer vaccines, e.g., talimogene laherparepvec; cell based immunotherapeutic agents, e.g., dendritic cell vaccines, adoptive T-cells, or chimeric antigen receptor-modified T-cells; and therapeutic (bispecific) antibodies, or other ADCs, that can trigger antibody-dependent cell-mediated cytotoxicity (ADCC), antibody-dependent cellular phagocytosis (ADCP) or complement-dependent cytotoxicity (CDC) via their Fc region when binding to membrane bound ligands on a cell.

In the context of the invention, treatment is preferably preventing, reverting, curing, ameliorating, and/or delaying the cancer, autoimmune or infectious disease. This may mean that the severity of at least one symptom of the cancer, autoimmune or infectious disease has been reduced, and/or at least a parameter associated with the cancer, autoimmune or infectious disease has been improved.

In the context of the invention, a subject may survive and/or may be considered as being disease free. Alternatively, the disease or condition may have been stopped or delayed. In the context of the invention, an improvement of quality of life and observed pain relief may mean that a subject may need less pain relief drugs than at the onset of the treatment. “Less” in this context may mean 5% less, 10% less, 20% less, 30% less, 40% less, 50% less, 60% less, 70% less, 80% less, 90% less. A subject may no longer need any pain relief drug. This improvement of quality of life and observed pain relief may be seen, detected or assessed after at least one week, two weeks, three weeks, four weeks, one month, two months, three months, four months, five months, six months or more of treatment in a subject and compared to the quality of life and observed pain relief at the onset of the treatment of said subject.

### General Definitions

Conjugates and linker-drugs according to the invention may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof. Accordingly, the chemical structures depicted herein encompass all possible tautomeric forms of the illustrated or identified compounds. It is also understood that some isomeric forms such as diastereomers, enantiomers and geometrical isomers can be separated by physical and/or chemical methods by those skilled in the art. When a structural formula or chemical name is understood by the skilled person to have chiral centers, yet no chirality is indicated, for each chiral center individual reference is made to all three of either the racemic mixture, the pure R enantiomer, and the pure S enantiomer. When the structure of a compound is depicted as a specific enantiomer, it is to be understood that the invention of the present application is not limited to that specific enantiomer. When two moieties are said to together form a bond, this implies the absence of these moieties as atoms, and compliance of valence being fulfilled by a replacing electron bond. All this is known in the art.

The compounds disclosed in this description and in the claims may further exist as exo and endo regioisomers. Unless stated otherwise, the description of any compound in the description and in the claims is meant to include both the individual exo and the individual endo regioisomer of a compound, as well as mixtures thereof. Furthermore, the compounds disclosed in this description and in the claims may exist as cis and trans isomers. Unless stated otherwise, the description of any compound in the description and in the claims is meant to include both the individual cis and the individual trans isomer of a compound, as well as mixtures thereof. As an example, when the structure of a compound is depicted as a cis isomer, it is to be understood that the corresponding trans isomer or mixtures of the cis and trans isomer are not excluded from the invention of the present application.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g., about 10) preferably means that the value may be the given value more or less 1% of the value.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature (RT) to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such charge more often than that it does not bear or carry such charge. As such, an atom that is indicated in this disclosure to be charged could be non-charged under specific conditions, and a neutral moiety could be charged under specific conditions, as is understood by a person skilled in the art.

All patent and literature references cited in the present specification are hereby incorporated by reference in their entirety.

The following Examples are offered for illustrative purposes only, and are not intended to limit the scope of the present invention in any way.

### EXAMPLES

### General Procedures

### Solvents:

All solvents used were reagent grade or HPLC grade from various vendors.

### NMR Spectra:

NMR spectra were recorded on a Bruker AVANCE400 (400 MHz for ¹H; 101 MHz for ¹³C).

### Chemical Shifts:

Chemical shifts are reported in ppm relative to tetramethylsilane as an internal standard, or residual undeuterated solvent.

### UPLC Characterization of Products:

Products were characterized on a Waters UPLC-MS (equipped with an SQD 2 detector) with a Waters ACQUITY UPLC BEH C18 Column (1.7 μm particle size, 2.1×50 mm) at a flow rate of 0.4 mL/min. (MeCN/Water×0.1% Formic acid).

### HPLC Purification:

Purifications by preparative HPLC were performed using a Shimadzu Prominence 20AP system equipped with a Waters SunFire Prep C18 OBD 5 μm colunn (19×150 mm) at a flow rate of 17 ml/min.

General Procedure XXA: Alkylation of Alcohols with Chloromethyl Carbamates.

Paraformaldehyde (3 eq.) and trimethylsilylchloride (TMSCl) (2.75 eq.) were sequentially added to a RT suspension of the carbamate (2.5 eq.) in dichloromethane (DCM) (0.45 M carbamate) under N₂. The mixture was stirred for 1 h and was then concentrated, coevaporated from DCM and dried under high vacuum for 2 min. The pale yellow oil was dissolved in DCM (0.6 M carbamate).

An aliquot of this solution, (typically 1.5 eq of the chloromethyl carbamate) was then added to a cooled (0° C.) mixture of the alcohol (1 eq.) in DCM (0.24 M). N,N-Diisopropylethylamine (DIPEA, 3 eq.) was added and after stirring for 5 min, the reaction was warmed to RT. UPLC-MS was used to assess conversion after 30-60 min, and more stock solution was added if incomplete. With each subsequent addition of chloromethyl carbamate, a stoichiometric amount of DIPEA was added to maintain a basic pH. Once full conversion was observed, the reaction was quenched with MeOH, concentrated and purified as indicated.

General Procedure XXB: Preparation of Phosphonic Acid Dichlorides from Phosphonate Diesters Trimethylsilylbromide (TMSBr, 10 eq.) was added over 5 min to a cooled (0° C.) solution of the phosphonate diester (1 eq.) in DCM (0.2 M) under N₂ atmosphere. After 30 min, the ice bath was removed and the reaction was stirred at RT for 3.5 h. The solution was concentrated using an N₂-purged rotary evaporator, and the crude was taken up in DCM (0.2 M) under N₂ atmosphere, and cooled to 0° C. Dimethylformamide (DMF, 2 drops) was added followed by the dropwise addition of oxalyl chloride (3 eq.). The cooling bath was allowed to warm to RT over 1 h, and stirring was continued for 16 h. The reaction was concentrated under N₂ atmosphere and coevaporated with DCM (3×10 mL) to give crude phosphonic dichloride that was used without further purification.
General Procedure XXC: Allylic Oxidation with SeO₂

Step 1: SeO₂ (0.7 eq.) and salicylic acid (0.1 eq.) were dissolved in DCM (0.9 M SeO₂) and t-BuOOH (4.5 eq.) was added at RT. After stirring vigorously for 15 min, the alkene (1 eq.) in DCM (0.82 M) was added. The resulting reaction mixture was stirred vigorously until UPLC-MS analysis indicated full consumption of the alkene (typically 16-48 h). The reaction mixture was cooled to 0° C. and was then carefully quenched with sat. aq. NaHCO₃ (10 mL per 1 mL tBuOOH used). The mixture was diluted with water to help solubilize any precipitated salts, and the product was extracted 3-6 times with DCM (or EtOAc for more polar compounds), until UPLC-MS analysis revealed no more product in the water phase. The combined organic layers were dried over Na₂SO₄, filtered and concentrated, to yield a mixture of the allylic alcohol and the corresponding aldehyde product.

Step 2: The crude was dissolved in EtOAc (0.2 M) and AcOH (5 eq.) was added, followed by NaBH(OAc)₃ (5 eq.). The reaction mixture was stirred at 50° C., until UPLC-MS analysis indicated full consumption of the aldehyde (typically for 1-3 h). Afterwards, the reaction mixture was cooled to RT and water (1-5 volumes) was added. The water layer was extracted with EtOAc (2-6×) until UPLC-MS analysis indicated no more product in the water phase. The combined organic layers were washed with a small volume of sat. aq. NaHCO₃, and brine, dried over Na₂SO₄, filtered and concentrated. Purification was performed as indicated.

### Example 1: Synthesis of Carbamate (XD5)

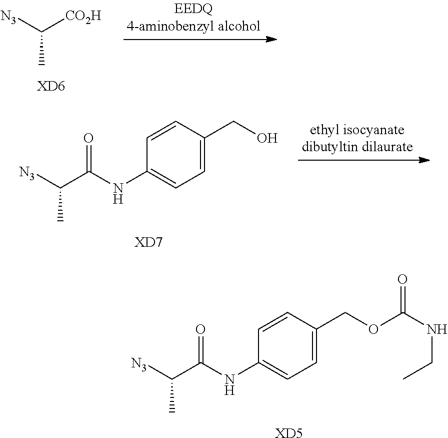

### (S)-2-Azido-N-(4-(hydroxymethyl)phenyl)propanamido (XD7)

(S)-2-azidopropanoic acid (6.73 g, 58.5 mmol) and (4-aminophenyl)methanol (10.0 g, 82 mmol) were dissolved in DCM (228 mL) and MeOH (75 mL). After cooling to 0° C., N-ethoxycarbonyl-2-ethoxy-1,2-dihydroquinoline (EEDQ, 28.9 g, 117 mmol) was added and the mixture was stirred at RT overnight and concentrated. Purification by flash chromatography (silica gel, 0-40% EtOAc in DCM) afforded azide XD7 (9.3 g, 72%) as a yellow liquid. MS (ESI⁺) calc. for C₁₀H₁₃N₄O₂⁺ [M+H]⁺ 221.10, found 221.11.

### (S)-4-(2-Azidopropanamido)benzyl ethylcarbamate (XD5)

To a solution of XD7 (3.8 g, 17.2 mmol) in tetrahydrofuran (THF, 100 mL) were added at 0° C. dibutyltin dilaurate (2.57 ml, 4.31 mmol) and ethyl isocyanate (2.05 mL, 25.9 mmol) and the mixture was stirred for 5 h at RT. The reaction mixture was concentrated on silica gel and purified by flash chromatography (silica gel, 0-50% diethyl ether in heptane, followed by 0-100% EtOAc in heptane, to give azide XD5 (4.25 g, 85%) as a white solid. ¹H NMR (400 MHz, DMSO-d₆) ppm=8.29 (br s, 1H), 7.52 (d, J=8.4 Hz, 2H), 7.30 (d, J=8.3 Hz, 2H), 5.04 (s, 2H), 4.92 (br s, 1H), 4.18 (q, J=7.0 Hz, 1H), 3.22 (quint, J=6.7 Hz, 2H), 1.61 (d, J=7.0 Hz, 3H), 1.12 (t, J=7.3 Hz, 3H). MS (ESI⁺) calc. for C₁₃H₁₇N₅NaO₃⁺ [M+Na]⁺ 314.1, found 314.1.

### Example 2: Synthesis of Linker-Drug Compound XD4 from pAg (Prodrug) Moiety XD1

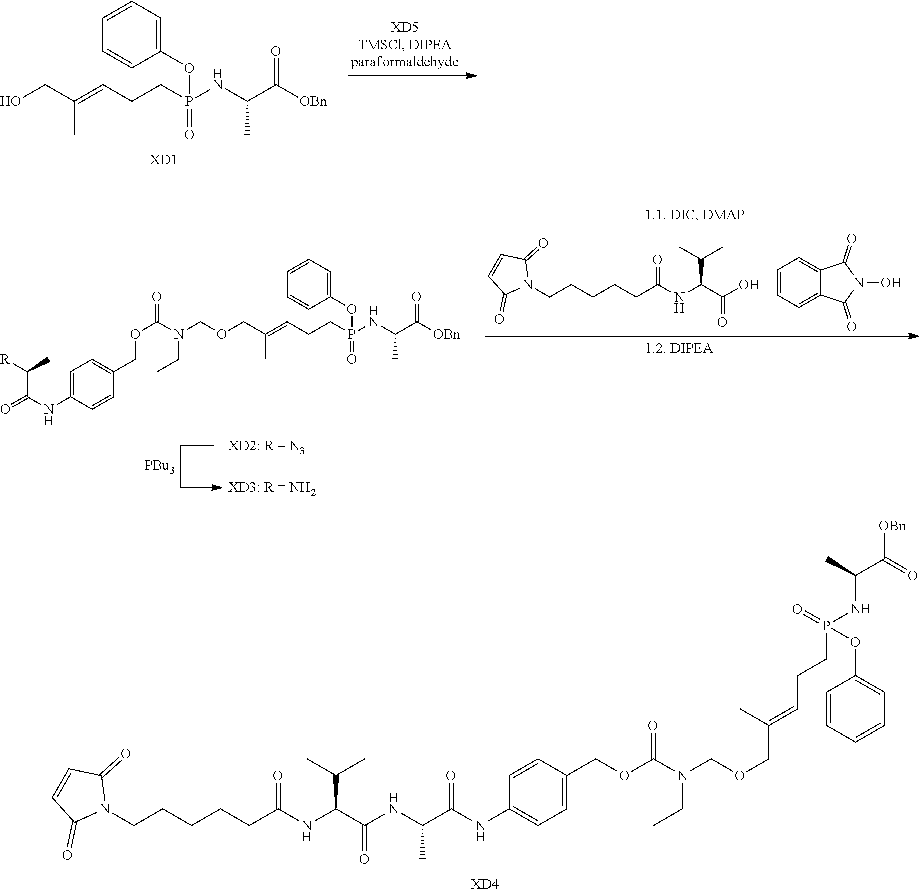

### Benzyl (((E)-5-(((((4-((S)-2-azidopropanamido)benzyl)oxy)carbonyl)(ethyl)amino)methoxy)-4-methylpent-3-en-1-yl)(phenoxy)phosphoryl)-L-alaninate (XD2)

XD1 (100 mg, 0.240 mmol, prepared as described in Kadri et al., J Med. Chem. 2020, 63, 11258-11270) was reacted with carbamate XD5 according to general procedure XXA. Purification by flash chromatography (silica gel, 0-5% MeOH in DCM) afforded XD2 (134 mg, 78%) as a colorless oil. MS (ESI⁺) calc. for C₃₆H₄₆N₆O₈P⁺ [M+H]⁺ 721.3, found 721.6.

### Benzyl (((E)-5-(((((4-((S)-2-aminopropanamido)benzyl)oxy)carbonyl)(ethyl)amino)methoxy)-4-methylpent-3-en-1-yl)(phenoxy)phosphoryl)-L-alaninate (XD3)

A solution of azide XD2 (134 mg, 0.186 mmol) in THF/water (2 mL, 9:1) was purged with N₂ for 15 min. Tributylphosphine (0.116 ml, 0.465 mmol) was added at RT and the mixture was stirred for 4 h. The reaction was concentrated and residual water was removed by coevaporation with MeCN (2×7 mL) and toluene (1×7 mL). Purification of the crude by flash chromatography (silica gel, 0-20% MeOH in DCM) afforded amine XD3 (97 mg, 75%). MS (ESI⁺) calc. for C₃₆H₄₈N₄O₈P⁺ [M+H]⁺ 695.3, found 695.5.

### Benzyl (((E)-5-(((((4-((S)-2-((S)-2-(6-(2,5-dioxo-2,5-dihydro-1H-pyrrol-1-yl)hexanamido)-3-methylbutanamido)propanamido)benzyl)oxy)carbonyl)(ethyl)amino)methoxy)-4-methylpent-3-en-1-yl)(phenoxy)phosphoryl)-L-alaninate (XD4)

N,N′-Diisopropylcarbodiimide (DIC; 0.013 mL, 0.085 mmol) was added to a RT suspension of (6-(2,5-dioxo-2,5-dihydro-1H-pyrrol-1-yl)hexanoyl)-L-valine (26.5 mg, 0.085 mmol, prepared as described in WO2013122823), DMAP (1.0 mg, 8.5 μmol) and N-hydroxyphthalimide (13.9 mg, 0.085 mmol) in THF (0.8 ml). After 3 h at RT, the reaction mixture was concentrated and suspended in DCM (˜1 mL). The orange/red supernatant was then added to a solution of XD3 (41 mg, 0.059 mmol) in DMF (0.5 ml) and stirred for 75 min at RT. After concentration, the crude was purified by RP-HPLC (water/MeCN, gradient 40% to 90%, no modifier added). Lyophilization of product fractions afforded XD4 (10.9 mg, 13%). MS (ESI⁺) calc. for C₅₁H₆₈N₆O₁₂P⁺ [M+H]⁺ 987.5, found 987.7.

Linker-drug compound XD4 was conjugated to antibodies to create conjugates ADC-XD4-r and ADC-XD4-i, as described in Example 22. Both were tested for their effect on gamma delta T-cells as described in Example 23.

### Example 3: Synthesis of Linker-Drug Compound XD13

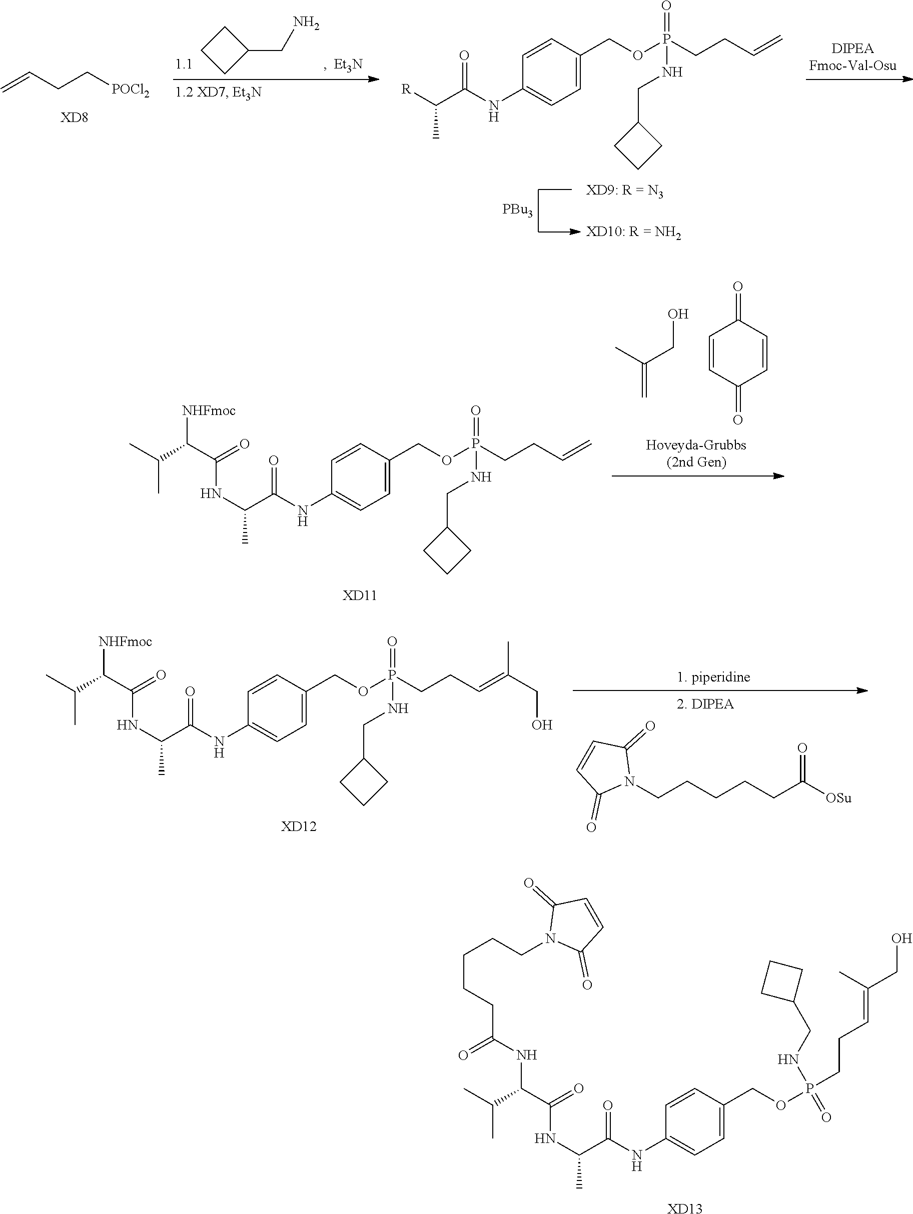

### 4-((S)-2-azidopropanamido)benzyl P-(but-3-en-1-yl)-N-(cyclobutylmethyl)phosphonamidate (XD9)

In the first step, but-3-en-1-ylphosphonic dichloride (XD8, 338 mg, 1.95 mmol, prepared as described in Kadri et al. J. Med. Chem. 2020, 63, 11258-11270) was added dropwise to a solution of cyclobutylmethanamine (166 mg, 1.95 mmol) and Et₃N (0.544 ml, 3.90 mmol) in DCM (3.8 ml) at −78° C. After 5 min, the cooling bath was removed and stirring was continued for 45 min.

In a separate flask, alcohol XD7 (429 mg, 1.95 mmol) and Et₃N (0.544 ml, 3.90 mmol) were dissolved in DCM (3.8 ml) under N₂, and the mixture was cooled to −78° C. The solution that was prepared in the first step, was then filtered directly into the solution containing alcohol XD7. DCM (2 mL) was used to complete the transfer. After 5 min, the reaction was warmed to RT and stirred for 5 h. The reaction was quenched with 1-methylpiperazine (0.1 mL). After concentration, the crude was taken up in EtOAc (50 mL) and washed with aq. HCl (0.1 M, 30 mL). The water layer was extracted with EtOAc (15 mL) and the combined org. layers were washed with sat. aq. NaHCO₃, water and brine, dried over MgSO₄, filtered and concentrated. Purification by flash chromatography (silica gel, 0-80% EtOAc in DCM) afforded azide XD9 (363 mg, 46%) as a colorless oil. ¹H NMR (400 MHz, CDCl₃) ppm=8.21 (br s, 1H), 7.56 (d, J=8.5 Hz, 2H), 7.35 (d, J=8.4 Hz, 2H), 5.85 (ddt, J=16.9, 10.3, 6.4 Hz, 1H), 5.09-4.96 (m, 3H), 4.89 (dd, J=11.9, 7.6 Hz, 1H), 4.24 (q, J=7.0 Hz, 1H), 2.88 (br s, 2H), 2.43-2.27 (m, 4H), 2.13-1.97 (m, 2H), 1.97-1.75 (m, 4H), 1.70-1.55 (m, 5H). MS (ESI⁺) calc. for C₁₉H₂₉N₅O₃P⁺ [M+H]⁺ calc: 406.2, found: 406.4.

### 4-((S)-2-Aminopropanamido)benzyl P-(but-3-en-1-yl)-N-(cyclobutylmethyl)phosphonamidate (XD10)

A solution of azide XD9 (244 mg, 0.602 mmol) in THF (1.8 ml)/Water (0.2 ml) was purged with N₂ for 15 min. Tributylphosphine (0.376 ml, 1.51 mmol) was added at RT, and the mixture was stirred for 22 h. The reaction was concentrated, coevaporated with MeCN (2×7 mL) and toluene (1×7 mL), and the crude purified by flash chromatography (silica gel, 0-20% MeOH in DCM) to give amine XD10 (182 mg, 80%). ¹H NMR (400 MHz, CD₃OD) ppm=7.63 (d, J=8.5 Hz, 2H), 7.38 (d, J=8.5 Hz, 2H), 5.89 (ddt, J=16.9, 10.3, 6.4 Hz, 1H), 5.07 (dq, J=17.0, 1.6 Hz, 1H), 5.02-4.89 (m, 3H), 3.58 (q, J=6.9 Hz, 1H), 2.99-2.85 (m, 2H), 2.50-2.39 (m, 1H), 2.38-2.27 (m, 2H), 2.12-2.01 (m, 2H), 1.98-1.77 (m, 4H), 1.77-1.66 (m, 2H), 1.37 (d, J=7.0 Hz, 3H). MS (ESI⁺) calc. for C₁₉H₃₁N₃O₃P⁺ [M+H]⁺ calc: 380.2, found: 380.3.

### (9H-Fluoren-9-yl)methyl ((2S)-1-(((2S)-1-((4-(((but-3-en-1-yl((cyclobutylmethyl)amino)phosphoryl)oxy)methyl)phenyl)amino)-1-oxopropan-2-yl)amino)-3-methyl-1-oxobutan-2-yl)carbamate (XD11)

Fmoc-Val-OSu (220 mg, 0.50 mmol) was added to a solution of amine XD10 (182 mg, 0.48 mmol) and DIPEA (0.079 ml, 0.46 mmol) in THF (4.8 ml) at RT. After 70 min a gel-like mixture formed. Ethyl acetate (4.0 ml) was added which broke up the gel and stirring was continued for 4 h. The reaction mixture was diluted with EtOAc/isopropyl alcohol (9:1) and washed with sat. aq. NaHCO₃ and brine. The org. layer was dried over Na₂SO₄, filtered and concentrated. Purification by flash chromatography (silica gel, 0-10% MeOH in DCM) afforded amide XD11 (279 mg, 83%). ¹H NMR (400 MHz, DMSO-d₆) ppm=10.00 (s, 1H), 8.17 (d, J=7.0 Hz, 1H), 7.89 (d, J=7.5 Hz, 2H), 7.74 (t, J=7.3 Hz, 2H), 7.58 (d, J=8.5 Hz, 2H), 7.46-7.38 (m, 3H), 7.37-7.27 (m, 4H), 5.87 (ddt, J=16.9, 10.4, 6.3 Hz, 1H), 5.08-4.91 (m, 2H), 4.84 (dd, J=12.1, 7.6 Hz, 1H), 4.77 (dd, J=12.1, 7.6 Hz, 1H), 4.57 (dt, J=11.1, 6.8 Hz, 1H), 4.43 (quint, J=7.0 Hz, 1H), 4.34-4.18 (m, 3H), 3.92 (dd, J=8.9, 7.1 Hz, 1H), 2.87-2.73 (m, 2H), 2.39-2.26 (m, 1H), 2.26-2.15 (m, 2H), 2.05-1.90 (m, 3H), 1.85-1.59 (m, 6H), 1.31 (d, J=7.1 Hz, 3H), 0.89 (d, J=6.9 Hz, 3H), 0.86 (d, J=6.8 Hz, 3H). MS (ESI⁺) calc. for C₃₉H₅₀N₄O₆P⁺ [M+H]⁺ calc: 701.4, found: 701.5.

### (9H-Fluoren-9-yl)methyl ((2S)-1-(((2S)-1-((4-(((((cyclobutylmethyl)amino)((E)-5-hydroxy-4-methylpent-3-en-1-yl)phosphoryl)oxy)methyl)phenyl)amino)-1-oxopropan-2-yl)amino)-3-methyl-1-oxobutan-2-yl)carbamate (XD12)

Amide XD11 (100 mg, 0.143 mmol), 2-methylprop-2-en-1-ol (0.126 ml, 1.50 mmol) and 1,4-benzoquinone (1.5 mg, 0.014 mmol) were suspended in 1,2-dichloroethane (1.2 ml) at RT under N₂. Hoveyda-Grubbs 2nd gen. catalyst (4.5 mg, 7.1 μmol, CAS:301224-40-8) was added at RT and the suspension was heated to 45° C. After 4 h, additional Hoveyda-Grubbs 2nd gen. catalyst (4.5 mg, 7.1 μmol) was added and stirring was continued at 45° C. overnight. More 1,4-benzoquinone (2.3 mg, 0.021 mmol) and Hoveyda-Grubbs 2nd gen. catalyst (8.9 mg, 0.014 mmol) was added and the reaction was continued for 5 h. The reaction was cooled to RT, 1,4-bis(3-isocyanopropyl)piperazine (SnatchCat, 12.6 mg, 0.057 mmol) was added, and the mixture was stirred for 30 min. Purification by flash chromatography (silica gel, 0-10% MeOH in DCM) afforded XD12 (39 mg, contaminated with the undesired Z-isomer as well as an impurity originating from double bond isomerization in the sm to the internal position, prior to cross metathesis (m/z 731.6)). The material was carried forward without any further purification at this stage. MS (ESI⁺) calc. for C₄₁H₅₄N₄O₇P⁺ [M+H]⁺ calc: 745.4, found: 745.6.

### 4-((S)-2-((S)-2-Amino-3-methylbutanamido)propanamido)benzyl N-(cyclobutylmethyl)-P-((E)-5-hydroxy-4-methylpent-3-en-1-yl)phosphonamidate (XD13)

Dipeptide XD12 (39 mg, 0.052 mmol) was dissolved in DMF (1 ml) at RT. Piperidine (0.39 ml, 3.9 mmol) was added and the mixture was stirred for 30 min. After concentration, ether (8 mL) was added, and the mixture was stirred for 15 min at RT. The product did not dissolve well and stuck to the flask. Ether was removed by pipette and the flask was rinsed with ether (1×). The residual oil was dried under vacuum to give a colourless oil (24.5 mg).

The material was dissolved in DMF (0.5 ml) at RT, and 2,5-dioxopyrrolidin-1-yl 6-(2,5-dioxo-2,5-dihydro-1H-pyrrol-1-yl)hexanoate (14.5 mg, 0.047 mmol) and DIPEA (0.025 ml, 0.141 mmol) were sequentially added. The reaction mixture was stirred at RT for 3 h. After concentration, the crude was purified by RP-HPLC (water/MeCN, gradient 70:30 to 50:50, no modifier added) to give pure XD13 (16.2 mg, 43%, 2 steps). ¹H NMR (400 MHz, DMSO-d₆) ppm=9.91 (s, 1H), 8.13 (d, J=7.0 Hz, 1H), 7.80 (d, J=8.6 Hz, 1H), 7.62-7.56 (m, 2H), 7.31 (d, J=8.5 Hz, 2H), 7.00 (s, 2H), 5.35 (td, J=7.2, 1.3 Hz, 1H), 4.83 (dd, J=12.3, 7.6 Hz, 1H), 4.76 (dd, J=12.1, 7.8 Hz, 1H), 4.63 (t, J=5.6 Hz, 1H), 4.54 (dt, J=11.0, 6.8 Hz, 1H), 4.39 (quint, J=7.0 Hz, 1H), 4.17 (dd, J=8.6, 6.9 Hz, 1H), 3.76 (d, J=5.8 Hz, 2H), 3.36 (t, J=7.1 Hz, 2H), 2.88-2.72 (m, 2H), 2.39-2.26 (m, 1H), 2.23-2.10 (m, 4H), 2.02-1.88 (m, 3H), 1.87-1.70 (m, 2H), 1.69-1.57 (m, 4H), 1.56-1.52 (m, 3H), 1.52-1.40 (m, 4H), 1.30 (d, J=7.1 Hz, 3H), 1.18 (quint, J=7.5 Hz, 2H), 0.86 (d, J=6.8 Hz, 3H), 0.82 (d, J=6.9 Hz, 3H). MS (ESI⁺) calc. for C₃₆H₅₅N₅O₈P⁺ [M+H]⁺ calc: 716.4, found: 716.6.

Linker-drug compound XD13 was conjugated to antibodies to create conjugates ADC-XD13-r and ADC-XD13-i, as described in Example 22. Both were tested for their effect on gamma delta T-cells as described in Example 23.

### Example 4: Synthesis of Carboxylic Acid XT2

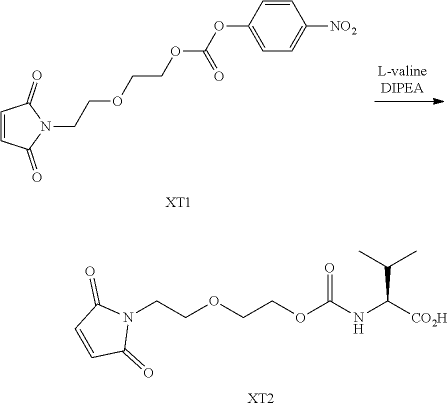

### ((2-(2-(2,5-Dioxo-2,5-dihydro-1H-pyrrol-1-yl)ethoxy)ethoxy)carbonyl)-L-valine (XT2)

To a solution of L-valine (167 mg, 1.4 mmol) and carbonate XT1 (500 mg, 1.4 mmol, prepared as described in Elgersma et al., Mol. Pharm., 2015, 12, 1813-1835) in DMF (5 ml) at 0° C. was added DIPEA (0.249 ml, 1.40 mmol) and the resulting mixture was stirred for 10 days at RT. The mixture was concentrated, taken up in EtOAc (25 ml) and washed with aq. HCl (1 M, 50 ml). The water layer was extracted with EtOAc (25 ml) and the combined organic layers were dried (MgSO₄), filtered and concentrated. Purification by flash chromatography (silica gel, 0-40% MeOH in DCM) afforded acid XT2 (250 mg, 53%) as a clear oil. MS (ESI⁺) calc. for C₁₄H₂₁N₂O₇⁺ [M+H]⁺ calc: 329.1, found: 329.2.

### Example 5: Synthesis of Linker-(Pro) Drug Compound XC4

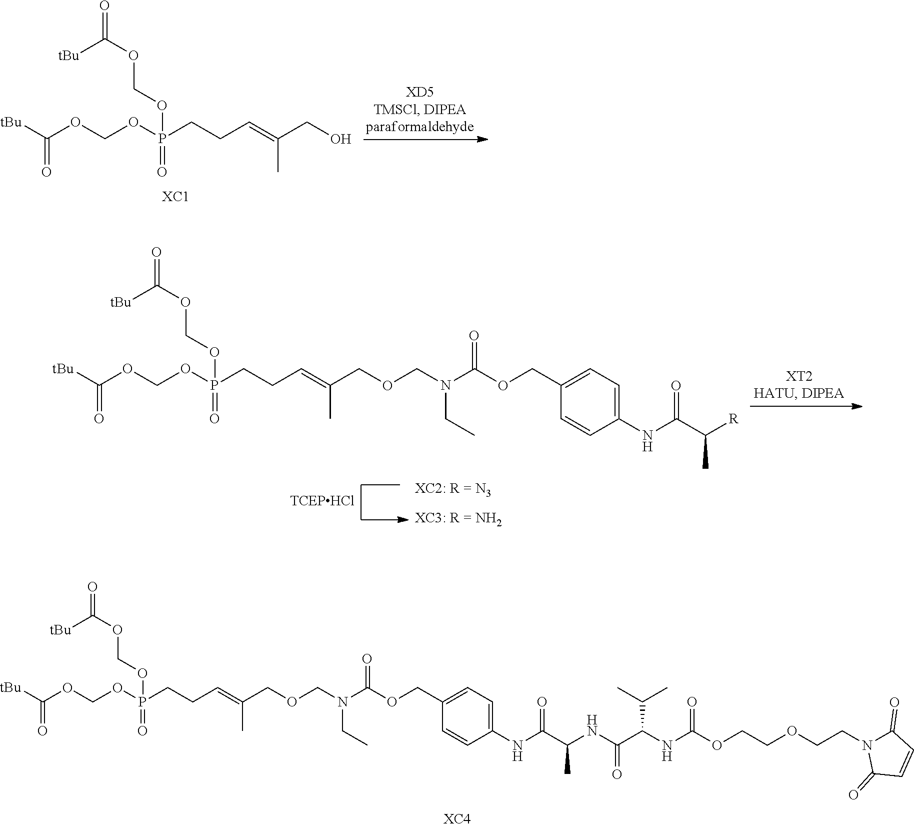

### (S,E)-(((5-(((((4-(2-Azidopropanamido)benzyl)oxy)carbonyl)(ethyl)amino)methoxy)-4-methylpent-3-en-1-yl)phosphoryl)bis(oxy))bis(methylene) bis(2,2-dimethylpropanoate) (XC2)

Alcohol XC1 (70 mg, 0.171 mmol, prepared as described in Wiemer, Chem. Biol. 2014, 21, 945-954) was reacted with carbamate XD5 according to general procedure XXA, described in Example 1. Once the reaction was complete, half of the solvent volume was removed by rotary evaporation. The crude mixture was then directly loaded on a silica gel column and purified by flash chromatography (silica gel, 0-100% EtOAc in heptane). Azide XC2 (140 mg, quant.) was obtained as an impure colorless oil that was carried forward without any further purification. MS (ESI⁺) calc. for C₃₂H₅₁N₅O₁₁P⁺ [M+H]⁺ 712.3, found 712.5.

### (S,E)-(((5-(((((4-(2-Aminopropanamido)benzyl)oxy)carbonyl)(ethyl)amino)methoxy)-4-methylpent-3-en-1-yl)phosphoryl)bis(oxy))bis(methylene) bis(2,2-dimethylpropanoate) (XC3)

To a solution of azide XC2 (70 mg, 0.098 mmol) in THF (1.85 mL)/water (0.093 mL) was added tris(2-carboxyethyl)phosphine hydrochloride (TCEP·HCl, 85 mg, 0.295 mmol) at RT, and the resulting mixture was stirred for 18 h. The suspension was filtered over cotton wool, rinsed with THF and the filtrate was concentrated on silica gel. Purification by flash chromatography (silica gel, 0-10% MeOH in DCM) afforded amine XC3 (15 mg, 22%) as a colorless oil. ¹H NMR (400 MHz, CD₃OD) ppm=7.51 (d, J=8.5 Hz, 2H), 7.27 (d, J=8.5 Hz, 2H), 5.61-5.52 (m, 4H), 5.35-5.13 (m, 1H), 5.02 (s, 2H), 4.65 (br s, 2H), 3.80 (q, J=7.0 Hz, 1H), 3.70 (br d, J=1.0 Hz, 2H), 3.27 (q, J=7.0 Hz, 2H), 2.27-2.09 (m, 2H), 1.91-1.71 (m, 2H), 1.58-1.45 (m, 3H), 1.42 (d, J=7.0 Hz, 3H), 1.13 (s, 18H), 1.09-1.00 (m, 3H). MS (ESI⁺) calc. for C₃₂H₅₃N₃O₁₁P⁺ [M+H]⁺ 686.3, found 686.7.

### ((((E)-5-(((((4-((2S,5S)-13-(2,5-Dioxo-2,5-dihydro-1H-pyrrol-1-yl)-5-isopropyl-2-methyl-4,7-dioxo-8,11-dioxa-3,6-diazatridecanamido)benzyl)oxy)carbonyl)(ethyl)amino)methoxy)-4-methylpent-3-en-1-yl)phosphoryl)bis(oxy))bis(methylene) bis(2,2-dimethylpropanoate) (XC4)

To amine XC3 (15 mg, 0.022 mmol) was added a solution of acid XT2 (7.2 mg, 0.022 mmol) in DMF (0.500 mL). The mixture was cooled in an ice bath, 1-[bis(dimethylamino)methylene]-1H-1,2,3-triazolo[4,5-b]pyridinium 3-oxide hexafluorophosphate (HATU, 10.0 mg, 0.026 mmol) and DIPEA (7.6 μl, 0.044 mmol) were subsequently added, and the resulting mixture was stirred whilst gradually warming to RT. After stirring for 1.5 h at RT, the reaction was concentrated in vacuo and the crude was purified by flash chromatography (silica gel, 0-8% MeOH in DCM) to give XC4 (18 mg) contaminated with residual XT2. The product was taken up in EtOAc and was then washed with sat aq. NaHCO₃/water (1:1), water and brine. The organic layer was dried over Na₂SO₄, filtered, concentrated and further purified by flash chromatography (silica gel, 0-8% MeOH in DCM) to give XC4 (10 mg, 45%) as a colorless oil. ¹H NMR (400 MHz, CDCl₃) ppm=8.80-8.51 (m, 1H), 7.65-7.52 (m, 2H), 7.33-7.28 (m, 2H), 7.14-6.94 (m, 1H), 6.70 (s, 2H), 5.67 (d, J=12.9 Hz, 4H), 5.39-5.27 (m, 1H), 5.16 (br s, 1H), 5.09 (s, 2H), 4.77 (br s, 1H), 4.74-4.63 (m, 2H), 4.30 (br s, 1H), 4.09 (br s, 1H), 4.03 (br t, J=5.5 Hz, 1H), 3.84 (br s, 1H), 3.76-3.60 (m, 5H), 3.55 (br s, 2H), 3.44-3.29 (m, 2H), 2.38-2.18 (m, 3H), 1.98-1.70 (m, 2H), 1.65-1.53 (m, 3H), 1.46 (br d, J=7.0 Hz, 3H), 1.23 (s, 18H), 1.18-1.10 (m, 3H), 1.01 (br d, J=6.6 Hz, 3H), 0.96 (br d, J=6.9 Hz, 3H). MS (ESI⁺) calc. for C₄₆H₇₁N₅O₁₇P⁺ [M+H]⁺ 996.5, found 996.7.

Linker-drug compound XC4 was conjugated to antibodies to create conjugates ADC-XC4-r and ADC-XC4-i, as described in Example 22. Both were tested for their effect on gamma delta T-cells as described in Example 23.

### Example 6: Preparation of Linker-Drug Compound XD18

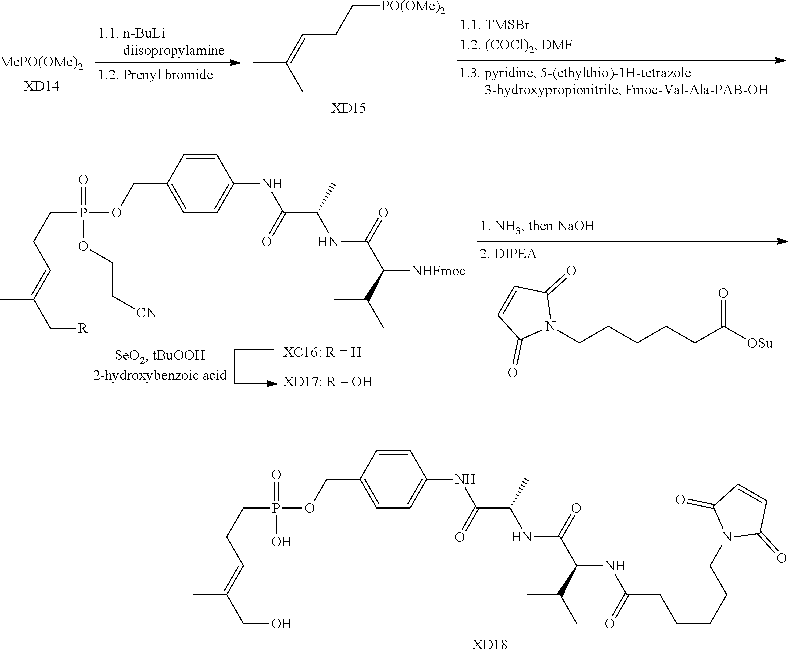

### Dimethyl (4-methylpent-3-en-1-yl)phosphonate (XD15)

To a stirred solution of diisopropylamine (13.0 ml, 92.7 mmol) in THF (280 ml) at −78° C., was added n-butyllithium (1.6 M in hexanes, 55.4 ml, 88.6 mmol) The resulting solution was stirred for 20 min at −78° C. Next, dimethyl methylphosphonate (8.73 ml, 80.6 mmol) was slowly added via a syringe and the mixture was stirred for 1 h. Prenyl bromide (11.6 ml, 101 mmol) was added slowly via a syringe and the solution was then allowed to warm to RT overnight. The reaction was cooled on ice, quenched with sat. aq. NH₄Cl (aq), and extracted with Et₂O (3×). The combined organic layers were washed with brine, dried over Na₂SO₄, filtered and concentrated. Purified by flash chromatography (silica gel, 0-3% MeOH in ether). Product fractions were concentrated and coevaporated with DCM (3×) to remove traces of methanol, to give phosphonic diester XD15 (12.88 g, 83%) as a yellow liquid. ¹H NMR (400 MHz, CDCl₃) ppm=5.14-5.07 (m, 1H), 3.74 (d, J=10.8 Hz, 6H), 2.33-2.22 (m, 2H), 1.83-1.71 (m, 2H), 1.69 (s, 3H), 1.62 (s, 3H). MS (ESI⁺) calc. for C₈H₁₈O₃P⁺ [M+H]⁺ 193.1, found 193.1.

### (9H-Fluoren-9-yl)methyl ((2S)-1-(((2S)-1-((4-((((2-cyanoethoxy)(4-methylpent-3-en-1-yl)phosphoryl)-oxy)methyl)phenyl)amino)-1-oxopropan-2-yl)amino)-3-methyl-1-oxobutan-2-yl)carbamate (XD16)

Step 1: To (4-methylpent-3-en-1-yl)phosphonic dichloride (97.0 mg, 0.485 mmol, prepared from phosphonic diester XD15 according to general procedure XXB) in DCM (1.8 mL) was added 5-(ethylthio)-1H-tetrazole (6.31 mg, 0.048 mmol). The solution was cooled to −78° C. and 3-hydroxypropanenitrile (0.033 ml, 0.485 mmol) and pyridine (0.047 ml, 0.582 mmol) were added. After stirring for 30 min at −78 C, the reaction was warmed to RT and stirred for 2.5 h.

Step 2: In a separate flask, Fmoc-Val-Ala-PAB (250 mg, 0.485 mmol) was taken up in pyridine (1.0 ml). After cooling to 0° C., the phosphonic chloride solution in DCM, prepared in step 1, was cannulated dropwise into the pyridine solution. The reaction was stirred for 30 min at 0° C. and then 1 h at RT. UPLC-MS analysis indicated that the reaction stalled at 50% conversion. The reaction was stored at −30° C. overnight, and step 1 was then repeated with identical amounts but with overnight stirring at RT instead of 2.5 h. The next day, the resulting solution was added at 0° C. to the reaction mixture that was stored overnight. After stirring for 30 min at 0° C. and 1 h at RT complete conversion was observed. The solution was concentrated and the crude was dryloaded on silica gel and purified by flash chromatography (silica gel, 0-25% acetone in DCM) to give the mixed phosphonate diester XD16 (127 mg, 37%) as an impure white solid. MS (ESI⁺) calc. for C₃₉H₄₈N₄O₇P⁺ [M+H]⁺ 715.3, found 715.5.

### (9H-Fluoren-9-yl)methyl ((2S)-1-(((2S)-1-((4-((((2-cyanoethoxy)((E)-5-hydroxy-4-methylpent-3-en-1-yl)phosphoryl)oxy)methyl)phenyl)amino)-1-oxopropan-2-yl)amino)-3-methyl-1-oxobutan-2-yl)carbamate (XD17)

SeO₂ (71 mg, 0.64 mmol) and 2-hydroxybenzoic acid (17 mg, 0.12 mmol) were dissolved in DCM (0.7 ml) and t-BuOOH (70% in water, 0.66 ml, 4.81 mmol) was added at RT. After stirring vigorously for 15 min, the solution was added by pipette to a suspension of XD16 (127 mg, 0.178 mmol) in DCM (1.1 ml). The resulting mixture was stirred vigorously ON. The mixture was cooled to 0° C. and slowly quenched with sat. aq. NaHCO₃ until effervescence stopped. Water was added to solubilize the precipitated salts. The product was extracted with DCM (3×) and the combined organic layers were dried over Na₂SO₄ and concentrated on silica gel. Purification by flash chromatography (silica gel, 0-5% MeOH in DCM) afforded alcohol XD17 (45 mg, 35%) as a white solid that was sufficiently pure for the next step. MS (ESI⁺) calc. for C₃₉H₄₈N₄O₈P⁺ [M+H]⁺ 731.3, found 731.6.

### 4-((S)-2-((S)-2-(6-(2,5-Dioxo-2,5-dihydro-1H-pyrrol-1-yl)hexanamido)-3-methylbutanamido)propan-amido)benzyl hydrogen ((E)-5-hydroxy-4-methylpent-3-en-1-yl)phosphonate (XD18)

Step 1: Phosphonate XD17 (45 mg, 0.062 mmol) in THF (1.0 mL) was diluted with MeOH (9.0 mL). Ammonia in methanol (7 M, 2.35 mL) was then added at RT and the mixture was stirred for 3 h at RT. Next, aq. NaOH (2 M, 1.15 mL) was added at RT and the mixture was stirred for 15 min. The reaction was cooled on ice and aq. AcOH (1 M, 23.5 mL) was added. A cloudy solution formed that was then filtered over a syringe filter. The filtrate was concentrated under vacuum and taken up in dioxane/water (1:1, 1 mL). The solution was lyophilized to give 200 mg of a white solid (mixture of product and salts).

Step 2: The product was taken up in DMF (1 mL), DIPEA (0.049 mL, 0.281 mmol) and 6-maleimidohexanoic acid N-hydroxylsuccinimide ester (70.4 mg, 0.228 mmol) were added at RT, and the mixture was stirred for 30 min. Excess base was quenched with aq. AcOH (1 M, 0.52 mL) at 0° C., and the mixture was concentrated. The crude was purified by preparative RP-HPLC (water×0.1% TFA/MeCN×0.1% 2,2,2-trifluoroacetic acid (TFA)/MeCN, gradient 90:10 to 45:55). MeCN was removed by rotary evaporation and the aq. mixture was lyophilized to give XD18 (26.5 mg, 89% 2 steps). ¹H NMR (400 MHz, DMSO-d₆) ppm=9.92 (s, 1H), 8.14 (d, J=7.0 Hz, 1H), 7.80 (d, J=8.6 Hz, 1H), 7.58 (d, J=8.4 Hz, 2H), 7.30 (d, J=8.5 Hz, 2H), 6.99 (s, 2H), 5.33 (td, J=7.1, 1.0 Hz, 1H), 4.84 (br d, J=7.6 Hz, 2H), 4.61 (br s, 1H), 4.39 (quint, J=6.9 Hz, 1H), 4.17 (dd, J=8.5, 6.9 Hz, 1H), 3.74 (s, 2H), 3.36 (t, J=7.0 Hz, 2H), 2.26-2.06 (m, 4H), 2.02-1.91 (m, 1H), 1.66-1.54 (m, 2H), 1.50 (s, 3H), 1.51-1.39 (m, 4H), 1.30 (d, J=7.1 Hz, 3H), 1.26-1.12 (m, 3H), 0.86 (d, J=6.8 Hz, 3H), 0.82 (d, J=6.8 Hz, 3H). MS (ESI⁺) calc. for C₃₁H₄₆N₄O₉P⁺ [M+H]⁺ 649.3, found 649.6.

### Example 7: Synthesis of Linker-Drug Compound XC9

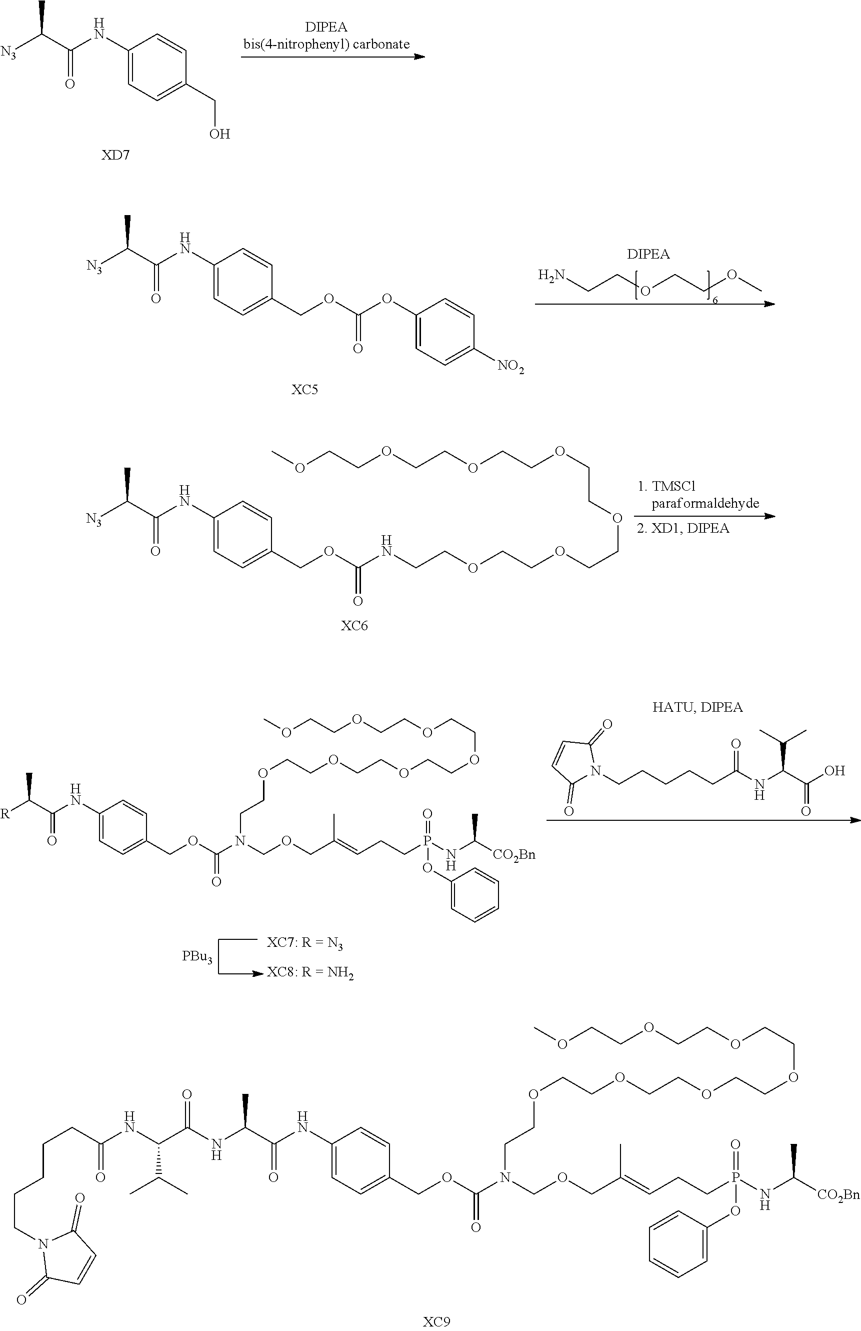

### (S)-4-(2-Azidopropanamido)benzyl (4-nitrophenyl) carbonate (XC5)

To a solution of alcohol XD7 (1.60 g, 7.27 mmol) in THF (20 mL), at 0° C., were added bis(4-nitrophenyl) carbonate (4.42 g, 14.5 mmol) and DIPEA (1.90 mL, 10.9 mmol). The resulting mixture was stirred for 18 h at RT and was then concentrated in vacuo. Purification by flash chromatography (silica gel, 0-5% EtOAc in DCM) afforded carbonate XC5 (1.97 g, 70%) as a pale yellow oil. MS (ESI⁺) calc. for C₁₇H₁₆N₅O₆⁺ [M+H]⁺ 386.1, found 386.2.

### (S)-4-(2-Azidopropanamido)benzyl (2,5,8,11,14,17,20-heptaoxadocosan-22-yl)carbamate (XC6)

To a cooled (0° C.) solution of carbonate XC5 (624 mg, 1.62 mmol) in THF (10.8 mL) were added 2,5,8,11,14,17,20-heptaoxadocosan-22-amine (550 mg, 1.62 mmol) and DIPEA (0.340 mL, 1.94 mmol), and the resulting bright yellow solution was stirred for 18 h whilst gradually warming to RT. After concentration, the crude was purified by flash chromatography (silica gel, 0-6% MeOH in DCM) afforded the carbamate XC6 (875 mg, 92%) as a pale yellow oil. MS (ESI⁺) calc. for C₂₆H₄₄N₅O₁₀⁺ [M+H]⁺ 586.3, found 586.4.

### Benzyl (((E)-23-(((4-((S)-2-azidopropanamido)benzyl)oxy)carbonyl)-27-methyl-2,5,8,11,14,17,20,25-octaoxa-23-azatriacont-27-en-30-yl)(phenoxy)phosphoryl)-L-alaninate (XC7)

Alcohol XD1 (100 mg, 0.240 mmol, prepared as described in Kadri, H. et al. J. Med. Chem. 2020, 63, 11258-11270) was reacted with carbamate XC6 according to general procedure XXA. Purification by flash chromatography (silica gel, 0-11% MeOH in DCM) afforded impure azide XC7 (313 mg) as a colorless oil, that was carried forward without any further purification. MS (ESI⁺) calc. for C₄₉H₇₁N₆NaO₁₅P⁺ 1037.5, found 1037.7

### Benzyl (((E)-23-(((4-((S)-2-aminopropanamido)benzyl)oxy)carbonyl)-27-methyl-2,5,8,11,14,17,20,25-octaoxa-23-azatriacont-27-en-30-yl)(phenoxy)phosphoryl)-L-alaninate (XC8)

A solution of azide XC7 (121 mg, 0.119 mmol) in THF (1.14 mL)/water (0.13 mL) was purged with N₂ for 15 min. Tributylphosphane (0.074 ml, 0.298 mmol) was added at RT and the mixture was stirred for 5.5 h at RT before being concentrated under vacuum. Purification of the crude by flash chromatography (silica gel, 0-15% MeOH in DCM) afforded amine XC8 (62 mg, 52%) as a yellow oil. MS (ESI⁺) calc. for C₄₉H₇₄N₄O₁₅P⁺ [M+H]⁺ 989.5, found 989.8.

### Benzyl (((E)-23-(((4-((S)-2-((S)-2-(6-(2,5-dioxo-2,5-dihydro-1H-pyrrol-1-yl)hexanamido)-3-methylbutanamido)propanamido)benzyl)oxy)carbonyl)-27-methyl-2,5,8,11,14,17,20,25-octaoxa-23-azatriacont-27-en-30-yl)(phenoxy)phosphoryl)-L-alaninate (XC9)

To a cooled (0° C.) solution of amine XC8 (62 mg, 0.063 mmol) and (6-(2,5-dioxo-2,5-dihydro-1H-pyrrol-1-yl)hexanoyl)-L-valine (19.5 mg, 0.063 mmol, prepared as described in WO2013122823) in DMF (1 mL) was added HATU (28.6 mg, 0.075 mmol) and DIPEA (0.022 mL, 0.125 mmol). The resulting yellow mixture was stirred at RT for 1.5 h before being concentrated in vacuo. The residue was dissolved in EtOAc and washed with sat. aq. NaHCO₃/water (1:1), water and brine, dried over Na₂SO₄, and concentrated. The crude was purified by flash chromatography (silica gel, 0-10% MeOH in DCM) to give a colorless oil. The oil was taken up in MeCN/MilliQ (1:1) and purified by preparative RP-HPLC (water/MeCN, gradient 60:40 to 10:90, no modifier used). Product fractions were pooled and MeCN was removed by rotary evaporation. The aqueous mixture was then lyophilized to yield maleimide XC9 (25 mg, 31%) as a colorless oil. MS (ESI⁺) calc. for C₆₄H₉₄N₆O₁₉P⁺ [M+H]⁺ 1281.6, found 1282.0.

### Example 8: Synthesis of Linker-Drug Compound XC13

### Preparation of benzyl (E)-((5-hydroxy-4-methylpent-3-en-1-yl)(4-iodophenoxy)phosphoryl)alaninate (XS4)

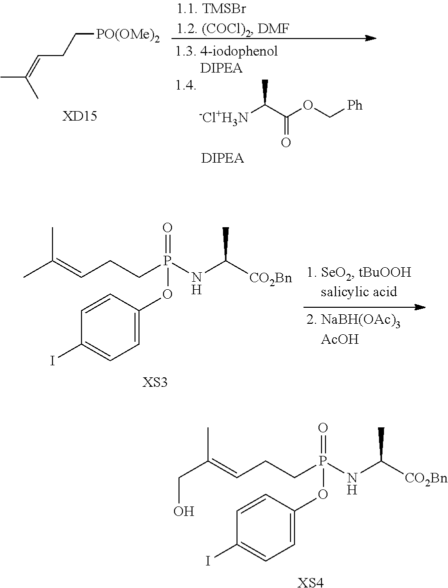

### Benzyl ((4-iodophenoxy)(4-methylpent-3-en-1-yl)phosphoryl)alaninate (XS3)

To (4-methylpent-3-en-1-yl)phosphonic dichloride (837 mg, 4.16 mmol, prepared from phosphonic diester XD15 (1.00 g, 5.20 mmol) according to general procedure XXB) in toluene (27 mL) was dropwise added a solution of 4-iodophenol (1.83 g, 8.33 mmol) and DIPEA (1.45 mL, 8.33 mmol) in toluene (27 mL) at −78° C. The reaction mixture was stirred at −78° C. for 30 min. Benzyl L-alaninate hydrochloride (1.89 g, 8.74 mmol) and DIPEA (3.05 mL, 17.5 mmol) were added and the reaction mixture was allowed to reach RT and was stirred for 2 h. The reaction mixture was concentrated and the crude was purified by flash chromatography (silica gel, 0-50% EtOAc in heptane), to yield XS3 (0.490 g, 22%, ˜3:2 diastereomeric mixture) as a yellow solid. ¹H NMR (400 MHz, CDCl₃) ppm=7.60-7.53 (m, 2H), 7.41-7.28 (m, 5H), 7.00-6.93 (m, 2H), 5.14-5.06 (m, 3H), 4.15-4.04 (m, 1H), 3.31 (t, J=10.4 Hz, 0.6H), 3.22 (t, J=10.5 Hz, 0.4H), 2.41-2.28 (m, 2H), 1.96-1.80 (m, 2H), 1.69 (s, 3H), 1.62 (s, 3H), 1.33 (d, J=7.1 Hz, 1.9H), 1.27 (d, J=7.1 Hz, 1.1H). MS (ESI⁺) calc. for C₂₂H₂₈INO₄P⁺ [M+H]⁺ 528.08, found 528.26.

### Benzyl (E)-((5-hydroxy-4-methylpent-3-en-1-yl)(4-iodophenoxy)phosphoryl)alaninate (XS4)

The allylic oxidation of alkene XS3 (0.434 g, 0.823 mmol) was performed according to general procedure XXC. The crude was purified by flash chromatography (silica gel, 40-100% EtOAc in heptane), to yield alcohol XS4 (0.254 g, 57%, ˜3:2 diastereomeric mixture) as a colorless oil. ¹H NMR (400 MHz, CDCl₃) ppm=7.61-7.54 (m, 2H), 7.40-7.28 (m, 5H), 7.00-6.93 (m, 2H), 5.47-5.39 (m, 1H), 5.15-5.07 (m, 2H), 4.17-4.02 (m, 1H), 4.00 (s, 2H), 3.36 (t, J=10.3 Hz, 0.6H), 3.27 (dd, J=11.6, 9.6 Hz, 0.4H), 2.49-2.35 (m, 2H), 2.01-1.83 (m, 2H), 1.68 (s, 3H), 1.34 (d, J=7.0 Hz, 1.8H), 1.25 (d, J=7.1 Hz, 1.2H). MS (ESI⁺) calc. for C₂₂H₂₈INO₅P⁺ [M+H]⁺ 544.07, found 544.32.

Linker-drug compound XC13 was synthesized from XS4 according to the following reaction scheme.

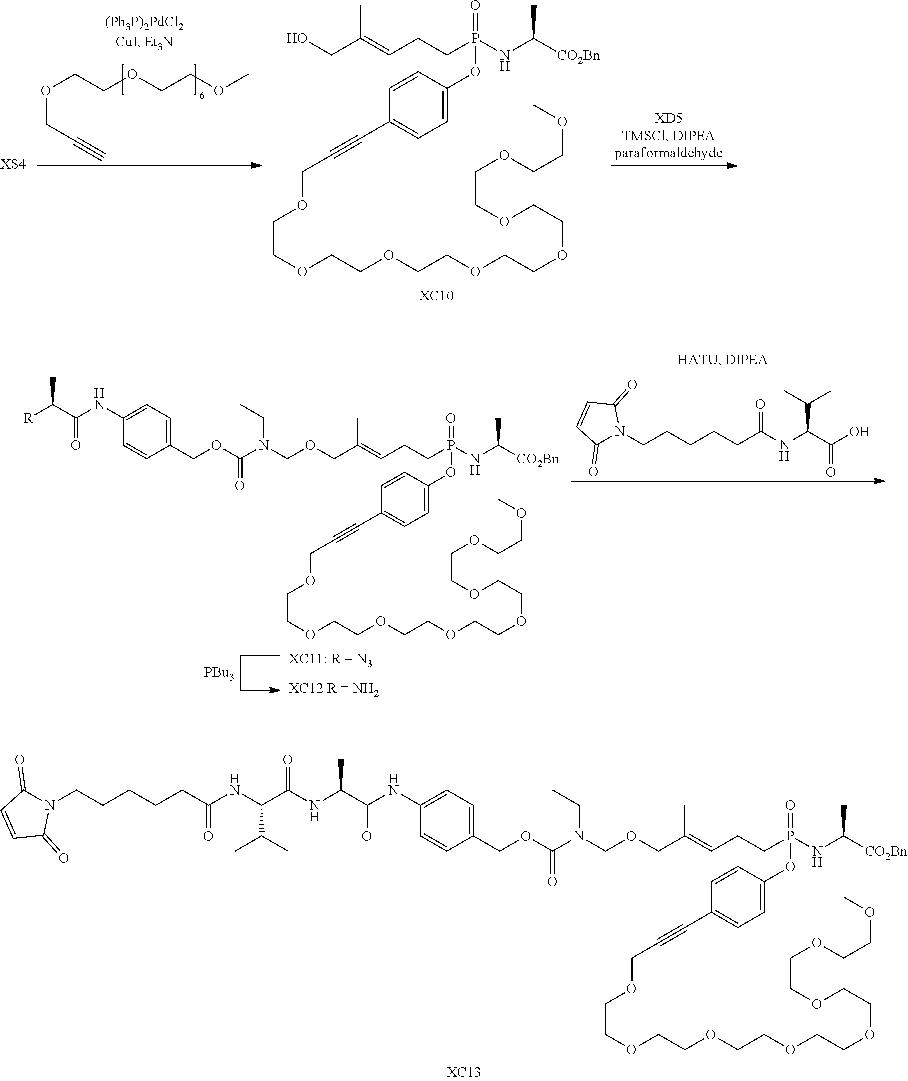

### Benzyl ((4-(2,5,8,11,14,17,20,23-octaoxahexacos-25-yn-26-yl)phenoxy)((E)-5-hydroxy-4-methylpent-3-en-1-yl)phosphoryl)-L-alaninate (XC10)

To XS4 (50 mg, 0.092 mmol), 2,5,8,11,14,17,20,23-octaoxahexacos-25-yne (34.8 mg, 0.092 mmol), bis(triphenylphosphino)palladium chloride (3.23 mg, 4.60 μmol) and Cu(I)I (1.753 mg, 9.20 μmol) was added degassed Et₃N (0.2 mL, 1.44 mmol). The mixture was stirred at RT for 4 h. After concentration, the crude was redissolved in DCM and again concentrated. Purification of the crude by flash chromatography (silica gel, 0-11% MeOH in DCM) afforded alcohol XC10 (67 mg, 83%) as a brown oil. MS (ESI⁺) calc. for C₄₀H₆₁NO₁₃P⁺ [M+H]⁺ 794.4, found 794.6.

### Benzyl ((4-(2,5,8,11,14,17, 20,23-octaoxahexacos-25-yn-26-yl)phenoxy)((E)-5-(((((4-((S)-2-azidopropanamido)benzyl)oxy)carbonyl)(ethyl)amino)methoxy)-4-methylpent-3-en-1-yl)phosphoryl)-L-alaninate (XC11)

Alcohol XC10 (67 mg, 0.076 mmol) was reacted with carbamate XD5 according to general procedure XXA. Purification by flash chromatography (silica gel, 0-8% MeOH in DCM) afforded azide XC11 (54 mg, 65%) as a pale brown oil. MS (ESI⁺) calc. for C₅₄H₇₈N₆O₁₆P⁺ [M+H]⁺ 1097.5, found 1097.8.

### Benzyl ((4-(2,5,8,11,14,17,20,23-octaoxahexacos-25-yn-26-yl)phenoxy)((E)-5-(((((4-((S)-2-aminopropanamido)benzyl)oxy)carbonyl)(ethyl)amino)methoxy)-4-methylpent-3-en-1-yl)phosphoryl)-L-alaninate (XC12)

A solution of azide XC11 (54 mg, 0.049 mmol) in THF (0.473 mL)/water (0.053 mL) was purged with N₂ for 15 min. Tributylphosphane (0.031 ml, 0.123 mmol) was added at RT and the mixture was stirred for 5.5 h at RT before being concentrated under vacuum. Purification by flash chromatography (silica gel, 0-15% MeOH in DCM) afforded amine XC12 (42 mg, 80%) as a pale yellow oil. MS (ESI⁺) calc. for C₅₄H₈₀N₄O₁₆P⁺ [M+H]⁺ 1071.5, found 1071.9

### Benzyl ((4-(2,5,8,11,14,17,20,23-octaoxahexacos-25-yn-26-yl)phenoxy)((E)-5-(((((4-((S)-2-((S)-2-(6-(2,5-dioxo-2,5-dihydro-JH-pyrrol-1-yl)hexanamido)-3-methylbutanamido)propanamido)benzyl)oxy)carbonyl)(ethyl)amino)methoxy)-4-methylpent-3-en-1-yl)phosphoryl)-L-alaninate (XC13)

To a cooled (0° C.) solution of amine XC12 (42 mg, 0.039 mmol) and (6-(2,5-dioxo-2,5-dihydro-1H-pyrrol-1-yl)hexanoyl)-L-valine (12.2 mg, 0.039 mmol, prepared as described in WO2013122823) in DMF (1 mL) was added HATU (17.9 mg, 0.047 mmol) and DIPEA (0.014 mL, 0.078 mmol). The resulting yellow mixture was stirred at RT for 1.5 h before being concentrated in vacuo. The residue was dissolved in EtOAc and washed with sat. aq. NaHCO₃/water (1:1), water and brine, dried over Na₂SO₄, and concentrated. The crude was purified by flash chromatography (silica gel, 0-10% MeOH in DCM) to give a colorless oil. The oil was purified by preparative RP-HPLC (water/MeCN, gradient 60:40 to 10:90, no modifier used). Product fractions were pooled and MeCN was removed by rotary evaporation. The aqueous mixture was then lyophilized to yield maleimide XC13 (21 mg, 40%) as a white solid. MS (ESI⁺) calc. for C₆₉H₁₀₀N₆O₂₀P⁺ [M+H]⁺ 1363.7, found 1364.1.

### Example 9: Synthesis of Linker-Drug Compound XS2

### Preparation of 2,5-dioxopyrrolidin-1-yl (6-(2,5-dioxo-2,5-dihydro-1H-pyrrol-1-yl)hexanoyl)glycylglycyl-L-phenylalaninate (XD20)

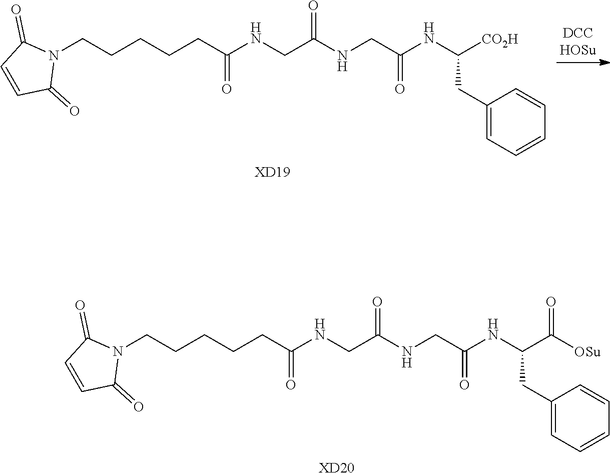

2,5-Dioxopyrrolidin-1-yl (6-(2,5-dioxo-2,5-dihydro-1H-pyrrol-1-yl)hexanoyl)glycylglycyl-L-phenylalaninate (XD20)

N,N′-Dicyclohexylcarbodiimide (DCC, 459 mg, 2.222 mmol) was added to a suspension of XD19 (1.05 g, 2.22 mmol) and 1-hydroxypyrrolidine-2,5-dione (256 mg, 2.22 mmol, synthesized as described in EP2907824) in THF (40 ml) at RT. After stirring for 3.5 h, the mixture was filtered and DCM was used to wash the residue thoroughly. The filtrate was diluted with EtOAc and was then concentrated. The white solid was suspended in a small volume of EtOAc and was then filtered to give OSu-ester XD20 (612 mg, 48%) as a white solid. MS (ESI⁺) calc. for C₂₇H₃₂N₅O₉+[M+H]⁺ 570.2, found 570.4.

### Preparation of benzyl (((E)-5-((2-((S)-2-(2-(2-(6-(2,5-dioxo-2,5-dihydro-1H-pyrrol-1-yl)hexanamido)acetamido)acetamido)-3-phenylpropanamido)acetamido)methoxy)-4-methylpent-3-en-1-yl)(phenoxy)phosphoryl)-L-alaninate (XS2)

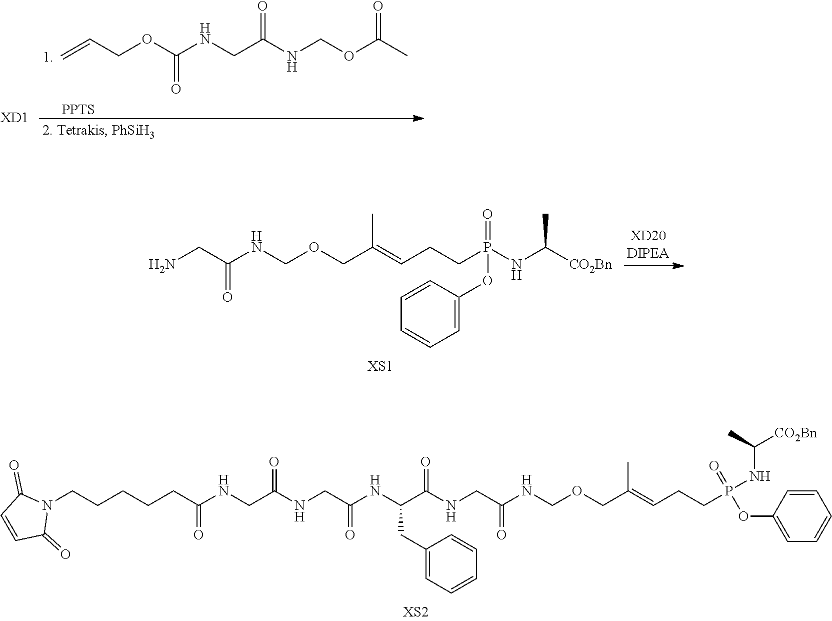

### Benzyl (((E)-5-((2-aminoacetamido)methoxy)-4-methylpent-3-en-1-yl)(phenoxy)phosphoryl)-L-alaninate (XS1)

Step 1: A flask was charged with (2-(((allyloxy)carbonyl)amino)acetamido)methyl acetate (0.152 g, 0.659 mmol, prepared as described by Brailsford et al. Tetrahedron, 2018, 74, 1951-1956) and pyridinium p-toluenesulfonate (PPTS; 10.6 mg, 0.042 mmol) under N₂. Alcohol XD1 (0.110 g, 0.264 mmol, prepared as described in Kadri et al. J. Med. Chem. 2020, 63, 11258-11270) in toluene (1.3 mL) was added and the reaction mixture was stirred at 80° C. for 1 h. After cooling to RT, Et₃N (4 drops) was added and the reaction mixture was concentrated and coevaporated with DCM (1 mL). The crude was purified by flash chromatography (silica gel, 20-100% EtOAc in DCM) to yield benzyl (E)-((5-((2-(((allyloxy)carbonyl)amino)acetamido)methoxy)-4-methylpent-3-en-1-yl)(phenoxy)phosphoryl-)alaninate (0.120 g, 68% yield) as a mixture of diastereoisomers. MS (ESI⁺) calc. for C₂₉H₃₉N₃O₈P⁺ [M+H]⁺ 588.25, found 588.42.

Step 2: A 10 mL vial was purged with N₂ (3× vacuum/N₂ cycles) and charged with Pd(PPh₃)₄ (4.2 mg, 0.0036 mmol). Next, the Alloc-protected amine (0.120 g, 0.180 mmol, prepared in step 1) in DCM (1.80 mL) was added, followed by PhSiH₃ (0.155 mL, 1.26 mmol). The reaction mixture was stirred for 1 h. The reaction mixture was diluted with DCM and purified by flash chromatography (silica gel, 5-15% MeOH in DCM) to yield XS1 (63.7 mg, 70%, ˜3:2 diastereomeric mixture) as a yellow oil. ¹H NMR (400 MHz, CDCl₃) ppm=8.01 (br s, 1H), 7.46-7.22 (m, 7H), 7.22-7.16 (m, 2H), 7.16-7.09 (m, 1H), 5.48 (q, J=7.2 Hz, 1H), 5.27-4.91 (m, 2H), 4.82-4.64 (m, 2H), 4.19-4.00 (m, 1H), 3.90 (s, 2H), 3.47 (t, J=10.2 Hz, 1H), 3.42-3.28 (m, 2H), 2.49-2.34 (m, 2H), 2.02-1.84 (m, 4H), 1.65 (s, 3H), 1.32 (d, J=7.0 Hz, 1.8H), 1.25 (d, J=7.1 Hz, 1.2H). MS (ESI⁺) calc. for C₂₅H₃₅N₃O₆P⁺ [M+H]⁺ 504.2, found 504.4.

### Benzyl (((E)-5-((2-((S)-2-(2-(2-(6-(2,5-dioxo-2,5-dihydro-1H-pyrrol-1-yl)hexanamido)acetamido)acetamido)-3-phenylpropanamido)acetamido)methoxy)-4-methylpent-3-en-1-yl)(phenoxy)phosphoryl)-L-alaninate (XS2)

To a solution of amine XS1 (25.7 mg, 0.051 mmol) in DMF (0.51 mL) was added OSu-ester XD20 (31.7 mg, 0.056 mmol), followed by DIPEA (0.027 mL, 0.153 mmol). The reaction mixture was stirred for 60 min at RT. More OSu-ester XD20 (5.81 mg, 10.2 μmol) was added, and after stirring for 40 min, a final portion of OSu-ester XD20 (5.81 mg, 10.2 μmol) was added, followed by stirring for 30 min. The reaction mixture was concentrated, coevaporated with toluene (2 mL) and dried in vacuo. The crude was dissolved in DCM (5 mL), filtered over a syringe filter and purified by flash chromatography (silica gel, 0-8% MeOH in DCM). The product was further purified by RP-HPLC (water/MeCN, gradient 70:30 to 45:55, no modifier added). Evaporation of MeCN and subsequent lyophilization afforded XS2 (14.8 mg, 30%, ˜3:2 diastereomeric mixture). ¹H NMR (400 MHz, DMSO-d₆) ppm=8.50-8.43 (m, 1H), 8.27 (t, J=5.8 Hz, 1H), 8.10 (d, J=8.0 Hz, 1H), 8.06 (t, J=5.8 Hz, 1H), 8.00 (t, J=5.8 Hz, 1H), 7.38-7.27 (m, 7H), 7.27-7.21 (m, 4H), 7.20-7.11 (m, 4H), 6.98 (s, 2H), 5.63 (dd, J=12.5, 10.4 Hz, 0.4H), 5.52 (dd, J=13.3, 10.0 Hz, 0.6H), 5.44-5.36 (m, 1H), 5.13-5.02 (m, 2H), 4.55-4.46 (m, 3H), 4.00-3.90 (m, 1H), 3.77 (s, 2H), 3.74-3.69 (m, 2H), 3.66 (d, J=5.8 Hz, 2H), 3.63-3.55 (m, 1H), 3.38-3.35 (m, 2H), 3.06 (dd, J=13.8, 4.5 Hz, 1H), 2.81 (dd, J=13.8, 9.8 Hz, 1H), 2.31-2.19 (m, 2H), 2.11 (t, J=7.5 Hz, 2H), 1.88-1.75 (m, 2H), 1.57-1.53 (m, 3H), 1.52-1.42 (m, 4H), 1.24-1.18 (m, 3H), 1.13 (d, J=7.1 Hz, 2H). MS (ESI⁺) calc. for C₄₈H₆₁N₇O₁₂P⁺ [M+H]⁺ 958.4, found 958.8.

### Example 10: Synthesis of Linker-Drug XS7

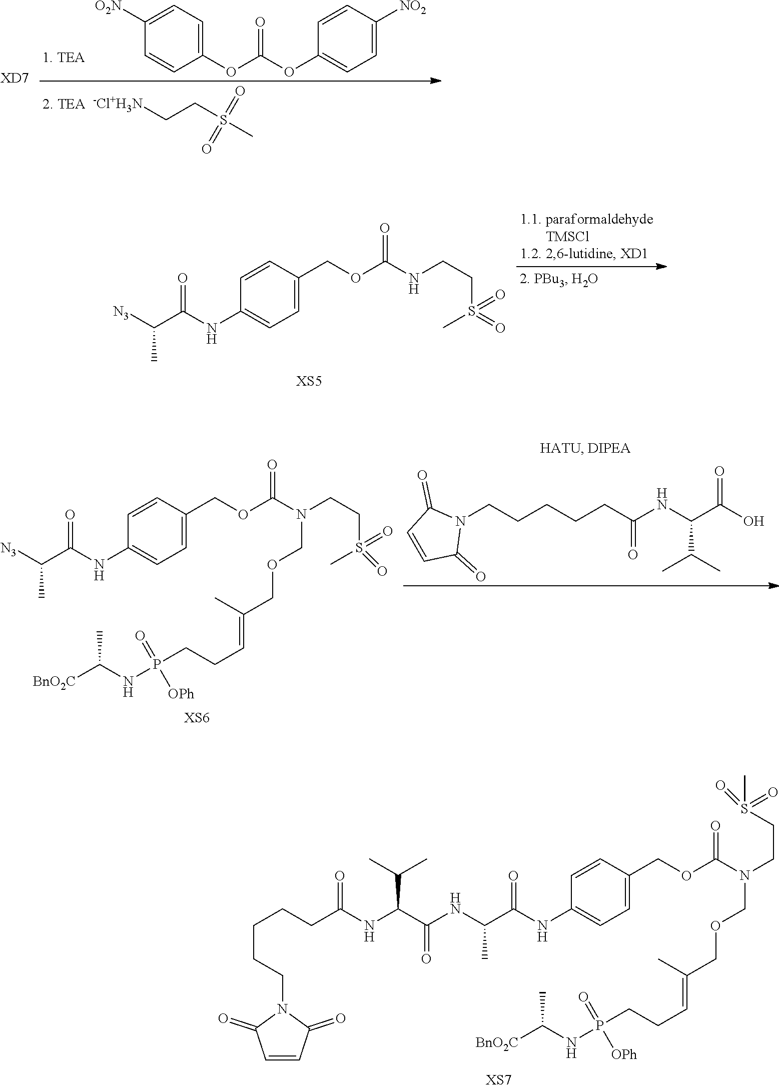

### (S)-4-(2-Azidopropanamido)benzyl (2-(methylsulfonyl)ethyl)carbamate (XS5)

Step 1: To a solution of XD7 (1.14 g, 5.18 mmol, prepared as described in example 1) in THF (17 mL) was added bis(4-nitrophenyl) carbonate (3.15 g, 10.4 mmol), followed by DIPEA (1.36 mL, 7.76 mmol). The reaction mixture was stirred overnight at RT. The reaction mixture was concentrated and the crude was stirred in Et₂O (20 mL) for 15 min and filtered. This process was repeated twice and the filtrates were combined and concentrated. Purification by flash chromatography (silica gel, 0-50% EtOAc in heptane), afforded the corresponding carbonate (1.57 g, 79%). MS (ESI⁺) calc. for C₁₇H₁₆N₅O₆⁺ [M+H]⁺ 386.1, found 386.2.

Step 2: The carbonate intermediate (0.340 g, 0.882 mmol) was dissolved in THF (4.4 mL) and 2-(methylsulfonyl)ethan-1-amine hydrochloride (0.148 g, 0.926 mmol) and TEA (0.258 mL, 1.85 mmol) were added at 0° C. The mixture was allowed to reach RT, and after stirring for 3 h, the reaction mixture was concentrated, taken up in EtOAc (30 mL) and washed with sat. aq. NaHCO₃ (3×20 mL) and brine (20 mL), dried over Na₂SO₄ and concentrated. The crude was purified by flash chromatography (silica gel, 0-100% EtOAc in DCM), to yield carbamate XS5 (0.260 g, 80%) as a white solid. ¹H NMR (400 MHz, CDCl₃) ppm=8.11 (br s, 1H), 7.57-7.51 (m, 2H), 7.33 (d, J=8.5 Hz, 2H), 5.42 (br s, 1H), 5.07 (s, 2H), 4.24 (q, J=7.0 Hz, 1H), 3.72 (q, J=6.1 Hz, 2H), 3.25 (t, J=5.9 Hz, 2H), 2.93 (s, 3H), 1.65 (d, J=7.0 Hz, 3H). MS (ESI⁺) calc. for C₁₄H₁₉N₅NaO₅S⁺ [M+Na]⁺392.1, found 392.1.

### Benzyl (((E)-5-(((((4-((S)-2-aminopropanamido)benzyl)oxy)carbonyl)(2-(methylsulfonyl)ethyl)amino)methoxy)-4-methylpent-3-en-1-yl)(phenoxy)phosphoryl)-L-alaninate (XS6)

Step 1: To a solution of carbamate XS5 (0.186 g, 0.503 mmol) in DCM (2.5 mL) was added paraformaldehyde (18 mg, 0.60 mmol). The reaction mixture was stirred for 5 min, after which TMSCl (0.070 mL, 0.55 mmol) was added. The reaction mixture was stirred for 1 h, concentrated, coevaporated with DCM (1 mL), dried in vacuo for 15 min and dissolved in DCM (2.5 mL) to give solution A. Allylic alcohol XD1 (84 mg, 0.20 mmol) was dissolved in DCM (1.3 mL), cooled to 0° C., and a portion of solution A (1.5 mL) was added, followed by 2,6-lutidine (0.070 mL, 0.60 mmol). The reaction mixture was allowed to reach RT and stirred for 2 h. More solution A (0.50 mL) and 2,6-lutidine (0.023 mL, 0.20 mmol) were added and the mixture was stirred for 1 h. More solution A (0.50 mL) and 2,6-lutidine (0.023 mL, 0.20 mmol) were added and the mixture was stirred overnight. A few drops of n-BuOH were added to quench the reaction and the mixture was concentrated, coevaporated with heptane (1 mL), toluene (1 mL) and DCM (1 mL). The crude was purified by flash chromatography (silica gel, 0-100% EtOAc in DCM), to yield both diastereomers of benzyl (((E)-5-(((((4-((S)-2-azidopropanamido)benzyl)oxy)carbonyl)(2-(methylsulfonyl)ethyl)amino)methoxy)-4-methylpent-3-en-1-yl)(phenoxy)phosphoryl)-L-alaninate (0.104 g, 65%) as a colorless oil. MS (ESI⁺) calc. for C₃₇H₄₈N₆O₁₀PS⁺ [M+H]⁺ 799.3, found 799.6.

Step 2: This intermediate (60 mg, 0.075 mmol) was dissolved in THF (0.68 mL)/water (0.075 mL) and the resulting solution was purged with N₂ for 15 min. Tributylphosphane (0.047 mL, 0.188 mmol) was added and the reaction mixture was stirred overnight at RT. The reaction mixture was concentrated, coevaporated with MeCN (2×2 mL) and dried in vacuo. The crude was purified by flash chromatography (silica gel, 0-20% MeOH in DCM), to yield amine XS6 (27.5 mg, 47%) as a mixture of diastereomers. MS (ESI⁺) calc. for C₃₇H₅₀N₄O₁₀PS⁺ [M+H]⁺ 773.3, found 773.6.

### Linker-Drug XS7

Amine XS6 (28 mg, 0.036 mmol) and (6-(2,5-dioxo-2,5-dihydro-1H-pyrrol-1-yl)hexanoyl)-L-valine (12 mg, 0.039 mmol, prepared as described in WO2013122823) were dissolved in DMF (0.36 mL), and HATU (15 mg, 0.039 mmol) and DIPEA (0.025 mL, 0.14 mmol) were added. The reaction mixture was stirred for 40 min at RT, concentrated and coevaporated with toluene (2×1 mL). The residue was dissolved in EtOAc (10 mL) and washed with sat. aq. NaHCO₃ (10 mL). The water layer was backextracted with EtOAc (2×10 mL) and the combined organic layers were washed with water (10 mL) and brine (10 mL), dried over Na₂SO₄ and concentrated. The crude was purified by preparative RP-HPLC (MilliQ/MeCN, gradient 70:30 to 20:80, no modifier added) to give after lyophilization XS7 (17.1 mg, 45%) as mixture of diastereoisomers. ¹H NMR (400 MHz, DMSO-d₆) ppm=9.94 (s, 1H), 8.14 (d, J=6.9 Hz, 1H), 7.79 (d, J=8.6 Hz, 1H), 7.59 (d, J=8.4 Hz, 2H), 7.38-7.27 (m, 9H), 7.20-7.10 (m, 3H), 6.99 (s, 2H), 5.62 (t, J=11.4 Hz, 0.4H), 5.56-5.47 (m, 0.6H), 5.45-5.29 (m, 1H), 5.13-5.01 (m, 4H), 4.74 (s, 2H), 4.38 (quint, J=7.0 Hz, 1H), 4.17 (dd, J=8.5, 6.9 Hz, 1H), 4.04-3.89 (m, 1H), 3.81-3.71 (m, 2H), 3.71-3.63 (m, 2H), 3.42-3.33 (m, 4H), 3.05-2.91 (m, 3H), 2.35-2.20 (m, 2H), 2.20-2.07 (m, 2H), 2.03-1.90 (m, 1H), 1.89-1.71 (m, 2H), 1.59-1.44 (m, 7H), 1.30 (d, J=7.0 Hz, 3H), 1.23-1.20 (m, 1H), 1.19-1.15 (m, 2H), 1.13 (d, J=7.3 Hz, 2H), 0.86 (d, J=6.8 Hz, 3H), 0.82 (d, J=6.8 Hz, 3H). MS (ESI⁺) calc. for C₅₂H₇₀N₆O₁₄PS⁺ [M+H]⁺ 1065.4, found 1065.9.

### Example 11: Synthesis of Linker-Drug XS25

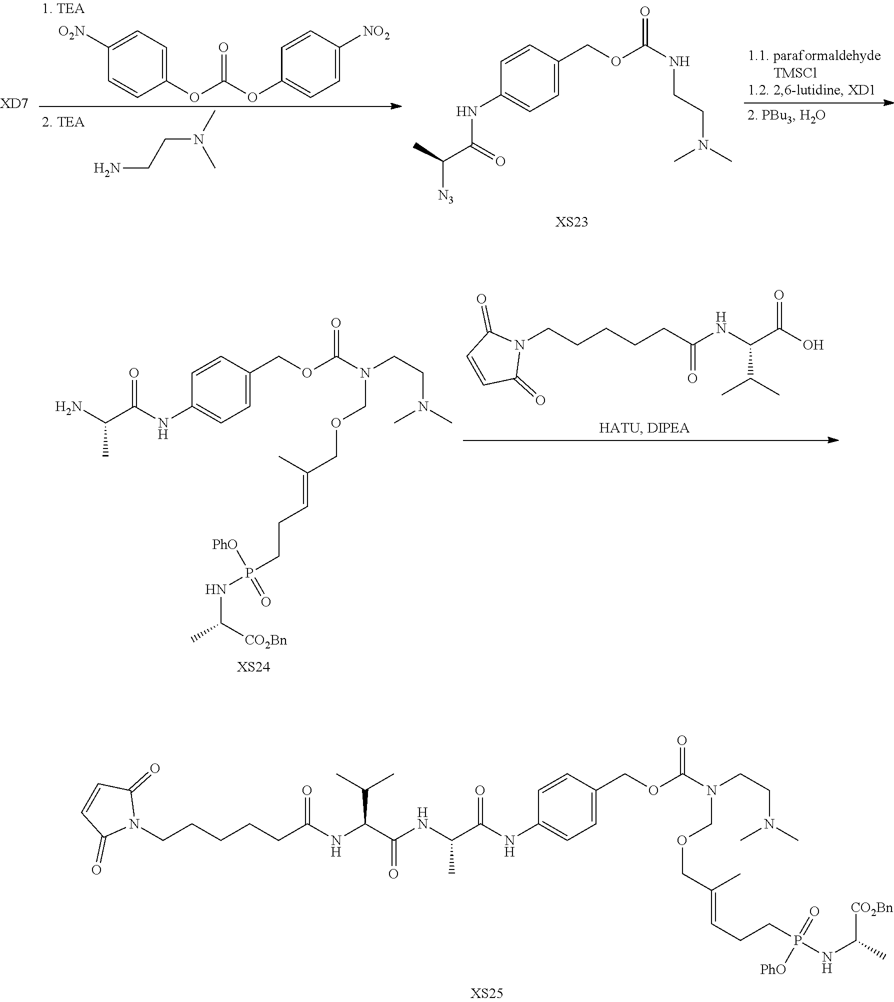

### (S)-4-(2-Azidopropanamido)benzyl (2-(dimethylamino)ethyl)carbamate (XS23)

The PNP-carbonate of XD7 was prepared as described in the synthesis of XS5. The carbonate (0.393 g, 1.02 mmol) was dissolved in THF (5.1 mL) and at 0° C. were added N,N-dimethylethane-1,2-diamine (0.137 mL, 1.25 mmol) and TEA (0.258 mL, 1.85 mmol). The mixture was allowed to reach RT and stirred for 4 h. The reaction mixture was concentrated, taken up in EtOAc (30 mL) and washed with sat. aq. NaHCO₃ (3×15 mL). The combined water layer was backextracted with EtOAc (25 mL) and the combined organic layer was washed with aq. NaOH (2×15 mL, 1 N), brine (25 mL) and dried over Na₂SO₄ and evaporated, to yield carbamate XS23 (0.299 g, 88%) as a pale yellow solid. ¹H NMR (400 MHz, CDCl₃) ppm=8.11 (br s, 1H), 7.56-7.51 (m, 2H), 7.37-7.32 (m, 2H), 5.26 (br s, 1H), 5.06 (s, 2H), 4.23 (q, J=7.0 Hz, 1H), 3.26 (q, J=5.6 Hz, 2H), 2.39 (t, J=6.0 Hz, 2H), 2.21 (s, 6H), 1.64 (d, J=7.0 Hz, 3H). MS (ESI⁺) calc. for C₁₅H₂₃N₆O₃⁺ [M+H]⁺ 335.2, found 335.3.

### Benzyl (((E)-5-(((((4-((S)-2-aminopropanamido)benzyl)oxy)carbonyl)(2-(dimethylamino)-ethyl)amino)methoxy)-4-methylpent-3-en-1-yl)(phenoxy)phosphoryl)-L-alaninate (XS24)

Step 1: To a solution of carbamate XS23 (0.160 g, 0.478 mmol) in DCM (4.8 mL) was added paraformaldehyde (20 mg, 0.67 mmol). The reaction mixture was stirred for 15 min, after which TMSCl (0.094 mL, 0.74 mmol) was added. The reaction mixture was stirred for 1 h, TMSCl (0.094 mL, 0.74 mmol) was added and stirring was continued for 2.5 h. The reaction mixture was then concentrated, coevaporated with DCM (1 mL) and dried in vacuo for 15 min. The crude intermediate was suspended in DCM (4.8 mL) and a solution of XD1 (0.493 g, 1.18 mmol) in DCM (4.8 mL) was added. The reaction mixture was stirred for 15 min, followed by the addition of 2,6-lutidine (0.167 mL, 1.44 mmol). After 20 min, MeOH (2 mL) was added and the reaction mixture was concentrated. The crude was purified by flash chromatography (silica gel, 0-20% MeOH in DCM), to yield a diastereomeric mixture of benzyl (((E)-5-(((((4-((S)-2-azidopropanamido)benzyl)oxy)carbonyl)(2-(dimethylamino)ethyl)amino)methoxy)-4-methylpent-3-en-1-yl)(phenoxy)phosphoryl)-L-alaninate (0.272 g, 74%) as a pale yellow oil. ¹H NMR (400 MHz, DMSO-d₆) ppm=10.34 (s, 1H), 7.63 (d, J=8.1 Hz, 2H), 7.40-7.26 (m, 9H), 7.20-7.10 (m, 3H), 5.63 (t, J=11.4 Hz, 0.6H), 5.53 (dd, J=13.1, 10.2 Hz, 0.4H), 5.46-5.30 (m, 1H), 5.06 (d, J=13.3 Hz, 4H), 4.71 (s, 2H), 4.09 (q, J=5.3 Hz, 2H), 4.07-4.02 (m, 1H), 4.02-3.92 (m, 1H), 3.86-3.69 (m, 2H), 3.17 (d, J=5.1 Hz, 4H), 2.73-2.59 (m, 4H), 2.33-2.16 (m, 2H), 1.87-1.72 (m, 2H), 1.59-1.49 (m, 3H), 1.49-1.40 (m, 3H), 1.28-1.15 (m, 2H), 1.13 (d, J=7.3 Hz, 1H). MS (ESI⁺) calc. for C₃₈H₅₁N₇O₈P⁺ [M+H]⁺ 764.4, found 764.7.

Step 2: This intermediate (75 mg, 0.098 mmol) was dissolved in THF (0.884 mL)/water (0.098 mL) and the resulting solution was purged with N₂ for 15 min. Tributylphosphane (61 μL, 0.245 mmol) was added and the reaction mixture was stirred overnight at RT. The reaction mixture was concentrated, coevaporated with MeCN (2×2 mL) and dried in vacuo. The crude was purified by flash chromatography (silica gel, 0-25% MeOH in DCM), to yield both diastereomers of amine XS24 (33 mg, 45%) as a yellow oil. MS (ESI⁺) calc. for C₃₈H₅₃N₅O₈P⁺ [M+H]⁺ 738.4, found 736.7.

### Linker-Drug XS25

Amine XS24 (33 mg, 0.044 mmol) and (6-(2,5-dioxo-2,5-dihydro-1H-pyrrol-1-yl)hexanoyl)-L-valine (14 mg, 0.046 mmol, prepared as described in WO2013122823) were dissolved in DMF (0.44 mL). HATU (18 mg, 0.049 mmol) and DIPEA (31 μL, 0.18 mmol) were added, and the reaction mixture was stirred at RT for 1 h, concentrated and coevaporated with toluene (1 mL). Water (1 mL) was added, the resulting supernatant was removed and the precipitate was washed with water. The crude was purified by preparative RP-HPLC (MilliQ×0.1% TFA/MeCN, gradient 80:20 to 30:70) to give, after lyophilization, XS25 (18 mg, 39%) as a diastereomeric mixture. ¹H NMR (400 MHz, DMSO-d₆) ppm=9.94 (s, 1H), 9.37 (br s, 1H), 8.14 (d, J=6.9 Hz, 1H), 7.79 (d, J=8.5 Hz, 1H), 7.60 (d, J=8.3 Hz, 2H), 7.38-7.27 (m, 9H), 7.19-7.10 (m, 3H), 6.99 (s, 2H), 5.63 (t, J=11.4 Hz, 0.7H), 5.52 (dd, J=13.1, 10.3 Hz, 0.3H), 5.46-5.30 (m, 1H), 5.10-5.06 (m, 2H), 5.06-5.04 (m, 2H), 4.71 (s, 2H), 4.38 (quint, J=7.0 Hz, 1H), 4.16 (dd, J=8.5, 6.9 Hz, 1H), 4.06-3.88 (m, 1H), 3.84-3.73 (m, 2H), 3.64-3.55 (m, 2H), 3.39-3.36 (m, 2H), 3.29-3.18 (m, 2H), 2.85-2.70 (m, 6H), 2.31-2.21 (m, 2H), 2.21-2.08 (m, 2H), 1.95 (dq, J=13.6, 6.8 Hz, 1H), 1.88-1.73 (m, 2H), 1.60-1.52 (m, 3H), 1.52-1.43 (m, 4H), 1.30 (d, J=7.0 Hz, 3H), 1.21 (d, J=7.0 Hz, 3H), 1.19-1.11 (m, 2H), 0.86 (d, J=6.8 Hz, 3H), 0.82 (d, J=6.8 Hz, 3H). MS (ESI⁺) calc. for C₅₃H₇₃N₇O₁₂P⁺ [M+H]⁺ 1030.5, found 1030.9.

### Example 12: Synthesis of Linker-Drug XS12

### A: Preparation of Azide XS9

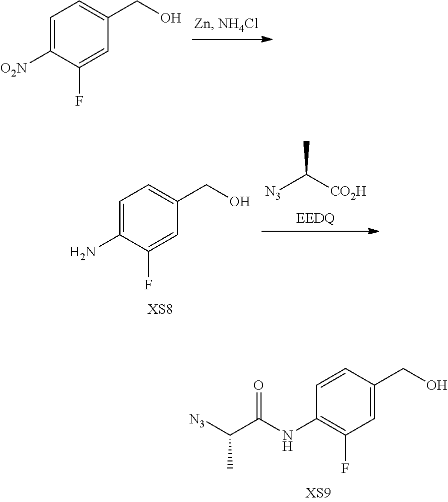

### (4-Amino-3-fluorophenyl)methanol (XS8)

To a solution of (3-fluoro-4-nitrophenyl)methanol (0.610 g, 3.56 mmol) in MeOH (8.9 mL) and THF (8.9 mL) were added zinc dust (2.33 g, 35.6 mmol) and ammonium chloride (1.91 g, 35.6 mmol). The reaction mixture was stirred at RT for 24 h, filtered over Celite and washed with MeOH (20 mL). The filtrate was concentrated and purified by flash chromatography (silica gel, 0-10% MeOH in DCM), to yield aniline XS8 (0.312 g, 62%) as an orange oil. ¹H NMR (400 MHz, CDCl₃) ppm=7.01 (dd, J=11.7, 1.9 Hz, 1H), 6.98-6.87 (m, 1H), 6.75 (dd, J=9.0, 8.1 Hz, 1H), 4.55 (s, 2H). MS (ESI⁺) calc. for C₇H₉FNO⁺ [M+H]⁺ 142.1, found 142.1.

### (S)-2-Azido-N-(2-fluoro-4-(hydroxymethyl)phenyl)propenamide (XS9)

A solution of (S)-2-azidopropanoic acid (0.180 g, 1.56 mmol) in MeOH (1.8 mL) was added to a solution of aniline XS8 (0.309 g, 2.19 mmol) in DCM (5.9 mL). The solution was cooled to 0° C. and EEDQ (0.774 g, 3.13 mmol) was added. After 15 min, the reaction mixture was allowed to reach RT and was stirred overnight. The reaction mixture was concentrated and purified by flash chromatography (silica gel, 0-100% EtOAc in DCM), to yield amide XS9 (0.416 g, 100%). ¹H NMR (400 MHz, CDCl₃) ppm=8.42-8.29 (m, 1H), 8.26 (t, J=8.2 Hz, 1H), 7.20-7.14 (m, 1H), 7.14-7.09 (m, 1H), 4.66 (d, J=5.6 Hz, 2H), 4.26 (q, J=7.1 Hz, 1H), 1.79 (t, J=5.9 Hz, 1H), 1.66 (d, J=7.0 Hz, 3H). MS (ESI⁺) calc. for C₁₀H₁₂FN₄O₂⁺ [M+H]⁺ 239.1, found 239.2.

### B: Preparation of Linker-Drug XS12

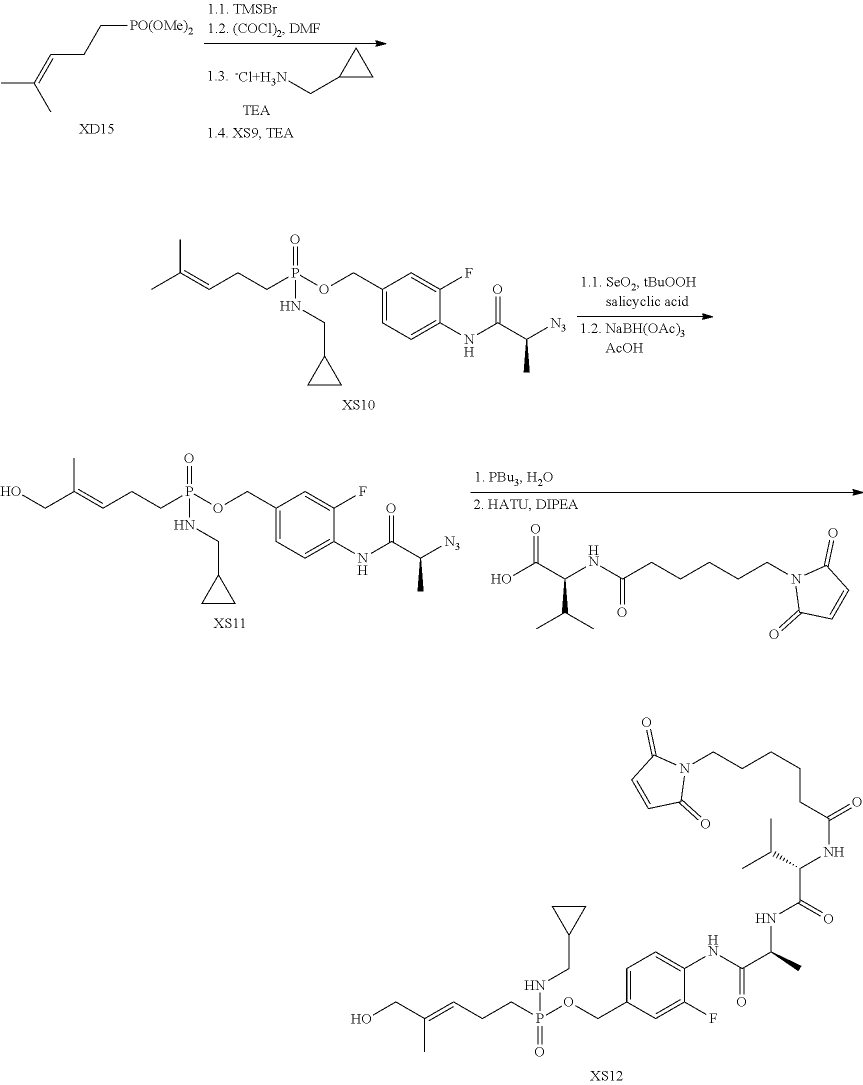

### 4-((S)-2-Azidopropanamido)-3-fluorobenzyl N-(cyclopropylmethyl)-P-(4-methylpent-3-en-1-yl)phosphonamidate (XS10)

Cyclopropylmethanamine hydrochloride (0.128 g, 1.19 mmol) was suspended in DCM (1.0 mL) and cooled to −78° C. A solution of (4-methylpent-3-en-1-yl)phosphonic dichloride (0.300 g, 1.19 mmol, prepared from phosphonic diester XD15 (0.323 g, 1.50 mmol) according to general procedure XXB) in DCM (2.0 mL) was added, followed by TEA (0.333 mL, 2.39 mmol). The reaction mixture was stirred at −78° C. for 1 h, allowed to reach RT and stirred for 2 h. The reaction mixture was cooled to −78° C. and a solution of benzylic alcohol XS9 (0.379 g, 1.43 mmol) in DCM (4.0 mL) was added, followed by TEA (0.200 mL, 1.43 mmol). The reaction mixture was allowed to reach RT and stirred for 2 h. TEA (0.100 mL, 0.717 mmol) was added, and the reaction mixture was stirred for 1 h and concentrated. The crude was partitioned between EtOAc (50 mL) and aq. HCl (15 mL, 1 M) and the water layer was backextracted with EtOAc (3×15 mL). The combined organic layer was washed with brine (10 mL), dried over Na₂SO₄ and concentrated. The crude was purified by flash chromatography (silica gel, 0-100% EtOAc in DCM), to yield both diastereomers of phosphonamidate XS10 (0.194 g, 37%) as a colorless oil. ¹H NMR (400 MHz, CDCl₃) ppm=8.39 (br s, 1H), 8.28 (t, J=8.2 Hz, 1H), 7.21-7.10 (m, 2H), 5.16-5.10 (m, 1H), 5.06-4.99 (m, 1H), 4.93-4.87 (m, 1H), 4.30-4.23 (m, 1H), 2.80-2.69 (m, 2H), 2.63-2.54 (m, 1H), 2.37-2.24 (m, 2H), 1.89-1.77 (m, 2H), 1.77-1.61 (m, 9H), 0.99-0.86 (m, 1H), 0.53-0.46 (m, 2H), 0.18-0.10 (m, 2H). MS (ESI⁺) calc. for C₂₀H₃₀FN₅O₃P⁺ [M+H]⁺ 438.2, found 438.4.

### 4-((S)-2-Azidopropanamido)-3-fluorobenzyl N-(cyclopropylmethyl)-P-((E)-5-hydroxy-4-methylpent-3-en-1-yl)phosphonamidate (XS11)

The allylic oxidation of alkene XS10 (0.183 g, 0.418 mmol) was performed according to general procedure XXC. The crude was purified by flash chromatography (silica gel, 0-5% MeOH in EtOAc), to yield both diastereomers of alcohol XS11 (80 mg, 44%). ¹H NMR (400 MHz, CDCl₃) ppm=8.38 (br s, 1H), 8.28 (t, J=8.1 Hz, 1H), 7.18 (dd, J=11.4, 1.9 Hz, 1H), 7.15-7.09 (m, 1H), 5.49-5.40 (m, 1H), 5.07-4.99 (m, 1H), 4.94-4.86 (m, 1H), 4.27 (q, J=7.0 Hz, 1H), 3.99 (br s, 2H), 2.75 (dt, J=8.5, 6.8 Hz, 2H), 2.64-2.55 (m, 1H), 2.43-2.31 (m, 2H), 1.92-1.74 (m, 2H), 1.68-1.65 (m, 6H), 1.62-1.51 (m, 1H), 0.98-0.86 (m, 1H), 0.53-0.47 (m, 2H), 0.15 (q, J=4.8 Hz, 2H). MS (ESI⁺) calc. for C₂₀H₃₀FN₅O₄P⁺ [M+H]⁺ 454.2, found 454.5.

### Linker-Drug XS12

Step 1: Azide XS11 (74 mg, 0.16 mmol) was dissolved in THF (1.5 mL)/water (0.16 mL) and the resulting solution was purged with N₂ for 15 min. Tributylphosphane (0.102 mL, 0.408 mmol) was added and the reaction mixture was stirred overnight at RT. The reaction mixture was concentrated, coevaporated with MeCN (3×1 mL) and dried in vacuo. The crude was purified by flash chromatography (silica gel, 0-20% MeOH in DCM), to yield both diastereomers of 4-((S)-2-aminopropanamido)-3-fluorobenzyl N-(cyclopropylmethyl)-P-((E)-5-hydroxy-4-methylpent-3-en-1-yl)phosphonamidate (51 mg, 73%) as an oil. MS (ESI⁺) calc. for C₂₀H₃₂FN₃O₄P⁺ [M+H]⁺ 428.2, found 428.4.

Step 2: The intermediate amine (47 mg, 0.11 mmol) and (6-(2,5-dioxo-2,5-dihydro-1H-pyrrol-1-yl)hexanoyl)-L-valine (36 mg, 0.12 mmol, prepared as described in WO2013122823) were dissolved in DMF (1.1 mL). HATU (46 mg, 0.12 mmol) and DIPEA (0.077 mL, 0.44 mmol) were added, and the reaction mixture was stirred at RT for 30 min. HATU (4.2 mg, 0.011 mmol) was added and the reaction mixture was stirred for 15 min, concentrated and coevaporated with toluene (1 mL). The residue was partitioned between EtOAc (10 mL) and sat. aq. NaHCO₃ (10 mL). The water layer was backextracted with EtOAc (2×10 mL) and the combined organic layer was washed with water (10 mL)/brine (5 mL) and brine (10 mL), dried over Na₂SO₄ and concentrated. The crude was purified by preparative RP-HPLC (MilliQ/MeCN, gradient 90:10 to 40:60, no modifier added) to give, after lyophilization, a diastereomeric mixture of XS12 (30 mg, 38%) as a white solid. ¹H NMR (400 MHz, DMSO-d₆) ppm=9.67 (s, 1H), 8.22 (d, J=7.0 Hz, 1H), 7.86 (t, J=8.3 Hz, 1H), 7.80 (d, J=8.9 Hz, 1H), 7.28 (dd, J=11.7, 1.8 Hz, 1H), 7.17 (dd, J=8.4, 1.5 Hz, 1H), 7.00 (s, 2H), 5.40-5.33 (m, 1H), 4.92-4.78 (m, 2H), 4.73 (dt, J=11.4, 6.9 Hz, 1H), 4.64 (t, J=5.5 Hz, 1H), 4.52 (quint, J=7.0 Hz, 1H), 4.22-4.15 (m, 1H), 3.79-3.73 (m, 2H), 3.37 (t, J=7.1 Hz, 2H), 2.73-2.63 (m, 2H), 2.25-2.06 (m, 4H), 2.02-1.89 (m, 1H), 1.73-1.62 (m, 2H), 1.53 (s, 3H), 1.52-1.43 (m, 4H), 1.31 (d, J=7.1 Hz, 3H), 1.23-1.13 (m, 2H), 0.94-0.86 (m, 1H), 0.84 (d, J=6.8 Hz, 3H), 0.81 (d, J=6.8 Hz, 3H), 0.42-0.35 (m, 2H), 0.17-0.11 (m, 2H). MS (ESI⁺) calc. for C₃₅H₅₂FN₅O₈P⁺ [M+H]⁺ 720.4, found 720.7.

### Example 13: Synthesis of Linker-Drug XS17

### A: Preparation of Azide XS14

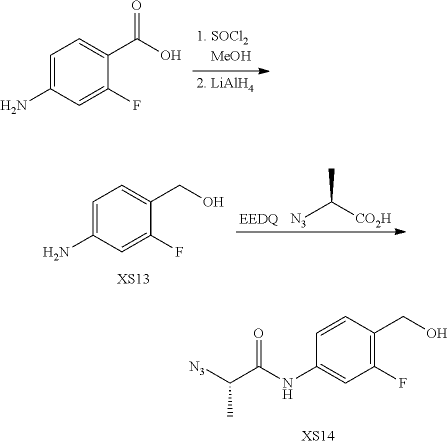

### (4-Amino-2-fluorophenyl)methanol (XS13)

Step 1: 4-Amino-2-fluorobenzoic acid (1.00 g, 6.45 mmol) was suspended in MeOH (3.2 mL) and cooled to 0° C. Thionyl chloride (0.706 mL, 9.67 mmol) was dropwise added after which the reaction mixture was refluxed for 1 h. MeOH (5.0 mL) was added and the reaction mixture was stirred at RT for 2 h. The mixture was added to sat. aq. NaHCO₃ (100 mL) and the product was extracted with EtOAc (3×75 mL). The combined organic layer was washed with brine (2×50 mL), dried over Na₂SO₄ and concentrated, coevaporated with MeOH (10 mL) and dried in vacuo, to yield methyl 4-amino-2-fluorobenzoate (1.01 g, 93%) as a brown solid. ¹H NMR (400 MHz, CDCl₃) ppm=7.76 (t, J=8.4 Hz, 1H), 6.41 (dd, J=8.6, 2.3 Hz, 1H), 6.33 (dd, J=12.9, 2.3 Hz, 1H), 4.15 (br s, 2H), 3.86 (s, 3H). MS (ESI⁺) calc. for C₈H₉FNO₂⁺[M+H]⁺ 170.1, found 170.2.

Step 2: To a 0° C. solution of the intermediate ester (0.486 g, 2.87 mmol) in THF (19 mL) was dropwise added LiAlH₄ in THF (3.59 mL, 8.62 mmol). The reaction mixture was allowed to reach RT and stirred for 3 h. The reaction mixture was cooled to 0° C. and quenched by portion wise addition of a mixture of Na₂SO₄·10 H₂O (3.5 g) and Celite (3.5 g). The mixture was filtered and the residue was washed with THF (10 mL). The filtrate was concentrated in vacuo to yield benzylic alcohol XS13 (0.367 g, 91%) as an off-white solid. ¹H NMR (400 MHz, CDCl₃) ppm=7.13 (t, J=8.3 Hz, 1H), 6.45-6.40 (m, 1H), 6.40-6.35 (m, 1H), 4.61 (d, J=5.4 Hz, 2H), 3.82-3.68 (m, 2H), 1.61 (t, J=5.9 Hz, 1H). MS (ESI⁺) calc. for C₇H₉FNO⁺ [M+H]⁺ 142.1, found 142.1.

### (S)-2-Azido-N-(3-fluoro-4-(hydroxymethyl)phenyl)propenamide (XS14)

A solution of (S)-2-azidopropanoic acid (0.210 g, 1.83 mmol) in MeOH (2.1 mL) was added to a solution of aniline XS13 (0.361 g, 2.55 mmol) in DCM (6.8 mL). The solution was cooled to 0° C. and EEDQ (0.902 g, 3.65 mmol) was added. After 15 min, the reaction mixture was allowed to reach RT and was stirred overnight. The reaction mixture was concentrated and purified by flash chromatography (silica gel, 0-100% EtOAc in DCM), to yield amide XS14 (0.902 g, quant.). ¹H NMR (400 MHz, CDCl₃) ppm=8.21-8.08 (m, 1H), 7.55 (dd, J=11.8, 2.1 Hz, 1H), 7.37 (t, J=8.3 Hz, 1H), 7.16 (dd, J=8.3, 2.2 Hz, 1H), 4.75-4.69 (m, 2H), 4.29-4.19 (m, 1H), 1.81-1.74 (m, 1H), 1.65 (d, J=7.0 Hz, 3H). MS (ESI⁺) calc. for C₁₀H₁₂FN₄O₂⁺ [M+H]⁺ 239.1, found 239.2.

### B: Preparation of Linker-Drug XS17

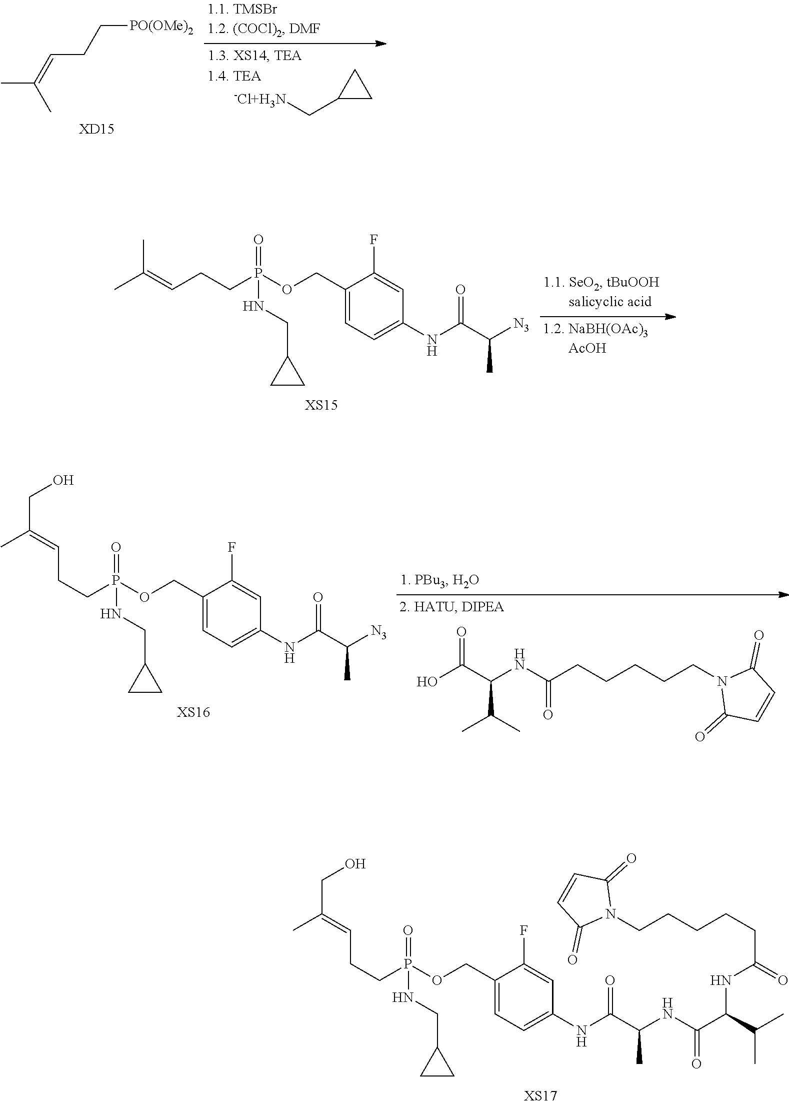

### 4-((S)-2-Azidopropanamido)-2-fluorobenzyl N-(cyclopropylmethyl)-P-(4-methylpent-3-en-1-yl)phosphonamidate (XS15)

Cyclopropylmethanamine hydrochloride (0.128 g, 1.19 mmol) was suspended in DCM (1.0 mL) and cooled to −78° C. A solution of (4-methylpent-3-en-1-yl)phosphonic dichloride (0.300 g, 1.19 mmol, prepared from phosphonic diester XD15 (0.323 g, 1.50 mmol) according to general procedure XXB) in DCM (2.0 mL) was added, followed by TEA (0.333 mL, 2.39 mmol). The reaction mixture was stirred at −78° C. for 1 h, allowed to reach RT and stirred for 2 h. The reaction mixture was cooled to −78° C. and a solution of benzylic alcohol XS14 (0.379 g, 1.43 mmol) in DCM (4.0 mL) was added, followed by TEA (0.200 mL, 1.43 mmol). The reaction mixture was allowed to reach RT and stirred for 2 h. TEA (0.030 mL, 0.215 mmol) was added, and the reaction mixture was stirred for 1 h and concentrated. The remainder was partitioned between EtOAc (50 mL) and aq. HCl (15 mL, 1 M) and the water layer was backextracted with EtOAc (3×15 mL). The combined organic layer was washed with brine (10 mL), dried over Na₂SO₄ and concentrated. The crude was purified by flash chromatography (silica gel, 0-100% EtOAc in DCM), to yield a diastereomeric mixture of phosphonamidate XS15 (0.294 g, 56%) as a colorless oil. ¹H NMR (400 MHz, CDCl₃) ppm=8.27 (s, 1H), 7.60 (dd, J=11.8, 2.1 Hz, 1H), 7.40 (t, J=8.3 Hz, 1H), 7.15 (dd, J=8.3, 2.1 Hz, 1H), 5.14-5.09 (m, 1H), 5.09-4.96 (m, 2H), 4.27-4.20 (m, 1H), 2.83-2.69 (m, 2H), 2.64-2.52 (m, 1H), 2.35-2.22 (m, 2H), 1.88-1.76 (m, 2H), 1.75-1.62 (m, 9H), 1.00-0.88 (m, 1H), 0.54-0.45 (m, 2H), 0.18-0.12 (m, 2H). MS (ESI⁺) calc. for C₂₀H₃₀FN₅O₃P⁺ [M+H]⁺ 438.2 found 438.4.

### 4-((S)-2-Azidopropanamido)-2-fluorobenzyl N-(cyclopropylmethyl)-P-((E)-5-hydroxy-4-methylpent-3-en-1-yl)phosphonamidate (XS16)

The allylic oxidation of alkene XS15 (0.288 g, 0.658 mmol) was performed according to general procedure XXC. The crude was purified by flash chromatography (silica gel, 0-5% MeOH in EtOAc), to yield XS16 (64 mg, 32%) as a diastereomeric mixture. ¹H NMR (400 MHz, CDCl₃) ppm=8.25 (s, 1H), 7.60 (dd, J=11.7, 2.1 Hz, 1H), 7.39 (t, J=8.3 Hz, 1H), 7.15 (dd, J=8.3, 2.1 Hz, 1H), 5.46-5.40 (m, 1H), 5.08-4.97 (m, 2H), 4.24 (q, J=7.0 Hz, 1H), 3.98 (s, 2H), 2.81-2.71 (m, 2H), 2.68-2.52 (m, 1H), 2.41-2.30 (m, 2H), 1.91-1.71 (m, 2H), 1.68-1.63 (m, 6H), 1.59-1.52 (m, 1H), 1.01-0.86 (m, 1H), 0.54-0.45 (m, 2H), 0.18-0.13 (m, 2H). MS (ESI⁺) calc. for C₂₀H₃₀FN₅O₄P⁺ [M+H]⁺ 454.2, found 454.5.

### Linker-Drug XS17

Step 1: Azide XS16 (61 mg, 0.14 mmol) was dissolved in THF (1.2 mL)/water (0.14 mL) and the resulting solution was purged with N₂ for 15 min. Tributylphosphane (84 μL, 0.336 mmol) was added and the reaction mixture was stirred overnight at RT. The reaction mixture was concentrated, coevaporated with MeCN (3×1 mL) and dried in vacuo. The crude was purified by flash chromatography (silica gel, 0-20% MeOH in DCM), to yield both diastereomers of 4-((S)-2-aminopropanamido)-2-fluorobenzyl N-(cyclopropylmethyl)-P-((E)-5-hydroxy-4-methylpent-3-en-1-yl)phosphonamidate (44 mg, 77%) as an oil. MS (ESI⁺) calc. for C₂₀H₃₂FN₃O₄P⁺ [M+H]⁺ 428.2, found 428.6.

Step 2: The intermediate amine (44 mg, 0.10 mmol) and (6-(2,5-dioxo-2,5-dihydro-1H-pyrrol-1-yl)hexanoyl)-L-valine (34 mg, 0.11 mmol, prepared as described in WO2013122823) were dissolved in DMF (1.0 mL). HATU (43 mg, 0.11 mmol) and DIPEA (72 μL, 0.41 mmol) were added, and the reaction mixture was stirred at RT for 90 min, concentrated and coevaporated with toluene (1 mL). The remainder was partitioned between EtOAc (10 mL) and sat. NaHCO₃ (10 mL). The water layer was backextracted with EtOAc (2×10 mL) and the combined organic layer was washed with water (10 mL) and brine (10 mL), dried over Na₂SO₄ and concentrated. The crude was purified by preparative RP-HPLC (MilliQ/MeCN, gradient 90:10 to 40:60, no modifier added). Evaporation of MeCN and subsequent lyophilization afforded XS17 (32 mg, 43%) as a diastereomeric mixture. ¹H NMR (400 MHz, DMSO-d₆) ppm=10.13 (s, 1H), 8.19 (d, J=6.8 Hz, 1H), 7.80 (d, J=8.6 Hz, 1H), 7.59 (dd, J=12.6, 1.9 Hz, 1H), 7.40 (t, J=8.4 Hz, 1H), 7.31 (dd, J=8.4, 2.0 Hz, 1H), 6.99 (s, 2H), 5.38-5.31 (m, 1H), 4.91-4.79 (m, 2H), 4.70 (dt, J=11.4, 6.8 Hz, 1H), 4.66-4.60 (m, 1H), 4.36 (quint, J=7.0 Hz, 1H), 4.16 (dd, J=8.5, 6.9 Hz, 1H), 3.75 (d, J=5.6 Hz, 2H), 3.36 (t, J=7.1 Hz, 2H), 2.73-2.59 (m, 2H), 2.22-2.07 (m, 4H), 1.96 (dq, J=13.6, 6.8 Hz, 1H), 1.70-1.58 (m, 2H), 1.52 (s, 3H), 1.51-1.42 (m, 4H), 1.30 (d, J=7.1 Hz, 3H), 1.23-1.13 (m, 2H), 0.90-0.88 (m, 1H), 0.86 (d, J=6.6 Hz, 3H), 0.82 (d, J=6.8 Hz, 3H), 0.41-0.34 (m, 2H), 0.18-0.09 (m, 2H). MS (ESI⁺) calc. for C₃₅H₅₂FN₅O₈P⁺ [M+H]⁺ 720.4, found 720.7.

### Example 14: Synthesis of Linker-Drug XS22

### A: Preparation of Azide XS19

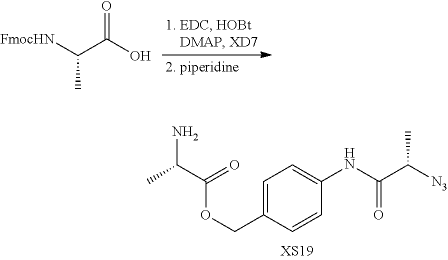

### 4-((S)-2-Azidopropanamido)benzyl L-alaninate (XS19)

Step 1: Fmoc-Ala-OH (0.386 g, 1.240 mmol) was dissolved in DCM (12 mL) and cooled to 0° C. DMAP (12 mg, 0.099 mmol) was added, followed by EDC (0.291 g, 1.52 mmol) and HOBt (0.155 g, 1.01 mmol). After stirring for 15 min, Alcohol XD7 (0.300 g, 1.36 mmol) was added and the reaction mixture was allowed to reach RT and stirred overnight. The reaction mixture was added to water (15 mL) and the water layer was extracted with DCM (2×15 mL). The combined organic layer was washed with brine (15 mL), dried over Na₂SO₄ and concentrated. The crude was purified by flash chromatography (silica gel, 0-80% EtOAc in heptane) to yield 4-((S)-2-azidopropanamido)benzyl (((9H-fluoren-9-yl)methoxy)carbonyl)-L-alaninate (0.406 g, 64%) as a white solid. MS (ESI⁺) calc. for C₂₈H₂₈N₅O₅⁺ [M+H]⁺ 514.2, found 514.5.

Step 2: The intermediate ester (0.404 g, 0.786 mmol) was dissolved in DMF (5.3 mL) and piperidine (0.389 mL, 3.93 mmol) was added. The reaction mixture was stirred for 20 min, then concentrated and coevaporated with toluene (2×5 mL). The crude was purified by flash chromatography (silica gel, 0-15% MeOH in DCM) to yield amine XS19 (0.219 g, 96%) as a colorless oil. ¹H NMR (400 MHz, DMSO-d₆) ppm=10.22 (s, 1H), 7.60 (d, J=8.6 Hz, 2H), 7.34 (d, J=8.5 Hz, 2H), 5.05 (d, J=2.3 Hz, 2H), 4.03 (q, J=6.9 Hz, 1H), 3.44 (q, J=6.9 Hz, 1H), 1.79 (br s, 2H), 1.45 (d, J=6.9 Hz, 3H), 1.17 (d, J=7.0 Hz, 3H). MS (ESI⁺) calc. for C₁₃H₁₈N₅O₃⁺[M+H]⁺ 292.1, found 292.3.

### B: Preparation of Linker-Drug XS22

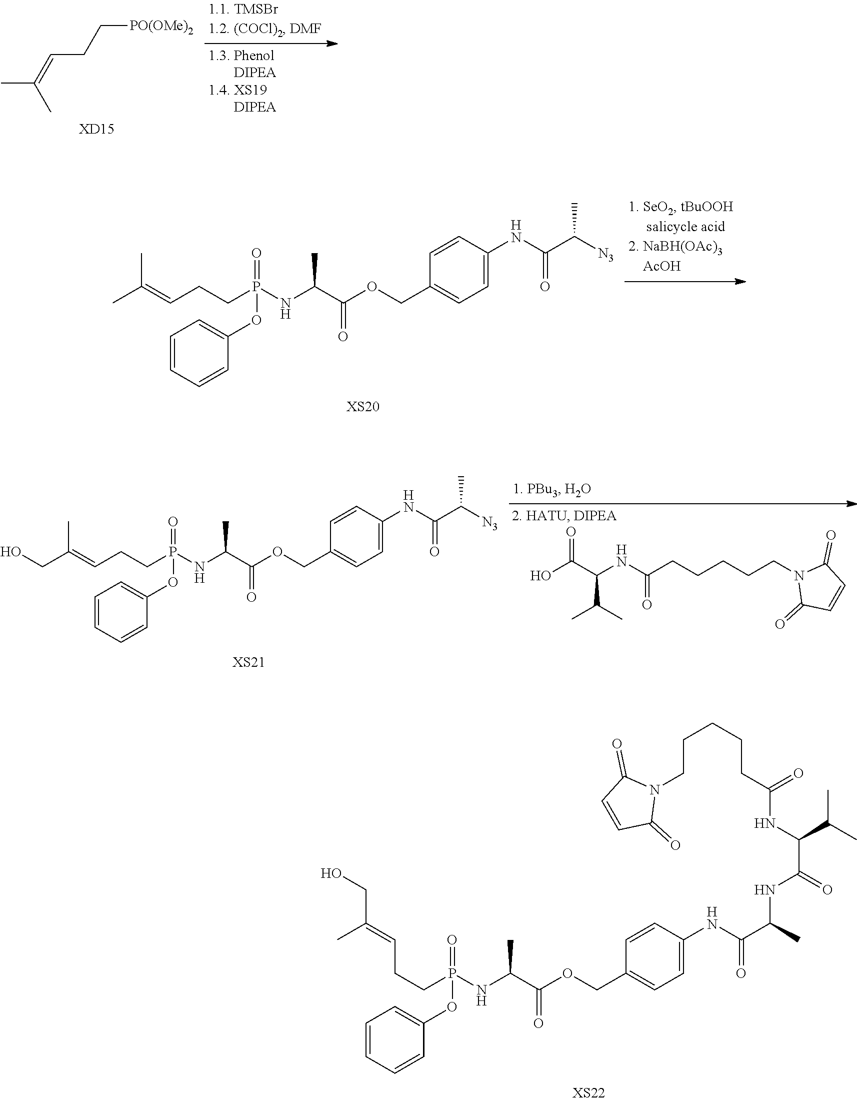

### 4-((S)-2-Azidopropanamido)benzyl ((4-methylpent-3-en-1-yl)(phenoxy)phosphoryl)-L-alaninate (XS20)

To (4-methylpent-3-en-1-yl)phosphonic dichloride (0.176 g, 0.700 mmol, prepared from phosphonic diester XD15 (0.189 g, 0.875 mmol) according to general procedure XXB) in toluene (4.5 mL) was dropwise added a solution of phenol (66 mg, 0.70 mmol) and DIPEA (0.122 mL, 0.700 mmol) in toluene (4.5 mL) at −78° C. The reaction mixture was allowed to reach RT and stirred for 2 h and 30 min. Amine XS19 (0.214 g, 0.735 mmol) and DIPEA (0.128 mL, 0.735 mmol) were added at −78° C. and the reaction mixture was allowed to reach RT and was stirred for 2 h. DIPEA (0.128 mL, 0.735 mmol) was added and the reaction mixture was stirred for 90 min and concentrated. The crude was purified by flash chromatography (silica gel, 0-100% EtOAc in DCM), to yield a diastereomeric mixture of phosphonamidate XS20 (0.100 g, 28%) as a pale yellow oil. ¹H NMR (400 MHz, CDCl₃) ppm=8.13 (br s, 1H), 7.57-7.51 (m, 2H), 7.37-7.27 (m, 4H), 7.22-7.08 (m, 3H), 5.17-5.08 (m, 1H), 5.07-5.01 (m, 2H), 4.24 (qd, J=7.0, 1.6 Hz, 1H), 4.16-4.04 (m, 1H), 3.29 (t, J=10.1 Hz, 0.5H), 3.18 (td, J=10.4, 3.4 Hz, 0.5H), 2.40-2.30 (m, 2H), 1.96-1.81 (m, 2H), 1.77-1.73 (m, 3H), 1.69 (br s, 3H), 1.65 (d, J=7.0 Hz, 3H), 1.25 (q, J=7.0 Hz, 3H. MS (ESI⁺) calc. for C₂₅H₃₃N₅O₅P⁺ [M+H]⁺ 514.2, found 514.5.

### 4-((S)-2-Azidopropanamido)benzyl (((E)-5-hydroxy-4-methylpent-3-en-1-yl)(phenoxy)phosphoryl)-L-alaninate (XS21)

The allylic oxidation of alkene XS20 (0.100 g, 0.195 mmol) was performed according to general procedure XXC. The crude was purified by flash chromatography (silica gel, 0-100% EtOAc in DCM), to yield both diastereomers of alcohol XS21 (27 mg, 26%). ¹H NMR (400 MHz, CDCl₃) ppm=8.48-8.34 (m, 1H), 7.58-7.51 (m, 2H), 7.32-7.23 (m, 4H), 7.22-7.16 (m, 2H), 7.15-7.09 (m, 1H), 5.44-5.35 (m, 1H), 5.12-5.01 (m, 2H), 4.22-4.16 (m, 1H), 4.16-3.99 (m, 1H), 3.98 (s, 2H), 3.45 (t, J=10.2 Hz, 0.5H), 3.30 (dd, J=11.4, 10.3 Hz, 0.5H), 2.49-2.32 (m, 2H), 2.00-1.80 (m, 2H), 1.65 (d, J=6.0 Hz, 3H), 1.62 (d, J=7.0 Hz, 3H), 1.29 (d, J=7.1 Hz, 1.4H), 1.21 (d, J=7.1 Hz, 1.6H). MS (ESI⁺) calc. for C₂₅H₃₃N₅O₆P⁺ [M+H]⁺ 530.2, found 530.5.

### Linker-Drug XS22

Step 1: Azide XS21 (27 mg, 0.050 mmol) was dissolved in THF (0.45 mL)/water (0.050 mL) and the resulting solution was purged with N₂ for 15 min. Tributylphosphane (0.032 mL, 0.126 mmol) was added and the reaction mixture was stirred overnight at RT. The reaction mixture was concentrated, coevaporated with MeCN (3×1 mL) and dried in vacuo. The crude was purified by flash chromatography (silica gel, 0-20% MeOH in DCM), to yield both diastereomers of 4-((S)-2-aminopropanamido)benzyl (((E)-5-hydroxy-4-methylpent-3-en-1-yl)(phenoxy)phosphoryl)-L-alaninate (15 mg, 58%) as a colorless oil. MS (ESI⁺) calc. for C₂₅H₃₅N₃O₆P⁺ [M+H]⁺ 504.2, found 504.6.

Step 2: The intermediate amine (15 mg, 0.029 mmol) and (6-(2,5-dioxo-2,5-dihydro-1H-pyrrol-1-yl)hexanoyl)-L-valine (9.9 mg, 0.032 mmol, prepared as described in WO2013122823) were dissolved in DMF (0.29 mL). HATU (13 mg, 0.035 mmol) and DIPEA (0.020 mL, 0.12 mmol) were added, and the reaction mixture was stirred at RT for 75 min, concentrated and coevaporated with toluene (1 mL). The remainder was partitioned between EtOAc (10 mL) and sat. NaHCO₃ (10 mL). The water layer was backextracted with EtOAc (2×10 mL) and the combined organic layer was washed with water (10 mL) and brine (10 mL), dried over Na₂SO₄ and concentrated. The crude was purified by preparative RP-HPLC (MilliQ/MeCN, gradient 80:20 to 30:70, no modifier added) to give, after lyophilization, a diastereomeric mixture of linker-drug XS22 (10 mg, 44%) as a white solid. ¹H NMR (400 MHz, DMSO-d₆) ppm=9.94 (s, 1H), 8.14 (d, J=6.9 Hz, 1H), 7.79 (d, J=8.6 Hz, 1H), 7.57 (d, J=8.5 Hz, 2H), 7.37-7.22 (m, 4H), 7.17-7.10 (m, 3H), 6.99 (s, 2H), 5.58 (dd, J=12.5, 10.4 Hz, 0.5H), 5.48 (dd, J=13.2, 10.1 Hz, 0.5H), 5.35 (t, J=7.1 Hz, 1H), 5.07-4.94 (m, 2H), 4.65 (td, J=5.6, 0.8 Hz, 1H), 4.38 (quint, J=7.0 Hz, 1H), 4.17 (dd, J=8.5, 6.9 Hz, 1H), 4.01-3.87 (m, 1H), 3.77 (d, J=5.5 Hz, 2H), 3.36 (t, J=7.1 Hz, 2H), 2.31-2.20 (m, 2H), 2.20-2.07 (m, 2H), 2.03-1.91 (m, 1H), 1.87-1.74 (m, 2H), 1.55-1.52 (m, 3H), 1.52-1.43 (m, 4H), 1.30 (d, J=7.0 Hz, 3H), 1.21-1.06 (m, 5H), 0.86 (d, J=6.8 Hz, 3H), 0.82 (d, J=6.8 Hz, 3H). MS (ESI⁺) calc. for C₄₀H₅₅N₅O₁₀P⁺ [M+H]⁺ 796.4, found 796.6.

### Example 15: Synthesis of Linker-Drug XD24

### A: Preparation of Carbamate XD26

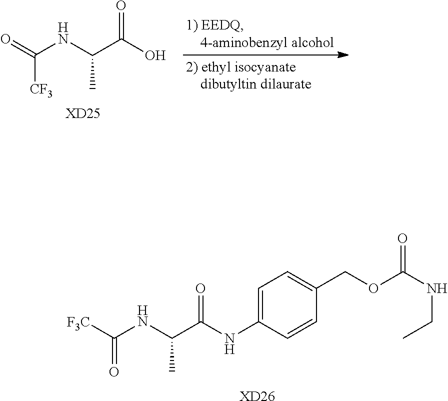

### (S)-4-(2-(2,2,2-Trifluoroacetamido)propanamido)benzyl ethylcarbamate (XD26)

Step 1: A solution of amide XD25 (11.4 g, 61.6 mmol, prepared as described by Z. P. Tachrim et al. Molecules, 2007, 22, 1748) in DCM (275 ml) and MeOH (175 ml) was cooled to 0° C. and EEDQ (30.5 g, 123 mmol) and (4-aminophenyl)methanol (10.6 g, 86.0 mmol) were added. After 1 h, the orange solution was allowed to warm to RT and stirred overnight. The reaction was concentrated and the crude was stirred in ether (500 mL) at RT for 1 h. The mixture was filtered and the solid was washed with ether to give a first crop of (S)—N-(4-(hydroxymethyl)phenyl)-2-(2,2,2-trifluoroacetamido)propanamide (10.8 g, 61%) as a white solid. The filtrate was concentrated and the crude was taken up in EtOAc (150 mL). The solution was washed with aq HCl (2 M, 90 mL) and the water layer was backextracted with EtOAc (3×125 mL). The combined org. layers were washed with brine (10 mL), dried over Na₂SO₄, filtered and concentrated. The crude was suspended in a minimal volume of DCM, stirred for 30 min, and the thick cake was then filtered. The solid was collected and dried under vacuum to give a second crop of (S)—N-(4-(hydroxymethyl)phenyl)-2-(2,2,2-trifluoroacetamido)propanamide (4.66 g, 26% yield) as a cream solid. MS (ESI⁺) calcd. for C₁₂H₁₄F₃N₂O₃ [M+H]⁺ 291.10, found 291.24.

Step 2: To a solution of (S)—N-(4-(hydroxymethyl)phenyl)-2-(2,2,2-trifluoroacetamido)propanamide (3.24 g, 11.2 mmol) in THF (80 ml) was added at 0° C. dibutyltin dilaurate (1.66 ml, 2.79 mmol) and ethyl isocyanate (1.33 ml, 16.7 mmol). The cooling bath was removed and the mixture was stirred at RT for 3 h. The reaction mixture was concentrated on silica gel and purified by flash chromatography (stannane impurities were first removed with a 0-80% gradient of ether in heptane, followed by elution of the product with a 0-100% gradient of EtOAc in heptane), to give carbamate XD26 (3.62 g, 78% 2 steps) as a white solid. ¹H NMR (400 MHz, DMSO-d₆) ppm=10.18 (s, 1H), 9.71 (s, 1H), 7.58 (d, J=8.6 Hz, 2H), 7.30 (d, J=8.5 Hz, 2H), 7.16 (br t, J=5.4 Hz, 1H), 4.95 (s, 2H), 4.62-4.42 (m, 1H), 3.02 (qd, J=7.2, 5.6 Hz, 2H), 1.42 (d, J=7.3 Hz, 3H), 1.01 (t, J=7.3 Hz, 3H). MS (ESI⁺) calcd. for C₁₅H₁₈F₃N₃NaO₄⁺ [M+Na]⁺384.1 found 384.3.

### B: Preparation of Linker-Drug XD24

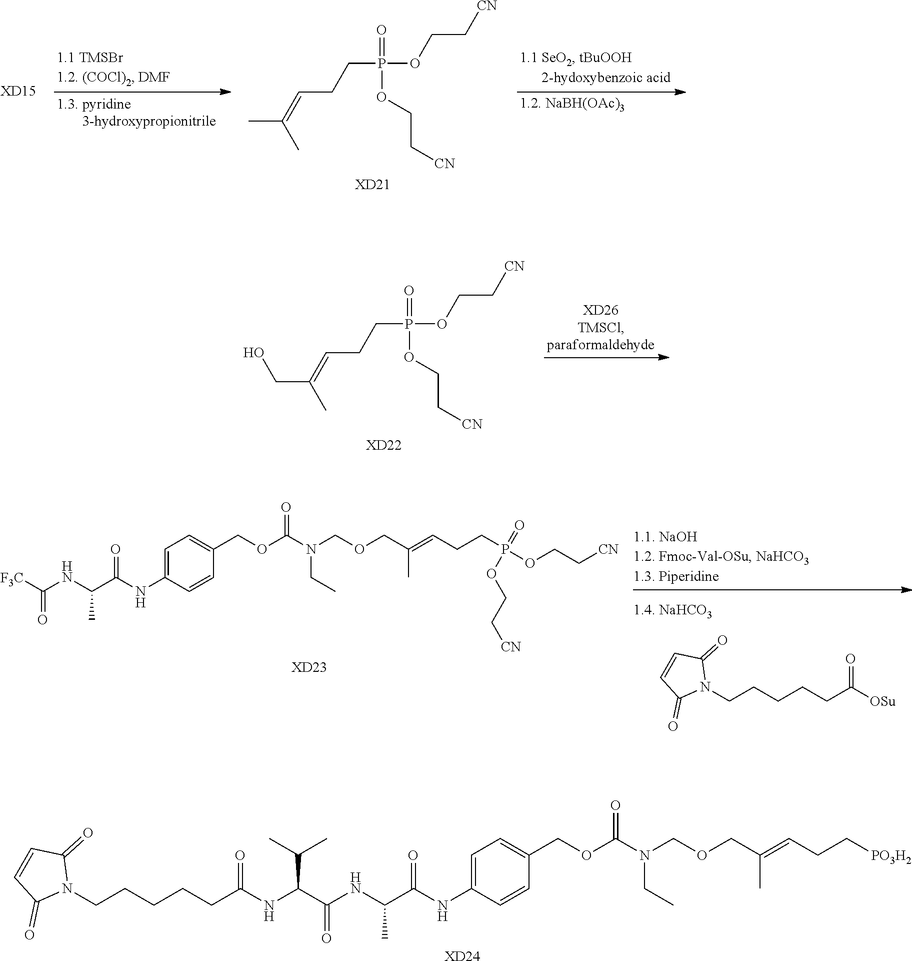

### Bis(2-cyanoethyl) (4-methylpent-3-en-1-yl)phosphonate (XD21)

A solution of (4-methylpent-3-en-1-yl)phosphonic dichloride (1.04 g, 5.20 mmol, prepared from phosphonic diester XD15 according to general procedure XXB) in DCM (3.3 ml) was added dropwise to a cooled (−78° C.) solution of 3-hydroxypropanenitrile (0.746 ml, 10.9 mmol) and pyridine (0.883 ml, 10.9 mmol) in DCM (17 ml). After 30 min, the reaction was warmed to RT. More 3-hydroxypropanenitrile (0.267 ml, 3.90 mmol) and pyridine (0.315 ml, 3.90 mmol) was added after 45 min at RT, and stirring was continued for 3 h. The reaction was poured into a mixture of EtOAc (100 mL) and aq. HCl (1 M, 20 mL). The layers were separated and the water layer was extracted with EtOAc (3×30 mL). The combined org. layers were washed with brine (20 mL), dried over Na₂SO₄, filtered and concentrated. Purification flash chromatography (0-100% EtOAc in DCM) afforded dialkyl phosphonate XD21 (986 mg, 70%) as a pale yellow oil. ¹H NMR (400 MHz, CDCl₃) ppm=5.12 (t, J=7.1 Hz, 1H), 4.37-4.20 (m, 4H), 2.77 (t, J=6.1 Hz, 4H), 2.33 (dq, J=14.4, 7.4 Hz, 2H), 1.96-1.83 (m, 2H), 1.70 (s, 3H), 1.64 (s, 3H). MS (ESI⁺) calcd. for C₁₂H₂₀N₂O₃P⁺ [M+H]⁺ 271.1, found 271.2.

### Bis(2-cyanoethyl) (E)-(5-hydroxy-4-methylpent-3-en-1-yl)phosphonate (XD22)

The allylic oxidation of alkene XD21 (0.986 g, 3.65 mmol) was performed according to general procedure XXC. The crude was purified by flash chromatography (silica gel, 0-6% MeOH in DCM), to yield alcohol XD22 (0.586 g, 56%). ¹H NMR (400 MHz, CDCl₃) ppm=5.44 (td, J=7.1, 1.3 Hz, 1H), 4.34-4.24 (m, 4H), 4.01 (s, 2H), 2.77 (t, J=6.0 Hz, 4H), 2.41 (dq, J=15.5, 7.6 Hz, 2H), 2.01-1.87 (m, 2H), 1.69 (s, 3H). MS (ESI⁺) calcd. for C₁₂H₁₉N₂NaO₄P⁺ [M+Na]⁺309.1 found 309.2.

### (S)-4-(2-(2,2,2-Trifluoroacetamido)propanamido)benzyl (E)-(((5-(bis(2-cyanoethoxy)phos-phoryl)-2-methylpent-2-en-1-yl)oxy)methyl)(ethyl)carbamate (XD23)

Alcohol XD22 (100 mg, 0.349 mmol) was reacted with carbamate XD26 according to general procedure XXA with the following modification; 2,6-lutidine was used instead of DIPEA. Purification of the crude by flash chromatography (silica gel, 0-4% MeOH in DCM) afforded carbamate XD23 (217 mg, 94%) as a colorless oil. ¹H NMR (400 MHz, DMSO-d₆, recorded at 330 K) ppm=10.11 (s, 1H), 9.60 (br d, J=6.9 Hz, 1H), 7.59 (d, J=8.5 Hz, 2H), 7.33 (d, J=8.5 Hz, 2H), 5.40 (br s, 1H), 5.07 (s, 2H), 4.70 (s, 2H), 4.51 (quint, J=7.1 Hz, 1H), 4.23-4.10 (m, 4H), 3.78 (br s, 2H), 3.30 (q, J=7.0 Hz, 2H), 2.91 (t, J=5.9 Hz, 4H), 2.26 (dq, J=14.1, 7.2 Hz, 2H), 1.95-1.81 (m, 2H), 1.58 (br s, 3H), 1.43 (d, J=7.1 Hz, 3H), 1.09 (t, J=7.1 Hz, 3H). MS (ESI⁺) calcd. for C₂₈H₃₈F₃N₅O₈P⁺ [M+H]⁺ 660.2, found 660.6.

### Linker-Drug XD24

Step 1: Amide XD23 (44.5 mg, 0.067 mmol) was dissolved in MeOH (1.1 ml). Water (0.135 ml) was added and the mixture was cooled to 0° C. NaOH (2 M in water, 0.135 ml, 0.270 mmol) was added and after 2 min the ice bath was removed and stirring was continued at RT. More NaOH (2 M in water, 0.135 ml, 0.270 mmol) was added after 2 h, and the reaction was continued at RT for a total reaction time of 9 h. The reaction was cooled to 0° C. and aq. HCl (1 M, 0.304 mL) was added. The solution was then carried forward without any further purification.

Step 2: The solution of step 1 was treated with aq. AcOH (1 M, 0.170 mL) and the mixture was concentrated. The crude was taken up in water (0.4 mL) and solid NaHCO₃ (16.9 mg, 0.201 mmol) was added followed by the addition of Fmoc-Val-OSu (29.4 mg, 0.067 mmol) in THF (0.4 mL) at RT. The mixture was stirred for 24 h at RT and was then concentrated.

Step 3: The crude, prepared in step 3, was suspended in DMF (2.0 mL) and piperidine (0.8 mL) was added at RT. After stirring for 1 h, the reaction was concentrated and redissolved in DMF (2.0 mL). Et₃N (0.8 mL) was added and the mixture was stirred for 5 min to give a fine suspension. The mixture was concentrated and this process was repeated once more to ensure full removal of piperidine residue. The white solid was suspended in ether (5 mL) and the mixture was stirred for 30 min. The supernatant was carefully removed and this process was repeated until UPLC-MS analysis showed no more Fmoc residue in the supernatant. The solid was dried under vacuum.

Step 4: The solid was dissolved in water (0.4 mL), and solid NaHCO₃ (17.0 mg, 0.200 mmol) was added, followed by 2,5-dioxopyrrolidin-1-yl 6-(2,5-dioxo-2,5-dihydro-1H-pyrrol-1-yl)hexanoate (20.8 mg, 0.068 mmol) in THF (0.4 mL) at RT. After stirring for 3 h, most of the THF was removed by brief rotary evaporation at RT. The aqueous solution was then diluted with 10% MeCN in MilliQ (8 mL) and the clear solution was purified by preparative RP-HPLC (water×0.025% NH₄OH/MeCN, gradient 10-40%). Note that the product was collected in test tubes that were prefilled with aq. AcOH (1 M, 0.5 mL) to ensure direct acidification of the basic eluent. Product fractions were immediately frozen and subsequently lyophilized to give the title compound XD24 (11.5 mg) as a white solid. ¹H NMR (400 MHz, D₂O) ppm=7.49-7.39 (m, 2H), 7.39-7.30 (m, 2H), 6.73 (s, 2H), 5.53-5.30 (m, 1H), 5.09 (br s, 2H), 4.70 (s, 2H; obscured by the residual solvent peak of D₂O. Correlation observed in HSQC), 4.39 (q, J=6.8 Hz, 1H), 4.03 (br d, J=7.4 Hz, 1H), 3.87-3.72 (m, 2H), 3.36 (t, J=6.8 Hz, 2H), 3.34-3.26 (m, 2H), 2.24 (t, J=6.8 Hz, 2H), 2.20-2.08 (m, J=4.1 Hz, 2H), 2.01 (dq, J=13.6, 6.8 Hz, 1H), 1.64-1.37 (m, 12H), 1.24-1.11 (m, 2H), 1.06 (t, J=7.1 Hz, 3H), 0.96-0.81 (m, 6H). MS (ESI⁻) calcd. for C₃₅H₅₁N₅O₁₁P⁻ [M−H]⁻ 748.3, found 748.8.

### Example 17: Synthesis of Linker-Drug XD44, XD45 and XD46

### A: Preparation of bis((9H-fluoren-9-yl)methyl) phosphorochloridate (XD34)

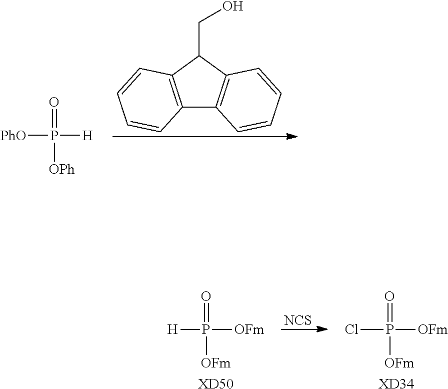

### bis((9H-Fluoren-9-yl)methyl) phosphonate (XD50)

(9H-Fluoren-9-yl)methanol (4.55 g, 23.2 mmol) was added to a solution of diphenyl phosphite (2.13 ml, 10.6 mmol) in dry pyridine (20 ml) at RT under N₂, and the mixture was stirred for 2 h. The reaction was concentrated and taken up in EtOAc (250 mL). The organic phase was washed with aq. HCl (2×, 1 M) and brine, dried over Na₂SO₄, filtered and concentrated on silica gel. Purification by flash chromatography (silica gel, 0-85% EtOAc/DCM (1:4) in heptane) afforded H-phosphonate XD50 (3.46 g, 75%) as a colorless wax. ¹H NMR (400 MHz, CDCl₃) ppm=7.76-7.66 (m, 4H), 7.58-7.45 (m, 4H), 7.42-7.31 (m, 4H), 7.31-7.22 (m, 4H), 7.19-7.12 (m, 1H), 6.68 (d, J=705.8 Hz, 1H), 4.34-4.21 (m, 4H), 4.15-4.08 (m, 2H). MS (ESI⁺) calcd. for C₂₈H₂₄O₃P⁺ [M+H]⁺ 439.2 found 439.3.

### bis((9H-Fluoren-9-yl)methyl) phosphorochloridate (XD34)

H-phosphonate XD50 (8.57 g, 19.6 mmol) was dissolved in toluene (98 ml) and the overhead space was purged with N₂. NCS (3.13 g, 23.5 mmol) was added at RT and the reaction mixture was then stirred at 40° C. for 2 h. After cooling to RT, the reaction mixture was filtered and concentrated. The residue was coevaporated with MeCN (10 mL) to afford a white solid. The solid was dissolved in MeCN (25 mL) using gentle heating with a heat gun to dissolve all the solid. The solution was gradually cooled down to −30° C. at which point a white solid started to precipitate. The flask was stored at −30° C. overnight and was then allowed to warm to RT before filtration. Ice-cold MeCN (10 mL) was used to wash the solid to give chloride XD34 (8.03 g, 87% yield) as a white solid. ¹H NMR (400 MHz, CDCl₃) ppm=7.76-7.71 (m, 4H), 7.56-7.48 (m, 4H), 7.43-7.36 (m, 4H), 7.33-7.25 (m, 4H), 4.46 (dt, J=9.7, 7.1 Hz, 2H), 4.36-4.28 (m, 2H), 4.25-4.19 (m, 2H). MS (ESI⁺) calcd. for C₂₈H₂₄ClO₄P⁺ [M+NH₄]⁺ 490.1 found 490.3.

### B: Preparation of Phosphonate XD37

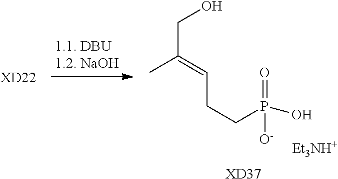

### Triethylammonium (E)-hydrogen (5-hydroxy-4-methylpent-3-en-1-yl)phosphonate (XD37)

DBU (0.401 ml, 2.66 mmol) was added to a solution of XD22 (305 mg, 1.07 mmol) in THF (10 ml) at 0° C. The cooling bath was removed after 2 min, and the reaction was stirred at RT for 4 h to give exclusively mono-deprotected product. The mixture was diluted with ether (30 mL) and after stirring for 15 min the emulsion was allowed to settle (15 min). The supernatant was decanted and the residue was taken up in MeOH (10 mL) and water (1.25 mL). NaOH (2 M in water, 3.20 mL, 6.40 mmol) was added at 0° C. and the mixture was stirred at this temperature for 30 min. The ice bath was removed and the reaction was continued at RT for 3 h and 30 min. The reaction mixture was then loaded on a DOWEX 50WX8-200 hydrogen form column (5 g, pre-washed with methanol until eluent is neutral and colorless) The product was eluted with methanol. Product fractions were pooled and Et₃N (0.148 mL, 1.07 mmol) was added. Methanol was removed by rotary evaporation and the aq. phase was diluted with MeCN and subsequently lyophilized to give XD37 (259 mg, 98%) as a colorless oil. ¹H NMR (400 MHz, CD₃OD) ppm=5.51-5.42 (m, 1H), 3.91 (s, 2H), 3.19 (q, J=7.3 Hz, 6H), 2.40-2.27 (m, 2H), 1.67 (s, 3H), 1.69-1.58 (m, 2H), 1.31 (t, J=7.3 Hz, 9H). MS (ESI⁻) calcd. for C₆H₁₂O4P⁻ [M−H]⁻ 179.1 found 179.1.

### C: Preparation of Phosphate XD38

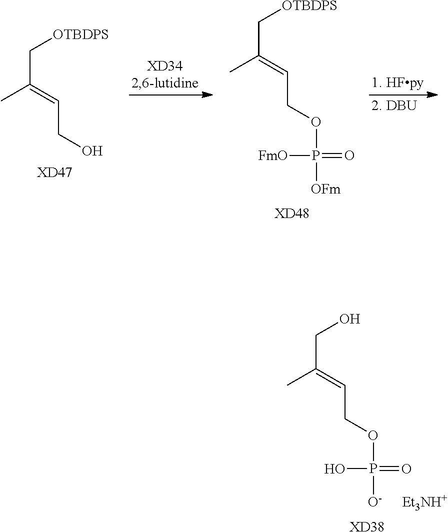

### (E)-bis((9H-Fluoren-9-yl)methyl) (4-((tert-butyldiphenylsilyl)oxy)-3-methylbut-2-en-1-yl) phosphate (XD48)

To a solution of alcohol XD47 (1.35 g, 3.97 mmol, synthesized as described by Serra, S. Tetrahedron: Asymmetry, 2014, 25, 1561-1572), 5-(ethylthio)-1H-tetrazole (0.043 g, 0.331 mmol) and 2,6-lutidine (1.54 ml, 13.2 mmol) in MeCN (5 mL) was added XD34 (1.45 g, 3.31 mmol) in MeCN/DCM (1:1, 5 mL) at 0° C. The cooling bath was removed and the reaction was stirred at RT for 75 min. The mixture was concentrated and the crude was taken up in EtOAc (100 mL). The suspension was washed with aq. HCl (1 M, ˜50 mL) and the water layer was backextracted with EtOAc (1×). The combined org. layers were washed with brine, dried over Na₂SO₄, filtered and concentrated. The crude was purified by flash chromatography (silica gel, 0-60% Et₂O/DCM (1:1) in heptane) to afford XD48 (1.95 g, 76%). ¹H NMR (400 MHz, CDCl₃) ppm=7.76-7.69 (m, 4H), 7.67-7.61 (m, 4H), 7.58-7.49 (m, 4H), 7.45-7.31 (m, 10H), 7.30-7.22 (m, 4H), 5.75-5.67 (m, 1H), 4.54 (t, J=7.6 Hz, 2H), 4.30-4.23 (m, 4H), 4.20-4.13 (m, 2H), 4.02 (s, 2H), 1.56 (s, 3H), 1.04 (s, 9H). MS (ESI⁺) calcd. for C₄₉H₄₉NaO₅PSi⁺ [M+H]⁺ 799.3 found 799.6.

### Triethylammonium (E)-4-hydroxy-3-methylbut-2-en-1-yl hydrogen phosphate (XD38)

Step 1: TBDPS-ether XD48 (911 mg, 1.17 mmol) was dissolved in THF/pyridine (1:1, 6 mL) in a Teflon tube under N₂. The mixture was cooled to 0° C., and HF•py (2 mL, 70% HF) was added. After stirring at 0° C. for 80 min, the reaction mixture was carefully added to a cooled (0° C.) mixture of sat. aq. NaHCO₃ and EtOAc under stirring. Once effervescence had stopped, the layers were separated and the aq. phase was extracted with EtOAc (3×). The combined org. layers were washed with aq. HCl (1 M) and brine, dried over Na₂SO₄, filtered and concentrated. Purification by flash chromatography (silica gel, 0-55% EtOAc in DCM) afforded the corresponding alcohol (418 mg, 66%).

Step 2: The alcohol (313 mg, 0.581 mmol) was taken up in THF (13 ml). The mixture was cooled to −78° C. and DBU (0.219 ml, 1.45 mmol) was added. After 5 min, the cooling bath was removed and the mixture was stirred at RT for 45 min. During this time a sticky precipitate formed. The reaction was diluted with ether (˜10 mL) and after stirring for 2 min the reaction was decanted. The residue was taken up in a minimal amount of MeCN (˜2 mL) and a small amount of MeOH was added to obtain a clear solution. The reaction was then diluted with ether (˜70 mL). The cloudy mixture was stirred for 5 min and was then left to settle overnight. The supernatant was removed by decantation and the process of dissolving in MeCN/MeOH and precipitating with ether was repeated three times. The residue was dried under vacuum to give 209 mg of an oil. The material was taken up in ethanol (2 ml) and loaded on a DOWEX 50WX8-200 hydrogen form column (5 g, pre-washed with ethanol until eluent is neutral and colorless) The product was eluted with ethanol. Product fractions were combined, Et₃N (0.083 mL) was added, and the colorless clear solution was concentrated to give XD38 (125 mg, 76%) as a colorless oil. ¹H NMR (400 MHz, CD₃OD) ppm=5.71-5.59 (m, 1H), 4.47 (t, J=6.8 Hz, 2H), 3.95 (s, 2H), 3.25-3.11 (m, 6H), 1.70 (s, 3H), 1.31 (t, J=7.3 Hz, 9H). MS (ESI⁻) calcd. for C₅H₁₀O₅P⁻ [M−H]⁻ 181.0 found 181.1.

### D: Preparation of Phosphorothioate XD39

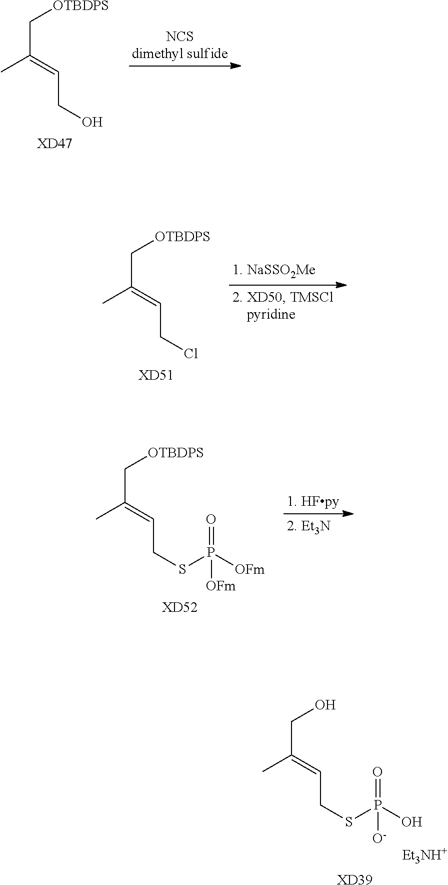

### (E)-tert-Butyl((4-chloro-2-methylbut-2-en-1-yl)oxy)diphenylsilane (XD51)

NCS (1.02 g, 7.63 mmol) was dissolved in dry DCM (25 ml) and the mixture was cooled to −40° C. DMS (0.695 ml, 9.40 mmol) was added dropwise under stirring, and the reaction was then warmed to 0° C. and stirred for 10 min. The reaction was cooled to −40° C. and alcohol XD47 (2.00 g, 5.87 mmol, synthesized as described by Serra, S. Tetrahedron: Asymmetry, 2014, 25, 1561-1572) dissolved in dry DCM (5 ml) was added. The reaction was allowed to warm to 0° C. over 2.5 h and was then stirred at 0° C. for an extra 90 min. Brine (30 mL) was added at 0° C. and the layers were separated. The aq. layer was extracted with DCM (40 mL) and the combined organic layers were dried over Na₂SO₄, filtered and concentrated in vacuo. The near colorless crude oil was purified by flash chromatography (silica gel, 0-20% DCM in heptane) to give chloride XD51 (1.93 g, 92%) of a colorless oil. ¹H NMR (400 MHz, CDCl₃) ppm=7.72-7.64 (m, 4H), 7.47-7.36 (m, 6H), 5.85 (tq, J=8.1, 1.5 Hz, 1H), 4.16 (d, J=8.1 Hz, 2H), 4.09 (s, 2H), 1.68 (s, 3H), 1.08 (s, 9H).

### (E)-O,O-bis((9H-Fluoren-9-yl)methyl)S-(4-((tert-butyldiphenylsilyl)oxy)-3-methylbut-2-en-1-yl) phosphorothioate (XD52)

Step 1: Sodium methanesulfonothioate (262 mg, 1.95 mmol) was added to a RT solution of chloride XD51 (700 mg, 1.95 mmol) in DMF (4 ml). After stirring for 5 h, the mixture was poured into water (50 mL) and the mixture was extracted with EtOAc/heptane (1:1, 2×30 mL) and the combined org layers were washed with water (2×30 mL) and brine (30 mL), dried over Na₂SO₄, filtered and concentrated. Purification by flash chromatography (silica gel, 0-15% EtOAc in heptane) afforded (E)-S-(4-((tert-butyldiphenylsilyl)oxy)-3-methylbut-2-en-1-yl) methanesulfonothioate (725 mg, 86%) as a colorless oil. ¹H NMR (400 MHz, CDCl₃) ppm=7.71-7.61 (m, 4H), 7.51-7.34 (m, 6H), 5.75 (tq, J=7.9, 1.4 Hz, 1H), 4.10 (s, 2H), 3.91 (d, J=7.9 Hz, 2H), 3.27 (s, 3H), 1.70 (s, 3H), 1.08 (s, 9H).

Step 2: XD50 (570 mg, 1.30 mmol) was taken up in MeCN (2.75 ml) and pyridine (5.6 ml) under N₂. The solution was cooled in an ice bath and TMSCl (0.825 ml, 6.51 mmol) was added dropwise. After 5 min, the cooling bath was removed and the reaction was stirred for 45 min at RT. (E)-S-(4-((tert-butyldiphenylsilyl)oxy)-3-methylbut-2-en-1-yl) methanesulfonothioate (706 mg, 1.62 mmol) was subsequently added and the mixture was stirred for 15 min at RT. Toluene (10 mL) was added and the mixture was concentrated. The residue was once more coevaporated with toluene (10 mL) before being purified by flash chromatography (silica gel, 0-50% “1:1 ether/DCM” in heptane), affording XD52 (818 mg, 79%) as a colorless wax. ¹H NMR (400 MHz, CDCl₃) ppm=7.73 (t, J=6.9 Hz, 4H), 7.65-7.59 (m, 4H), 7.59-7.52 (m, 4H), 7.44-7.32 (m, 10H), 7.32-7.24 (m, 4H), 5.63-5.50 (m, 1H), 4.51-4.36 (m, 2H), 4.33-4.15 (m, 4H), 3.96 (s, 2H), 3.40 (dd, J=12.0, 8.0 Hz, 2H), 1.53 (s, 3H), 1.02 (s, 9H).

### Triethylammonium (E)-S-(4-hydroxy-3-methylbut-2-en-1-yl)O-hydrogen phosphorothioate (XD39)

Step 1: TBDPS-ether XD52 (800 mg, 1.01 mmol) was reacted with HF•py analogous to the procedure for XD38, with a reaction time of 1 h and 45 min. Purification of the crude by flash chromatography (silica gel, 0-40% EtOAc in DCM) afforded the corresponding alcohol (452 mg, 81%) as a colorless oil. ¹H NMR (400 MHz, CDCl₃) ppm=7.75 (t, J=6.8 Hz, 4H), 7.61-7.53 (m, 4H), 7.45-7.36 (m, 4H), 7.36-7.27 (m, 4H), 5.45 (tq, J=8.0, 1.4 Hz, 1H), 4.48-4.39 (m, 2H), 4.31-4.19 (m, 4H), 3.89 (d, J=6.1 Hz, 2H), 3.36 (dd, J=13.6, 7.9 Hz, 2H), 1.59 (s, 3H). MS (ESI⁺) calcd. for C₃₃H₃₂O₄PS⁺ [M+H]⁺ 555.2 found 555.5.

Step 2: The alcohol (272 mg, 0.490 mmol) was dissolved in THF (3 ml) and Et₃N (0.75 ml, 5.38 mmol) was added at RT. After 7 h, an oily precipitate had formed and MeCN (2 ml) was added followed by triethylamine (0.5 ml, 3.59 mmol) to afford a clear solution. Stirring was continued overnight, and the clear solution was subsequently concentrated to ˜1 mL, and coevaporated with toluene (6 mL). The oily residue was taken up in MeCN (˜0.7 mL) and was then precipitated by the slow addition of ether (7 mL) under stirring. After stirring for 5 min, the emulsion was allowed to settle and the solution was then removed by decantation leaving an oily residue. The MeCN/ether treatment was repeated 4 times and the residue was then dried under vacuum to give the triethylamine salt XD39 (123 mg, 83%) as a colorless oil. ¹H NMR (400 MHz, CD₃OD) ppm=5.63 (tq, J=7.9, 1.3 Hz, 1H), 3.93 (s, 2H), 3.48 (dd, J=9.4, 8.4 Hz, 2H), 3.19 (q, J=7.4 Hz, 6H), 1.71 (d, J=0.8 Hz, 3H), 1.32 (t, J=7.3 Hz, 9H). MS (ESI⁻) calcd. for C₅H₁₀O₄PS⁻ [M−H]⁻ 197.0 found 197.0.

### E: Preparation of Alkyne Linker XD43

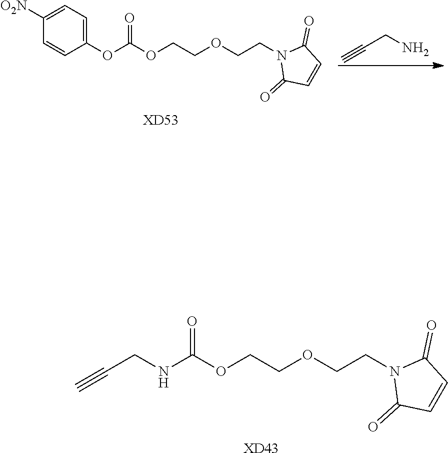

### 2-(2-(2,5-Dioxo-2,5-dihydro-1H-pyrrol-1-yl)ethoxy)ethyl prop-2-yn-1-ylcarbamate (XD43)

To PNP-carbonate XD53 (511 mg, 1.46 mmol, synthesized according to Elgersma, R. C. et al. Mol. Pharm. 2015, 12, 1813-1835) in THF (10 ml) at 0° C., propargylamine (0.093 ml, 1.46 mmol) was added. The cooling bath was removed and the mixture was stirred for 2 h at RT. The mixture was concentrated and the crude product was purified by flash chromatography (silica gel, 0-70% EtOAc in heptane), to give XD43 (265 mg, 68%) as a white solid. ¹H NMR (400 MHz, DMSO-d₆) ppm=7.60 (br t, J=5.5 Hz, 1H), 7.02 (s, 2H), 4.07-3.96 (m, 2H), 3.74 (dd, J=5.8, 2.4 Hz, 2H), 3.61-3.48 (m, 7H), 3.07 (t, J=2.5 Hz, 1H).

### F: General Procedure XXD: CDI Activation of Phosphates and Coupling with Phosphonates, Phosphates or Phosphorothioates

The mono-triethylamine salt of the phosphate (1.0 equiv.) was dissolved in DMF (0.15 M) under N₂, and CDI (2.1 equiv.) was added at RT. After stirring for 30 min, dry MeOH (1.0 equiv.) was added and the mixture was stirred for 15 min at RT before being concentrated. The residue was coevaporated with DMF to give crude A.

In a separate flask, the mono-triethylamine salt of the phosphonate, phosphate or phosphorothioate reactant (1.2 equiv.) was coevaporated with DMF and then redissolved in DMF (0.36 M) under N₂. The mixture was then cannulated into the flask containing crude A at RT. An identical volume of DMF was used to rinse the flask and complete the transfer. The mixture was stirred at RT under N₂, and once UPLC-MS analysis showed essentially complete conversion (typically 20-24 h) the reaction was concentrated and purified by preparative HPLC as indicated. Lyophilization of product fractions afforded the product.

### G: General Procedure XXE: Click-Reaction

Copper(II) sulfate pentahydrate (0.77 equiv.) in nitrogen purged water (0.034 M) was added to a flask containing solid azide (1.0 equiv) and alkyne (1.4 equiv.) at RT. An equal volume THF was added to give a homogeneous 1:1 water/THF solution. The headspace of the flask was briefly purged with N₂, and a solution of sodium ascorbate (1.5 equiv.) in nitrogen-purged water (0.13 M) was added. The reaction was stirred at RT until UPLC-MS analysis indicated full conversion (typically 1-2 h). Most of the THF was removed by brief rotary evaporation, and the aq. phase was taken up in MeCN/25 mM NH₄HCO₃ in MilliQ (1:9). Insoluble material was filter off using a syringe filter and the filtrate was purified by preparative HPLC as indicated. Lyophilization of product fractions afforded the product.

### H: Preparation of Linker-Drug XD44, XD45 and XD46

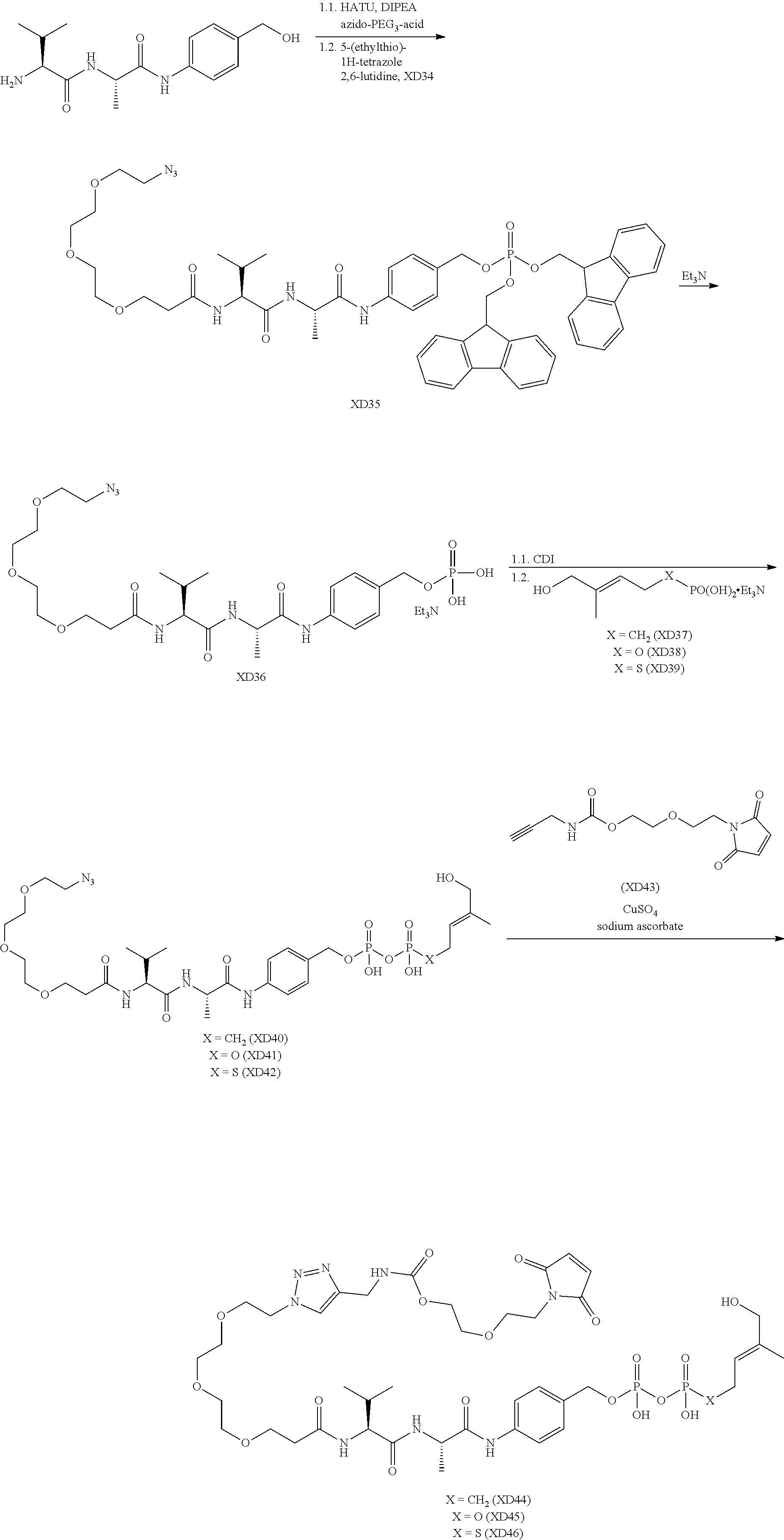

### bis((9H-Fluoren-9-yl)methyl) (4-((14S,17S)-1-azido-14-isopropyl-17-methyl-12,15-dioxo-3,6,9-trioxa-13,16-diazaoctadecan-18-amido)benzyl) phosphate (XD35)

Step 1: 3-(2-(2-(2-azidoethoxy)ethoxy)ethoxy)propanoic acid (142 mg, 0.574 mmol) was dissolved in DMF (1 ml). Val-Ala-PAB (160 mg, 0.545 mmol) in DMF (3.0 ml) was added, followed by the addition of HATU (228 mg, 0.600 mmol) and DIPEA (0.143 ml, 0.818 mmol) at RT. The reaction was stirred for 30 min before being concentrated. The crude was taken up in MeOH (1 mL) and basic impurities were removed by passing the solution through a short DOWEX 50WX8 plug that had been pre-washed with methanol. The product was eluted with methanol and the crude product was concentrated on silica gel. Purification by flash chromatography (silica gel, 0-8% MeOH in DCM) afforded the resulting amide (262 mg, 92%) as a cream solid. MS (ESI⁺) calcd. for C₂₄H₃₉N₆O₇⁺ [M+H]⁺ 523.3 found 523.6.

Step 2: To the amide product (977 mg, 1.87 mmol) and 5-(ethylthio)-1H-tetrazole (19 mg, 0.15 mmol) in MeCN (3.7 ml) under N₂ was added 2,6-lutidine (719 μl, 6.17 mmol) at RT followed by a solution of chloride XD34 (884 mg, 1.87 mmol) in DCM (3.7 mL), and the mixture was stirred at RT. More chloride XD34 was added after 80 min (88 mg, 0.187 mmol), and 140 min (177 mg, 0.374 mmol). After a total reaction time of 185 min, more 2,6-lutidine (218 μl, 1.87 mmol) was added and the reaction was continued for 2 h before being quenched with methanol (1 mL). The mixture was concentrated and the crude was taken up in EtOAc (80 mL) and aq. HCl (40 mL, 1 M). A small amount of MeCN (4 mL) was added to dissolve residual solids and the layers were separated. The water layer was extracted with EtOAc (80 mL) and the combined organic layers were washed with brine and dried over Na₂SO₄. The crude was purified by flash chromatography (silica gel, 0-5% MeOH in DCM) to yield phosphate ester XD35 (1.40 g, 66% yield). ¹H NMR (400 MHz, DMSO-d₆) ppm=9.94 (s, 1H), 8.18 (d, J=7.0 Hz, 1H), 7.89-7.82 (m, 5H), 7.55 (d, J=8.6 Hz, 2H), 7.52-7.44 (m, 4H), 7.42-7.34 (m, 4H), 7.30-7.24 (m, 4H), 7.09 (d, J=8.6 Hz, 2H), 4.60 (d, J=8.8 Hz, 2H), 4.40 (quint, J=7.0 Hz, 1H), 4.25-4.17 (m, 5H), 4.15-4.11 (m, 2H), 3.62-3.56 (m, 4H), 3.55-3.46 (m, 8H), 3.39-3.36 (m, 2H), 2.50-2.36 (m, 2H), 2.02-1.93 (m, 1H), 1.31 (d, J=7.1 Hz, 3H), 0.88 (d, J=6.8 Hz, 3H), 0.84 (d, J=6.8 Hz, 3H). MS (ESI⁺) calcd. for C₅₂H₅₉N₆O₁₀PNa⁺ [M+H]⁺ 981.4, found 981.8.

### 4-((14S,17S)-1-Azido-14-isopropyl-17-methyl-12,15-dioxo-3,6,9-trioxa-13,16-diazaoctadecan-18-amido)benzyl dihydrogen phosphate (XD36)

Triethylamine (0.25 ml) was added to a RT solution of phosphate XD35 (160 mg, 0.167 mmol) in MeCN (1 ml), and the reaction was stirred for 24 h. The reaction was diluted with toluene (8 mL) and then concentrated. The crude was suspended in ether (10 mL), filtered, and the solid was repetitively washed with ether to give alkyl phosphate XD36 (108 mg, 92%) as the mono triethylammonium salt. (Note: The product contained an impurity (m/z 606), potentially formed by elimination of the phosphate and trapping of the intermediate azaquinone methide with triethylamine. This impurity is unreactive in the next step and no further purification was required). MS (ESI⁻) calcd. for C₂₄H₃₈N₆O₁₀P⁻ [M−H]⁻ 601.2, found 601.7.

### Azide XD40

Alkyl phosphate XD36 (107 mg, 0.152 mmol) was reacted with phosphonate XD37 according to general procedure XXD. The crude was purified by preparative RP-HPLC (25 mM NH₄HCO₃ in MilliQ/MeCN, gradient 90:10 to 50:50), to give after lyophilization phosphonophosphate XD40 (60.5 mg, 50%) as a fluffy white solid. ¹H NMR (400 MHz, D₂O) ppm=7.44-7.39 (m, 4H), 5.38 (br t, J=7.1 Hz, 1H), 4.91 (d, J=7.0 Hz, 2H), 4.40 (q, J=7.1 Hz, 1H), 4.12 (d, J=7.1 Hz, 1H), 3.88 (s, 2H), 3.73 (t, J=6.0 Hz, 2H), 3.66-3.55 (m, 10H), 3.42 (t, J=4.9 Hz, 2H), 2.63-2.48 (m, 2H), 2.26-2.14 (m, 2H), 2.11-1.99 (m, 1H), 1.74-1.61 (m, 2H), 1.56 (s, 3H), 1.43 (d, J=7.3 Hz, 3H), 0.92 (d, J=6.6 Hz, 3H), 0.90 (d, J=6.6 Hz, 3H). MS (ESI⁻) calcd. for C₃₀H₄₉N₆O₁₃P₂⁻ [M−H]⁻ 763.3, found 763.7.

### Azide XD41

Alkyl phosphate XD36 (142 mg, 0.202 mmol) was reacted with alkyl phosphate XD38 according to general procedure XXD. The crude was purified by preparative RP-HPLC (25 mM NH₄HCO₃ in MilliQ/MeCN, gradient 90:10 to 50:50), to give after lyophilization pyrophosphate XD41 (65.9 mg, 41%) as a fluffy white solid. MS (ESI⁻) calcd. for C₂₉H₄₇N₆O₁₄P₂⁻ [M−H]⁻ 765.3, found 765.6.

### Azide XD42

Alkyl phosphate XD36 (142 mg, 0.202 mmol) was reacted with alkyl phosphate XD39 according to general procedure XXD. The crude was purified by preparative RP-HPLC (25 mM NH₄HCO₃ in MilliQ/MeCN, gradient 90:10 to 50:50), to give after lyophilization azide XD42 (88.6 mg, 54%) as a fluffy white solid. ¹H NMR (400 MHz, D₂O) ppm=7.48-7.39 (m, 4H), 5.51 (td, J=7.9, 1.1 Hz, 1H), 4.96 (d, J=6.9 Hz, 2H), 4.40 (q, J=7.2 Hz, 1H), 4.12 (d, J=7.1 Hz, 1H), 3.88 (s, 2H), 3.73 (t, J=6.0 Hz, 2H), 3.67-3.54 (m, 10H), 3.47-3.36 (m, 4H), 2.63-2.48 (m, 2H), 2.13-1.98 (m, 1H), 1.59 (s, 3H), 1.43 (d, J=7.1 Hz, 3H), 0.92 (br d, J=6.8 Hz, 3H), 0.91 (br d, J=6.6 Hz, 3H). MS (ESI⁻) calcd. for C₂₉H₄₇N₆O₁₃P₂S⁻ [M−H]⁻ 781.3, found 781.5.

### Linker-Drug XD44

Azide XD40 (18.5 mg, 0.023 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization phosphonophosphate XD44 (11.6 mg, 47%) as a fluffy white solid. ¹H NMR (400 MHz, D₂O) ppm=7.88 (s, 1H), 7.41 (s, 4H), 6.74 (s, 2H), 5.38 (br t, J=7.2 Hz, 1H), 4.91 (d, J=6.8 Hz, 2H), 4.53 (t, J=4.9 Hz, 2H), 4.38 (q, J=7.2 Hz, 1H), 4.31 (s, 2H), 4.17-4.04 (m, 3H), 3.92-3.83 (m, 4H), 3.70 (t, J=6.0 Hz, 2H), 3.66-3.57 (m, 6H), 3.57-3.43 (m, 8H), 2.62-2.47 (m, 2H), 2.27-2.14 (m, 2H), 2.10-1.97 (m, 1H), 1.74-1.61 (m, 2H), 1.55 (s, 3H), 1.41 (d, J=7.3 Hz, 3H), 0.90 (d, J=7.6 Hz, 3H), 0.88 (d, J=7.6 Hz, 3H). MS (ESI⁻) calcd. for C₄₂H₆₃N₈O₁₈P₂⁻ [M−H]⁻ 1029.4, found 1029.8.

### Linker-Drug XD45

Azide XD41 (30.2 mg, 0.038 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization pyrophosphate XD45 (36.2 mg, 90%) as a fluffy white solid. MS (ESI⁻) calcd. for C₄₁H₆₁N₈O₁₉P₂⁻ [M−H]⁻ 1031.4, found 1031.7.

### Linker-Drug XD46

Azide XD42 (27.1 mg, 0.033 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization linker-drug XD46 (22.6 mg, 63%) as a fluffy white solid. MS (ESI⁻) calcd. for C₄₁H₆₁N₈O₁₉P₂S⁻ [M−H]⁻ 1047.3, found 1047.7.

### Example 18: Synthesis of Linker-Drug XD58

### A: Preparation of Azide XD57

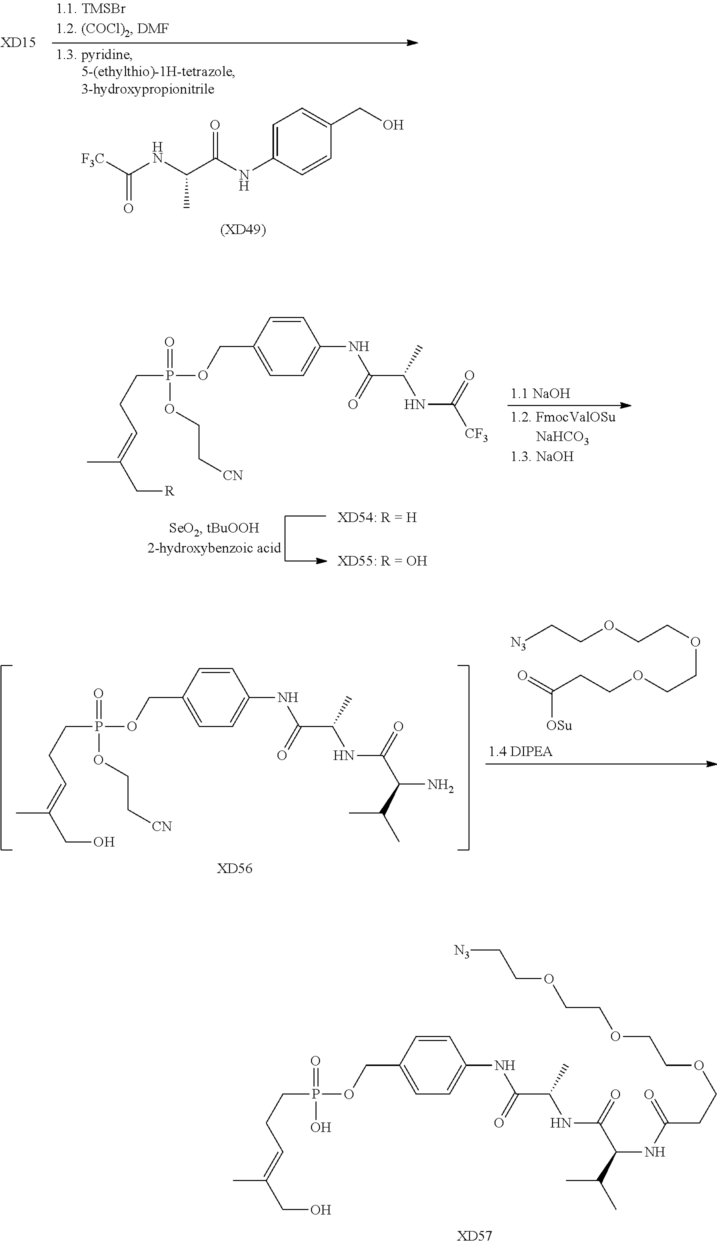

### 2-Cyanoethyl (4-((S)-2-(2,2,2-trifluoroacetamido)propanamido)benzyl) (4-methylpent-3-en-1-yl)phosphonate (XD54)

A solution of (4-methylpent-3-en-1-yl)phosphonic dichloride (540 mg, 2.69 mmol, prepared from phosphonic diester XD15 according to general procedure XXB) in DCM (4.3 mL) was added to a nitrogen purged vial charged with 5-(ethylthio)-1H-tetrazole (35.0 mg, 0.269 mmol). The solution was cooled to −78° C. and 3-hydroxypropanenitrile (0.184 ml, 2.69 mmol) and 2,6-lutidine (0.313 ml, 2.69 mmol) were sequentially added. After stirring for 30 min at −78 C, the reaction was warmed to RT and stirred for 2.5 h. A solution of XD49 (780 mg, 2.69 mmol, prepared as described for the synthesis of XD26) in THF/DCM (11 mL, 3:1, gentle heating with heat gun required to get a clear solution, then cooled back to RT) was then added rapidly to the reaction mixture at RT. After 3 h, more 2,6-lutidine (0.313 ml, 2.69 mmol) was added and the reaction was continued for 90 min. The mixture was diluted with EtOAc (50 mL) and was washed with aq. HCl (50 mL, 1 M). The water layer was backextracted with EtOAc (2×25 mL) and the combined organic layers were washed with brine (25 mL), dried over Na₂SO₄, filtered and concentrated. Purification by flash chromatography (silica gel, 0-40% acetone in DCM) afforded incomplete separation and the impure product was repurified by flash chromatography (silica gel, 0-4% MeOH in DCM) to give pure XD54 (630 mg, 48%). MS (ESI⁺) calc. for C₂₁H₂₇F₃N₃NaO₅P⁺ [M+Na]⁺512.2, found 512.5.

### 2-Cyanoethyl (4-((S)-2-(2,2,2-trifluoroacetamido)propanamido)benzyl) ((E)-5-hydroxy-4-methylpent-3-en-1-yl)phosphonate (XD55)

The allylic oxidation of alkene XD54 (0.625 g, 1.28 mmol) was performed according to general procedure XXC. The crude was purified by flash chromatography (silica gel, 0-8% MeOH in DCM), to yield alcohol XD55 (0.411 g, 64%). ¹H NMR (400 MHz, DMSO-d₆) ppm=10.22 (s, 1H), 9.73 (s, 1H), 7.61 (d, J=8.6 Hz, 2H), 7.36 (d, J=8.5 Hz, 2H), 5.39-5.29 (m, 1H), 5.05-4.91 (m, 2H), 4.66 (t, J=5.6 Hz, 1H), 4.48 (q, J=7.1 Hz, 1H), 4.18-4.02 (m, 2H), 3.76 (d, J=5.3 Hz, 2H), 2.88 (t, J=5.9 Hz, 2H), 2.26-2.13 (m, 2H), 1.90-1.76 (m, 2H), 1.52 (s, 3H), 1.41 (d, J=7.3 Hz, 3H). MS (ESI⁺) calc. for C₂₁H₂₇F₃N₃NaO₆P⁺ [M+Na]+528.2, found 528.4.

### Azide XD57

Step 1: Aq. NaOH (2 M, 1.31 ml, 2.62 mmol) was added to a cold (0° C.) solution of XD55 (396 mg, 0.655 mmol) in MeOH (4.6 ml)/water (0.6 ml). After 10 min, more aq. NaOH (2 M, 1.31 ml, 2.62 mmol) was added and the cooling bath was then removed. After 2 h, the reaction was cooled to 0° C., and aq. HCl (1 M, 2.95 mL, 4.5 eq) was added, followed by aq. AcOH (1 M, 1.97 mL, 3.0 eq). The mixture was then concentrated. MS (ESI⁺) calc. for C₁₆H₂₆N₂O₅P⁺ [M+H]⁺ 357.2, found 357.4.

Step 2: The crude product was taken up in water (5 mL) and NaHCO₃ (165 mg, 1.97 mmol) and iPrOH (5 mL) were added. Fmoc-Val-OSu (286 mg, 0.655 mmol) was added under stirring at RT, followed by the addition of THF (2.5 mL). After 2.5 h, the reaction was quenched with aq. AcOH (1 M, 2 mL) and concentrated. The crude was coevaporated with MeCN (3×) to remove traces of water. The resulting solid was repeatedly washed with EtOAc (20 mL) under stirring at 40° C., until no OSu ester was detected in the supernatant anymore, yielding a white solid.

Step 3: To a cooled (0° C.) solution of the crude solid in MeOH/water (10 mL, 9:1) was added aq. NaOH (2 M, 1.31 mL, 2.62 mmol), and the mixture was then stirred at RT for 45 min. The reaction was quenched with aq. AcOH (1 M, 3.9 mL) at 0° C. Methanol was then removed by rotary evaporation, and the aq suspension was diluted with water and filtered. The solid was washed with water and the aq. phase was lyophilized to give intermediate XD56 as white solid (840 mg). For subsequent reactions, quantitative conversion was assumed for step 1-3, corresponding to 35 wt % purity for crude XD56. MS (ESI⁺) calc. for C₂₁H₃₅N₃O₆P⁺ [M+H]⁺ 456.2, found 456.5.

Step 4: A portion of intermediate XD56 (490 mg crude, theoretic max. 0.365 mmol) was suspended in DMF (4 ml) at RT. DIPEA (0.254 ml, 1.46 mmol) and 2,5-dioxopyrrolidin-1-yl 3-(2-(2-(2-azidoethoxy)ethoxy)ethoxy)propanoate (146 mg, 0.424 mmol) in DMF (1 ml) were added and the mixture was stirred for 70 min. The mixture was concentrated and the crude was immediately purified by preparative RP-HPLC (25 mM NH₄HCO₃ in MilliQ/MeCN, gradient 90:10 to 50:50) to give, after lyophilization, azide XD57 (163.6 mg, 64%) as a white solid. ¹H NMR (400 MHz, D₂O) ppm=7.52-7.41 (m, 4H), 5.40 (br t, J=7.4 Hz, 1H), 4.94 (d, J=7.8 Hz, 2H), 4.45 (q, J=7.1 Hz, 1H), 4.17 (d, J=7.1 Hz, 1H), 3.91 (s, 2H), 3.78 (t, J=6.0 Hz, 2H), 3.71-3.61 (m, 10H), 3.51-3.44 (m, 2H), 2.69-2.55 (m, 2H), 2.28-2.16 (m, 2H), 2.16-2.04 (m, 1H), 1.77-1.65 (m, 2H), 1.58 (s, 3H), 1.49 (d, J=7.3 Hz, 3H), 0.98 (br d, J=6.6 Hz, 3H), 0.96 (br d, J=6.4 Hz, 3H). MS (ESI⁻) calc. for C₃₀H₄₈N₆O₁₀P⁻ [M−H]⁻ 683.3, found 683.6.

### B: Preparation of Linker-Drug XD58

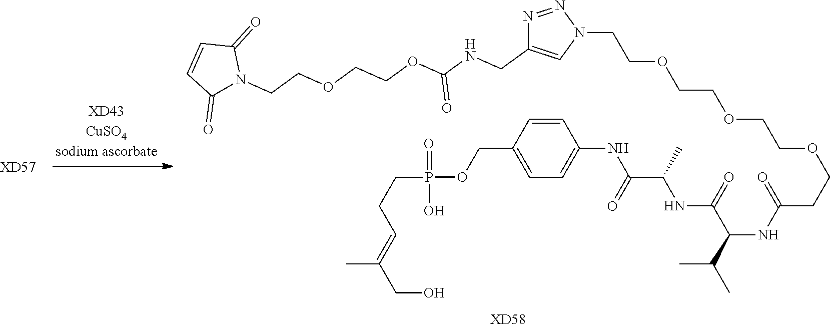

### Linker Drug XD58

Azide XD57 (21.4 mg, 0.030 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization linker-drug XD58 (19.7 mg, 67%) as a fluffy white solid. ¹H NMR (400 MHz, D₂O) ppm=7.92 (br s, 1H), 7.39 (d, J=8.5 Hz, 2H), 7.34 (d, J=8.5 Hz, 2H), 6.70 (s, 2H), 5.30 (br t, J=7.0 Hz, 1H), 4.83 (br d, J=4.6 Hz, 2H), 4.49 (br t, J=4.8 Hz, 2H), 4.35 (q, J=7.1 Hz, 1H), 4.27 (br s, 2H), 4.08 (d, J=7.0 Hz, 1H), 4.05 (br s, 2H), 3.87-3.82 (m, 2H), 3.81 (s, 2H), 3.67 (t, J=5.9 Hz, 2H), 3.63-3.53 (m, 6H), 3.53-3.40 (m, 8H), 2.61-2.42 (m, 2H), 2.11 (br s, 2H), 2.06-1.92 (m, 1H), 1.69-1.54 (m, 2H), 1.49 (s, 3H), 1.38 (d, J=7.1 Hz, 3H), 0.87 (d, J=7.6 Hz, 3H), 0.85 (d, J=7.4 Hz, 3H). MS (ESI⁻) calcd. for C₄₂H₆₂N₈O₁₅P⁻ [M−H]⁻ 949.4, found 949.8.

### Example 19: Synthesis of Linker-Drug XD59

### A: Preparation of Dialkyne Linker XS30

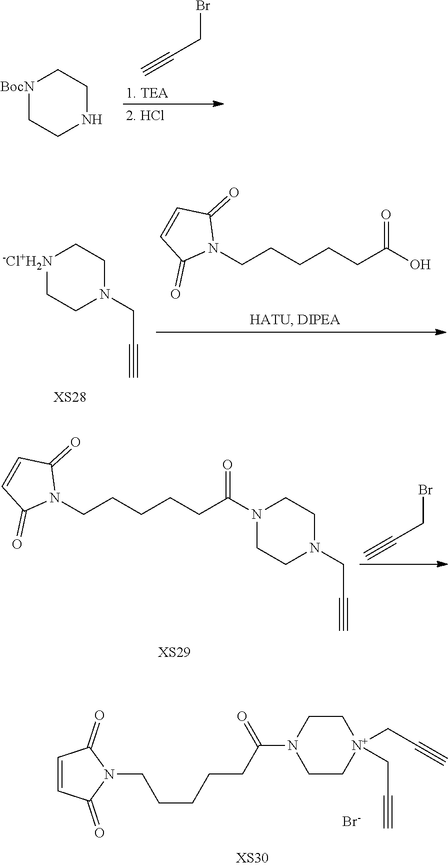

### 1-(Prop-2-yn-1-yl)piperazine (XS28)

Step 1: To a solution of tert-butyl piperazine-1-carboxylate (3.44 g, 18.5 mmol) in MeCN (17 mL) at 0° C., was added DIPEA (5.87 mL, 33.6 mmol) followed by propargyl bromide (80% in toluene, 1.80 mL, 16.8 mL). The reaction mixture was allowed to reach RT and stirred for 2 h. Then, it was partitioned between EtOAc (25 mL) and water (25 mL). The water layer was extracted with EtOAc (12 mL) and the combined organic layer was washed with brine (30 mL), dried over Na₂SO₄ and concentrated. Purification by flash chromatography (silica gel, 0-50% EtOAc in heptane), yielded tert-butyl 4-(prop-2-yn-1-yl)piperazine-1-carboxylate (3.56 g, 15.9 mmol, 94%) as a pale yellow oil. ¹H NMR (400 MHz, CDCl₃) ppm=3.47 (t, J=5.1 Hz, 4H), 3.32 (d, J=2.4 Hz, 2H), 2.51 (t, J=5.1 Hz, 4H), 2.26 (t, J=2.4 Hz, 1H), 1.46 (s, 9H). MS (ESI⁺) calc. for C₁₂H₂₁N₂O₂+[M+H]⁺ 225.2, found 225.2.

Step 2: A portion of the product (2.82 g, 12.6 mmol) was dissolved in DCM (6.3 mL) and a solution of 4 M HCl in dioxane (28.3 mL, 113 mmol) was added dropwise under stirring. The reaction mixture was stirred at RT for 4 h. The resulting suspension was filtered and the residue was washed with DCM (2×5 mL). The white solid was dried under vacuum to yield amine XS28 (2.48 g, quant.) as the hydrochloride salt. ¹H NMR (400 MHz, D₂O) ppm=3.98 (d, J=2.5 Hz, 2H), 3.52 (br s, 8H), 3.06 (t, J=2.5 Hz, 1H). MS (ESI⁺) calc. for C₇H₁₃N₂⁺[M+H]⁺ 125.1, found 125.1.

### Allyl ((S)-1-(((S)-1-((4-(hydroxymethyl)phenyl)amino)-1-oxo-5-ureidopentan-2-yl)amino)-3-methyl-1-oxobutan-2-yl)carbamate (XS29)

To a solution of 6-(2,5-dioxo-2,5-dihydro-1H-pyrrol-1-yl)hexanoic acid (1.90 g, 9.00 mmol) in DCM (45 mL) was added DIPEA (6.28 mL, 36.0 mmol), followed by HATU (3.59 g, 9.45 mmol). The reaction mixture was stirred for 1 h, after which amine XS28 (1.87 g, 9.45 mmol) was added. The reaction mixture was stirred for 30 min, after which is was partitioned between EtOAc (50 mL) and sat. aq. NaHCO₃ (50 mL). The aq. layer was extracted with EtOAc (2×50 mL) and the combined organic layers were washed with water (50 mL) and brine (50 mL), dried over Na₂SO₄, filtered and concentrated. The crude was purified by flash chromatography (silica gel, 0-0.1% Et₃N in EtOAc), to yield amide XS29 (2.20 g, 77%) as a pale yellow oil. ¹H NMR (400 MHz, DMSO-d₆) ppm=7.00 (s, 2H), 3.43 (d, 4H), 3.38 (t, J=7.1 Hz, 2H), 3.29 (d, J=2.4 Hz, 2H), 3.16 (t, J=2.4 Hz, 1H), 2.45-2.33 (m, 4H), 2.26 (t, J=7.5 Hz, 2H), 1.54-1.42 (m, 4H), 1.29-1.25 (m, 2H). MS (ESI⁺) calc. for C₁₇H₂₄N₃O₃⁺[M+H]⁺ 318.2, found 318.3.

### 4-(6-(2,5-dioxo-2,5-dihydro-1H-pyrrol-1-yl)hexanoyl)-1,1-di(prop-2-yn-1-yl)piperazin-1-ium bromide (XS30)

To a solution of amine XS29 (0.528 g, 1.66 mmol) in MeCN (3.3 mL) at 0° C. was added propargyl bromide (80% in toluene 0.926 mL, 8.32 mmol). The reaction mixture was allowed to reach RT and was stirred overnight, after which it was dropwise added to ether (45 mL) under stirring. An oily precipitate formed and the supernatant was removed by decantation. The residue was washed with ether (5 mL) and was subsequently taken up in a MeCN/toluene (4:1) mixture and concentrated, to give quaternary amine XS30 (0.710 g, 79%) as a pale yellow oil. ¹H NMR (400 MHz, DMSO-d₆) ppm=7.01 (s, 2H), 4.59 (d, J=2.1 Hz, 4H), 4.15 (t, J=2.2 Hz, 2H), 3.90-3.79 (m, 4H), 3.60-3.48 (m, 4H), 3.39 (t, J=7.1 Hz, 2H), 2.34 (t, J=7.4 Hz, 2H), 1.55-1.44 (m, 4H), 1.30-1.25 (m, 2H). MS (ESI⁺) calc. for C₂₀H₂₆N₃O₃⁺[M]⁺356.2, found 356.4.

### B: Preparation of Linker-Drug XD59

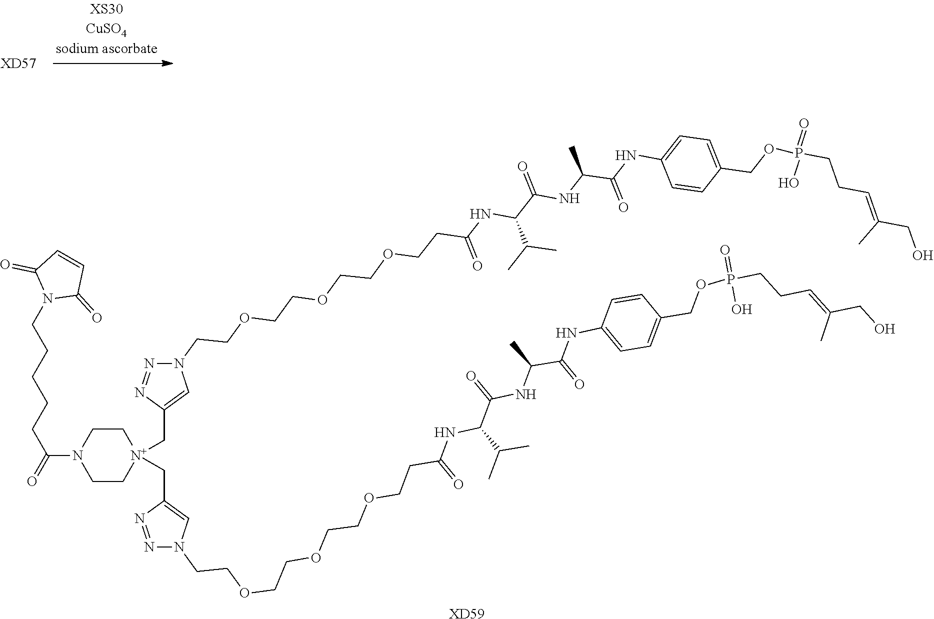

### Linker-Drug XD59

Water was purged with N₂ under stirring for 20 min before use. Alkyne XS30 (9.2 mg, 0.017 mmol) in THF/water (1:10, 0.55 mL) was added to solid XD57 (30.1 mg, 0.043 mmol) under N₂ at RT. Next, copper(II) sulfate pentahydrate (8.3 mg, 0.033 mmol) in water (1.0 mL) was added to give a clear solution. The headspace of the vial was purged with N₂, and subsequently sodium ascorbate (0.013 g, 0.064 mmol) in water (0.48 mL) was added at RT to give a turbid suspension. More alkyne XS30 (10 mg in THF/water (1:10, 0.540 mL)) was added in 4 portions over 3 h, at which point UPLC-MS analysis showed complete conversion. THF was removed by brief rotary evaporation and the aq. solution was diluted with 10% MeCN in 25 mM NH₄HCO₃ (10 mL) and purified by preparative RP-HPLC (25 mM NH₄HCO₃ in MilliQ/MeCN, gradient 90:10 to 50:50) to give, after lyophilization linker-drug XD59 (23.0 mg) as a white solid. ¹H NMR (400 MHz, D₂O) ppm=8.56 (s, 2H), 7.46 (d, J=8.5 Hz, 4H), 7.41 (d, J=8.5 Hz, 4H), 6.81 (s, 2H), 5.39 (br t, J=6.9 Hz, 2H), 4.89 (br d, J=7.0 Hz, 4H), 4.74 (s, 4H), 4.69 (br t, J=4.8 Hz, 4H), 4.42 (q, J=7.1 Hz, 2H), 4.16 (d, J=6.9 Hz, 2H), 4.10-4.01 (m, 4H), 3.98 (br t, J=4.7 Hz, 4H), 3.90 (s, 4H), 3.74 (t, J=5.9 Hz, 4H), 3.68-3.62 (m, 4H), 3.62-3.52 (m, 14H), 3.52-3.43 (m, 4H), 2.68-2.52 (m, 4H), 2.42 (br t, J=7.4 Hz, 2H), 2.26-2.14 (m, 4H), 2.09 (dq, J=13.6, 6.8 Hz, 2H), 1.67 (dt, J=16.3, 8.2 Hz, 4H), 1.60-1.51 (m, 10H), 1.46 (d, J=7.3 Hz, 6H), 1.34-1.20 (m, 2H), 0.96 (d, J=7.1 Hz, 6H), 0.93 (d, J=7.1 Hz, 6H). MS (ESI⁻) calcd. for C₈₀H₁₂₂N₁₅O₂₃P₂⁻ [M−H]⁻ 1722.8, found 1723.2.

### Example 20: Synthesis of Linker-Drug XD63

### A: Preparation of Phosphonate XD61

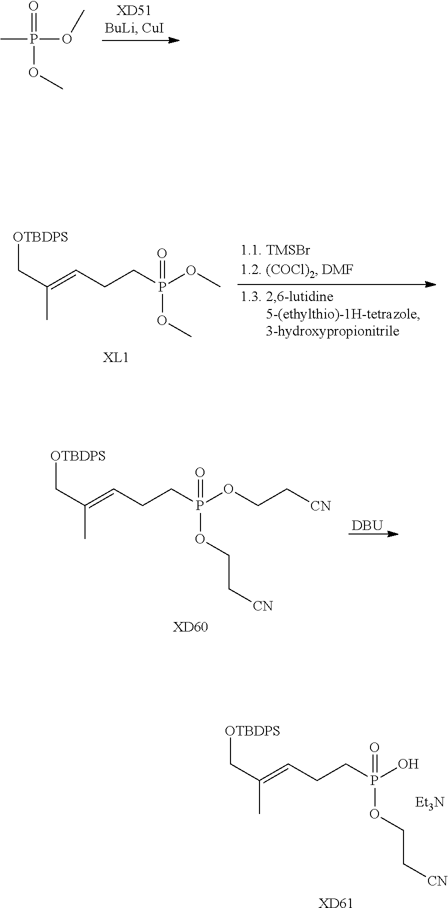

Dimethyl (E)-(5-((tert-butyldiphenylsilyl)oxy)-4-methylpent-3-en-1-yl)phosphonate (XL1) Dimethyl methylphosphonate (13.1 ml, 121 mmol) was dissolved in THF (440 ml) and cooled to −78° C., followed by the addition of n-butyllithium (75 ml, 121 mmol). The mixture was stirred at −78° C. for 1 h and was then warmed to −50° C. followed by the addition of CuI (11.5 g, 60.4 mmol) and stirring at this temperature for 1 h. The mixture was again cooled to −78° C. and XD51 (19.7 g, 54.9 mmol) dissolved in THF (110 ml) was added. The reaction was allowed to warm to rt overnight and was then quenched with sat. aq. NH₄Cl (500 mL). The mixture was extracted with EtOAc (2×500 mL), combined organic layers were washed with brine, dried over MgSO₄, filtered and concentrated in vacuo. The crude material was purified by flash chromatography (silica gel, 0-100% EtOAc in heptane) to afford XL1 (23.2 g, 95% yield) as a yellow oil. ¹H NMR (400 MHz, CDCl₃) ppm=7.69-7.63 (m, J=7.9, 1.5 Hz, 4H), 7.44-7.34 (m, 6H), 5.46-5.41 (m, 1H), 4.04 (br s, 2H), 3.75 (s, 3H), 3.73 (s, 3H), 2.38-2.28 (m, 2H), 1.83-1.73 (m, 2H), 1.60 (s, 3H), 1.06 (s, 9H). MS (ESI⁺) calcd. for C₂₄H₃₆O₄PSi⁺ [M+H]⁺ 447.2 found 447.4.

### B: Preparation of Linker-Drug XD63

### bis(2-Cyanoethyl) (E)-(5-((tert-butyldiphenylsilyl)oxy)-4-methylpent-3-en-1-yl)phosphonate (XD60)

Phosphonate XL1 (580 mg, 1.30 mmol) was converted to the phosphonic dichloride as described in general procedure XXB. The crude product was then reacted with 3-hydroxypropionitrile as described for the synthesis of XD21, with the exception that 2,6-lutidine was used instead of pyridine. Purification of the crude by flash chromatography (silica gel, 20-100% EtOAc in heptane) afforded phosphonate XD60 (360 mg, 53%) as a colorless oil. ¹H NMR (400 MHz, CDCl₃) ppm=7.69-7.62 (m, 4H), 7.46-7.34 (m, 6H), 5.44 (br t, J=7.0 Hz, 1H), 4.34-4.20 (m, 4H), 4.05 (s, 2H), 2.74 (t, J=6.1 Hz, 4H), 2.45-2.33 (m, 2H), 1.97-1.83 (m, 2H), 1.61 (s, 3H), 1.06 (s, 9H). MS (ESI⁺) calc. for C₂₈H₃₇N₂NaO₄PSi⁺ [M+H]⁺ 547.2, found 547.5.

### Triethylamine 2-cyanoethyl (E)-(5-((tert-butyldiphenylsilyl)oxy)-4-methylpent-3-en-1-yl)phosphonate (XD61)

DBU (0.053 ml, 0.349 mmol) was added to a solution of phosphonate XD60 (122 mg, 0.233 mmol) in THF (2 ml) at RT. After stirring for 30 min, the mixture was concentrated to ˜0.5 mL and the solution was diluted with MeOH (1 mL). DBU was then removed by passing the solution through a short DOWEX 50WX8 (H⁺ form) plug, using methanol to elute the product.

Triethylamine (0.032 ml, 0.233 mmol) was added to the eluent and the mixture was concentrated and coevaporated with MeCN (2×). Phosphonate XD61 (120 mg, 97%) was isolated as the triethylamine salt in a 1:0.6 ratio of phosphonate:Et₃N. ¹H NMR (400 MHz, CD₃OD) ppm=7.71-7.62 (m, 4H), 7.46-7.36 (m, 6H), 5.43 (td, J=7.3, 1.3 Hz, 1H), 4.08 (q, J=6.7 Hz, 2H), 4.05 (s, 2H), 3.20 (q, J=7.3 Hz, 4H), 2.78 (t, J=6.1 Hz, 2H), 2.38-2.28 (m, 2H), 1.65 (s, 3H), 1.71-1.61 (m, 2H), 1.31 (t, J=7.3 Hz, 5H), 1.04 (s, 9H). MS (ESI⁻) calcd. for C₂₅H₃₃NO₄PSi⁻ [M−H]⁻ 470.2, found 470.4.

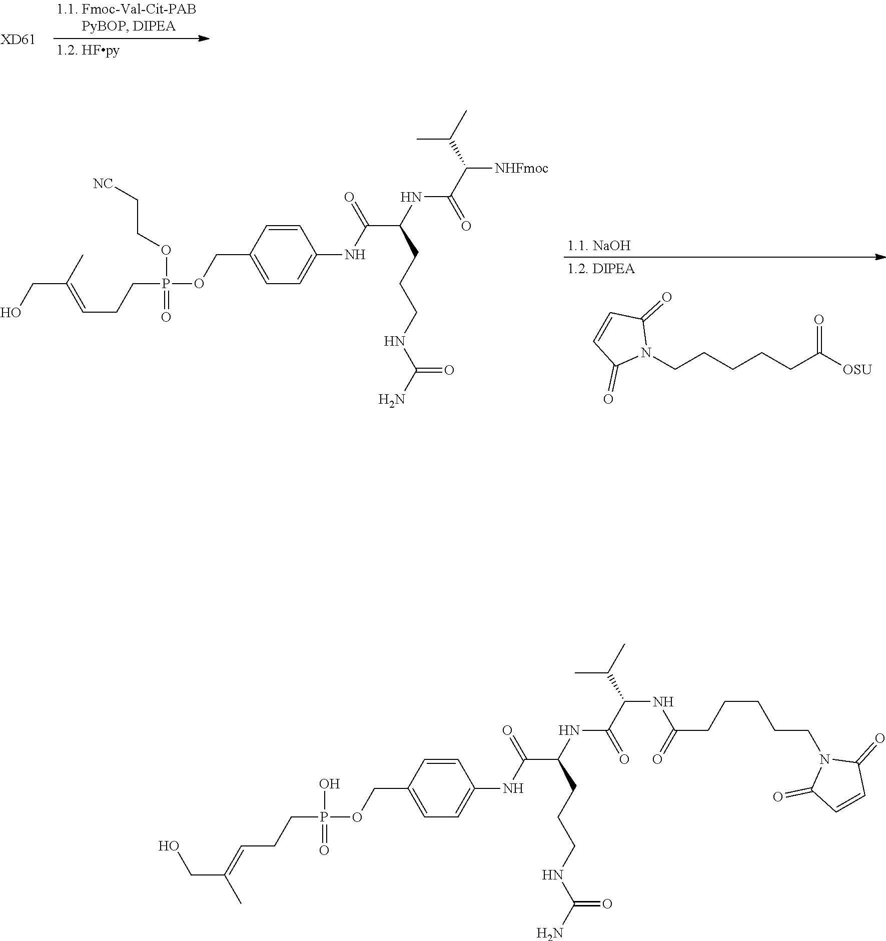

### (9H-Fluoren-9-yl)methyl ((2S)-1-(((2S)-1-((4-((((2-cyanoethoxy)((E)-5-hydroxy-4-methylpent-3-en-1-yl)phosphoryl)oxy)methyl)phenyl)amino)-1-oxo-5-ureidopentan-2-yl)amino)-3-methyl-1-oxobutan-2-yl)carbamate (XD62)

Step 1: Phosphonate XD61 (187 mg, 0.326 mmol) and Fmoc-Val-Cit-PAB (295 mg, 0.490 mmol) were combined in a round-bottom flask and coevaporated with DMF (3×8 mL). DMF (3.2 ml) was then introduced under N₂, followed by the addition of PyBOP (255 mg, 0.490 mmol) and DIPEA (0.057 ml, 0.326 mmol) at RT. More DIPEA (0.114 ml, 0.653 mmol) was added after 5 min and the mixture was stirred for 2 h. The reaction mixture was then slowly and dropwise added to water (70 mL, 0° C.). Stirring should be gentle to avoid gel formation. The white suspension was gently stirred for 5 min and was then filtered. The solid was collected and coevaporated with MeCN (2×) to remove traces of water. Purification of the solid by flash chromatography (silica gel, 0-12% MeOH in DCM) afforded the product (263 mg, 76%) as a white solid. MS (ESI⁺) calc. for C₅₈H₇₂N₆O₉PSi⁺[M+H]⁺ 1055.5, found 1056.0.

Step 2: A portion of the product (257 mg, 0.244 mmol) was suspended in THF (3.8 ml) and pyridine (0.370 ml) in a PFA vial under N₂. The vial was cooled to 0° C. and HF•pyridine (0.25 ml, 70%) was introduced. The mixture was stirred at this temperature for 6 h and the cold suspension was then carefully added to a cold (0° C.) sat. aq. NaHCO₃/10% iPrOH in EtOAc mixture. After stirring for 5 min, the layers were separated and the org. phase was washed with aq. HCl (1 M) and brine, dried over Na₂SO₄, filtered and concentrated on silica gel. Purification by flash chromatography (silica gel, 0-15% MeOH in DCM) afforded alcohol XD62 (148 mg, 74%) as a white solid. ¹H NMR (400 MHz, DMSO-d₆) ppm=10.10 (s, 1H), 8.12 (br d, J=7.5 Hz, 1H), 7.88 (d, J=7.5 Hz, 2H), 7.74 (t, J=7.8 Hz, 2H), 7.61 (d, J=8.5 Hz, 2H), 7.45-7.37 (m, 3H), 7.36-7.29 (m, 4H), 5.97 (br t, J=5.7 Hz, 1H), 5.40 (s, 2H), 5.34 (td, J=7.1, 1.0 Hz, 1H), 5.03-4.91 (m, 2H), 4.67 (t, J=5.6 Hz, 1H), 4.46-4.37 (m, 1H), 4.34-4.18 (m, 3H), 4.14-4.04 (m, 2H), 3.98-3.87 (m, 1H), 3.75 (d, J=5.5 Hz, 2H), 3.07-2.90 (m, 2H), 2.88 (t, J=5.9 Hz, 2H), 2.27-2.12 (m, 2H), 2.05-1.93 (m, 1H), 1.91-1.77 (m, 2H), 1.75-1.54 (m, 2H), 1.51 (s, 3H), 1.49-1.29 (m, 2H), 0.88 (d, J=6.8 Hz, 3H), 0.85 (d, J=6.8 Hz, 3H). MS (ESI⁺) calc. for C₄₂H₅₄N₆O₉P⁺ [M+H]⁺ 817.4, found 817.8.

### Linker-Drug XD63

Deprotection of phosphonate XD62 with NaOH, subsequent amide coupling with 2,5-dioxopyrrolidin-1-yl 6-(2,5-dioxo-2,5-dihydro-1H-pyrrol-1-yl)hexanoate and purification by RP-HPLC was performed analogous to step 3 and 4 in the synthesis of XD57 with the following modifications. Step 3: 5 eq. NaOH (2 M) were used and a reaction time of 2 h. Step 4: 2 eq of OSu ester were used. Linker-drug XD63 (47.9 mg, 36%) was obtained as a white solid. ¹H NMR (400 MHz, D₂O) ppm=7.46 (s, 4H), 6.82 (s, 2H), 5.40 (br t, J=7.1 Hz, 1H), 4.90 (br d, J=7.5 Hz, 2H), 4.45 (br t, J=6.9 Hz, 1H), 4.10 (br d, J=7.9 Hz, 1H), 3.91 (s, 2H), 3.46 (br t, J=6.9 Hz, 2H), 3.14 (br t, J=6.8 Hz, 2H), 2.31 (br t, J=6.6 Hz, 2H), 2.24-2.13 (m, 2H), 2.11-2.00 (m, 1H), 1.99-1.75 (m, 2H), 1.71-1.47 (m, 11H), 1.31-1.19 (m, 2H), 0.95 (br dd, J=6.4, 2.8 Hz, 6H). MS (ESI⁺) calc. for C₃₄H₅₂N₆O₁₀P⁺ [M+H]⁺ 735.4, found 735.7.

### Example 21: Preparation of Linker-Drug XD65

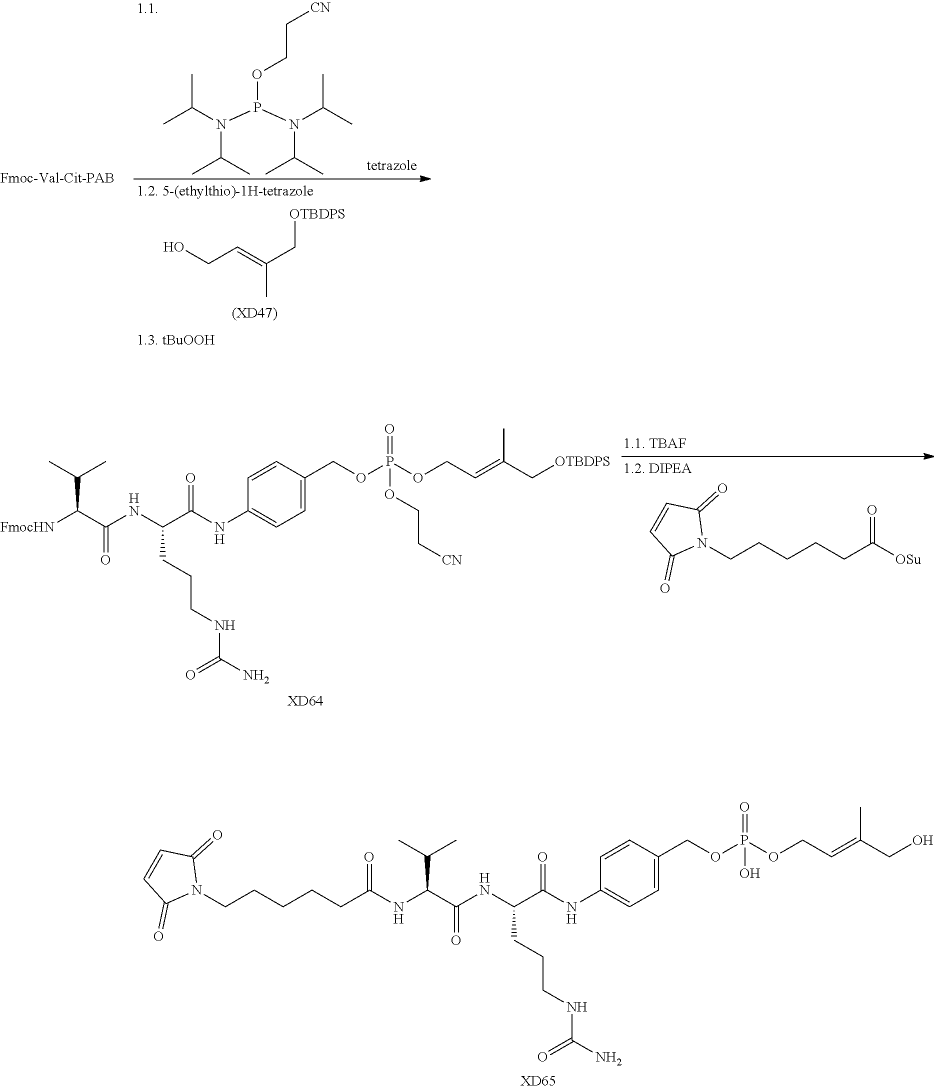

### (9H-Fluoren-9-yl)methyl ((2S)-1-(((2S)-1-((4-((((((E)-4-((tert-butyldiphenylsilyl)oxy)-3-methylbut-2-en-1-yl)oxy)(2-cyanoethoxy)phosphoryl)oxy)methyl)phenyl)amino)-1-oxo-5-ureidopentan-2-yl)amino)-3-methyl-1-oxobutan-2-yl)carbamate (XD64)

Fmoc-Val-Cit-PAB (300 mg, 0.499 mmol) was dissolved in DMF (7.0 ml) under N₂ at RT, and (3-((bis(diisopropylamino)phosphaneyl)oxy)propanenitrile (0.174 ml, 0.548 mmol) was added followed by the dropwise addition of tetrazole in MeCN (0.45 M, 1.22 ml, 0.548 mmol). The mixture was stirred for 2 h at RT. Meanwhile, alcohol XD47 (282 mg, 0.828 mmol, synthesized as described by Serra, S. Tetrahedron: Asymmetry, 2014, 25, 1561-1572) and 5-(ethylthio)-1H-tetrazole (143 mg, 1.097 mmol) were loaded in a 10 mL round-bottom flask and coevaporated with dry MeCN. The residue was taken up in DMF (0.5 ml) under N₂ and the mixture was then added via a cannula to the reaction mixture at RT. After stirring overnight, tBuOOH (5.5 M in decane, 0.199 ml, 1.097 mmol) was added at 0° C., and after 2 min, the reaction was warmed to RT and stirred for 90 min. The reaction was then poured into ice cold water (70 mL) under stirring, and after 5 min the suspension was filtered, and washed with a small amount of water (2×6 mL). The solid was then purified by flash chromatography (silica gel, 0-10% MeOH in DCM) to afford a mixture of product and Fmoc-Val-Cit-PAB (354 mg). The material was carried forward without further purification. MS (ESI⁺) calc. for C₅₇H₆₉N₆NaO₁₀PSi⁺[M+Na]⁺1079.5, found 1079.9.

### Linker-Drug XD65

(9H-fluoren-9-yl)methyl ((2S)-1-(((2S)-1-((4-((((((E)-4-((tert-butyldiphenylsilyl)oxy)-3-methylbut-2-en-1-yl)oxy)(2-cyanoethoxy)phosphoryl)oxy)methyl)phenyl)amino)-1-oxo-5-ureidopentan-2-yl)amino)-3-methyl-1-oxobutan-2-yl)carbamate (162 mg, 0.153 mmol) was dissolved in DMF (3.0 ml). TBAF (1.0 M in THF, 458 μl, 0.458 mmol) was added at RT. After 35 min, more TBAF (1.0 M in THF, 1.30 mL, 1.30 mmol) was added and the reaction was continued for 45 min. Ether (˜50 mL) was added to give an oil with a cloudy supernatant. The supernatant was removed and the residual oil was treated twice with ether (addition of ether, swirling, then decantation). The oily residue was taken up in DMF (0.3 mL) and acetic acid (0.044 mL, 0.764 mmol) was added followed by DIPEA (0.080 mL, 0.458 mmol) and 2,5-dioxopyrrolidin-1-yl 6-(2,5-dioxo-2,5-dihydro-1H-pyrrol-1-yl)hexanoate (70.6 mg, 0.229 mmol) at RT. After 25 min, complete conversion was observed. Acetic acid (0.044 mL, 0.764 mmol) was added and the mixture was concentrated. The crude was purified by preparative RP-HPLC (MilliQ×0.1% TFA/MeCN, gradient 90:10 to 40:60) and product fractions lyophilized. Partial decomposition was observed upon lyophilization under these acidic conditions. A portion of the impure product was repurified by preparative RP-HPLC (10 mM NH₄HCO₃ in MilliQ/MeCN, gradient 90:10 to 65:35) to give after lyophilization linker-drug XD65 (15.3 mg). ¹H NMR (400 MHz, DMSO-d₆) ppm=10.09 (s, 1H), 8.12 (br d, J=7.6 Hz, 1H), 7.85 (br d, J=8.8 Hz, 1H), 7.59 (d, J=8.6 Hz, 2H), 7.27 (d, J=8.6 Hz, 2H), 7.00 (s, 2H), 6.11-6.03 (m, 1H), 5.58-5.50 (m, 1H), 5.45 (br s, 2H), 4.75 (br d, J=6.8 Hz, 2H), 4.43-4.28 (m, 3H), 4.19 (dd, J=8.7, 6.9 Hz, 1H), 3.78 (s, 2H), 3.39-3.34 (m, 2H), 2.97 (dtt, J=18.9, 12.8, 6.3 Hz, 2H), 2.23-2.06 (m, 2H), 2.02-1.91 (m, 1H), 1.78-1.65 (m, 1H), 1.65-1.29 (m, 10H), 1.22-1.12 (m, 2H), 0.85 (d, J=6.9 Hz, 3H), 0.81 (d, J=6.8 Hz, 3H). MS (ESI⁻) calc. for C₃₃H₄₈N₆O₁₁P⁻ [M−H]⁻ 735.3, found 735.9.

### Example 22: Synthesis of Conjugates from Linker-Drug Compounds (LDs)

ADC numbers used in Table 1 reflect the corresponding linker-prodrugs synthesized as disclosed in the Examples, as well as antibody used.

The antibodies used in the conjugates reflected in Table 1 were:
Anti-CD20 monoclonal antibody (MoAb) rituximab (r)
Anti-Her2 MoAb trastuzumab (t), or trastuzumab-41C (t_{41C}), wherein the amino acid on the 41 position in the heavy chain (HC) of the antibody has been replaced by a cysteine.
anti-CD123 MoAb (CD123, which is a proprietary MoAb, Byondis B.V.
anti-5T4-Moab (5T4), which is the anti-5T4 antibody that is disclosed in WO2015/177360 as H8-HC41C (the heavy chain comprises the amino acid sequence of SEQ ID NO:8 and the light chain comprises the amino acid sequence of SEQ ID NO:11)
Anti-PSMA MoAb SYD1030 41C (p_{41C}), which is the anti-PSMA antibody having an engineered cysteine at position 41 of the heavy chain (i.e. HC41C) that is disclosed in WO2015/177360 as SYD1030 (the heavy chain comprises the amino acid sequence of SEQ ID NO:2 and the light chain comprises the amino acid sequence of SEQ ID NO:5).
isotype control antibody (i). The isotype control antibody used contains the variable domain sequences of human anti-HIV-1 pg120 antibody B12 with accession no. 2NY7 (Zhou et al, 2007, Nature, 445, 732-737) and has been used as an IgG1 kappa isotype control antibody (accession no. P01857 and P01834, respectively).

Respective conjugates were synthesized according to the methods described in example 22a-c. All conjugates with a DAR below 8, reflected in Table 1 were synthesized according to the procedure described in example 22a, except for conjugate with DAR2, which were made by site-specific conjugation, as described in example 22b. Conjugates with higher DAR (8 and above) were made by the procedure described in example 22c. DAR (pAg to antibody ratio in the conjugate) is also indicated in Table 1.

### Example 22a: Synthesis of Conjugates with DAR 2

To a solution of antibody (10-12 mg/mL) was added TRIS (1 vol %, 1 M, pH 8), EDTA (4 vol %, 25 mM) and TCEP (5 mM in water). The resulting solution was incubated at RT for 2 h. After incubation, the reduced antibody was rebuffered to 4.2 mM histidine, 50 mM trehalose pH 6 and treated with dimethylacetamide (DMA) and linker-drug compound (LD) (10 mM in DMA, >1.5 eq/SH). Final DMA content was ˜10 vol %. The resulting mixture was roller mixed in the dark at RT overnight. Activated carbon was added and the suspension was roller mixed in the dark for 1 h, filtered, washed with 4.2 mM histidine, 50 mM trehalose pH 6. The solution was rebuffered to 4.2 mM histidine, 50 mM trehalose pH 6 and sterile filtered.

### Example 22b: Synthesis of Conjugates with DAR2 by Site-Specific Conjugation

Some conjugates with DAR2 were synthesized by site-specific conjugation (ADC-XD18-CD123_{41C}, ADC-XD18-5T4₄₁, as reflected in Table 1), where the linker drug molecule is only linked to two engineered cysteines on position 41 in the antibody heavy chain according to the Kabat numbering system (“41C”). These conjugates were prepared according to the method disclosed in WO2015177360 and WO2017137628.

### Example 22c: Synthesis of Conjugates with Higher DAR (DAR8, DAR16 and DAR10, DAR20)

To a solution of antibody (12 mg/mL in 4.2 mM histidine, 50 mM trehalose, pH 6), EDTA (25 mM in water, 4% v/v) and TRIS (1 M in water, pH 8, 2% v/v) were added.

For conjugates with DAR 8 or 16, a wild type antibody was used. For conjugates with DAR 10 or 20, a 41C modified antibody was used, wherein the amino acid on position 41, according to the Kabat numbering system, in the heavy chains was replaced by a cysteine. This modification results in the introduction of 2 additional cysteines in the amino acid sequence of the antibody, that can be reduced in the next step with TCEP, resulting in a total of 10 potential linking positions for the linker drug (LD).

TCEP (10 mM in water, 30 eq) was added and the resulting mixture was incubated at RT overnight. The reactants were removed by a centrifugal concentrator (Vivaspin filter, 30 kDa cut-off, PES) using 4.2 mM histidine, 50 mM trehalose, pH 6.

DMA was added, followed by a solution of the appropriate linker-drug. For conjugates with DAR=8 and DAR=16, 10 mM in DMA, 16 eq was added. For conjugates with DAR=10 or 20, 10 mM in DMA, 20 eq was added.

The conjugates with DAR16 and DAR20 were made with linker drugs based on a branched linker, wherein each branched linker carries two pAg moieties (linker drug XD59, as reflected in Table 1). The final concentration of DMA was 10%.

The resulting mixture was incubated at RT in the absence of light for 3 h or overnight. In order to remove the excess of linker-drug, activated charcoal was added and the mixture was incubated at RT for 1 h. The coal was removed using a 0.2 m PES or PVDF filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.2 μm PVDF filter.

In order to approximate the DAR (pAg to antibody ration) of the conjugates with a target DAR of 2, synthesized as described in example 22a or 22b, surrogate conjugation was performed with the hydrophobic seco-DUBA payload (SYD980,described in a.o. WO2015/177360), that allows facile DAR determination via HIC.

The resulting approximate DAR for conjugates where the target DAR is 2, is reflected in table 1. For conjugates with higher DAR, synthesized as described in example 22c, table 1 reflects the target DAR (indicated with “target”. The actual DAR may deviate somewhat from this value (this means that the DAR could not be measured a standard (HIC) technique—due to either overlapping peaks (for target DAR 2 ADCs) or due to the fact that the ADCs being made were fully reduced/conjugated (for target DAR 8/10/16/20 ADCs).

When multiple values are given for a DAR or % HMW, separated by a comma, these refer to different batches of the same ADCs.

In Table 1, “<LOD” stands for “below Limit Of Detection”.

TABLE 1
Synthesized Conjugates with antibody and Linker-drug (LD) compound used in synthesis.

| | | | | Approx. | |
|---|---|---|---|---|---|
| LD | LD structure | Conjugate | MoAb | DAR | % HMW |
| XC4 | 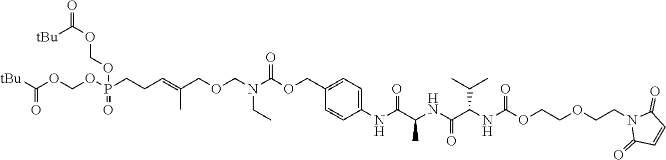 [SMILES: CCN(COC/C(C)=C/CCP(=O)(OCOC(=O)C(C)(C)C)OCOC(=O)C(C)(C)C)C(=O)OCc1ccc(NC(=O)[C@H](C)NC(=O)[C@@H](NC(=O)OCCOCCN2C(=O)C=CC2=O)C(C)C)cc1] | ADC-XC4-r ADC-XC4-i | rituximab (anti-CD20) Isotype (Anti-HIV) | 2 1.7 | 0.4 0.5 |
| XD13 | 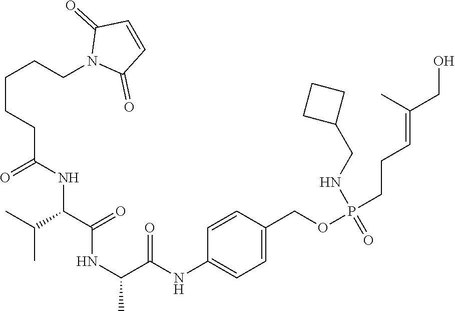 [SMILES: C/C(=C\CCP(=O)(NCC1CCC1)OCc1ccc(NC(=O)[C@H](C)NC(=O)[C@@H](NC(=O)CCCCCN2C(=O)C=CC2=O)C(C)C)cc1)CO] | ADC-XD13-r ADC-XD13-i | rituximab isotype | 2 1.7 | 0.3 0.1 |
| XD4 | 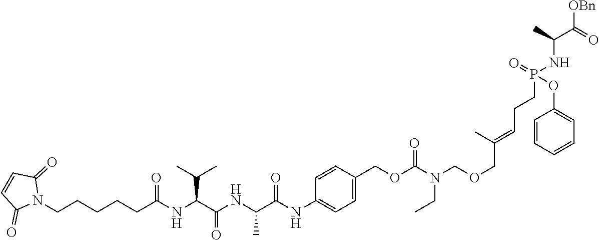 [SMILES: CCN(COC/C(C)=C/CCP(=O)(N[C@@H](C)C(=O)OCc1ccccc1)Oc1ccccc1)C(=O)OCc1ccc(NC(=O)[C@H](C)NC(=O)[C@@H](NC(=O)CCCCCN2C(=O)C=CC2=O)C(C)C)cc1] | ADC-XD4-r ADC-XD4-i | rituximab isotype | 1.9, 2.0 1.6 | 0.3, 1.4 0.1 |
| XS2 | 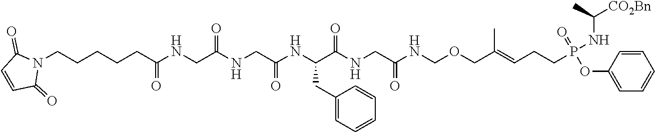 [SMILES: C/C(=C\CCP(=O)(N[C@@H](C)C(=O)OCc1ccccc1)Oc1ccccc1)COCNC(=O)CNC(=O)[C@H](Cc1ccccc1)NC(=O)CNC(=O)CNC(=O)CCCCCN1C(=O)C=CC1=O] | ADC-XS2-r ADC-XS2-t (or i) | rituximab trastuzumab as isotype | 2.1 2.2 | 0.0 0.7 |
| XD18 | 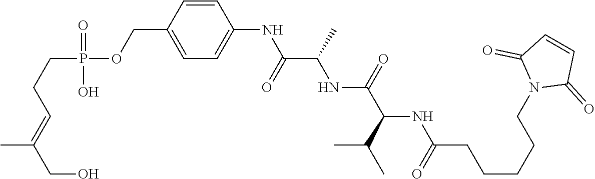 [SMILES: C/C(=C\CCP(=O)(O)OCc1ccc(NC(=O)[C@H](C)NC(=O)[C@@H](NC(=O)CCCCCN2C(=O)C=CC2=O)C(C)C)cc1)CO] | ADC-XD18-r ADC-XD18-t ADC-XD18-i ADC₈-XD18-r ADC₈-XD18-i ADC-XD18- CD123_{41C} ADC-XD18-5T4_{41C} ADC₁₀-XD18- CD123_{41C} | rituximab trastuzumab isotype rituximab isotype anti-CD123 41C MoAb Anti-5T4 41C MoAb anti-CD123 41C MoAb | 2.1 2.2, 2.2 1.9, 2.1, 2.0 target 8 target 8 2.1 1.9 target 10 | 0.2 0.7, 1.0 <LOD, <LOD, <LOD 0.8 1.3 1.2 1.01 1.7 |
| | | ADC₁₀-XD18-_{p41C} | Anti-PSMA | target 10 | 0.8 |
| | | | SYD1030 41C | | |
| | | ADC-XD18-_{p41C} | Anti-PSMA | 1.8 | 1.1 |
| | | | SYD1030 41C | | |
| XD65 | 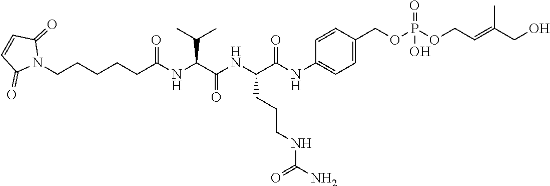 [SMILES: C/C(=C\COP(=O)(O)OCc1ccc(NC(=O)[C@H](CCCNC(N)=O)NC(=O)[C@@H](NC(=O)CCCCCN2C(=O)C=CC2=O)C(C)C)cc1)CO] | ADC-XD65-r ADC-XD65-i ADC8-XD65-r ADC8-XD65-i | rituximab isotype rituximab isotype | 2.1 2.1 target 8 target 8 | <LOD <LOD 0.6 0.7 |
| XS12 | 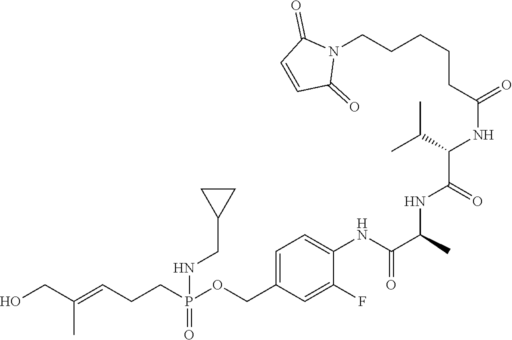 [SMILES: C/C(=C\CCP(=O)(NCC1CC1)OCc1ccc(NC(=O)[C@H](C)NC(=O)[C@@H](NC(=O)CCCCCN2C(=O)C=CC2=O)C(C)C)c(F)c1)CO] | ADC-XS12-r ADC-XS12-i | rituximab isotype | 2.0 1.5 | <LOD 0.09 |
| XS17 | 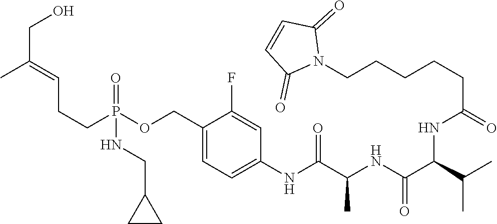 [SMILES: C/C(=C\CCP(=O)(NCC1CC1)OCc1ccc(NC(=O)[C@H](C)NC(=O)[C@@H](NC(=O)CCCCCN2C(=O)C=CC2=O)C(C)C)cc1F)CO] | ADC-XS17-r ADC-XS17-i | rituximab isotype | 2.0 1.5 | 0.2 0.08 |
| XS7 | 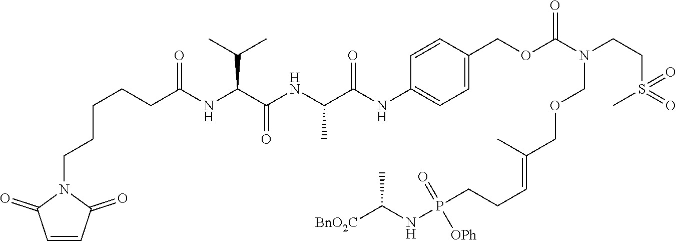 | ADC-XS7-r ADC-XS7-i | rituximab isotype | 2.6 1.2 | 0.1 <LOD |
| XS25 | 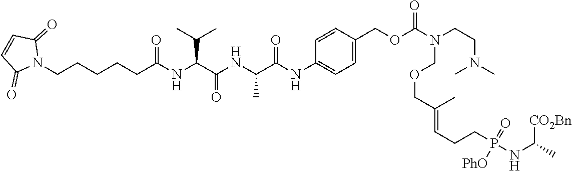 [SMILES: C/C(=C\CCP(=O)(N[C@@H](C)C(=O)OCc1ccccc1)Oc1ccccc1)COCN(CCN(C)C)C(=O)OCc1ccc(NC(=O)[C@H](C)NC(=O)[C@@H](NC(=O)CCCCCN2C(=O)C=CC2=O)C(C)C)cc1] | ADC-XS25-r ADC-XS25-i | rituximab isotype | target 2 target 2 | n.d. n.d. |
| XC9 | 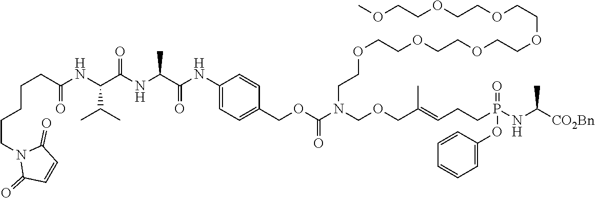 [SMILES: COCCOCCOCCOCCOCCOCCOCCN(COC/C(C)=C/CCP(=O)(N[C@@H](C)C(=O)OCc1ccccc1)Oc1ccccc1)C(=O)OCc1ccc(NC(=O)[C@H](C)NC(=O)[C@@H](NC(=O)CCCCCN2C(=O)C=CC2=O)C(C)C)cc1] | ADC-XC9-r ADC-XC9-t | rituximab trastuzumab | 2.1 2.2 | 0.1 0.7 |
| XC13 | 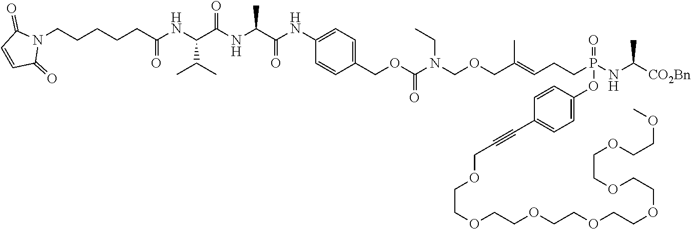 [SMILES: CCN(COC/C(C)=C/CCP(=O)(N[C@@H](C)C(=O)OCc1ccccc1)Oc1ccc(C#CCOCCOCCOCCOCCOCCOCCOCCOC)cc1)C(=O)OCc1ccc(NC(=O)[C@H](C)NC(=O)[C@@H](NC(=O)CCCCCN2C(=O)C=CC2=O)C(C)C)cc1] | ADC-XC13-r ADC-XC13-t | rituximab trastuzumab | 2.1 2.2 | 0.1 0.7 |
| XS22 | 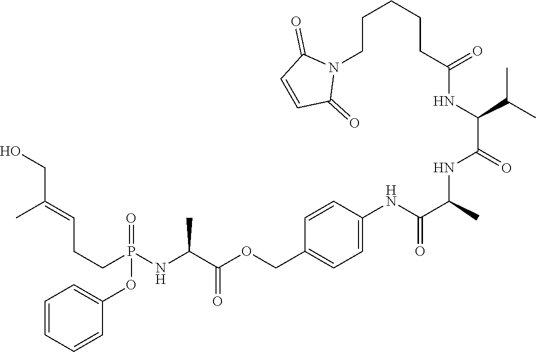 [SMILES: C/C(=C\CCP(=O)(N[C@@H](C)C(=O)OCc1ccc(NC(=O)[C@H](C)NC(=O)[C@@H](NC(=O)CCCCCN2C(=O)C=CC2=O)C(C)C)cc1)Oc1ccccc1)CO] | ADC-XS22-r ADC-XS22-i | rituximab isotype | Target 2 Target 2 | 0.9 0.8 |
| XD24 | 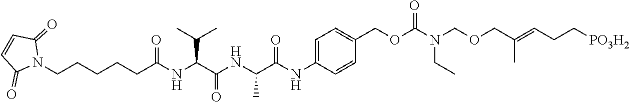 | ADC-XD24-r ADC-XD24-i | rituximab isotype | 1.9 1.6 | 0.3 0.14 |
| XD44 | 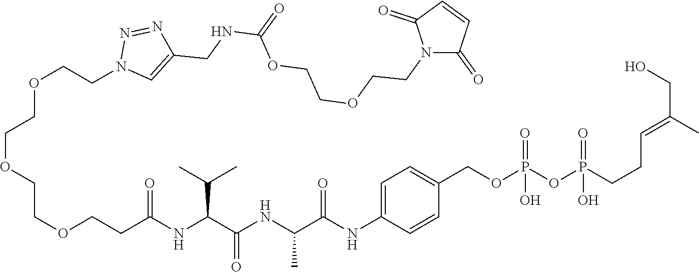 [SMILES: C/C(=C\CCP(=O)(O)OP(=O)(O)OCc1ccc(NC(=O)[C@H](C)NC(=O)[C@@H](NC(=O)CCOCCOCCOCCn2cc(CNC(=O)OCCOCCN3C(=O)C=CC3=O)nn2)C(C)C)cc1)CO] | ADC-XD44-r ADC-XD44-i | rituximab isotype | 2.2 2.2 | <LOD <LOD |
| XD45 | 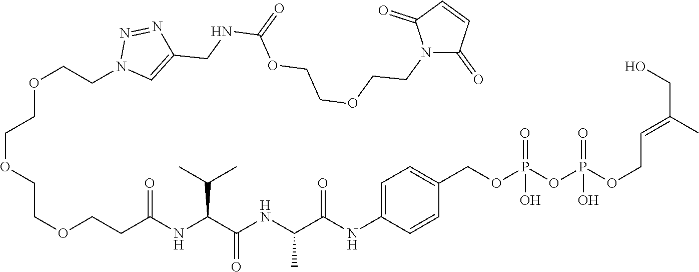 [SMILES: C/C(=C\COP(=O)(O)OP(=O)(O)OCc1ccc(NC(=O)[C@H](C)NC(=O)[C@@H](NC(=O)CCOCCOCCOCCn2cc(CNC(=O)OCCOCCN3C(=O)C=CC3=O)nn2)C(C)C)cc1)CO] | ADC-XD45-r ADC-XD45-i | rituximab isotype | 2.2 2.1 | <LOD <LOD |
| XD46 | 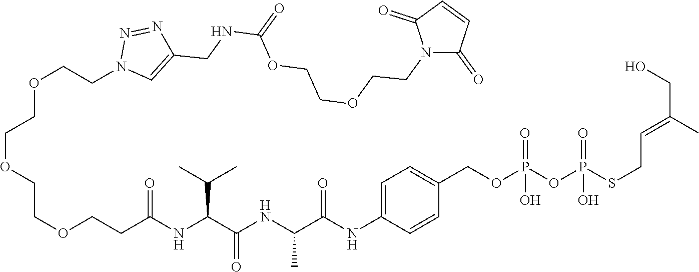 [SMILES: C/C(=C\CSP(=O)(O)OP(=O)(O)OCc1ccc(NC(=O)[C@H](C)NC(=O)[C@@H](NC(=O)CCOCCOCCOCCn2cc(CNC(=O)OCCOCCN3C(=O)C=CC3=O)nn2)C(C)C)cc1)CO] | ADC-XD46-r ADC-XD46-i | rituximab isotype | 2.5 2.4 | <LOD <LOD |
| XD58 | 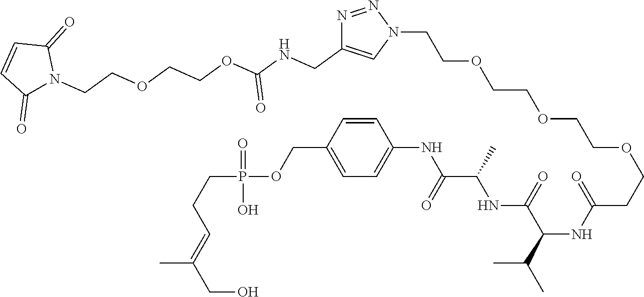 [SMILES: C/C(=C\CCP(=O)(O)OCc1ccc(NC(=O)[C@H](C)NC(=O)[C@@H](NC(=O)CCOCCOCCOCCn2cc(CNC(=O)OCCOCCN3C(=O)C=CC3=O)nn2)C(C)C)cc1)CO] | ADC-XD58-r ADC-XD58-i | rituximab isotype | 2.0 2.2 | <LOD <LOD |
| XD59 | 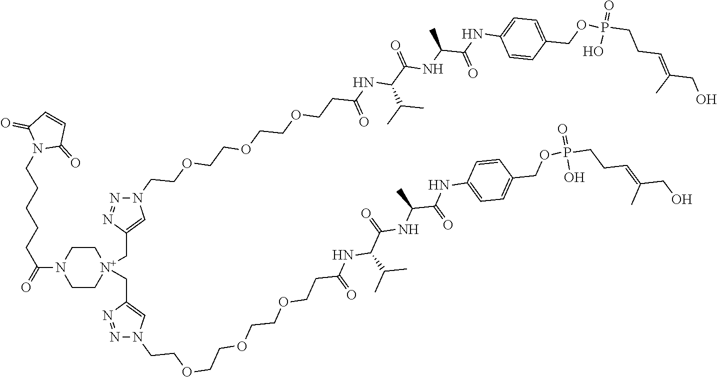 [SMILES: C/C(=C\CCP(=O)(O)OCc1ccc(NC(=O)[C@H](C)NC(=O)[C@@H](NC(=O)CCOCCOCCOCCn2cc(C[N+]3(Cc4cn(CCOCCOCCOCCC(=O)N[C@H](C(=O)N[C@@H](C)C(=O)Nc5ccc(COP(=O)(O)CC/C=C(\C)CO)cc5)C(C)C)nn4)CCN(C(=O)CCCCCN4C(=O)C=CC4=O)CC3)nn2)C(C)C)cc1)CO] | ADC16-XD59-r ADC16-XD59-i ADC20-XD59-_{c41C} | rituximab isotype anti-CD123 41C MoAb | target 16 target 16 target 20 | 0.5 1.1 1.6 |
| XD63 | 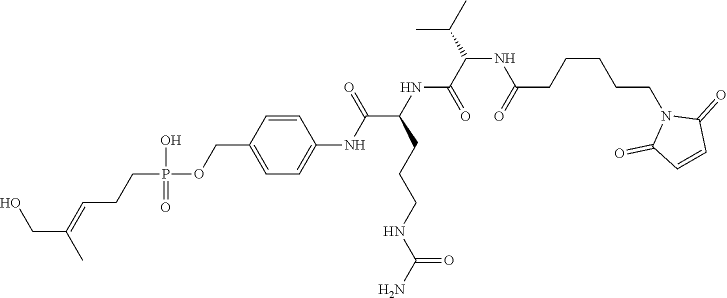 | ADC-XD63-r ADC-XD63-i | rituximab isotype | 2.2 1.9 | 0.75 1.7 |

### Example 23: Activity of Prodrugs and Conjugates on Gamma Delta T-Cells

To determine (unconjugated) phosphoantigen prodrug activity, Raji target cells were pre-incubated with XC1 and XD1, prior to co-culture with peripheral blood mononuclear cells (PBMCs) containing effector cells. Selective gammadelta T-cell activation was studied in vitro after co-culture of PBMCs with tumor cells (from the CD20-positive Burkitt's Lymphoma human tumor cell line Raji) pretreated with phosphoantigen HMBPP, phosphoantigen prodrugs XC1, XD1, or the conjugates listed in Table 1. Structural formulas of prodrugs tested are listed in Table 2 below.

TABLE 2
Phosphoantigen prodrugs; structural formula and code.

| structural formula | Exp code |
|---|---|
| 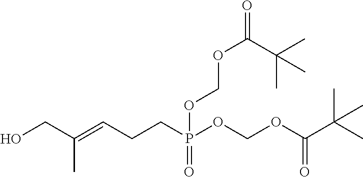 [SMILES: C/C(=C\CCP(=O)(OCOC(=O)C(C)(C)C)OCOC(=O)C(C)(C)C)CO] | XC1 |
| 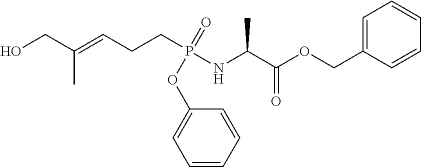 [SMILES: C/C(=C\CCP(=O)(N[C@@H](C)C(=O)OCc1ccccc1)Oc1ccccc1)CO] | XD1 |

As a source of immune cells, PBMCs of a healthy human donor were used.

Once activated, Vγ9Vδ2 gammadeltaT-cells produce cytokines and release cytotoxic granules (degranulation), leading to immune activation and target cell killing, respectively. While only Vγ9Vδ2 gammadelta T-cells are known to sense fluctuations in phosphoantigen levels, some of the effector mechanisms induced are shared with other immune cells, including CD8+ T-cells, NK cells and other subsets of gammadelta T-cells. These immune cell populations are all present in PBMCs, isolated from blood of healthy donors. PBMCs therefore represent a good source of cells for performing in vitro experiments to determine selective activation of gammadelta T-cells.

Identification of different immune cell populations can be achieved by specific staining with fluorescently-labeled monoclonal antibodies. When monensin and/or brefeldin A are added during co-culture of PBMCs and targets, produced IFNγ will be trapped in activated cells. Staining with fluorescently-labeled antibodies in the presence of saponin, allowing anti-IFNγ antibodies to enter the cell, will identify IFNγ-producing cells. Fluorescently-labeled antibodies against CD107a can also be added during co-culture and will stain cells that have undergone degranulation. Thus, by combining fluorescently-labeled immune-cell specific markers and CD107a- and IFNγ-markers, it is possible to determine the activation status of the gammadelta T-cells and/or other immune cell subsets after co-culture with pretreated target cells.

### Materials and Methods

The CD20-positive Burkitt's Lymphoma human tumor cell line Raji (DSMZ, the German collection of Microorganisms and cell cultures GmbH (Leibniz Institute, Germany)) was used for in vitro experiments. Raji cells were cultured in complete growth medium (CGM): RPMI-1640 (Lonza, Walkersville, MD, USA) supplemented with 10% Heat-inactivated (HI) Fetal Bovine Serum (FBS) (Gibco-Life Technologies; Carlsbad, CA) and 80 U/mL Penicillin-Streptomycin solution (Lonza Group Ltd, Basel Switzerland). Raji cells were maintained at 37° C. in a humidified incubator containing 5% CO2 and sub-cultured twice a week.

For stimulation with conjugates according to the invention (ADCs), unconjugated phosphoantigen prodrugs and HMBPP, Raji cells were harvested, diluted to a concentration of 5×10⁶ cells/mL and 50 μL (equivalent to 250.000 cells/well) of this cell suspension was seeded into a 96-well plate. A 2-times concentrated, 10-fold serial dilution of the prodrugs and ADC was prepared in CGM. Plated Raji cells were incubated overnight (O/N) in a humidified incubator with 5% CO2 at 37° C. with 50 μL/well of the serially-diluted compounds.

The following day, the 96 well plate with Raji cells and compounds (ADCs, unconjugated phosphoantigen prodrugs or HMBPP) was washed by adding 100 μL/well CGM, centrifugation at 300×g for 3 minutes at room temperature (RT), and removal of supernatant in order to remove excessive unbound compound.

As a source of immune cells, frozen peripheral blood mononuclear cells (PBMCs) of a healthy human donor were thawed, resuspended in CGM and placed O/N in a humidified incubator with 5% CO2 at 37° C. to let the cells recover.

The recovered PBMCs were harvested, counted and diluted to a concentration of 10×10⁶ cells/mL in CGM, and 50 μL/well (equivalent to 0.5×10⁶ cells/well) was added to the Raji cells. A 2-times concentrated anti-CD107a-AlexaFluor 647 solution was prepared in CGM, containing GolgiStop (Monensin) and GolgiPlug (Brefeldin A) (BD Biosciences, San Jose, CA, USA), and 50 μL/well was added to the Raji-PBMCs co-culture. In all experiments, 1% Phytohemagglutinin (PHA-M, Gibco-ThermoFisher), known as an aspecific activator of immune cells, was also included in a well to serve as a positive control. Samples were incubated for 6 hours in a humidified incubator with 5% CO2 at 37° C.

For specific staining of immune cell subsets, a multicolor antibody staining cocktail was prepared in Brilliant Stain buffer, containing anti-CD3 BUV396 (not included in all occasions), anti-CD8 BV421, anti-CD56 PE-Cy7, Fixable Viability Stain 780 (BD Biosciences, San Jose, CA, USA), anti-TCR Vδ1 PerCP-Vio700, FcR Blocking Reagent (Miltenyi Biotec, Bergisch Gladbach, Germany) and anti-TCR Vδ2 BV711 (Biolegend, San Diego, USA). After the 6 hours incubation period, the plate was centrifuged at 300×g for 3 minutes at RT and supernatant was discarded. The pellet was re-suspended in 50 μL antibody cocktail and incubated for 30 minutes on ice, protected from light. The plate was washed twice by adding 100 μL ice-cold FACS buffer (PBS 1×, 0.1% v/w BSA, 0.02% v/v Sodium Azide (NaN3)), followed by centrifugation at 300× g for 3 minutes and discarding of the supernatant. Cells were fixed and permeabilized using 100 μL/well Cytofix/Cytoperm Solution (BD bioscience, San Jose, CA, USA) and were incubated for 20 minutes on ice, protected from light. Cells were washed three times by adding 150 μL BD Perm/wash solution containing saponin (dilute 10× BD Perm/Wash buffer in distilled H₂O, to make a 1× solution prior to use), followed by centrifugation at 300×g for 3 minutes and discarding of the supernatant. Finally, cells were re-suspended in FACS buffer and stored O/N in the fridge at 4° C., protected from light.

On the third day, stained PBMCs/Raji cells were washed once in 150 μL BD Perm/wash solution followed by centrifugation at 300×g for 3 minutes and discarding of the supernatant. The pellet was re-suspended in a mix of 50 μL anti-IFNγ BV650 (BD Biosciences, San Jose, CA, USA) diluted in Perm/Wash solution and incubated for 30 minutes on ice, protected from light. After incubation the plate was washed once with 150 μL ice-cold FACS buffer, followed by centrifugation at 300×g for 3 minutes and discarding of the supernatant. Thereafter, the cell pellet was resuspended in 150 μL FACS buffer and samples were analyzed using the BD FACSymphony A3 Cell analyzer (BD Biosciences, San Jose, CA, USA) with corresponding High Throughput Sampler in order to analyze samples in the 96 well plate.

### Gating Strategy

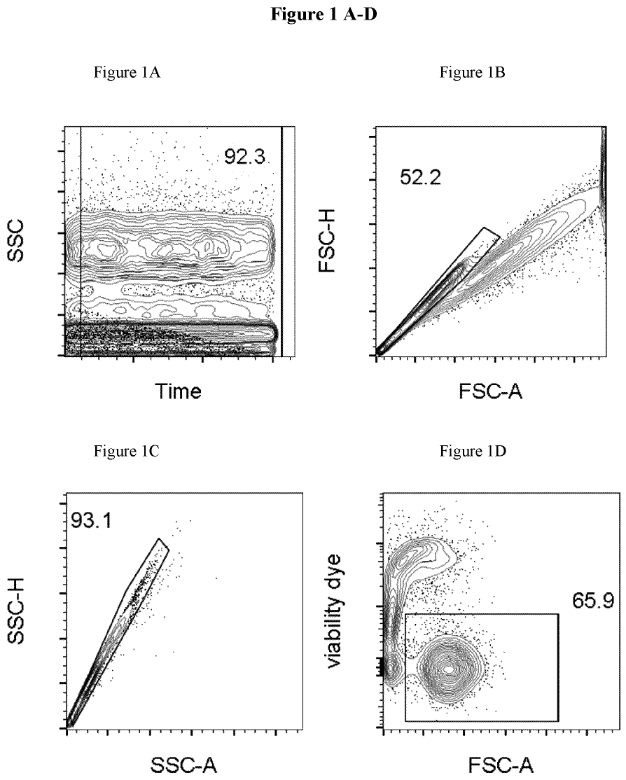

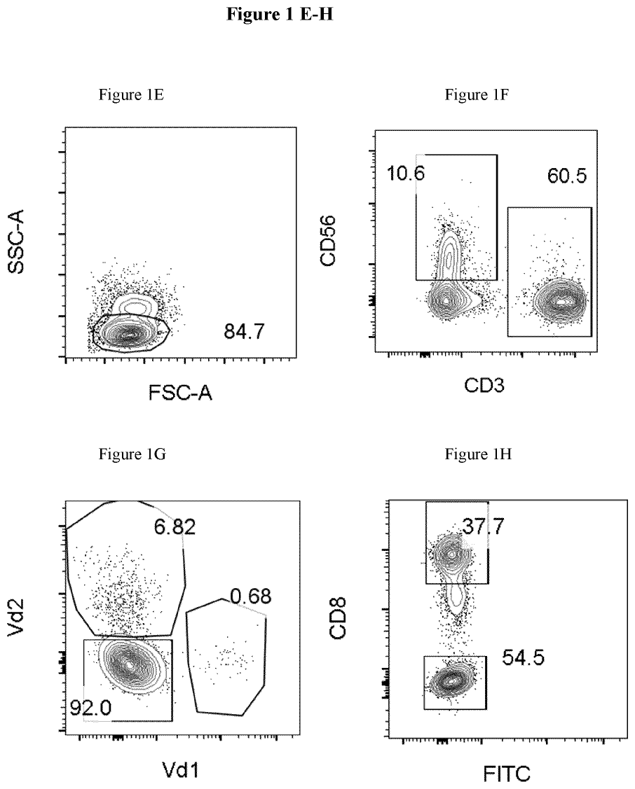

Analysis was performed using FlowJo V10.7., and the acquired samples were subjected to electronical gating to define various immune cell populations (FIG. 1). First, a time gate was applied to assure a constant flow (FIG. 1A) and to exclude potential irregularities, then doublets and dead cells were excluded on the FSC-A versus FSC-H and SSC-A versus SSC-H plots (FIGS. 1B and C).

Viable cells were then selected (FIG. 1D), followed by selection of lymphocytes based on FSC/SSC (FIG. 1E). CD3-negative, CD56-positive cells were then identified as NK cells (FIG. 1 F). CD3-positive cells were further divided into Vδ2 and Vδ1 positive cells (FIG. 1G). Lymphocytes were also subdivided into CD8-positive cytotoxic T-cells (FIG. 1H), resulting in 4 cell families:
NK cells were defined as CD3⁻CD56⁺ cells,
CD8⁺ T-cells were defined as CD3⁺VδV1⁻Vδ2⁻CD8⁺,
Vδ1 γδ T-cells were defined as CD3⁺Vδ1+Vδ2⁻ and
Vδ2 γδT-cells were defined as CD3⁺Vδ1⁻Vδ2⁺.

Of note, the B6 clone used here to stain Vδ2 γδ T-cells solely stains Vγ9Vδ2 γδ T-cells and not Vγ9⁻ populations of Vδ2 γδ T-cells. For these four immune cell populations, proportions of CD107a⁺ or IFNγ⁺ cells were determined. In addition, the median fluorescent intensities of the CD107⁺ or IFNγ⁺ cell populations were determined as a measure of activity per cell. CD107a has been described as a marker for degranulation and strongly correlates with target-cell killing. IFNγ accumulation, caused by brefeldinA/monensin treatment preventing excretion, is a measure for IFNγ cytokine production.

### Results/Conclusion

### Cell-Membrane-Permeable Prodrugs Induce Activation of Vδ2 γδ T-Cells

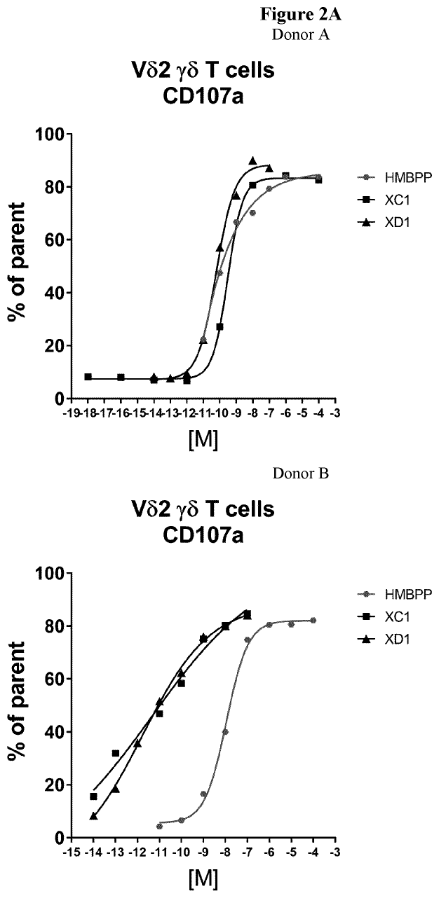

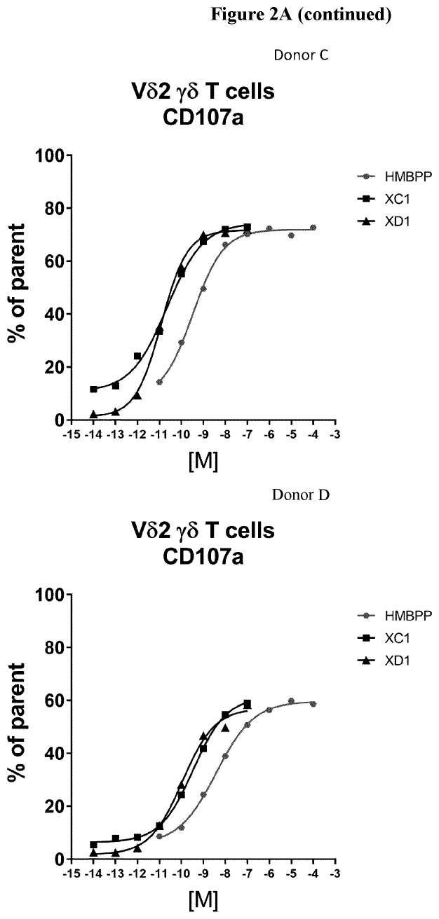

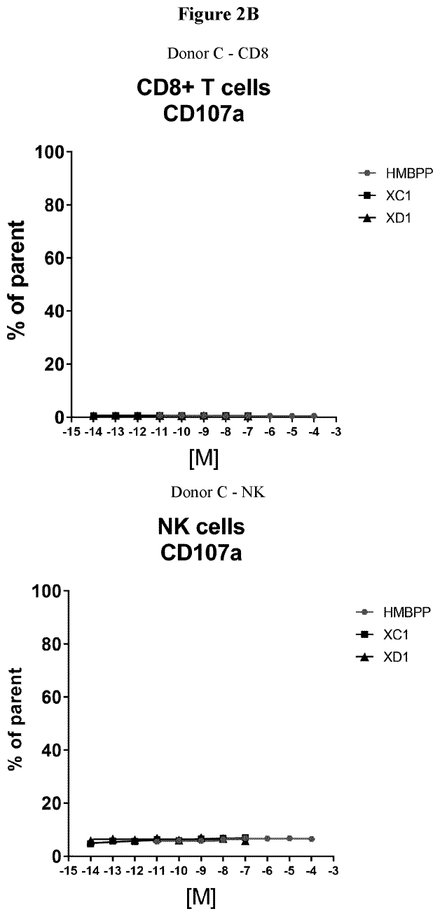

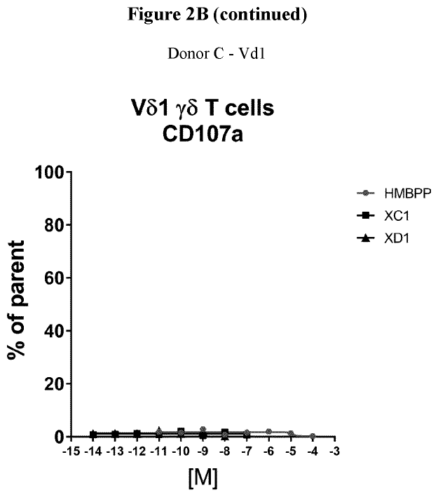

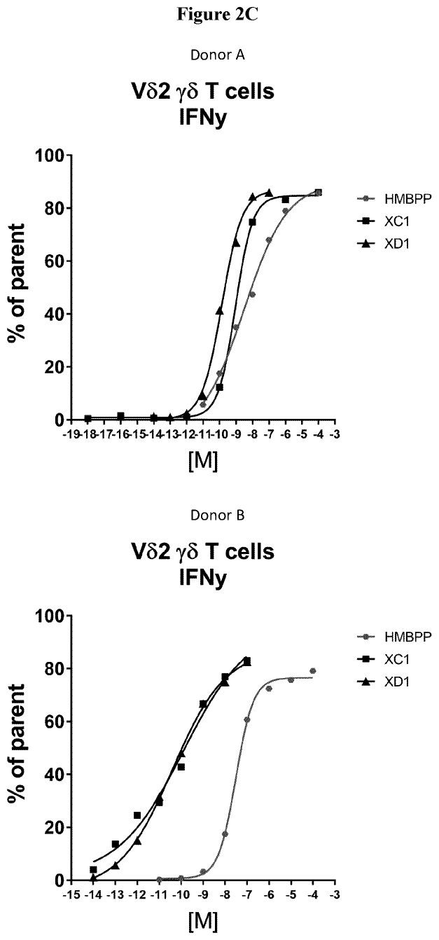

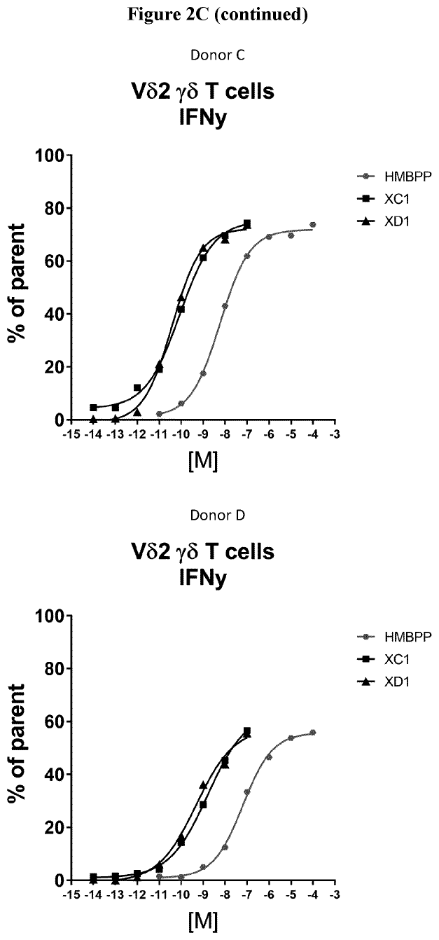

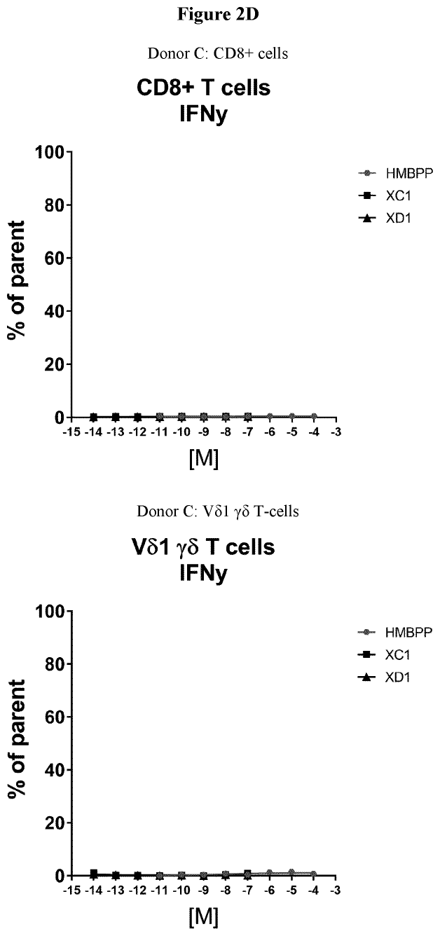

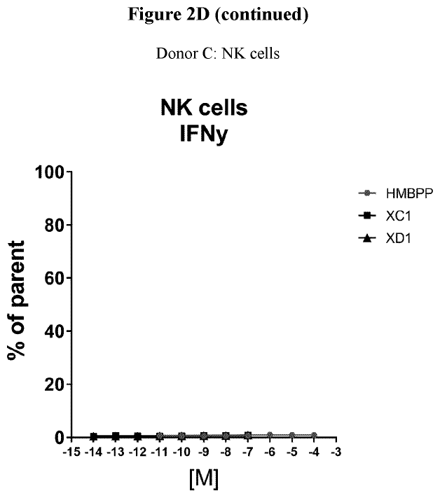

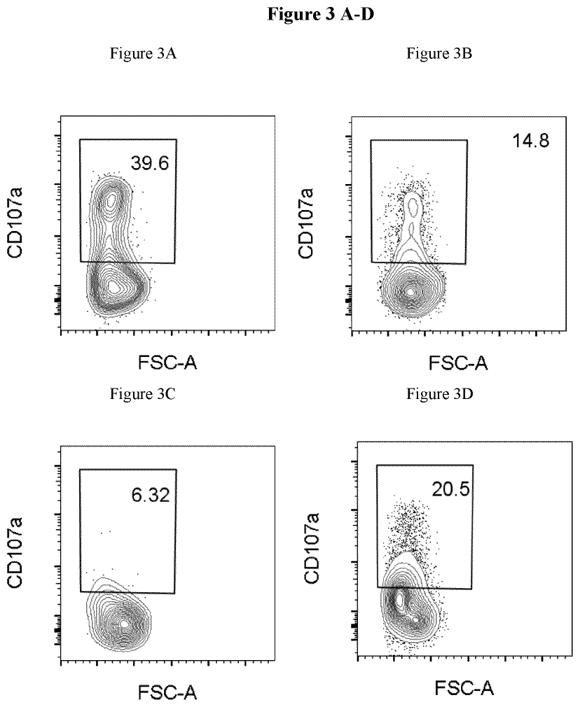

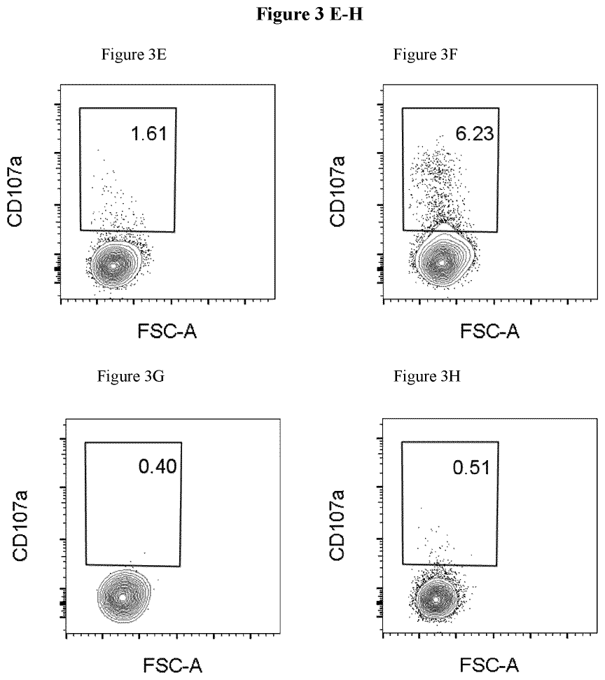

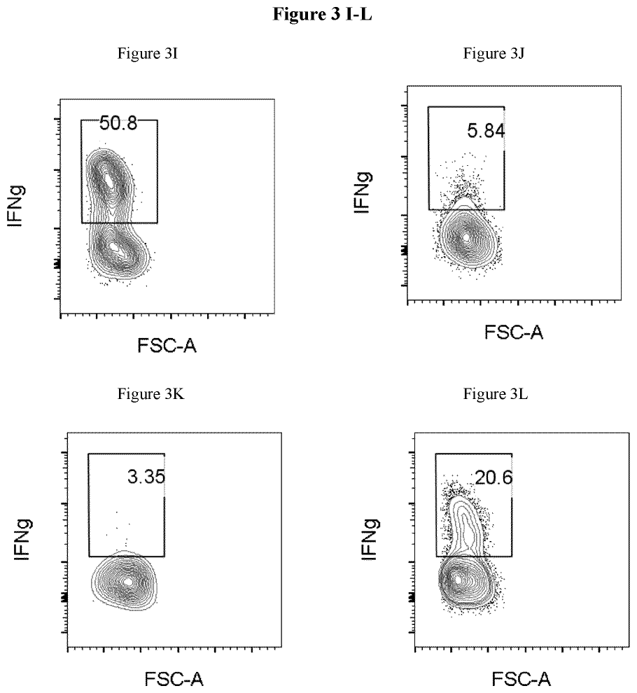

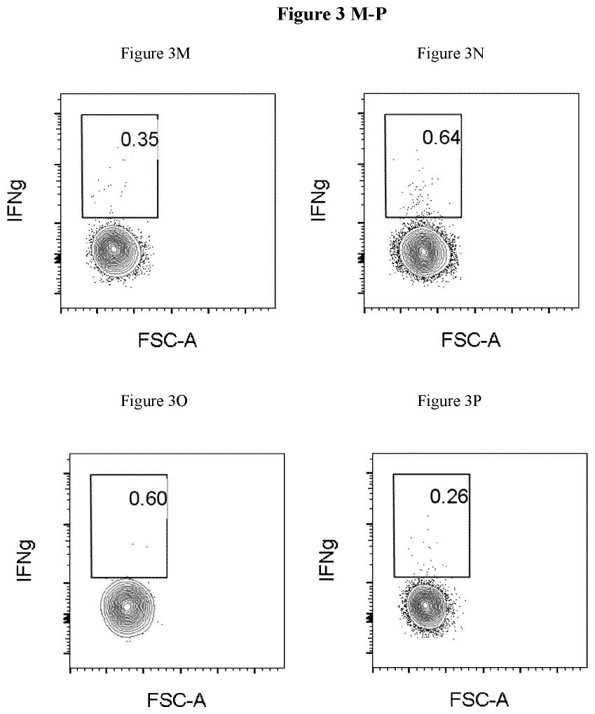

Both prodrugs XC1 and XD1 dose-dependently induced IFNγ production and degranulation (i.e. CD107a) of the Vδ2 γδ T cell population in primary human PBMCs (FIG. 2 A, C), without inducing direct activation of other immune cells (NK cells, CD8+ T-cells, Vδ1 γδ T-cells) (FIG. 2 B, D). The positive control phytohaemagglutinin (PHA-M) was able to activate all immune cells (FIG. 3A-D, I-L), indicating that they retained their immunogenic potential. IFNγ production and degranulation of Vδ2 γδ T-cells was more potently induced by Raji cells that were pre-treated with XC1 and XD1 compared to pretreatment with HMBPP (FIG. 2 A, C).

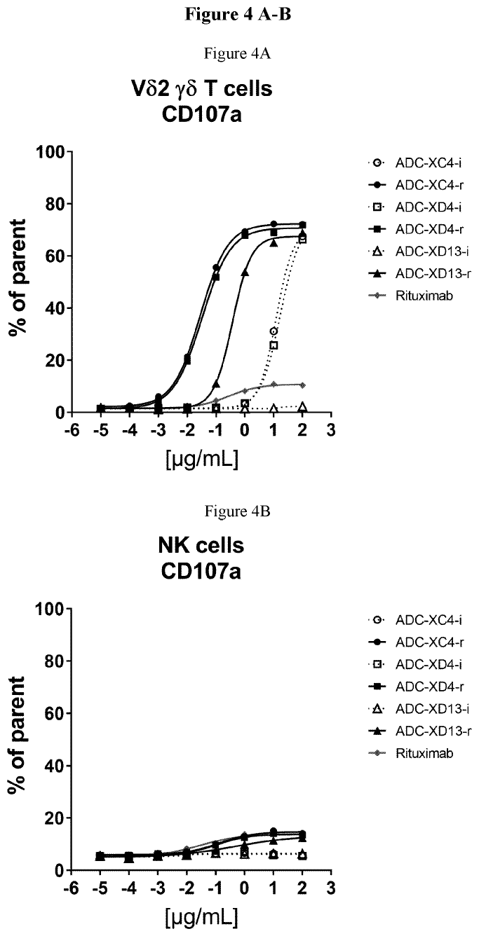

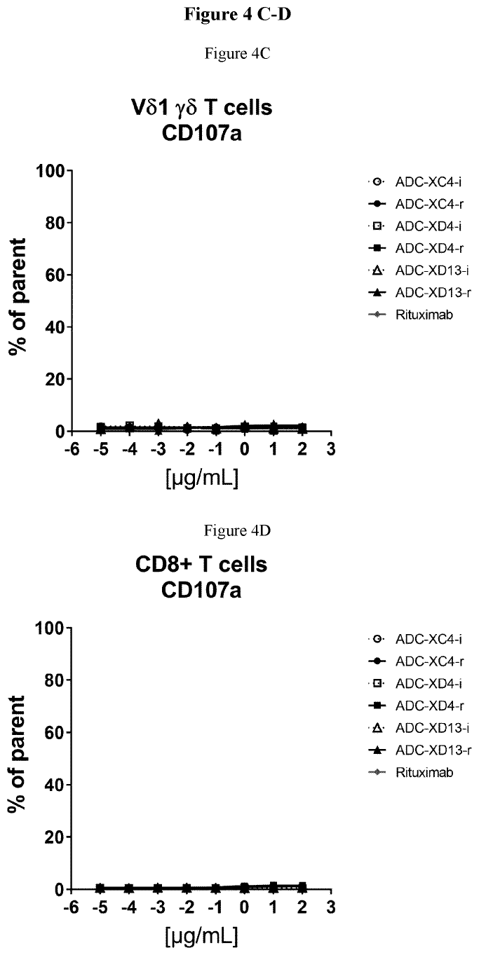

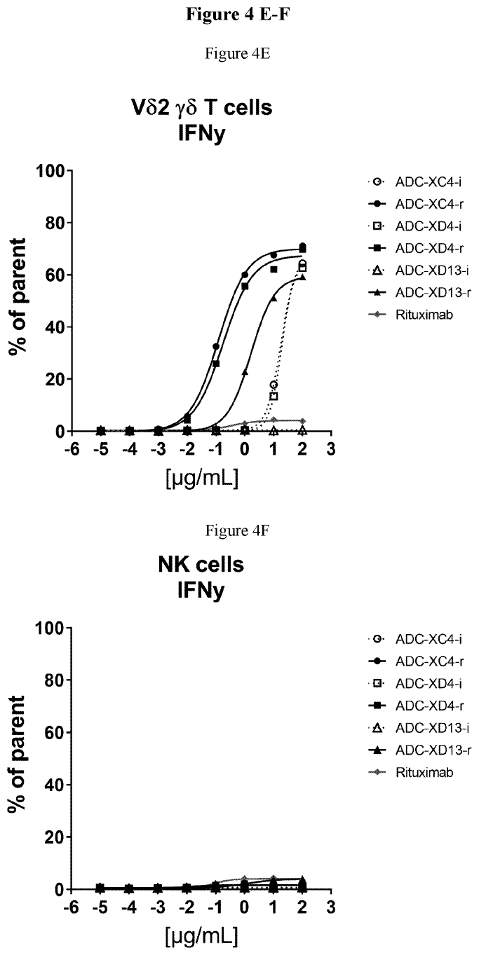

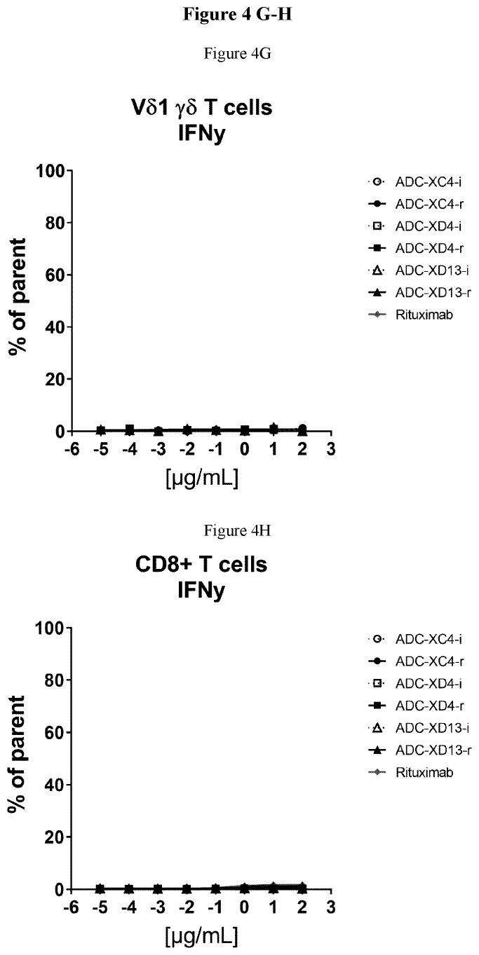

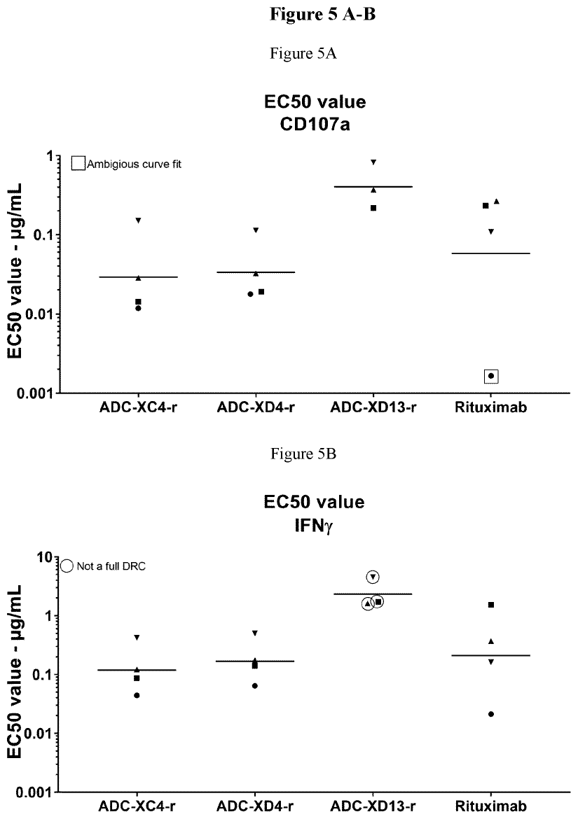

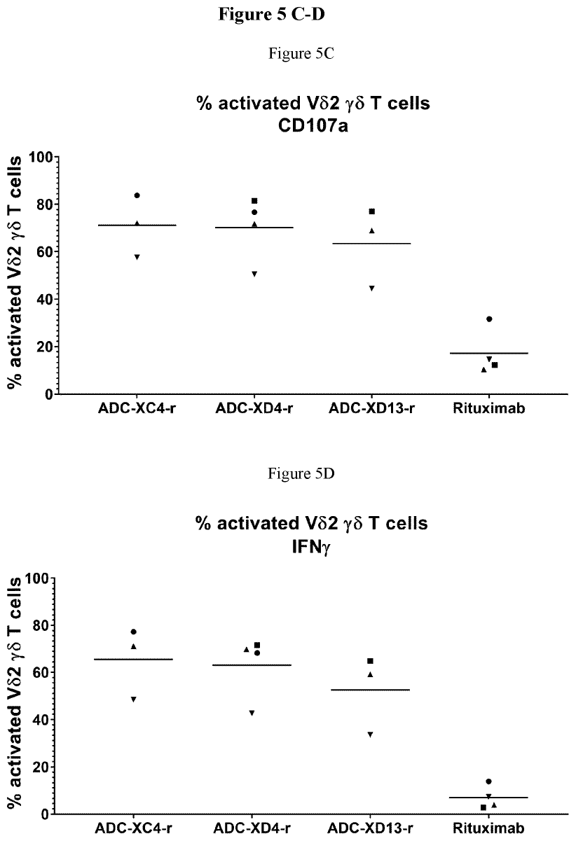

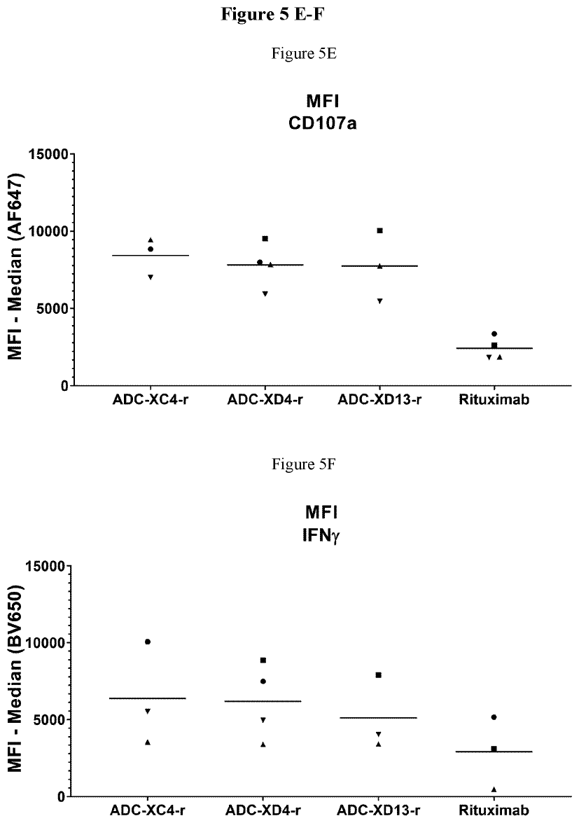

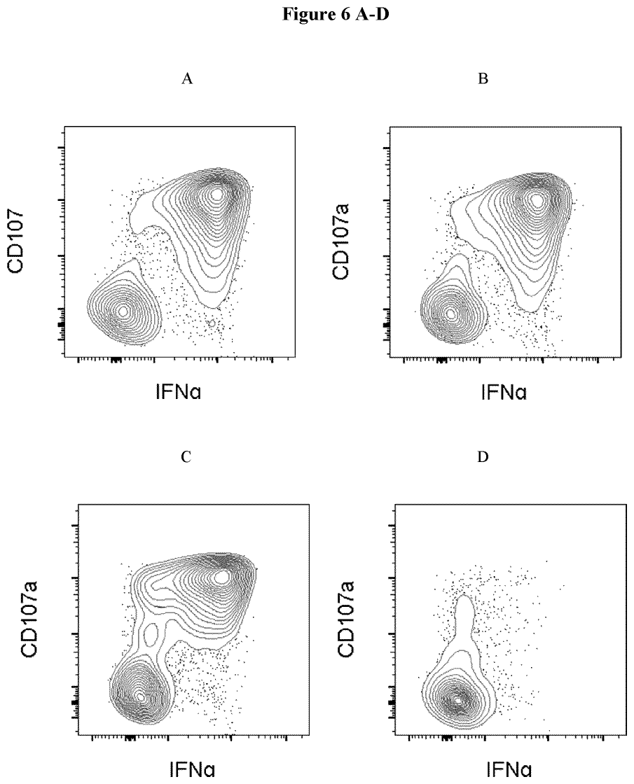

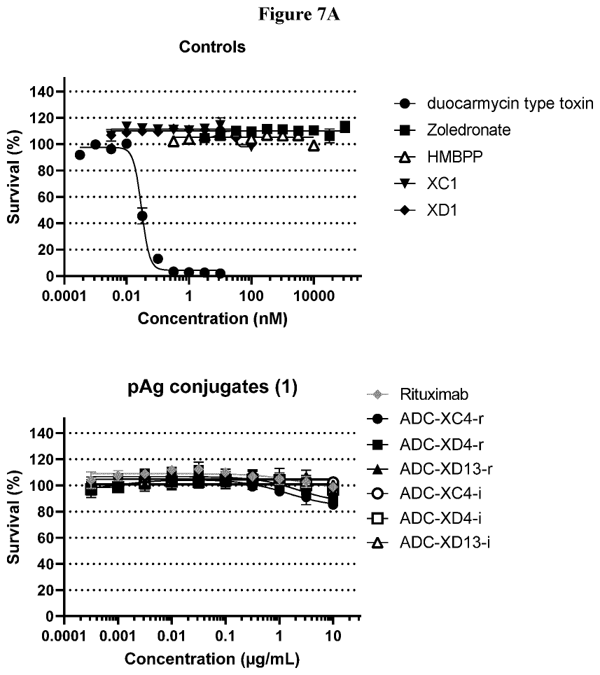

Three prodrugs were conjugated to rituximab or a non-binding isotype control through a cleavable linker as described in Example 4. Raji cells pre-incubated with the CD20-targeting ADC-XC4-r, ADC-XD4-r and ADC-XD13-r dose-dependently induced IFNγ production by Vδ2 γδ T cell with an average EC₅₀ of 169, 220, 2622 ng/ml, and degranulation (CD107a) with an average EC₅₀ of 51.3, 45.5, 470 ng/ml, respectively (FIGS. 4, 5A, B, 6, table 3). Potent Vδ2 γδ T cell activation relied on target-cell mediated ADC activation, as the respective non-binding control ADC-XC4-i and ADC-XD4-i induced Vδ2 γδ T cell activation with low potency, and the non-binding control ADC-XD13-i did not induce IFNγ production or degranulation of Vδ2 γδ T-cells (FIG. 4).

As expected, Raji cells pretreated with the CD20-binding antibody rituximab also activated Vδ2 γδ T-cells. However, the rituximab-ADCs induced degranulation and IFNγ-production in more Vδ2 γδ T-cells than rituximab itself (FIG. 5C,D), and also induced higher CD107a and IFNγ expression per cell (FIG. 5E, F, 6).

Rituximab pre-treated Raji cells also activated NK cells, most likely through FcγRs that are well-known to be expressed by NK cells. ADC-pretreated Raji cells did not further enhance the proportion of activated NK cells (FIG. 4 B, F). These results showed that pretreatment of tumor cells with the described CD20-binding ADCs led to dose dependent, more potent induction of IFNγ and degranulation of Vδ2 γδ T-cells than with rituximab, and depended on target binding and internalization indicating that our ADCs can improve the anti-tumor immune response. The ADCs have an active Fc tail that activated other immune cells, most likely through well-defined FcγR interactions.

TABLE 3
IFNγ or CD107a expression by indicated immune cell
populations induced by prodrug-pre-treated Raji cells.

| donor | | | | | | | |
|---|---|---|---|---|---|---|---|
| A | | B | | C | | D | |
| EC₅₀ | CI | EC₅₀ | CI | EC₅₀ | CI | EC₅₀ | CI |

ADC-XC4-r

| | | | | | | | | |
|---|---|---|---|---|---|---|---|---|
| IFNγ | 44 | 21-95 | 86 | 62-122 | 122 | 98-153 | 425 | 252-799 |
| CD107a | 12 | 8-17 | 14 | 8-26 | 29 | 26-32 | 151 | 61-517 |

| Donor | | | | | | | |
|---|---|---|---|---|---|---|---|
| A | | B | | C | | D | |
| EC₅₀ | CI | EC₅₀ | CI | EC₅₀ | CI | EC₅₀ | CI |

ADC-XD4-r

| | | | | | | | | |
|---|---|---|---|---|---|---|---|---|
| IFNγ | 64 | 52-78 | 141 | 102-197 | 176 | 110-296 | 500 | 306-904 |
| CD107a | 18 | 8-39 | 19 | 7-49 | 33 | 27-39 | 113 | 87-148 |

ADC-XD13-r

| | | | | | | | | |
|---|---|---|---|---|---|---|---|---|
| IFNγ | n.d. | n.d. | 1711 | 1665-1760 | 1621 | 1268-2136 | 4534 | 3328-6483 |
| CD107a | n.d. | n.d. | 217 | 193-244 | 371 | 304-450 | 823 | 596-1198 |

rituximab

| | | | | | | | | |
|---|---|---|---|---|---|---|---|---|
| IFNγ | 21 | n.d. | 1532 | 830-3535 | 371 | 141-858 | 162 | 112-252 |
| CD107a | 2 | # | 233 | 171-321 | 267 | 151 to 481 | 109 | 38 to 353 |

EC₅₀ (ng/ml) values represent the concentration at which 50% of the activation is achieved.
n.d. = not determined
# = Could not be determined by GraphPad prism

### Example 24: Vδ2 γδ T-Cell Activity Induced by Different pAg Conjugates

Multiple synthesized phosphoantigen (pAg) conjugates were linked to rituximab (anti-CD20) and tested for their ability to selectively activate Vδ2 γδ T-cells after overnight incubation with CD20-positive Raji cells. The tested conjugates are within the list reflected in Table 1. The pretreated Raji cells were cocultured with PBMCs and activation (IFNγ and TNFα production) and degranulation (CD107a) of Vδ2 γδ T-cells, Vδ1 γδ T-cells, CD8 positive T-cells and NK-cells was determined using multicolor flow cytometry as described in Example 23.

### Material and Methods

### Cellular Binding

Raji cells were cultured as described in Example 23. For cellular binding in a 96 well plate, 100,000 Raji cells/well were washed twice with ice-cold FACS buffer (PBS 1×, 0.1% v/w BSA, 0.02% v/v Sodium Azide (NaN3)), followed by the addition of a concentration range of 50 μL/well of a pAg conjugate, naked antibody (e.g. rituximab) or non-binding isotype control pAg conjugate diluted in ice-cold FACS buffer. After an incubation time of 30 minutes at 4° C., the cells were washed twice with ice-cold FACS buffer. Then, 50 μL/well APC-conjugated secondary F(ab′)₂ goat anti-Human IgG (Fc fragment specific, Jackson Immuno research, 109-136-098, 1:6000 or 1:500) was added. After 30 minutes at 4° C., cells were washed twice and resuspended in 150 μL ice-cold FACS buffer. Fluorescence intensities were determined by flow cytometry using the FACSVerse or FACSymphony (BD Biosciences) and indicated as the median fluorescence intensity (MFI). Curves were fitted by nonlinear regression with a variable slope (four parameters) in GraphPad Prism version 9. EC₅₀ values were calculated in GraphPad Prism as the concentration in μg/mL that gives a response halfway between bottom and top of the curve. Binding experiments were performed N=2 or N=3 times.

### Determination of Direct Compound-Related Cell Death

Raji cells were plated in CGM (complete growth medium, RPMI-1640 (Gibco-Life Technologies; Carlsbad, CA) supplemented with 10% Heat-inactivated (HI) Fetal Bovine Serum (FBS) (Gibco-Life Technologies; Carlsbad, CA) and 80 U/mL Penicillin-Streptomycin solution (Gibco-Life Technologies; Carlsbad, CA) in 96 well plates (90 μL/well, 1000 cells/well) or 384 well plates (45 μL/well, 500 cells/well) and incubated in a humidified incubator containing 5% CO₂ at 37° C. After an overnight incubation, 10 μL or 5 μL of a concentration range of antibody (e.g. rituximab), pAg conjugate or control compound (Toxin (duocarmycin type): Cyclopropyl DC1) was added. Metabolic activity was assessed after 6 days, using the CellTiter-Glo™ (CTG) luminescent assay kit from Promega Corporation (Madison, WI) according to the manufacturer's instructions. Cell viability was expressed as the percentage survival relative to the average mean of untreated cells or vehicle-treated cells (only growth medium or 1% DMSO) multiplied with 100. The efficacy was calculated by subtracting the bottom of the dose response curve (DRC) from 100%.

### Functional Assay (Determining γδ T-Cell Activity Induced by Different pAg Conjugates).

The functional assay was performed as disclosed in Example 23. In addition to anti-IFNγ BV650, also anti-TNFα PE (BD Biosciences, San Jose, CA, USA, clone Mab11) was included in most of the experiments during the intracellular cytokine staining step. The highest compound concentration used for pretreatment of Raji cells was 150 μg/mL for antibodies/conjugates. EC₅₀ values were calculated in GraphPad Prism as the concentration in μg/mL that gives a response half way between bottom and top of the curve. In the summary graphs, the ‘% activated (cells)’ and the CD107a, IFNγ and TNFα MFI was determined from samples cocultured with Raji cells pretreated with the highest compound concentration (e.g. 150 μg/mL for antibodies and pAg conjugates, or 30 or 6 μg/mL when data from the highest compound suffered from technical problems).

### Results/Conclusion

Rituximab-Conjugates Activate Vδ2 γδ T-Cells with Higher Efficacy and Potency than Rituximab

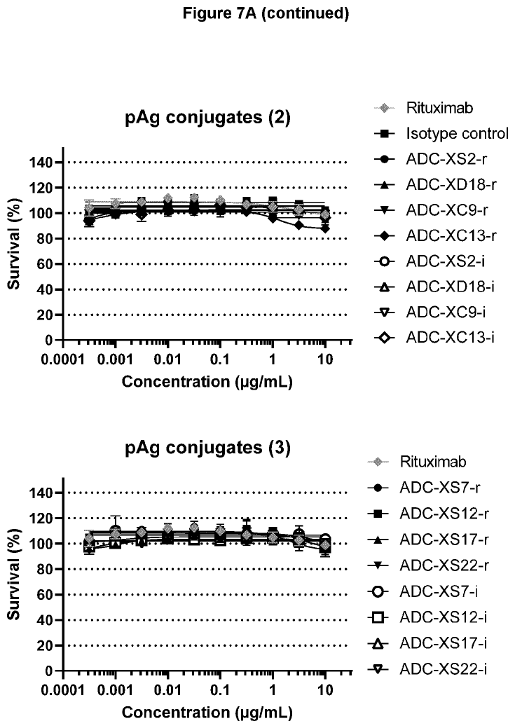

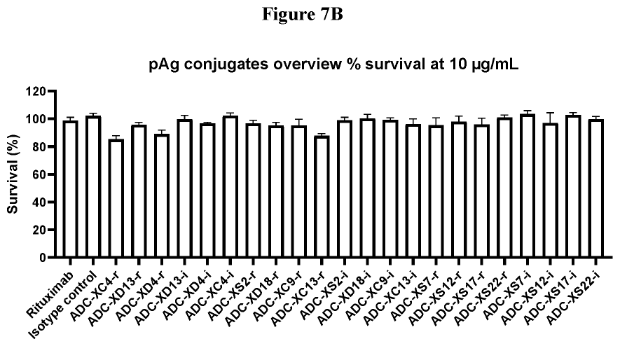

Multiple pAg conjugates linked to rituximab and non-binding controls were generated with a drug-to-antibody-ratio of ˜2. Their binding to Raji cells was comparable to naked rituximab (Table 4) and the non-binding isotype controls did not show binding. It was also determined if the pAg conjugates have direct cytotoxic effects to CD20-positive Raji tumor cells. A concentration range of the pAg conjugates ADC-XC4-r, ADC-XD4-r, ADC-XD-13-r, ADC-XS2-r, ADC-XD18-r, ADC-XC9-r, ADC-XC13-r, ADC-XS7-r, ADC-XS12-r, ADC-XS17 and ADC-XS22-r and respective non-binding control pAg conjugates were incubated for 6 days with Raji cells and cell survival was determined using CellTiter-Glo®. None of the tested pAg conjugates, HEMBPP and zoledronate, induced substantial (>15%) direct compound related cell death while the positive control, a duocarmycin type toxin, was very effective. (FIG. 7).

Therefore, other pAg conjugates were not tested anymore.

TABLE 4
Binding of rituximab pAg conjugates to Raji cells, compared
to naked rituximab. EC₅₀ (μg/ml) values represent the
concentration at which 50% of the activation is achieved.

| | | EC₅₀ | |
|---|---|---|---|
| pAg conjugate | Compound | (μg/mL) | Confidence interval |
| Experiment 1 and 2 | Rituximab | 0.6 | 0.3452-1.118 |
| | ADC-XC4-r | 0.41 | 0.2405-0.6843 |
| | ADC-XC4-i | >150 | N/A |
| | ADC-XD13-r | 0.76 | 0.4916-1.202 |
| | ADC-XD13-i | >150 | N/A |
| | ADC-XD4-r | 0.42 | 0.2274-0.7149 |
| | ADC-XD4-i | >150 | N/A |
| | ADC-XS2-r | 0.56 | 0.2949-1.053 |
| | ADC-XS2-i | >150 | N/A |
| | ADC-XD18-r | 0.55 | 0.2754-1.188 |
| | ADC-XD18-i | >150 | N/A |
| | ADC-XC9-r | 0.50 | 0.2419-0.966 |
| | ADC-XC9-i | >150 | N/A |
| | ADC-XC13-r | 0.40 | 0.2307-0.6524 |
| | ADC-XC13-i | >150 | N/A |
| | ADC-XS7-r | 0.31 | 0.1551-0.5681 |
| | ADC-XS7-i | >150 | N/A |
| | ADC-XS12-r | 0.48 | 0.3316-0.6948 |
| | ADC-XS12-i | >150 | N/A |
| | ADC-XS17-r | 0.59 | 0.392-0.8666 |
| | ADC-XS17-i | >150 | N/A 0 |
| | ADC-XS22-r | 0.61 | 0.465-0.7931 |
| | ADC-XS22-i | >150 | N/A |
| Experiment 3 and 4 | Rituximab | 1.221 | 0.8106-1.877 |
| | ADC-XD44-r | 1.858 | 0.8823-5.609 |
| | ADC-XD44-i | >150 | N/A |
| | ADC-XD45-r | 1.507 | 0.7492-3.601 |
| | ADC-XD45-i | >150 | N/A |
| | ADC-XD46-r | 2.629 | 1.775-4.294 |
| | ADC-XD46-i | >150 | N/A |
| Experiment 5, 6 and 7 | Rituximab | 3.451 | 1.977-7.527 |
| | ADC-XS24-r | 1.494 | † |
| | ADC-XS24-i | >150 | N/A |
| | ADC-XS25-r | 1.87 | † |
| | ADC-XS25-i | >150 | † |
| | ADC-XD65-r | 2.321 | 0.9457-16.12 |
| | ADC-XD65-i | >150 | N/A |
| | ADC₈-XD65-r | 1.888 | 0.8547-5.452 |
| | ADC₈-XD65-i | >150 | N/A |
| | ADC-XD58-r | 2.485 | 1.216-7.962 |
| | ADC-XD58-i | >150 | N/A |
| | ADC-XD63-r | 4.351 | 2.662-8.947 |
| | ADC-XD63-i | >150 | N/A |

† Confidence interval not reliable;
>150 = No dose-dependent response up to 150 μg/mL;
N/A = not applicable

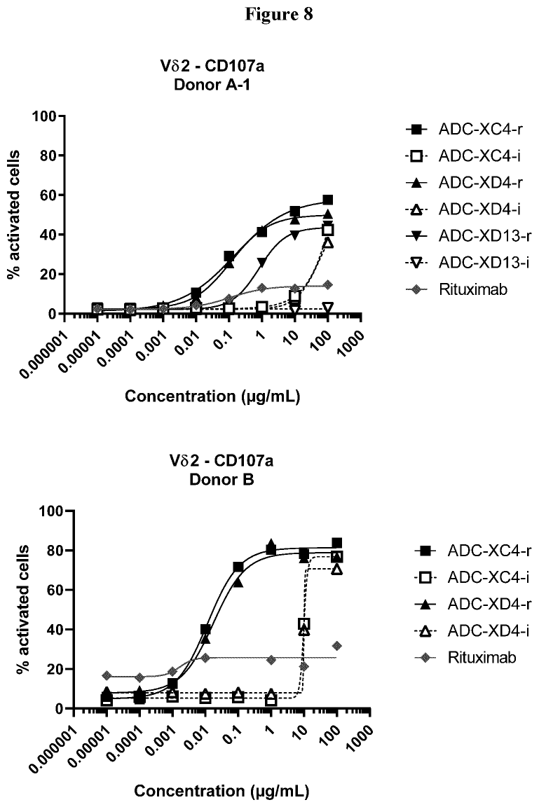

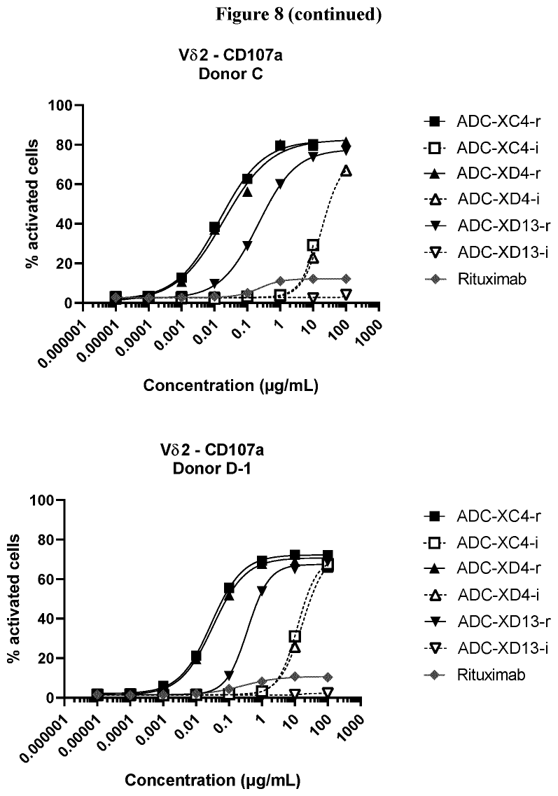

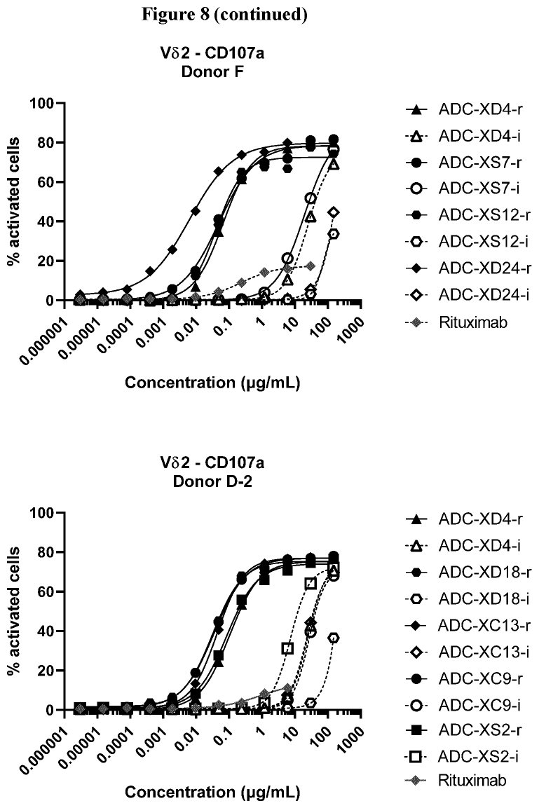

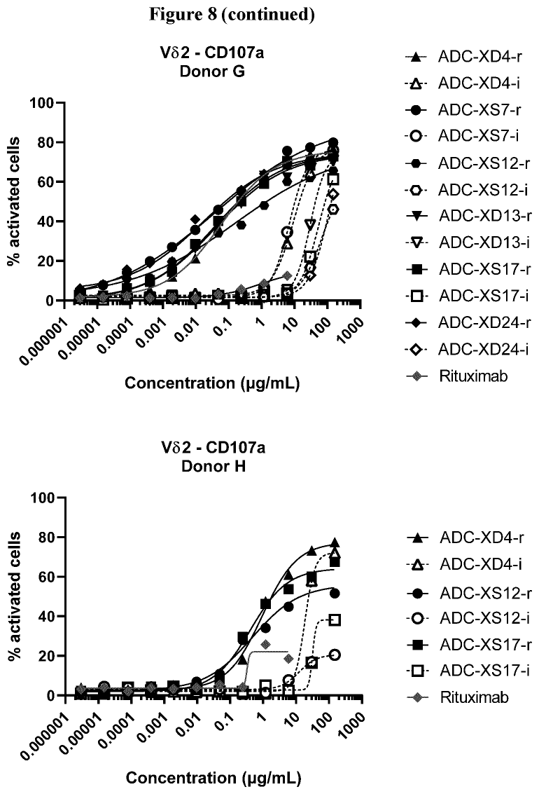

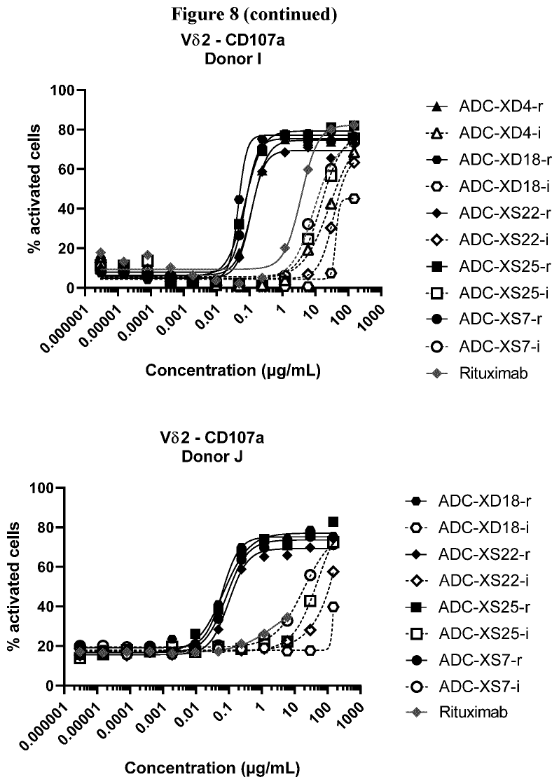

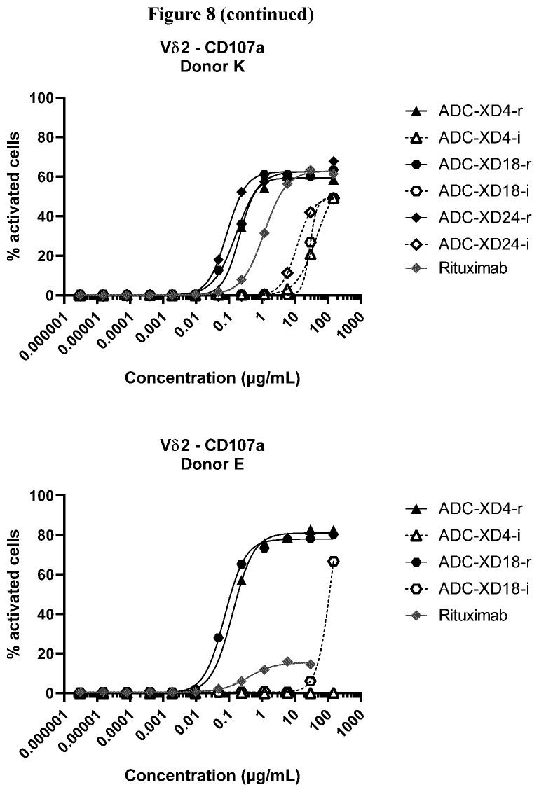

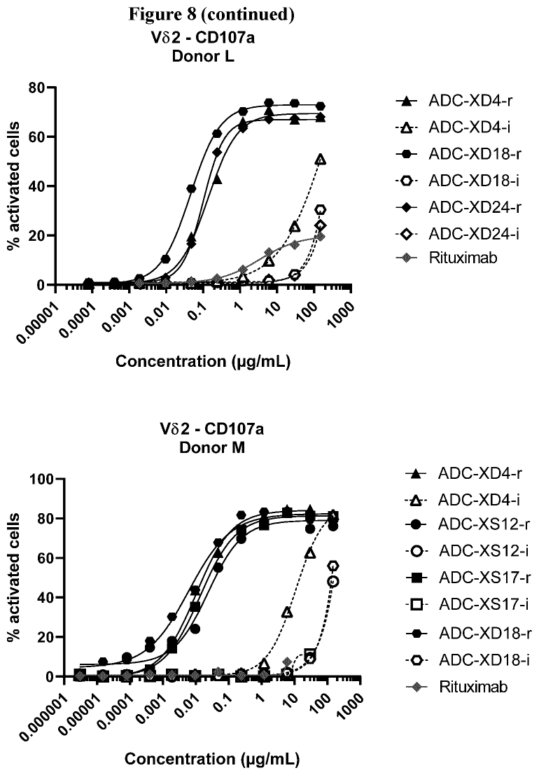

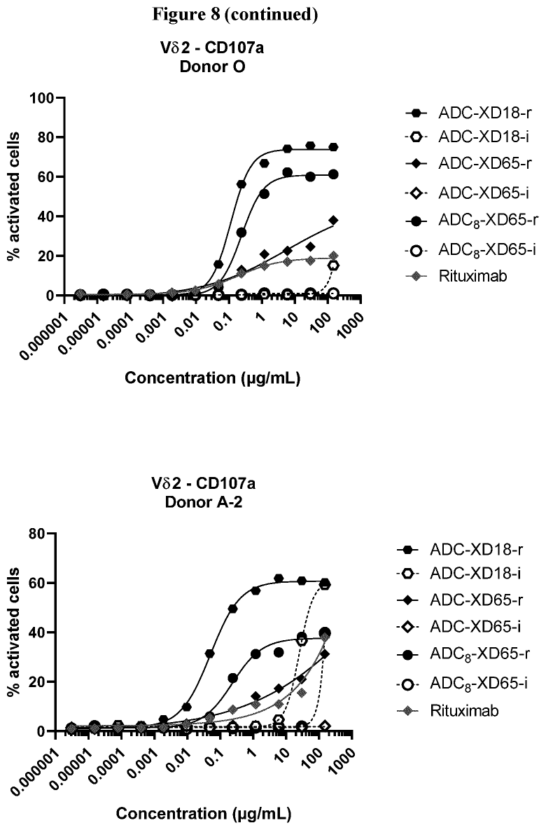

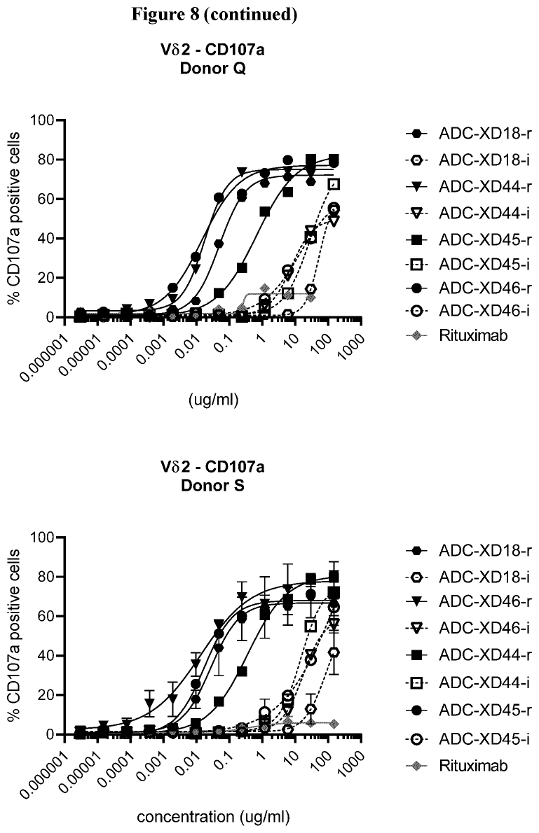

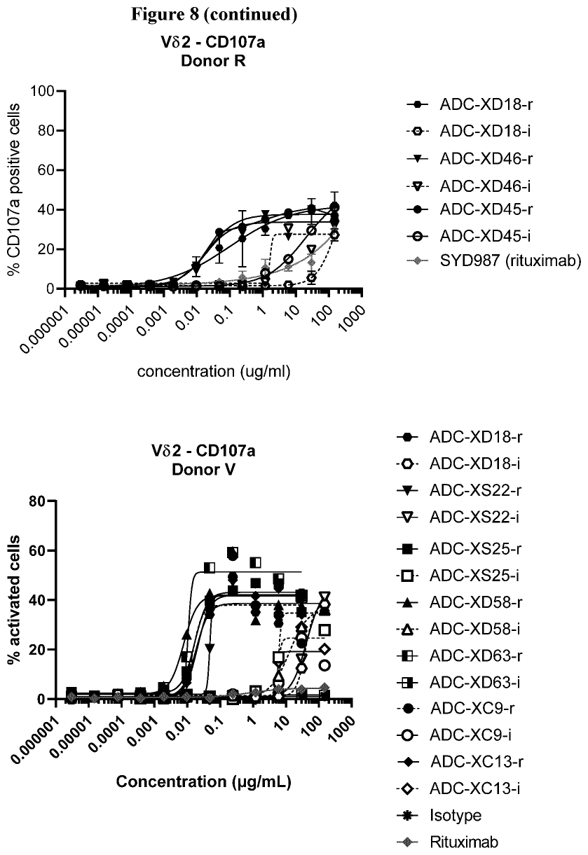

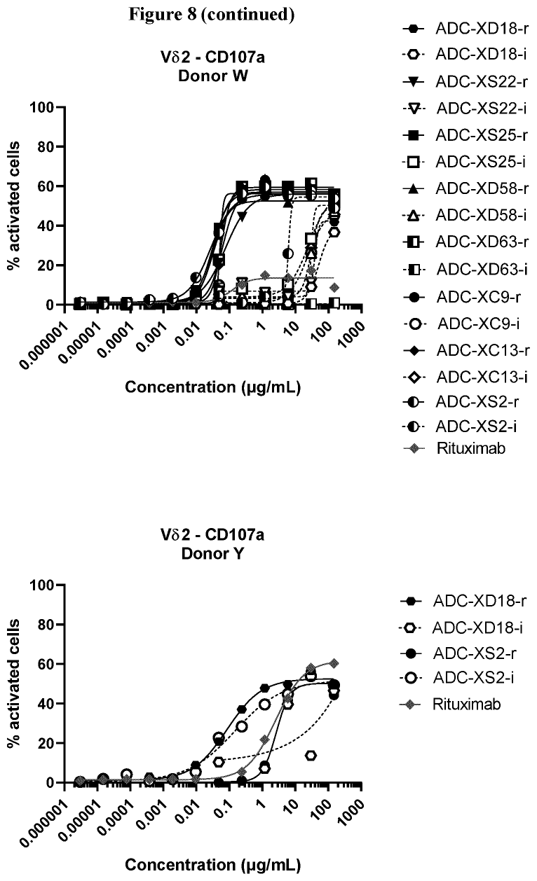

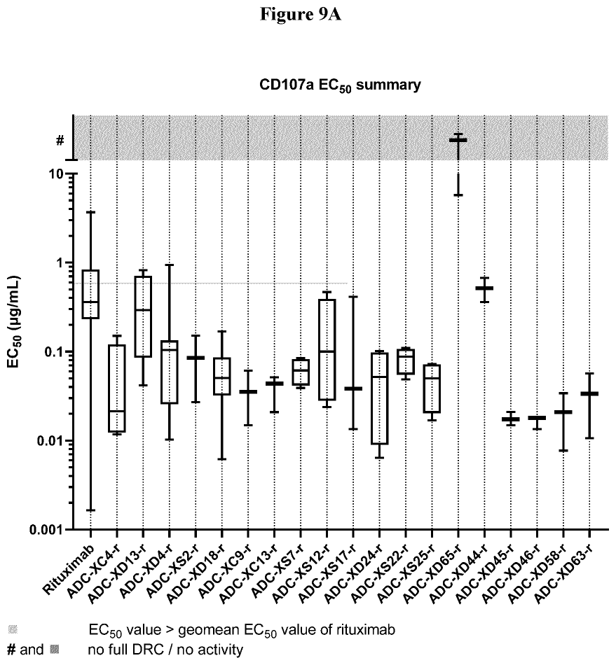

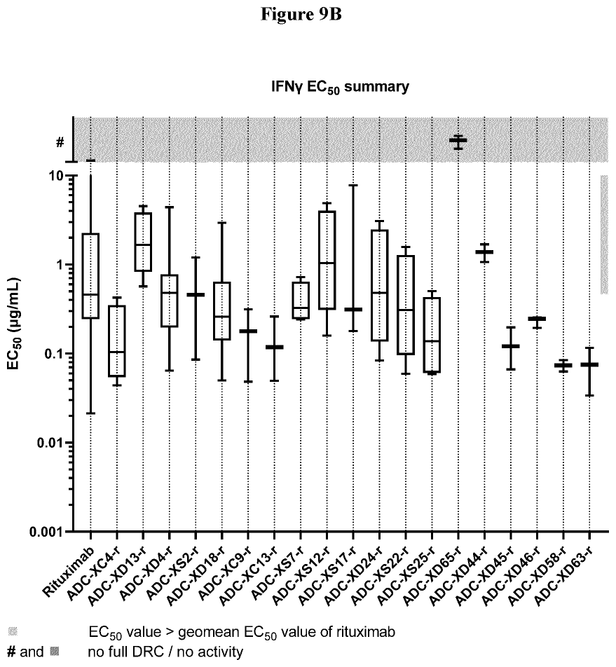

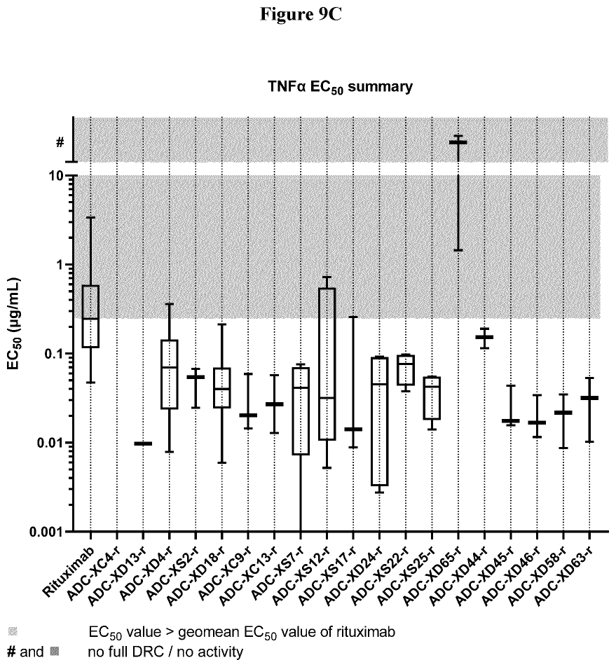

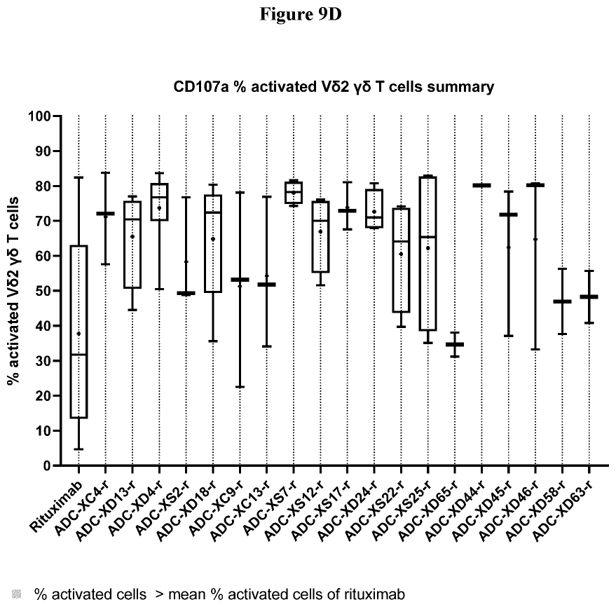

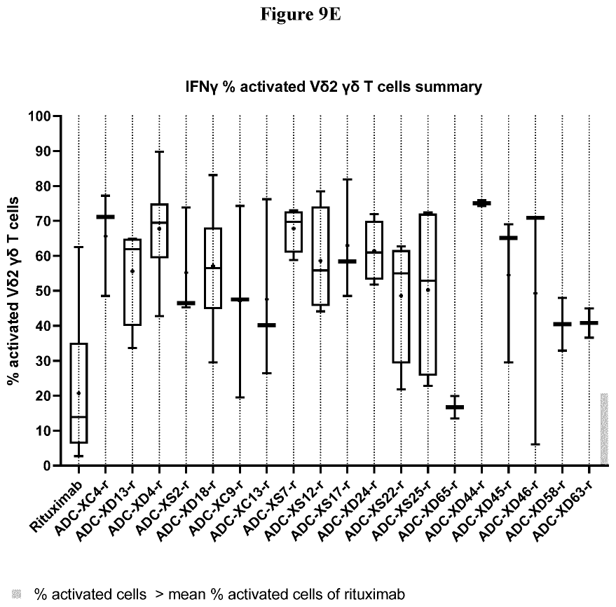

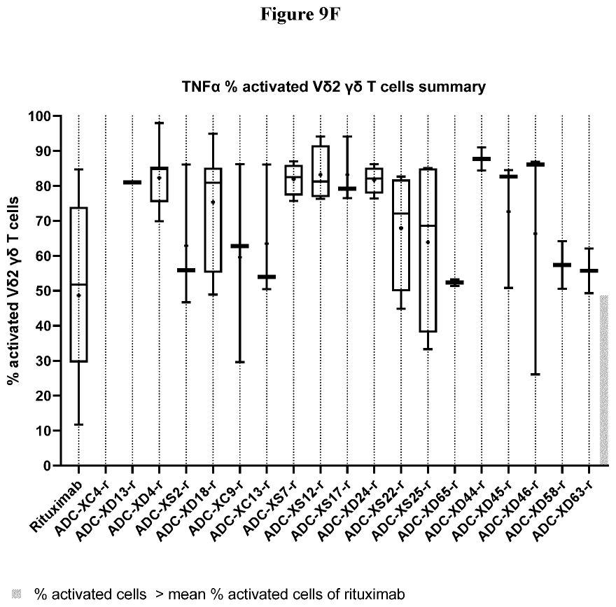

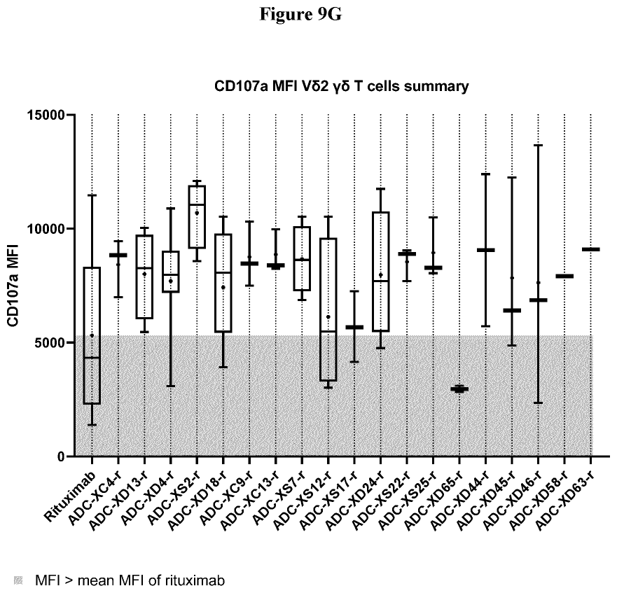

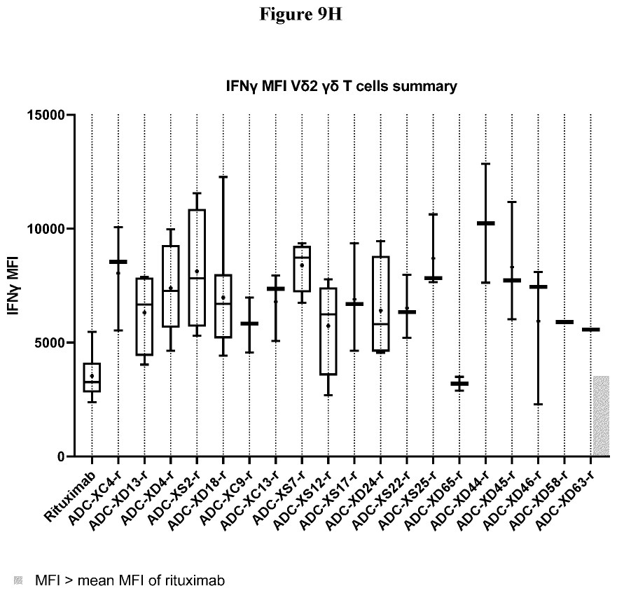

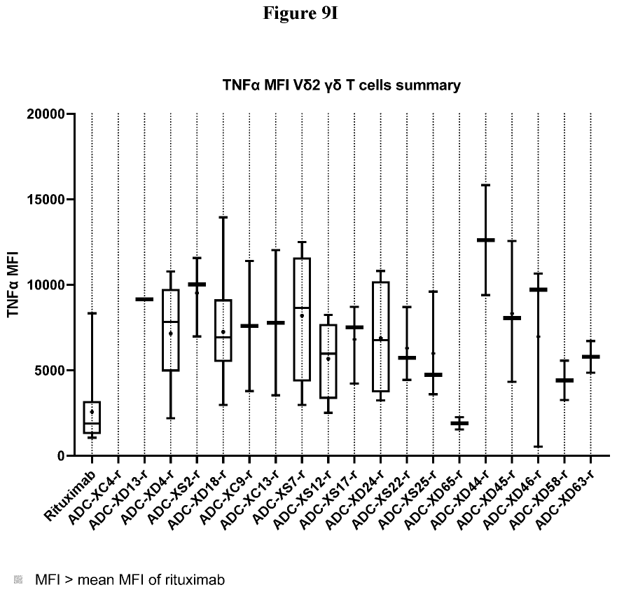

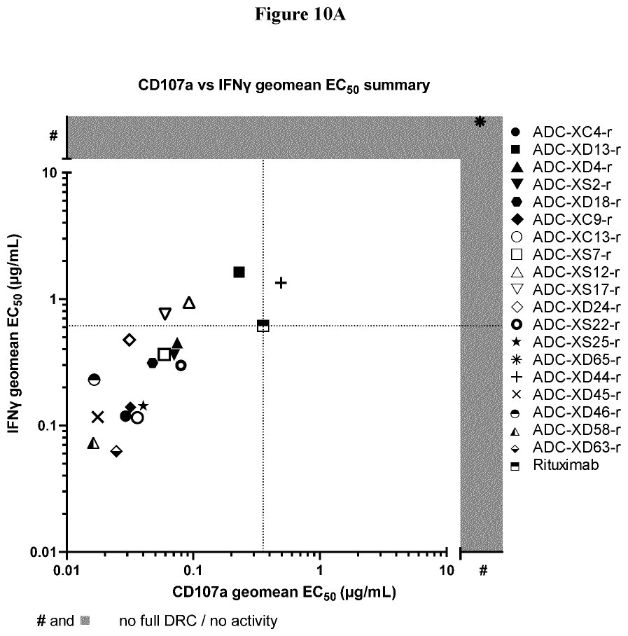

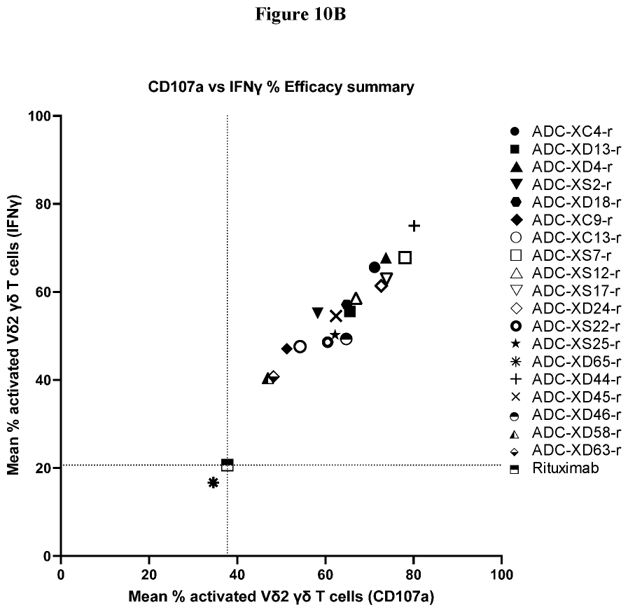

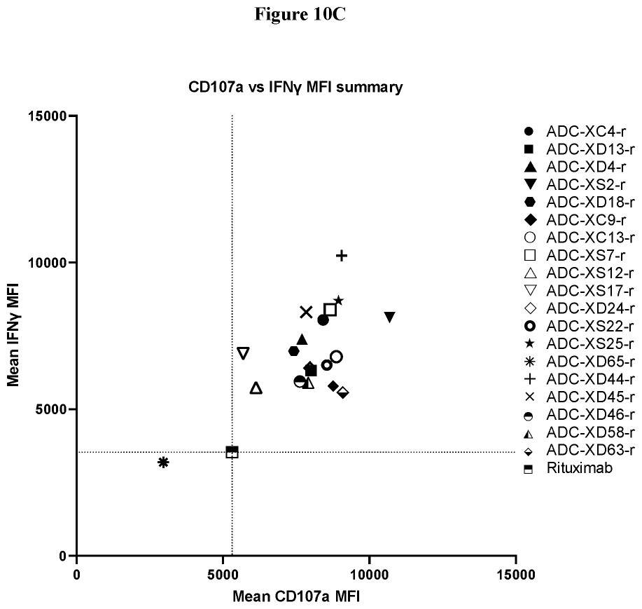

The generated pAg conjugates were tested for their ability to induce selective Vδ2 γδ T-cell activation after overnight incubation with Raji cells, followed by a 6 hour coculture with Vδ2 γδ T-cell containing PBMCs. Dose-response curves for Vδ2 γδ T-cell degranulation, IFNγ and TNFα production were generated (exemplified by CD107a production in FIG. 8) and these results showed that Raji cells pretreated with non-binding isotype controls pAg conjugates activated Vδ2 γδ T-cells with low potency and EC₅₀ values could not be calculated reliably. For the rituximab pAg conjugates, EC₅₀ values and efficacies were calculated (Table 5-10). Box plots depict an overview of CD107a, IFNγ and TNFα EC₅₀ values, efficacies and MFIs (FIG. 9). Degranulation induced by pAg conjugates correlated with IFNγ production (FIG. 10). Since TNFα production was not assessed in every experiment, correlations graphs were not generated for this cytokine.

The results depicted in FIG. 8-10 and Table 5-10 show that pAg conjugate pretreated Raji cells induced dose-dependent CD107a, IFNγ and TNFα production, which was more potent (lower EC₅₀) for most pAg conjugates when compared to rituximab pretreatment, except for ADC-XD65-r (higher CD107a, IFNγ and TNFα EC₅₀ values), ADC-XD44-r (higher CD107a and IFNγ EC₅₀ values) and ADC-XD13-r and ADC-XS12-r (higher IFNγ EC₅₀ values). Except for ADC-XD65-r, all pAg conjugates activated more Vδ2 γδ T-cells (% activity) than rituximab and the amount of produced cytokines and degranulation (MFI) was higher. Overall, these results showed that Raji cells preincubated with pAg conjugates potently and efficaciously activated Vδ2 γδ T-cells.

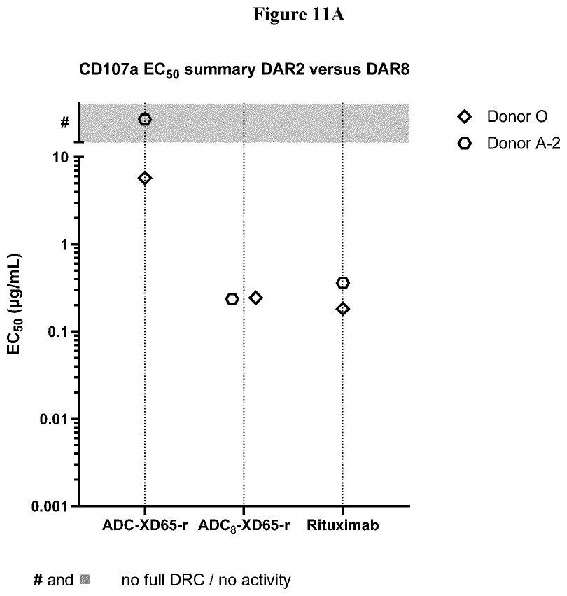

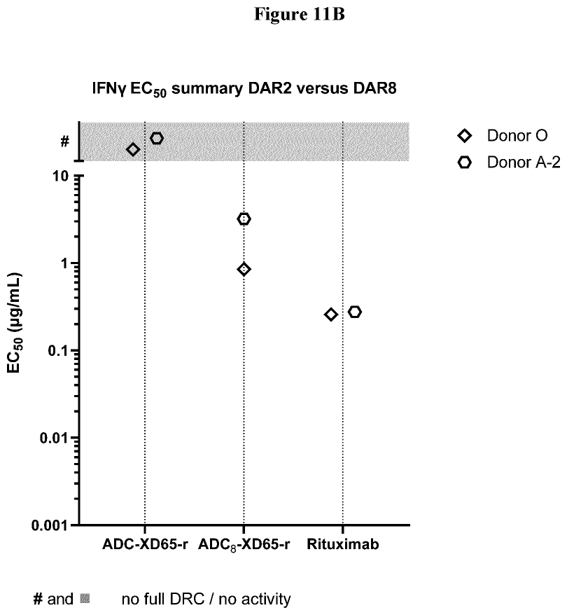

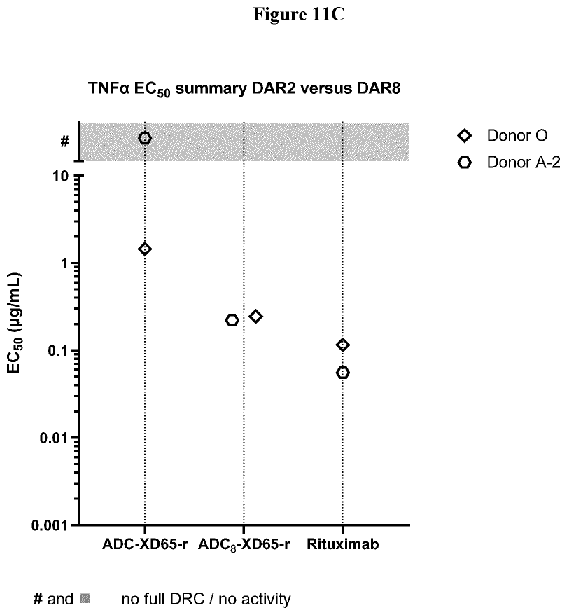

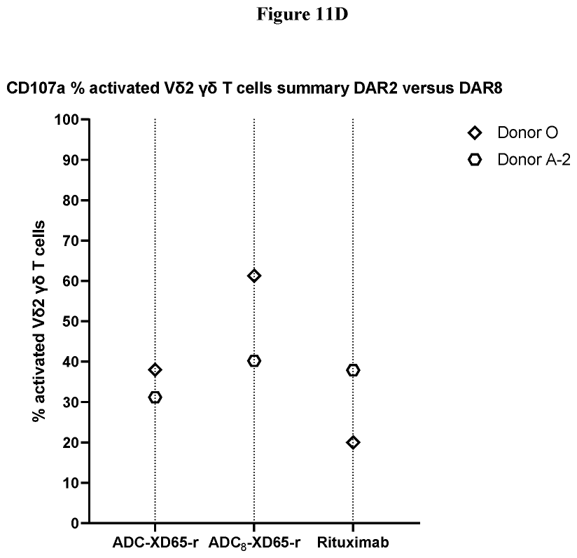

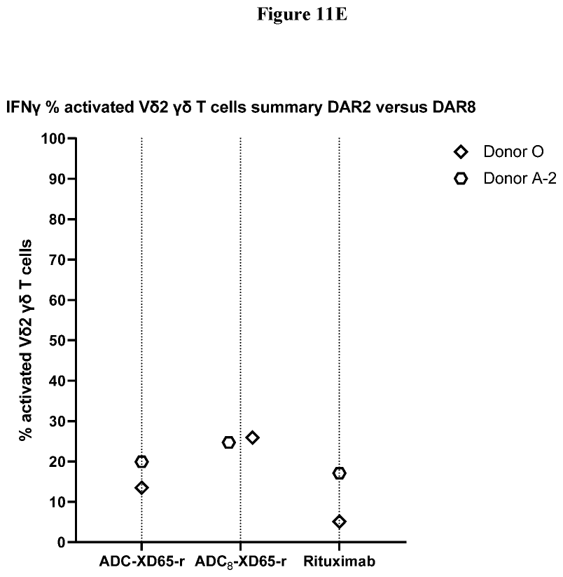

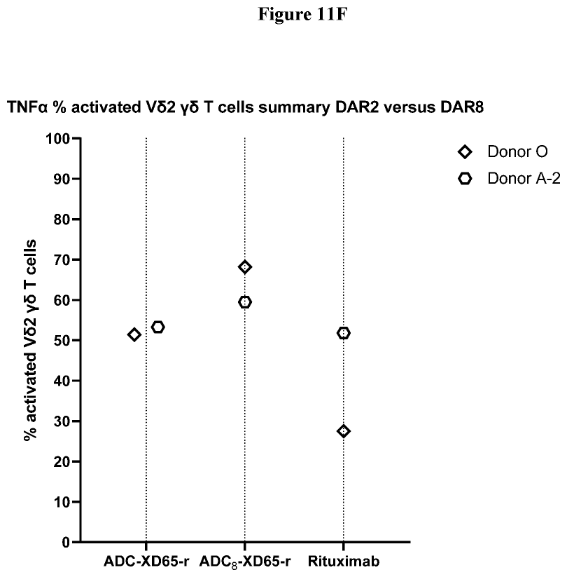

While most pAg conjugates were potent and efficacious in inducing Vδ2 γδ T-cell activity, ADC-XD65-r was less potent and efficacious than rituximab. Therefore, a DAR8 of this compound was generated. When pretreated with Raij cells, ADCs-XD65-r was more potent and efficacious in inducing Vδ2 γδ T-cell activation as measured by CD107a, IFNγ and TNFα production compared to ADC-XD65-r (FIG. 11). Thus, increasing the DAR leads to improved activity of low-potent pAg conjugates.

TABLE 5
CD107a EC₅₀ (μg/mL) by Vδ2 γδ T-cells induced by rituximab pAg conjugate
or rituximab pretreated Raji cells. When fields are empty, no EC₅₀ values were determined
(not tested). The letters indicate the donors that were used. When the same donor was
tested in two different experiments this was indicated with ‘1’ or ‘2’. Rmab = rituximab.

| | ADC- | | | | | | | | | |
|---|---|---|---|---|---|---|---|---|---|---|
| | XC4-r | XD13-r | XD4-r | XS2-r | XD18-r | XC9-r | XC13-r | XS7-r | XS12-r | XS17-r |
| A1 | 0.15 | 0.82 | 0.11 | | | | | | | |
| B | 0.01 | | 0.02 | | | | | | | |
| C | 0.01 | 0.22 | 0.02 | | | | | | | |
| D1 | 0.03 | 0.37 | 0.03 | | | | | | | |
| F | | | 0.07 | | | | | 0.05 | 0.04 | |
| D2 | | | 0.10 | 0.08 | 0.03 | 0.04 | 0.04 | | | |
| G | | 0.04 | 0.06 | | | | | 0.04 | 0.16 | 0.04 |
| H | | | 0.94 | | | | | | 0.47 | 0.41 |
| I | | | 0.12 | | 0.05 | | | 0.07 | | |
| J | | | | | 0.06 | | | 0.08 | | |
| K | | | 0.21 | | 0.17 | | | | | |
| E | | | 0.13 | | 0.07 | | | | | |
| L | | | 0.14 | | 0.05 | | | | | |
| M | | | 0.01 | | 0.01 | | | | 0.02 | 0.01 |
| O | | | | | 0.12 | | | | | |
| A2 | | | | | 0.05 | | | | | |
| Q | | | | | 0.05 | | | | | |
| R | | | | | 0.10 | | | | | |
| S | | | | | 0.02 | 0.01 | 0.02 | | | |
| V | | | | 0.03 | 0.03 | 0.06 | 0.05 | | | |
| W | | | | 0.15 | 0.09 | | | | | |
| Y | | | | | 0.03 | | | | | |
| | ADC- | | | | | | | | | |
| | XD24-r | XS22-r | XS25-r | XD65-r | XD44-r | XD45-r | XD46-r | XD58-r | XD63-r | Rmab |
| A1 | | | | | | | | | | 0.11 |
| B | | | | | | | | | | 0.002 |
| C | | | | | | | | | | 0.23 |
| D1 | | | | | | | | | | 0.27 |
| F | 0.01 | | | | | | | | | 0.24 |
| D2 | | | | | | | | | | 0.83 |
| G | 0.02 | | | | | | | | | † |
| H | | | | | | | | | | 0.33 |
| I | | 0.11 | 0.07 | | | | | | | 3.68 |
| J | | 0.10 | 0.07 | | | | | | | † |
| K | 0.09 | | | | | | | | | 1.18 |
| E | | | | | | | | | | 0.38 |
| L | 0.10 | | | | | | | | | 3.39 |
| M | | | | | | | | | | † |
| O | | | | 5.76 | | | | | | 0.18 |
| A2 | | | | † | | | | | | 0.36 |
| Q | | | | | 0.67 | 0.01 | 0.02 | | | 0.26 |
| R | | | | | | 0.02 | 0.02 | | | 0.39 |
| S | | 0.05 | 0.02 | | | | | 0.01 | 0.01 | 0.82 |
| V | | 0.08 | 0.03 | | | | | 0.03 | 0.06 | 0.12 |
| W | | | | | | | | | | 2.58 |
| Y | | | | | 0.36 | 0.02 | 0.01 | | | 0.43 |

† = EC₅₀ value could not be determined reliably

TABLE 6
IFNγ EC₅₀ (μg/mL) by Vδ2 γδ T-cells induced by rituximab pAg conjugate
or rituximab pretreated Raji cells. When fields are empty, no EC₅₀ values were determined
(not tested). The letters indicate the donors that were used. When the same donor was
tested in two different experiments this was indicated with ‘1’ or ‘2’. Rmab = rituximab.

| | ADC- | | | | | | | | | |
|---|---|---|---|---|---|---|---|---|---|---|
| | XC4-r | XD13-r | XD4-r | XS2-r | XD18-r | XC9-r | XC13-r | XS7-r | XS12-r | XS17-r |
| A1 | 0.43 | 4.53 | 0.50 | | | | | | | |
| B | 0.04 | | 0.06 | | | | | | | |
| C | 0.09 | 1.71 | 0.14 | | | | | | | |
| D1 | 0.12 | 1.62 | 0.18 | | | | | | | |
| F | | | 0.48 | | | | | 0.40 | 0.16 | |
| D2 | | | 0.86 | 1.20 | 0.26 | 0.31 | 0.26 | | | |
| G | | 0.57 | 0.22 | | | | | 0.24 | 0.76 | 0.18 |
| H | | | 4.41 | | | | | | 4.91 | 7.78 |
| I | | | 0.30 | | 0.14 | | | 0.25 | | |
| J | | | | | 0.64 | | | 0.72 | | |
| K | | | 2.55 | | 2.93 | | | | | |
| E | | | 0.61 | | 0.41 | | | | | |
| L | | | 0.69 | | 0.37 | | | | | |
| M | | | 0.33 | | 0.12 | | | | 1.31 | 0.31 |
| O | | | | | 1.25 | | | | | |
| A2 | | | | | 0.24 | | | | | |
| Q | | | | | 0.18 | | | | | |
| R | | | | | 2.24 | | | | | |
| S | | | | | 0.05 | 0.05 | 0.05 | | | |
| V | | | | 0.09 | 0.07 | 0.18 | 0.12 | | | |
| W | | | | 0.46 | 0.31 | | | | | |
| Y | | | | | 0.17 | | | | | |
| | ADC- | | | | | | | | | |
| | XD24-r | XS22-r | XS25-r | XD65-r | XD44-r | XD45-r | XD46-r | XD58-r | XD63-r | Rmab |
| A1 | | | | | | | | | | 0.16 |
| B | | | | | | | | | | 0.02 |
| C | | | | | | | | | | 1.53 |
| D1 | | | | | | | | | | 0.37 |
| F | 0.08 | | | | | | | | | 0.18 |
| D2 | | | | | | | | | | 0.46 |
| G | 0.30 | | | | | | | | | † |
| H | | | | | | | | | | 0.24 |
| I | | 0.41 | 0.21 | | | | | | | 7.80 |
| J | | 1.57 | 0.50 | | | | | | | † |
| K | 3.08 | | | | | | | | | 2.26 |
| E | | | | | | | | | | 0.62 |
| L | 0.67 | | | | | | | | | 6.26 |
| M | | | | | | | | | | † |
| O | | | | 39.26 | | | | | | 0.26 |
| A2 | | | | † | | | | | | 0.28 |
| Q | | | | | 1.69 | 0.07 | 0.25 | | | 0.26 |
| R | | | | | | 0.20 | 0.26 | | | 0.48 |
| S | | 0.06 | 0.06 | | | | | 0.06 | 0.03 | 11.47 |
| V | | 0.21 | 0.07 | | | | | 0.08 | 0.12 | 0.13 |
| W | | | | | | | | | | 3.54 |
| Y | | | | | 1.07 | 0.12 | 0.19 | | | 0.76 |

† = EC₅₀ value could not be determined reliably

TABLE 7
TNFα EC₅₀ (μg/mL) by Vδ2 γδ T-cells induced by rituximab pAg conjugate
or rituximab pretreated Raji cells. When fields are empty, no EC₅₀ values were determined
(not tested). The letters indicate the donors that were used. When the same donor was
tested in two different experiments this was indicated with ‘1’ or ‘2’. Rmab = rituximab.

| | ADC- | | | | | | | | | |
|---|---|---|---|---|---|---|---|---|---|---|
| | XC4-r | XD13-r | XD4-r | XS2-r | XD18-r | XC9-r | XC13-r | XS7-r | XS12-r | XS17-r |
| A1 | | | | | | | | | | |
| B | | | | | | | | | | |
| C | | | | | | | | | | |
| D1 | | | | | | | | | | |
| F | | | 0.04 | | | | | 0.03 | 0.04 | |
| D2 | | | 0.05 | 0.05 | 0.02 | 0.02 | 0.03 | | | |
| G | | 0.01 | 0.01 | | | | | 0.000 | 0.01 | 0.01 |
| H | | | 0.36 | | | | | | 0.72 | 0.26 |
| I | | | 0.08 | | 0.04 | | | 0.06 | | |
| J | | | | | 0.07 | | | 0.08 | | |
| K | | | 0.19 | | 0.14 | | | | | |
| E | | | 0.10 | | 0.06 | | | | | |
| L | | | 0.07 | | 0.03 | | | | | |
| M | | | 0.01 | | 0.01 | | | | 0.03 | 0.01 |
| O | | | | | 0.10 | | | | | |
| A2 | | | | | 0.04 | | | | | |
| Q | | | | | 0.03 | | | | | |
| R | | | | | 0.21 | | | | | |
| S | | | | | 0.02 | 0.01 | 0.01 | | | |
| V | | | | 0.02 | 0.03 | 0.06 | 0.06 | | | |
| W | | | | 0.07 | 0.05 | | | | | |
| Y | | | | | 0.02 | | | | | |
| | ADC- | | | | | | | | | |
| | XD24-r | XS22-r | XS25-r | XD65-r | XD44-r | XD45-r | XD46-r | XD58-r | XD63-r | Rmab |
| A1 | | | | | | | | | | |
| B | | | | | | | | | | |
| C | | | | | | | | | | |
| D1 | | | | | | | | | | |
| F | 0.005 | | | | | | | | | 0.05 |
| D2 | | | | | | | | | | 0.33 |
| G | 0.003 | | | | | | | | | † |
| H | | | | | | | | | | 0.15 |
| I | | 0.09 | 0.06 | | | | | | | 3.37 |
| J | | 0.10 | 0.05 | | | | | | | † |
| K | 0.09 | | | | | | | | | 0.59 |
| E | | | | | | | | | | 0.29 |
| L | 0.09 | | | | | | | | | 1.39 |
| M | | | | | | | | | | † |
| O | | | | 1.45 | | | | | | 0.12 |
| A2 | | | | † | | | | | | 0.06 |
| Q | | | | | 0.19 | 0.02 | 0.02 | | | 0.20 |
| R | | | | | | 0.04 | 0.03 | | | 0.16 |
| S | | 0.04 | 0.01 | | | | | 0.01 | 0.01 | 0.25 |
| V | | 0.06 | 0.03 | | | | | 0.03 | 0.05 | 0.06 |
| W | | | | | | | | | | 2.35 |
| Y | | | | | 0.12 | 0.02 | 0.01 | | | 0.34 |

† = EC₅₀ value could not be determined reliably

TABLE 8
% CD107a positive Vδ2 γδ T-cells induced by rituximab pAg conjugate
or rituximab pretreated Raji cells. Empty fields were not tested. The letters
indicate the donors that were used. When the same donor was tested in two
different experiments this was indicated with ‘1’ or ‘2’. Rmab = rituximab.

| | ADC- | | | | | | | | | |
|---|---|---|---|---|---|---|---|---|---|---|
| | XC4-r | XD13-r | XD4-r | XS2-r | XD18-r | XC9-r | XC13-r | XS7-r | XS12-r | XS17-r |
| A1 | 58 | 45 | 51 | | | | | | | |
| B | 84 | | 77 | | | | | | | |
| C | | 77 | 82 | | | | | | | |
| D1 | 72 | 69 | 72 | | | | | | | |
| F | | | 80 | | | | | 82 | 74 | |
| D2 | | | 78 | 77 | 78 | 78 | 77 | | | |
| G | | 72 | 76 | | | | | 80 | 66 | 73 |
| H | | | 78 | | | | | | 52 | 68 |
| I | | | 75 | | 76 | | | 77 | | |
| J | | | | | 76 | | | 74 | | |
| K | | | 59 | | 64 | | | | | |
| E | | | 82 | | 80 | | | | | |
| L | | | 68 | | 72 | | | | | |
| M | | | 84 | | 79 | | | | 76 | 81 |
| O | | | | | 75 | | | | | |
| A2 | | | | | 60 | | | | | |
| Q | | | | | 78 | | | | | |
| R | | | | | 42 | | | | | |
| S | | | | | 36 | 23 | 34 | | | |
| V | | | | 49 | 42 | 53 | 52 | | | |
| W | | | | 49 | 49 | | | | | |
| Y | | | | | 66 | | | | | |
| | ADC- | | | | | | | | | |
| | XD24-r | XS22-r | XS25-r | XD65-r | XD44-r | XD45-r | XD46-r | XD58-r | XD63-r | Rmab |
| A1 | | | | | | | | | | 15 |
| B | | | | | | | | | | 32 |
| C | | | | | | | | | | 12 |
| D1 | | | | | | | | | | 5 |
| F | 81 | | | | | | | | | 36 |
| D2 | | | | | | | | | | 72 |
| G | 74 | | | | | | | | | 65 |
| H | | | | | | | | | | 68 |
| I | | 73 | 82 | | | | | | | 82 |
| J | | 74 | 83 | | | | | | | 65 |
| K | 68 | | | | | | | | | 61 |
| E | | | | | | | | | | 26 |
| L | 68 | | | | | | | | | 20 |
| M | | | | | | | | | | †† |
| O | | | | 38 | | | | | | 20 |
| A2 | | | | 31 | | | | | | 38 |
| Q | | | | | 80 | 78 | 80 | | | 58 |
| R | | | | | | 37 | 33 | | | 30 |
| S | | 40 | 35 | | | | | 38 | 41 | 5 |
| V | | 56 | 49 | | | | | 56 | 56 | 9 |
| W | | | | | | | | | | 60 |
| Y | | | | | 80 | 72 | 81 | | | 0 |

†† = Excluded due to technical problems

TABLE 9
% IFNγ positive Vδ2 γδ T-cells induced by rituximab pAg conjugate
or rituximab pretreated Raji cells. Empty fields were not tested. The letters
indicate the donors that were used. When the same donor was tested in two
different experiments this was indicated with ‘1’ or ‘2’. Rmab = rituximab.

| | ADC- | | | | | | | | | |
|---|---|---|---|---|---|---|---|---|---|---|
| | XC4-r | XD13-r | XD4-r | XS2-r | XD18-r | XC9-r | XC13-r | XS7-r | XS12-r | XS17-r |
| A1 | 49 | 34 | 43 | | | | | | | |
| B | 77 | | 68 | | | | | | | |
| C | | 65 | 72 | | | | | | | |
| D1 | 71 | 59 | 70 | | | | | | | |
| F | | | 82 | | | | | 72 | 61 | |
| D2 | | | 73 | 74 | 68 | 74 | 76 | | | |
| G | | 65 | 67 | | | | | 73 | 51 | 58 |
| H | | | 69 | | | | | | 44 | 49 |
| I | | | 70 | | 65 | | | 68 | | |
| J | | | | | 63 | | | 59 | | |
| K | | | 51 | | 53 | | | | | |
| E | | | 77 | | 72 | | | | | |
| L | | | 52 | | 57 | | | | | |
| M | | | 90 | | 83 | | | | 79 | 82 |
| O | | | | | 55 | | | | | |
| A2 | | | | | 53 | | | | | |
| Q | | | | | 72 | | | | | |
| R | | | | | 30 | | | | | |
| S | | | | | 38 | 20 | 26 | | | |
| V | | | | 47 | 40 | 48 | 40 | | | |
| W | | | | 45 | 45 | | | | | |
| Y | | | | | 64 | | | | | |
| | ADC- | | | | | | | | | |
| | XD24-r | XS22-r | XS25-r | XD65-r | XD44-r | XD45-r | XD46-r | XD58-r | XD63-r | Rmab |
| A1 | | | | | | | | | | 7 |
| B | | | | | | | | | | 14 |
| C | | | | | | | | | | 3 |
| D1 | | | | | | | | | | 4 |
| F | 72 | | | | | | | | | 14 |
| D2 | | | | | | | | | | 42 |
| G | 64 | | | | | | | | | 36 |
| H | | | | | | | | | | 43 |
| I | | 63 | 72 | | | | | | | 63 |
| J | | 58 | 71 | | | | | | | 32 |
| K | 58 | | | | | | | | | 42 |
| E | | | | | | | | | | 9 |
| L | 52 | | | | | | | | | 7 |
| M | | | | | | | | | | †† |
| O | | | | 14 | | | | | | 5 |
| A2 | | | | 20 | | | | | | 17 |
| Q | | | | | 74 | 69 | 71 | | | 31 |
| R | | | | | | 30 | 6 | | | 12 |
| S | | 22 | 23 | | | | | 33 | 37 | 6 |
| V | | 52 | 35 | | | | | 48 | 45 | 11 |
| W | | | | | | | | | | 34 |
| Y | | | | | 76 | 65 | 71 | | | 3 |

†† = Excluded due to technical problems

TABLE 10
% TNFα positive Vδ2 γδ T-cells induced by rituximab pAg conjugate
or rituximab pretreated Raji cells. Empty fields were not tested. The letters
indicate the donors that were used. When the same donor was tested in two
different experiments this was indicated with ‘1’ or ‘2’. Rmab = rituximab.

| | ADC- | | | | | | | | | |
|---|---|---|---|---|---|---|---|---|---|---|
| | XC4-r | XD13-r | XD4-r | XS2-r | XD18-r | XC9-r | XC13-r | XS7-r | XS12-r | XS17-r |
| A1 | | | | | | | | | | |
| B | | | | | | | | | | |
| C | | | | | | | | | | |
| D1 | | | | | | | | | | |
| F | | | 85 | | | | | 87 | 84 | |
| D2 | | | 86 | 86 | 85 | 86 | 86 | | | |
| G | | 81 | 81 | | | | | 83 | 76 | 79 |
| H | | | 85 | | | | | | 79 | 77 |
| I | | | 81 | | 84 | | | 82 | | |
| J | | | | | 81 | | | 76 | | |
| K | | | 70 | | 79 | | | | | |
| E | | | 85 | | 85 | | | | | |
| L | | | 70 | | 78 | | | | | |
| M | | | 98 | | 95 | | | | 94 | 94 |
| O | | | | | 82 | | | | | |
| A2 | | | | | 74 | | | | | |
| Q | | | | | 87 | | | | | |
| R | | | | | 54 | | | | | |
| S | | | | | 49 | 30 | 51 | | | |
| V | | | | 56 | 54 | 63 | 54 | | | |
| W | | | | 47 | 55 | | | | | |
| Y | | | | | 87 | | | | | |
| | ADC- | | | | | | | | | |
| | XD24-r | XS22-r | XS25-r | XD65-r | XD44-r | XD45-r | XD46-r | XD58-r | XD63-r | Rmab |
| A1 | | | | | | | | | | |
| B | | | | | | | | | | |
| C | | | | | | | | | | |
| D1 | | | | | | | | | | |
| F | 86 | | | | | | | | | 43 |
| D2 | | | | | | | | | | 76 |
| G | 82 | | | | | | | | | 73 |
| H | | | | | | | | | | 76 |
| I | | 83 | 85 | | | | | | | 85 |
| J | | 79 | 85 | | | | | | | 62 |
| K | 82 | | | | | | | | | 75 |
| E | | | | | | | | | | 33 |
| L | 76 | | | | | | | | | 32 |
| M | | | | | | | | | | †† |
| O | | | | 51 | | | | | | 28 |
| A2 | | | | 53 | | | | | | 52 |
| Q | | | | | 84 | 85 | 86 | | | 63 |
| R | | | | | | 51 | 26 | | | 35 |
| S | | 45 | 33 | | | | | 51 | 49 | 12 |
| V | | 65 | 52 | | | | | 64 | 62 | 15 |
| W | | | | | | | | | | 59 |
| Y | | | | | 91 | 83 | 87 | | | 12 |

†† = Excluded due to technical problems

### Example 25: Efficacious Killing of Raji Cells by Vδ2 γδ T-Cells after Pre-Treatment with pAg ADC-XD18-r

Vδ2 γδ T-cells can lyse tumor cells opsonized with therapeutic antibodies like rituximab (Sabrina Braza et al., 2011, Haematologica, 96(3), 400-407), most likely through CD16 expressed on the γδ T-cells (classical antibody dependent cellular cytotoxicity, ADCC). However, not all γδ T-cells express CD16 (Sabrina Braza et al, supra). Vδ2 γδ T-cells can also potently kill tumor cells that have high level of pAgs. These pAgs intracellularly induce a conformational change of the BTN3A1/BTN2A1 receptor complex, leading to γδ-T cell activation and killing of the target cells (Rigau et al., supra). It was here determined if a tumor-targeting antibody can be used as a vehicle to deliver pAgs into the tumor cell, leading to specific tumor cell killing by the Vδ2 γδ-T cells. For this, Raji cells were pretreated with a rituximab pAg conjugate and cytotoxicity was studied after a 1 hour co-culture with Vδ2 γδ-T cells.

### Material and Methods

### γδ T-Cell Expansion

To obtain large numbers of Vδ2 γδ T-cells, a standard protocol was used to expand Vδ2 γδ T-cells with IL-2 and zoledronate (Kondo et al., 2008, Cytotherapy, 10(8):842-56.

doi: 10.1080/14653240802419328). For this, frozen PBMCs isolated from buffy coats of healthy donors (Sanquin, Nijmegen, The Netherlands) were thawed, seeded at 12.5 million cells in 10 mL CTS™ OpTmizer™ T-Cell Expansion Serum Free Medium (CTS medium, Gibco, A3705001; basal medium and concentrated medium are pre-mixed before usage according to manufacturer's instructions and 10% Heat-inactivated (HI) Fetal Bovine Serum (FBS) (Gibco) and 80 U/mL Penicillin-Streptomycin solution (Gibco) and glutamax (Gibco) were added) plus 1000 international units (IU) recombination human (rh)IL-2 (Miltenyi, 130-097-746) and 5 μM zoledronate (Merck) in a T25 and cultured for 3 days in a humidified incubator containing 5% CO₂ at 37° C. After 3 days, the cells were transferred to a T75 flask and CTS medium plus 1000 rhIL-2 was added. On day 8, the cells were transferred to a T175 flask and CTS medium plus 1000 IU rhIL-2 was added. After 13 or 14 days, the purity of the cells and their phenotype was assessed by flow cytometry. The Vδ2 γδ T-cell purity was 67.3, 81.7, 82.8, 84.5, 87, 90.6, 91.2, 92 and 95.4% of life cells for the 9 different healthy donors used. The expanded Vδ2 γδ T-cells were used in the killing assay after 14 days. For this, the cells were pelleted and resuspended to 2 million cells/mL in complete growth medium (CGM; RPMI-1640 (Gibco) supplemented with 10% Heat-inactivated (HI) Fetal Bovine Serum (FBS) (Gibco) and 80 U/mL Penicillin-Streptomycin solution (Gibco)).

For phenotyping and purity determination of expanded Vδ2 γδ T cells, 500,000 cells/well were added to a U-bottom 96 well plate, washed twice with ice-cold FACS buffer (PBS 1×, 0.1% v/w BSA, 0.02% v/v Sodium Azide (NaN3)), and the cell pellet was resuspended in 100 μL antibody cocktail diluted in ice-cold FACS buffer: anti-Vδ2 BV711 (clone B6, Biolegend, 1:300), anti-CD56 AlexaFluor647 (clone B159, BD Bioscience, 1:200), anti-CD16 FITC (clone 3G8, BD Biosciences, 1:50), fixable viability stain 780 (ThermoFisher Scientific, 1:1000). After incubation for 30 minutes on ice in the dark, the cells were then washed twice by adding ice-cold FACS buffer, followed by centrifugation at 300×g for 3 minutes and discarding of the supernatant. The pellet was resuspended in 150 μL ice-cold FACS buffer and analyzed using the BD FACSymphony A3 Cell analyzer (BD Biosciences, San Jose, CA, USA). Further analysis was performed using FlowJo V10.7. Vδ2 γδ T-cells were defined as live Vδ2+ cells.

### Killing Assay

Raji cells were cultured as described in Example 23.

For the killing assay, Raji cells were first washed twice with PBS (Gibco, 2326202) and then labeled with 10 μM Cell Proliferation Dye eFluor 450 (Thermofisher Scientific, 65-0842-85) for 10 minutes at 37° C. in the dark. After addition of 4-5 volumes of CGM for 5 minutes on ice, the cells were washed 3 times with RPMI-1640 plus 10% HI-FBS, diluted to 200.000 cells/mL in CGM and 50 μL/well plated in a 96-well plate (Greiner Bio-one, 650185, U-bottom). Then, 50 μL/well of a concentration range of a rituximab-pAg conjugate, rituximab or 0.1 mM or 0.013 mM HMBPP diluted in CGM was added and incubated for 16 hours in a humidified incubator containing 5% CO₂ at 37° C. Of note, the concentrations of HMBPP used was shown to induce maximal efficacy (data not shown). After 16 hours, 100 μl CGM/well was added and the cells were pelleted by centrifugation at 300×g, the supernatant was removed and 100,000 expanded Vδ2 γδ T-cells (day 14 of culture) were added to each well in a volume of 50 μL/well. The plates were placed in a humidified incubator containing 5% CO₂ at 37° C. and incubated for 1 hour. The cells were then pelleted by centrifugation for 3 minutes at 300×g and the supernatant was removed. The cells were resuspended in 50 μL fixable viability stain 780 (BD Biosciences, 1000× diluted in ice-cold FACS buffer) plus anti-CD19 FITC (Miltenyi 130-113-645, incubated for 30 minutes on ice in the dark, and the cells were washed by addition of 150 μL ice-cold FACS buffer and centrifugation for 3 minutes at 300×g. The cell pellets were then resuspended in 50 μL BD cytofix solution (554655), and after incubation for 15 minutes on ice in the dark the cells were washed twice by addition of 150 μL ice-cold FACS buffer, centrifugation (300×g, 3 minutes) and removal of supernatant. Finally, the cells were resuspended in 150 μL ice-cold FACS buffer and analyzed using the BD FACSymphony A3 Cell analyzer (BD Biosciences, San Jose, CA, USA). Raji cells were gated using the eFluor450 dye and the % dead Raji cells was determined using the viability stain. Further analysis was performed using FlowJo V10.7. The efficacy=% dead Raji cells pretreated with the highest concentration of compound−% dead Raji cells pretreated with no compound.

### Results/Conclusion

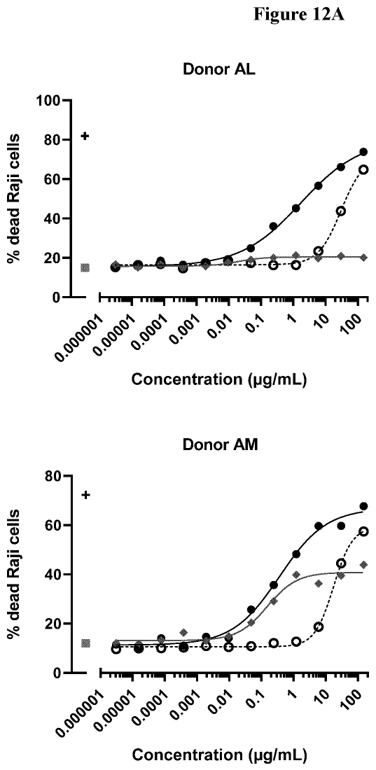

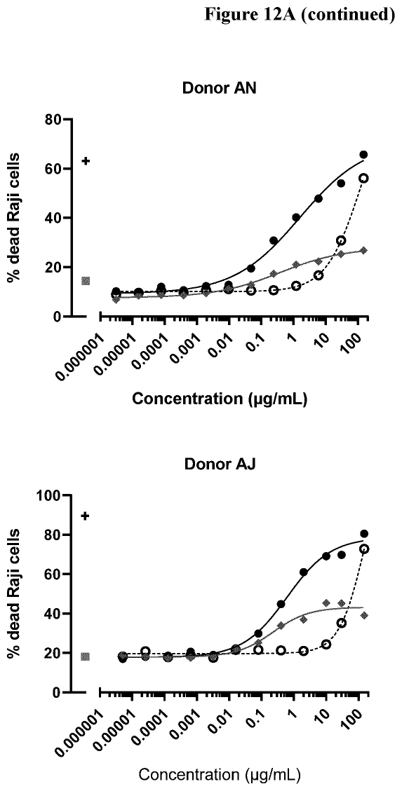

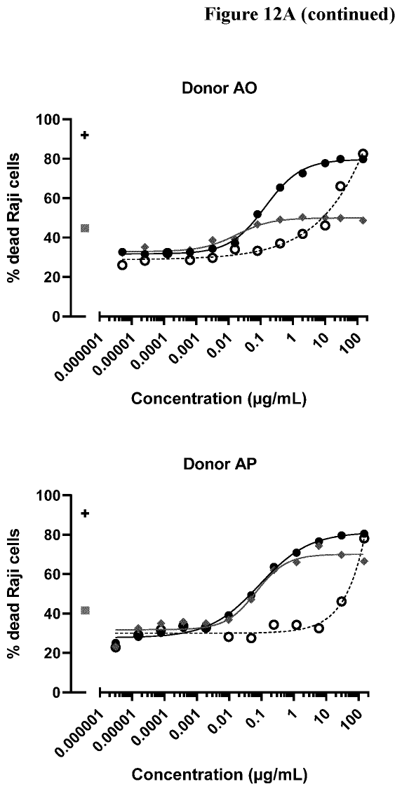

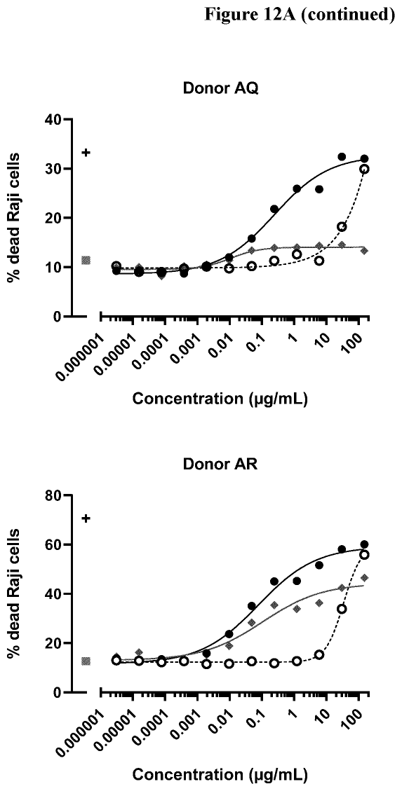

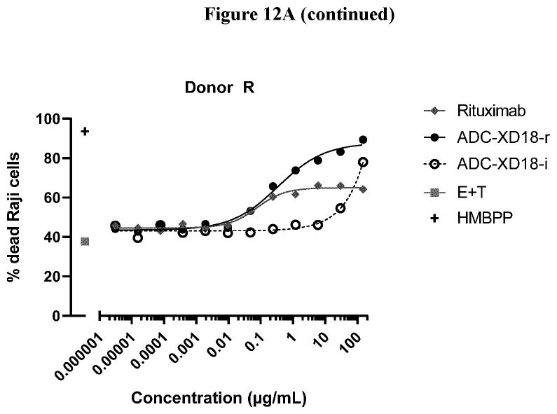

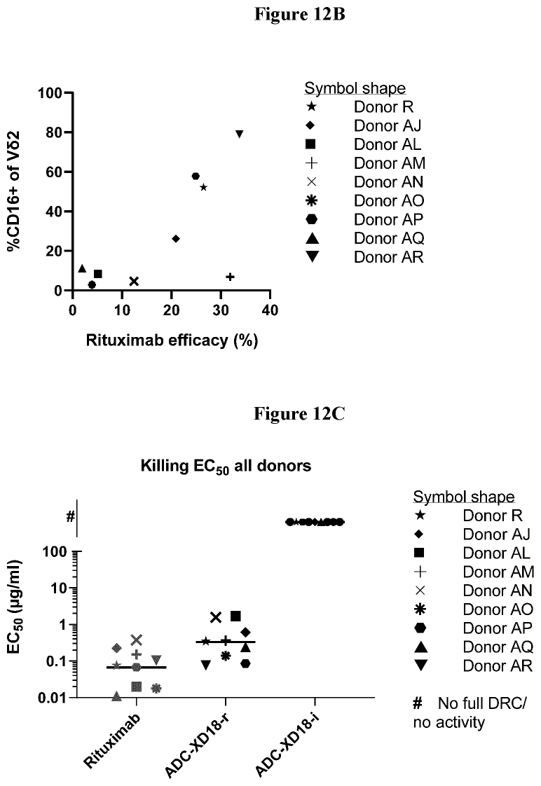

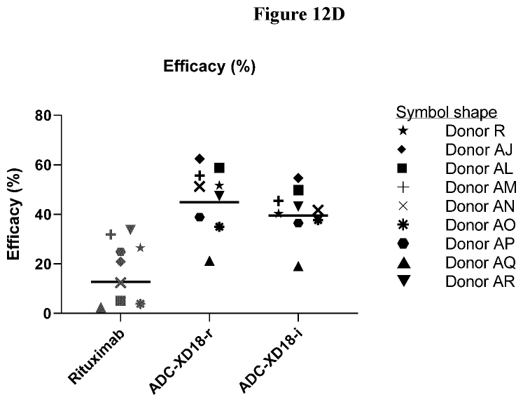

When Raji cells were pretreated with rituximab, dose-dependent killing was detected by expanded Vδ2 γδ T-cells from most donors (FIG. 12). The rituximab efficacy was variable and as expected, correlated with the expression of CD16 on the expanded Vδ2 γδ T-cells, which also showed high donor-to-donor variations (FIG. 12B). The efficacy induced by rituximab pretreatment was always lower compared to HMBPP pretreatment. When expanded Vδ2 γδ T-cells were exposed to Raji cells pretreated with ADC-XD18-r, dose-dependent killing of Raji cells was observed, and the efficacy was in a similar range compared to HMBPP-pretreated Raji cells and higher than for rituximab (FIGS. 12A and D). The potency of non-binding isotype pAg conjugates was low and no reliable EC₅₀ calculation for Vδ2 γδ T-cell activation was possible (FIGS. 12A and C). Overall, these results show that a rituximab pAg conjugate can potently and efficaciously confer tumor cells into targets for destruction by Vδ2 γδ T-cells in an TAA-specific manner.

### Example 26: CD20-Positive Cell Lines Derived from Various B-Cell Malignancies Potently and Efficaciously Activate Vδ2 γδ T-Cells after Preincubation with ADC-XD18-r

The activity of ADC-XD18-r was tested with multiple CD20 positive cell lines representing different B-cell malignancies (CLL, NHL) with varying CD20 expression levels (Table 11). For this, the B-cell lines were first pretreated with compounds for 16 hours and then subsequently co-cultured with PBMCs. Vδ2 γδ T-cell activation was assessed by determining the level of degranulation (CD107a production).

### Material and Methods

### Functional Assay

Raji cells were cultured as described in Example 23. The human tumor cell lines MEC-1, HG-3, SU-DHL-4 and SU-DHL-8 cells were from the German collection of Microorganisms and cell cultures GmbH (DSMZ, Leibniz Institute, Germany)). HG-3 and SU-DHL-4 cells were cultured in CGM. SU-DHL-8 was cultured in RPMI-1640 (Lonza) supplemented with 20% Heat-inactivated (HI) Fetal Bovine Serum (FBS) (Gibco) and 80 U/mL Penicillin-Streptomycin solution (Lonza). MEC-1 cells were cultured in IMDM (12-722F, IMDM, Lonza) supplemented with 10% Heat-inactivated (HI) Fetal Bovine Serum (FBS) (Gibco-Life Technologies; Carlsbad, CA) and 80 U/mL Penicillin-Streptomycin solution (Lonza Group Ltd, Basel Switzerland)). All cells were maintained at 37° C. in a humidified incubator containing 5% CO₂ and sub-cultured twice a week.

For the material and method of the functional assay, see example 23, with the exception that degranulation levels were determined only and not IFNγ expression levels. Thus, on the third experimental days, cells were directly analyzed on the BD FACSymphony A3 Cell analyzer (BD Biosciences, San Jose, CA, USA), before incubation with BD Perm/wash. The highest compound concentration used for pretreatment of Raji cells was 150 μg/mL for antibodies/ADCs and 0.1 mM for HMBPP. EC₅₀ values were calculated in GraphPad Prism as the concentration in μg/mL that gives a response half way between bottom and top of the curve. In the summary graphs, the ‘% activated Vδ2 γδ T-cells’ is determined from samples co-cultured with Raji cells pretreated with the highest compound concentration (e.g. 150 μg/mL for antibodies and ADCs).

### CD20 Expression Level Determination

CD20 receptor expression levels were determined using the human calibrator kit (Biocytex, CP010). Target cells (100,000 cells/well in a 96-well plate) were washed twice with ice-cold FACS buffer (PBS+0.1% v/w BSA+0.02% v/v Sodium Azide (NaN3)), followed by the addition of a concentration range of 50 μL/well rituximab (anti-CD20) diluted in ice-cold FACS buffer. After an incubation time of 30 minutes at 4° C., the cells were washed twice with ice-cold FACS buffer and resuspended in 50 μL FACS buffer. Then, 50 μL beads from the human calibrator kit were added to a separate well of the 96-well plate. A twice concentrated stock of APC-conjugated secondary F(ab′)₂ goat anti-Human IgG (Fc fragment specific, Jackson ImmunoResearch, 1:3000, 1:6000 final) was generated and 50 μL/well was added to the cells and beads. After 30 minutes incubation at 4° C. in the dark, cells and beads were washed twice in ice-cold FACS buffer and resuspended in 150 μL ice-cold FACS buffer. Fluorescence intensities were determined by flow cytometry using the FACSymphony (BD Biosciences) and absolute numbers of receptors were determined according to manufacturer's instructions. The experiment was performed N=2 times.

### Results/Conclusion

The B-cell lines used in this example expressed varying levels of CD20 (Table 11).

TABLE 11
CD20 expression level on multiple CD20 positive cell lines representing
different B-cell malignancies (CLL, NHL), and CD107a EC₅₀ values
(μg/mL) of Vδ2 γδ T-cell after coculture with indicated
ADC-XD18-r or rituximab pretreated cell lines using CD107a as read-out.

| | | | CD20 | CD107a EC₅₀ (μg/mL) | | | |
|---|---|---|---|---|---|---|---|
| | | | expression | ADC-XD18-r | | Rituximab | |
| | B-cell | Lymphoma | level | Donor | Donor | Donor | Donor |
| Cell line | malignancy | type | (sABC/cell) | Q | AH | Q | AH |
| Raji | NHL | Burkitt's | 176680 | 0.100 | 0.094 | 0.479 | † |
| HG-3 | CLL | N/A | 168919 | 0.059 | 0.061 | 0.177 | † |
| MEC-1 | CLL | N/A | 170964 | 0.031 | 0.039 | 0.414 | 3.646 |
| SU-DHL-4 | NHL | GCB | 326856 | 0.021 | 0.042 | † | 2.638 |
| SU-DHL-8 | NHL | GCB | 7205 | 0.186 | 0.228 | † | † |

† = Incomplete curve saturation (no EC₅₀ calculation possible)

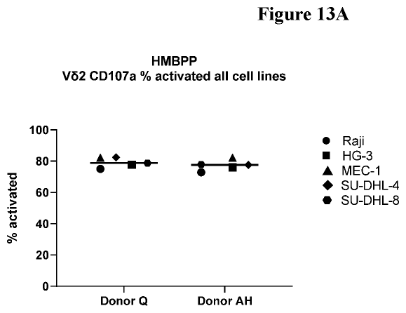

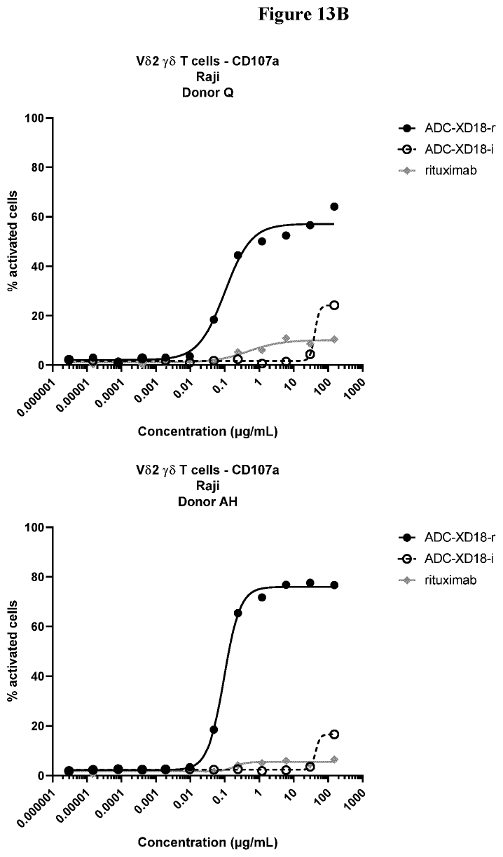

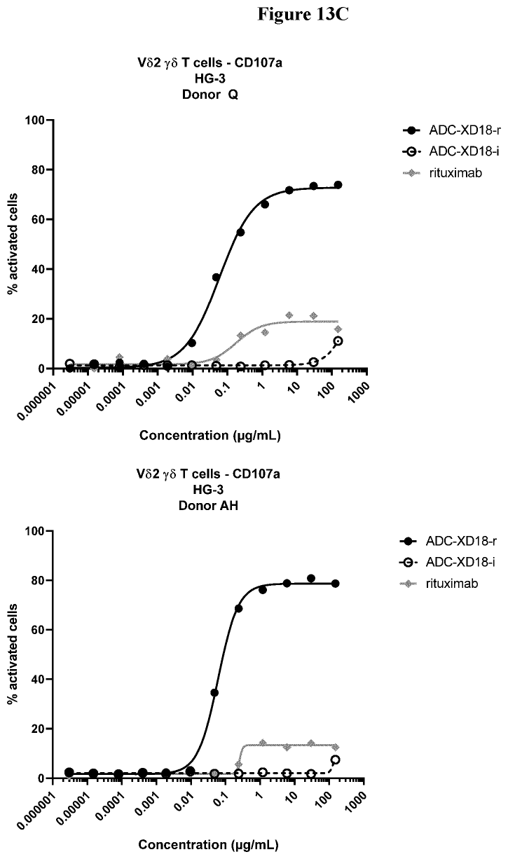

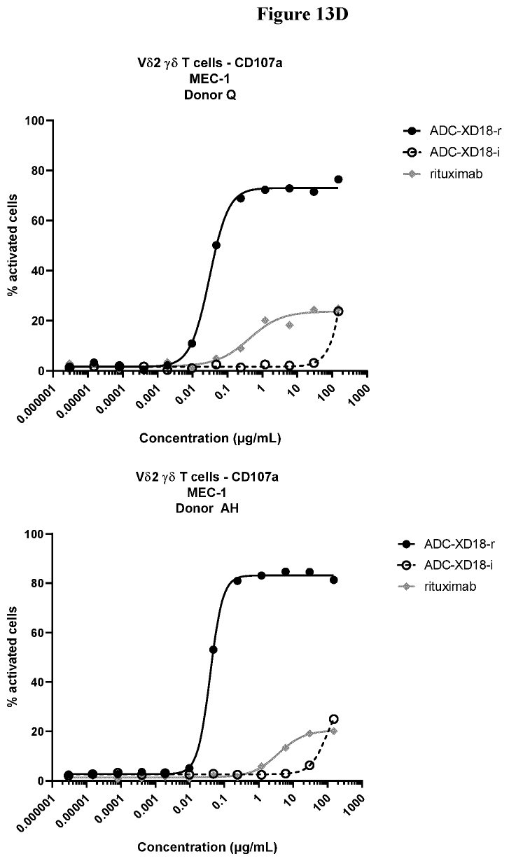

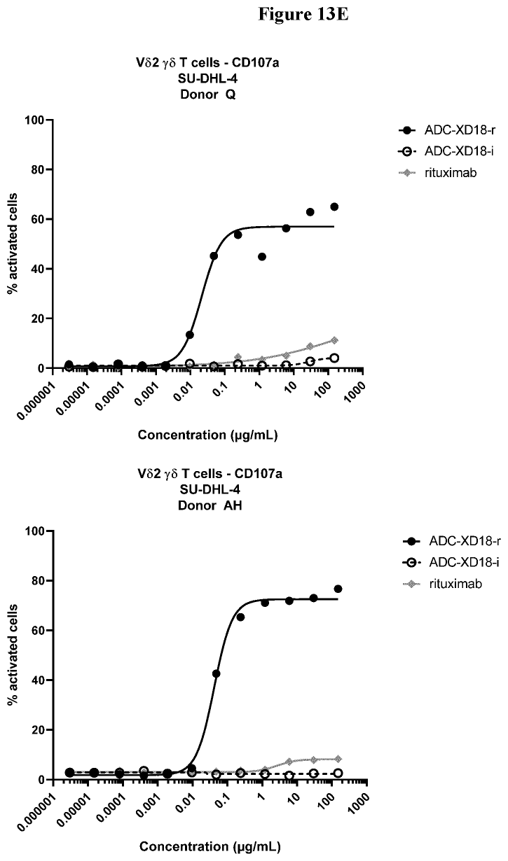

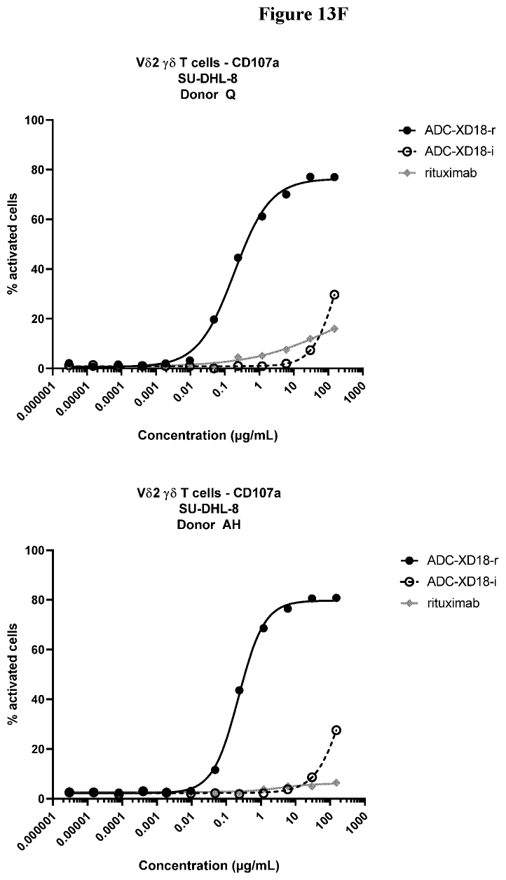

All tested B-cell lines had the ability to activate Vδ2 γδ T-cells, as HMBPP pretreatment induced degranulation of Vδ2 γδ T-cells (FIG. 13A). When the B-cell lines were pretreated with a concentration range of ADC-XD18-r, they all induced potent activation of Vδ2 γδ T-cells with higher efficacy (% activity) and potency than rituximab itself (FIG. 13B-F, Table 11). Non-binding control ADCs induced Vδ2 γδ T-cell activation with low/negligible potency and efficacy. These results showed that ADC-XD18-r accomplished activation of Vδ2 γδ T-cells when preincubated with multiple B-cell malignancies with low and high CD20 expression levels, while pretreatment with the non-binding pAg conjugates failed to induce Vδ2 γδ T-cell activation.

### Example 27: Trastuzumab-ADC Pretreated HER2^{high} Cells Induce Vδ2 γδ T-Cell Activation

To show activity of the pAg conjugate concept beyond rituximab, the linker drug XD18 was conjugated to trastuzumab, to create ADC-XD18-t. Upon pretreatment of HER2-positive cell lines (reflected in Table 12) with these novel ADCs and coincubation with PBMCs, Vδ2 γδ T-cell activity was determined.

### Material and Methods

### Functional Assay

The functional assay was performed as disclosed in Example 23. In addition to anti-IFNγ BV650, also anti-TNFαPE (BD Biosciences, San Jose, CA, USA, clone Mab11) was included in most of the experiments during the intracellular cytokine staining step. The highest compound concentration used for pretreatment of Raji cells was 150 μg/mL for antibodies/ADCs and 0.1 mM for HMBPP. EC₅₀ values were calculated in GraphPad Prism as the concentration in μg/mL that gives a response half way between bottom and top of the curve. In the summary graphs, the ‘% activated Vδ2 γδ T-cells’ is determined from samples cocultured with Raji cells pretreated with the highest compound concentration (e.g. 150 μg/mL for antibodies and pAg conjugates).

Human tumor cell lines SK-BR-3, BT-474, SK-OV-3 were obtained from American Type Culture Collection (ATCC, Rockville, MD), the HCT-116 from the German collection of Microorganisms and cell cultures GmbH (DSMZ, Leibniz Institute, Germany)). The BT-474 (ATCC; ATCC-HTB-20) was cultured in CGM and was maintained at 37° C. in a humidified incubator containing 5% CO₂ and sub-cultured twice a week. SK-BR-3, SK-OV-3 and HCT-116 were maintained in McCoys 5A medium (Lonza) containing 10% v/w FBS HI 80 U/mL and Penicillin-Streptomycin solution (Lonza).

### HER2 Expression Level Determination

See example 25, with the adjustment that trastuzumab was used to determine HER2 levels on the cells. The experiment was performed N=2 times and a mean sABC is reported.

### Results/Conclusion

The cell lines used here expressed either high (BT-474, SK-BR-3 and SK-OV-3) or low (HCT-116) levels of HER2 (Table 12).

TABLE 12
HER2 expression level on selected cell lines

| Cell line | Malignancy | HER2 expression level (sABC/cell) |
|---|---|---|
| BT-474 | Breast cancer | 3.300,000 |
| SK-BR-3 | Breast cancer | 3,000,000 |
| SK-OV-3 | Ovarian cancer | 2,400,000 |
| HCT-116 | Colorectal cancer | 21,000 |

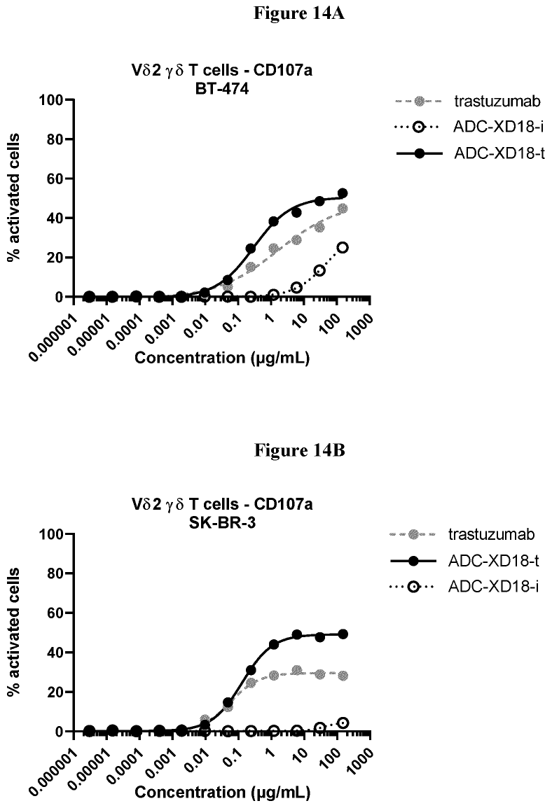

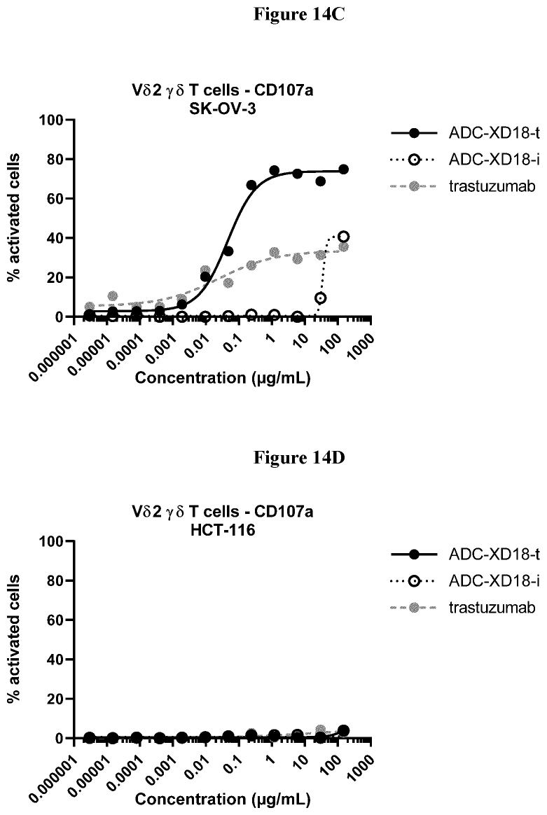

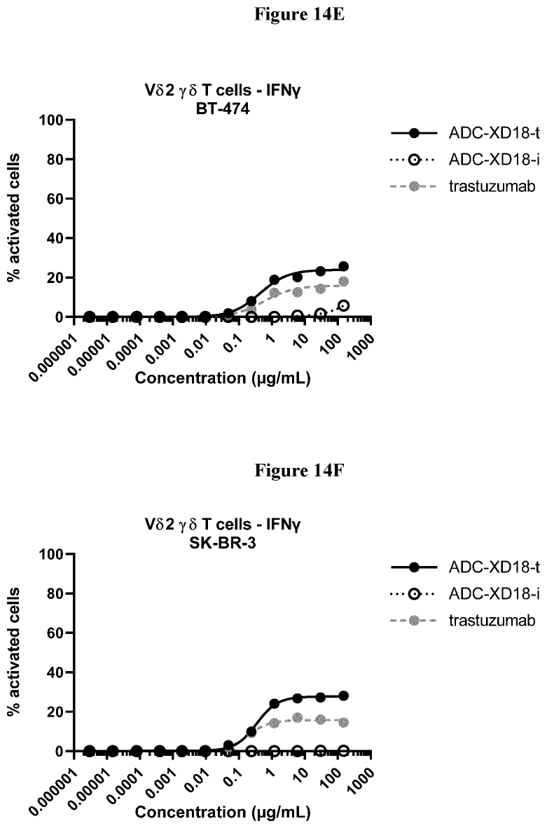

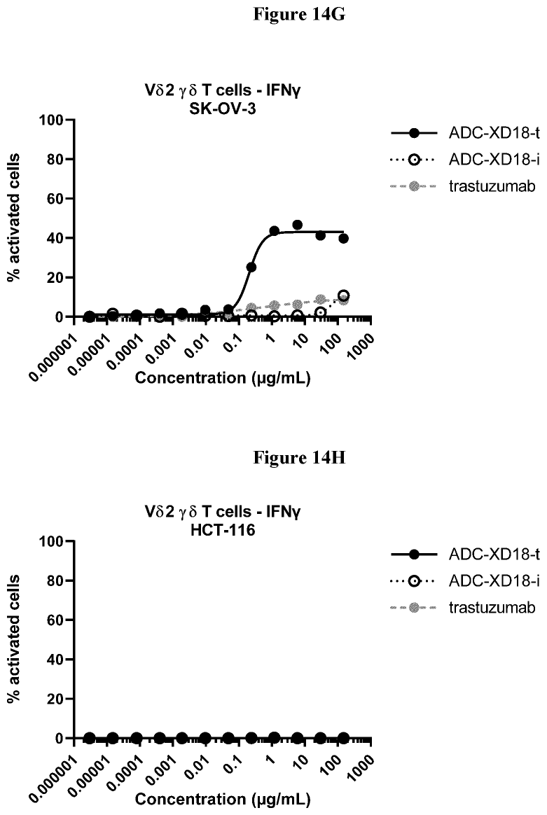

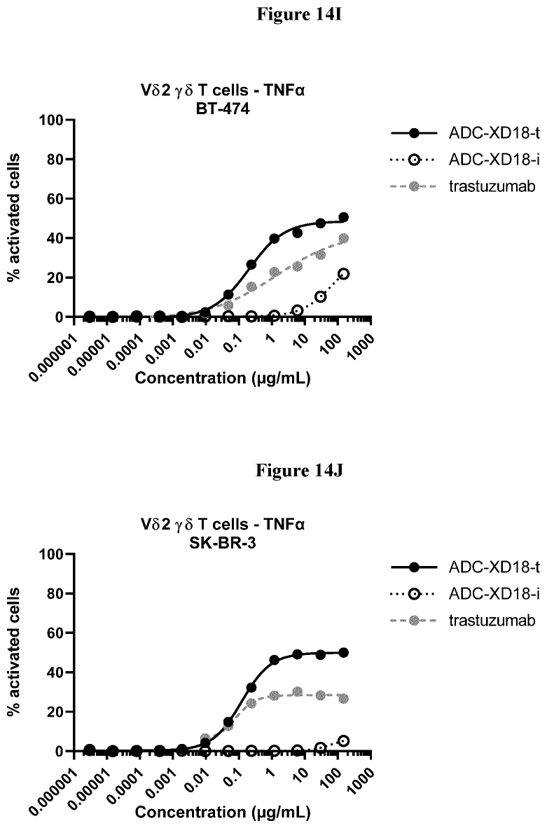

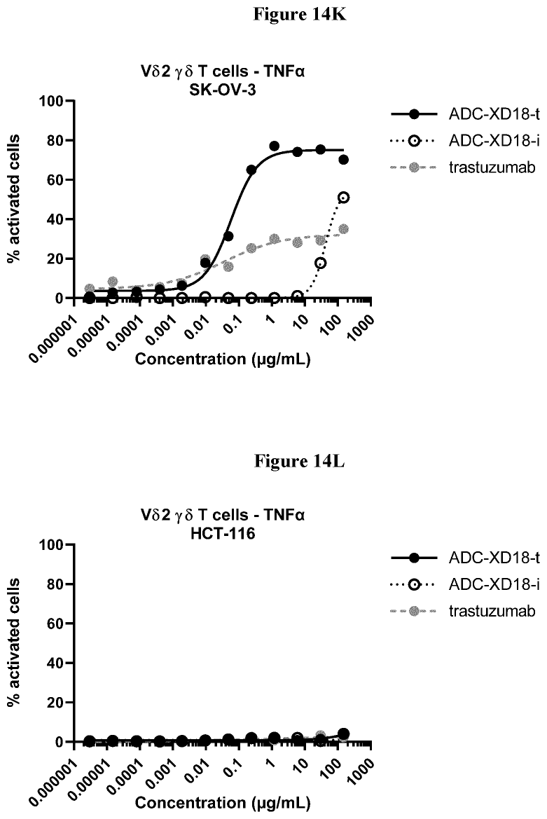

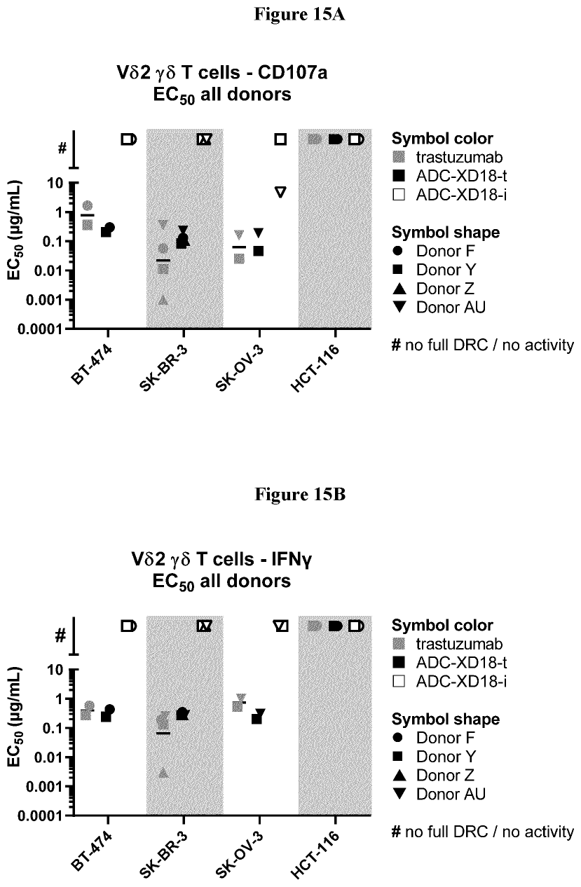

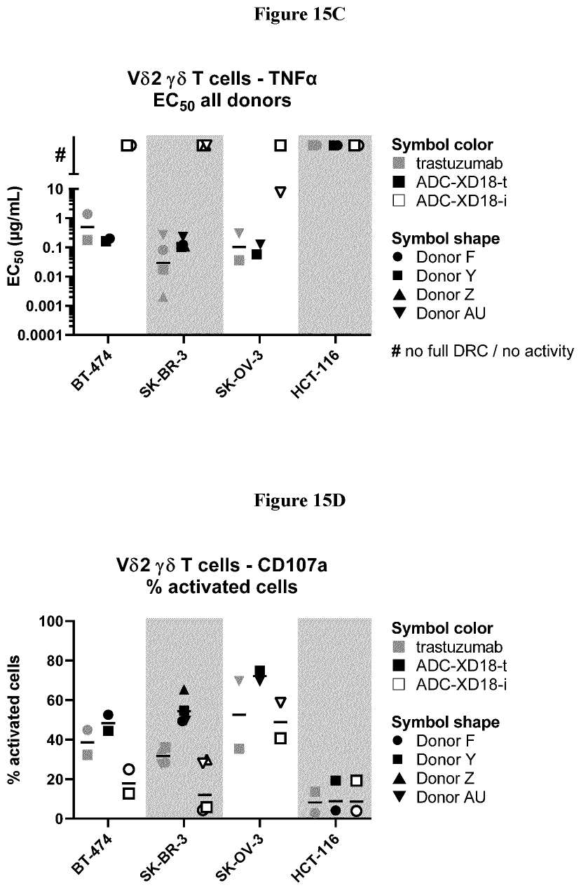

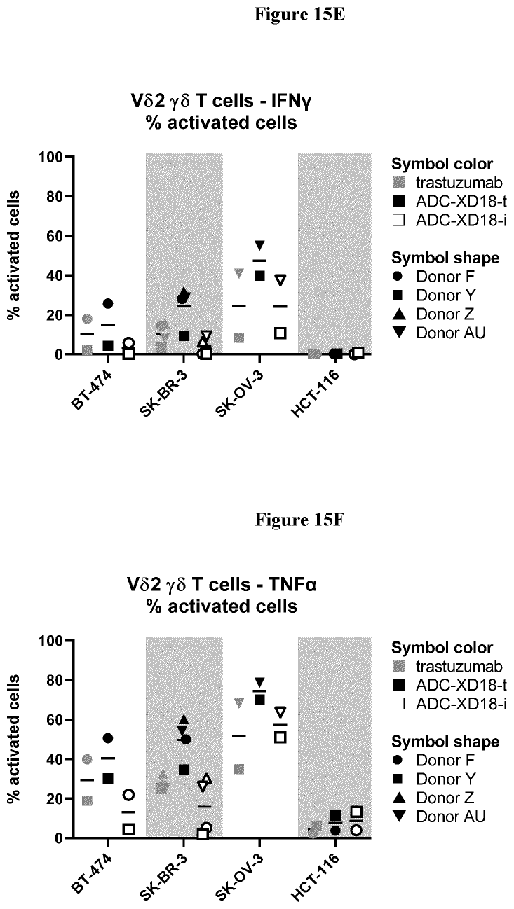

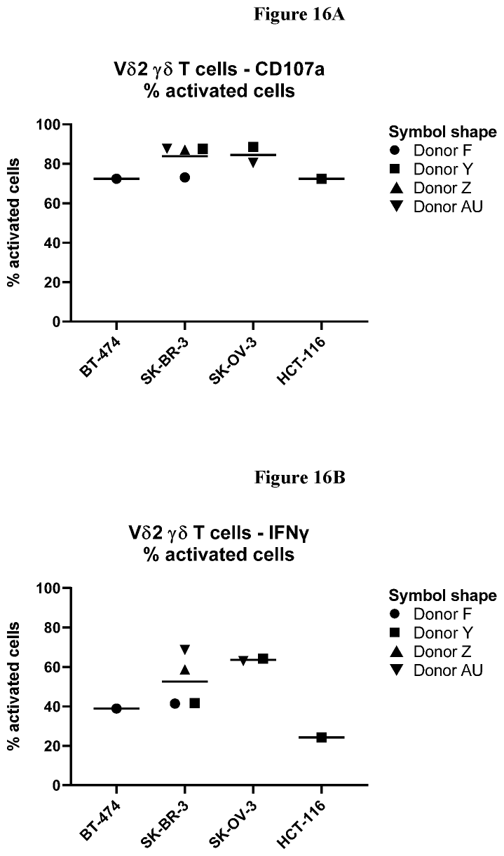

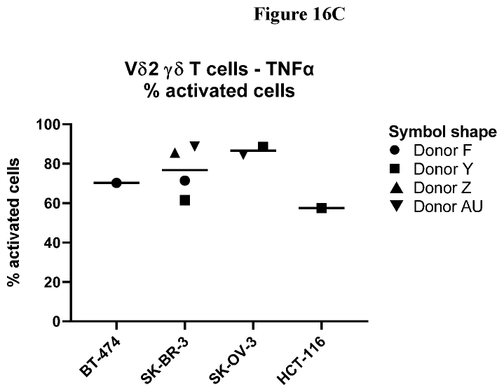

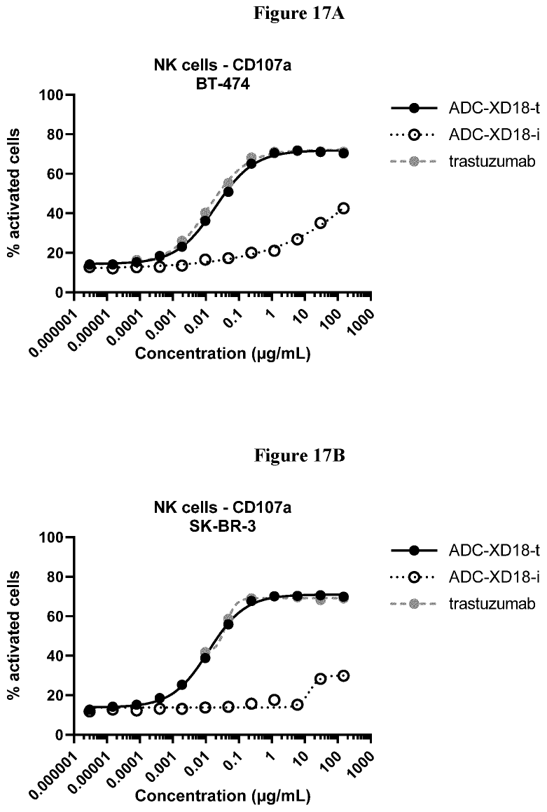

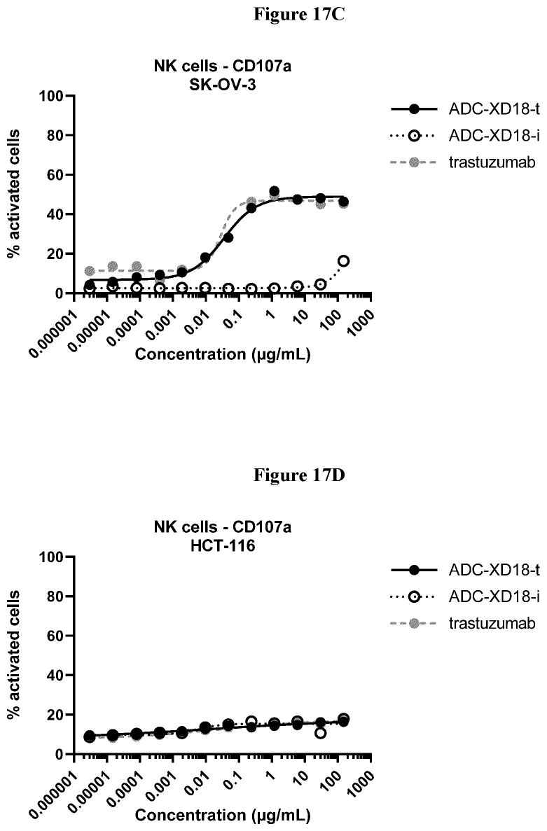

The four different cell lines were first preincubated with ADC-XD18-t, ADC-XD18-i or trastuzumab and then cocultured with Vδ2 γδ T-cell containing PBMCs and immune cell activation was determined. Results from representative donors are depicted in FIG. 14 and potency (EC₅₀) and efficacies of all tested donors were summarized in FIG. 15. BT-474, SK-BR-3 and SK-OV-3 cells preincubated with trastuzumab induced activation of Vδ2 γδ T-cells in a dose-dependent manner, most likely through trastuzumab-FcγRs interaction. HCT-116 cells pretreated with trastuzumab failed to induce notable activation of Vδ2 γδ T-cells, presumably due to the low numbers of HER2 receptors on the cell surface of HCT-116 cells (Table 12). When preincubated with BT-474, SK-BR-3 and SK-OV-3 cells, ADC-XD18-t induced degranulation (CD107a), IFNγ and TNFαproduction in more Vδ2 γδ T-cells than trastuzumab itself. HCT-116 cells failed to activate Vδ2 γδ T-cells when preincubated with ADC-XD18-t. This was not due to an inability of HCT-116 cells to activate Vδ2 γδ T-cells, as HMBPP pretreated HCT-116 were able to activate Vδ2 γδ T-cells (FIG. 16). Pretreatment of BT-474, SK-BR-3, SK-OV-3 and HCT-116 cells with non-binding isotype control-ADCs led to negligible or low potent activation of Vδ2 γδ T-cells (exemplified in FIG. 14). Trastuzumab pretreated BT-474, SK-BR-3 and SK-OV-3 also activated NK-cells, most likely through FcγRs that are well-known to be expressed by NK-cells. Trastuzumab pAg conjugate-pretreated BT-474, SK-BR-3 and SK-OV-3 cells were also able to activate NK-cells to a similar extend (FIG. 17), showing that trastuzumab-induced effector functions were still intact after addition of a pAg conjugate. HCT-116 cells did not display notable NK-cell activation after preincubation with trastuzumab or trastuzumab-ADC, again indicating that the HER2 expression level is too low on this cell line to trigger immune cell activation. Overall, these results show that pAg conjugates can be linked to various tumor-targeting antibodies to target multiple malignancies to induce Vδ2 γδ T-cell activation.

### Example 28: A Higher pAg Drug-to-Antibody-Ratio (DAR) Improves Vδ2 γδ T-Cell Activity Towards Cells with Low Expression of Tumor Associated Antigen (TAAs)

It was investigated if augmenting the DAR leads to more potent and efficacious Vδ2 γδ T-cell activation, especially for cell lines with a low number of TAAs.

### Material and Methods

### Vδ2 γδ T Cell Activation Assay

### Functional Assay

The functional assay was performed as disclosed in Example 23, with the exception that Raji and MOLM-13 cells were pretreated for 16 or 40 hours with antibodies/ADCs/controls (Table 13). Furthermore, CD107a degranulation levels were determined only and not IFNγ expression levels. Thus, on the third experimental days, cells were directly analyzed on the BD FACSymphony A3 Cell analyzer (BD Biosciences, San Jose, CA, USA), before incubation with BD Perm/wash. The highest compound concentration used for pretreatment of Raji and MOLM-13 cells was 150 μg/mL for antibodies/ADCs and 0.1 mM for HMBPP. EC₅₀ values were calculated in GraphPad Prism as the concentration in μg/mL that gives a response half way between bottom and top of the curve. In the summary graphs, the ‘% activated Vδ2 γδ T cells’ is determined from samples co-cultured with Raji or MOLM-13 cells pretreated with the highest compound concentration (e.g. 150 μg/mL for antibodies and ADCs).

TABLE 13
overview of compounds used for pretreatment of Raji
or MOLM-13 cells before co-culture with PBMCs

| Tested cell line | pAg conjugate (see Table 1 for details) |
|---|---|
| MOLM-13 | ADC-XD18-CD123_{41C} |
| MOLM-13 | ADC₁₀-XD18-CD123_{41C} |
| MOLM-13 | ADC₂₀-XD59-CD123_{41C} |
| MOLM-13 | ADC-XD18-i_{41C} |
| MOLM-13 | ADC₈-XD18-i |
| MOLM-13 | ADC₁₆-XD59-i |
| MOLM-13 | anti-CD123 41C MoAb |
| Raji | ADC-XD18-r |
| Raji | ADC₈-XD18-r |
| Raji | ADC₁₆-XD59-r |
| Raji | ADC-XD18-i |
| Raji | ADC₈-XD18-i |
| Raji | ADC₁₆-XD59-i |
| Raji | Rituximab |

### CD123 Expression Level Determination

CD123 receptor expression levels were determined using the Qifi kit (DAKO, agilent, USA). Cells (100,000 cells/well in a 96-well plate) were washed twice with ice-cold FACS buffer (PBS+0.1% v/w BSA+0.02% v/v Sodium Azide (NaN3)), followed by the addition of a concentration range of 50 μL/well anti-CD123 (clone 6H6, ThermoFisher Scientific) diluted in ice-cold FACS buffer. After an incubation time of 30 min at 4° C., the cells were washed twice with ice-cold FACS buffer and resuspended in 50 μL APC-conjugated secondary F(ab′)₂ goat anti-Human IgG (Fc fragment specific, Jackson ImmunoResearch, 1:6000 final) in ice-cold FACS buffer. The dilution of secondary antibody was also added to pelleted beads from the Qifi kit. Incubation with the secondary antibody was performed for 30 minutes at 4° C. in the dark on ice, and afterwards the cells and beads were washed twice in ice-cold FACS buffer and resuspended in 150 μL ice-cold FACS buffer. Fluorescence intensities were determined by flow cytometry using the FACSVerse or FACSymphony (BD Biosciences) and absolute numbers of receptors were determined according to manufacturers instructions.

### MOLM-13 and Raji Cell Culture

Raji cells were cultured as described in Example 23. The CD123-positive acute monocytic leukemia cell line MOLM-13 (DSMZ, ACC 554, the German collection of Microorganisms and cell cultures GmbH (Leibniz Institute, Germany)) was cultured in CGM and was maintained at 37° C. in a humidified incubator containing 5% CO₂ and sub-cultured twice a week.

### Results/Conclusion

The MOLM-13 cell line expressed low levels of CD123 (Table 14). CD20 expression levels on Raji cells was shown in Table 12.

TABLE 14
CD123 expression level on MOLM-13 cells

| Cell line | Malignancy | CD123 Expression level (sABC/cell) |
|---|---|---|
| MOLM-13 | Acute monocytic | 9664 |
| | leukemia | |

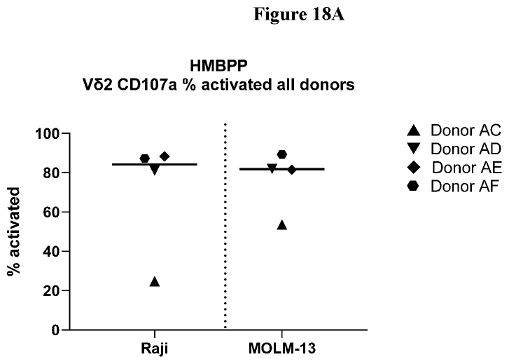

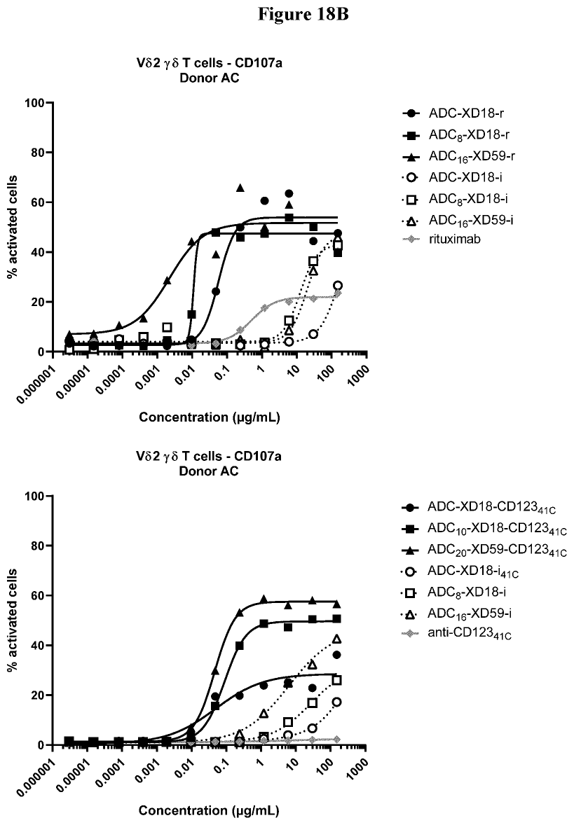

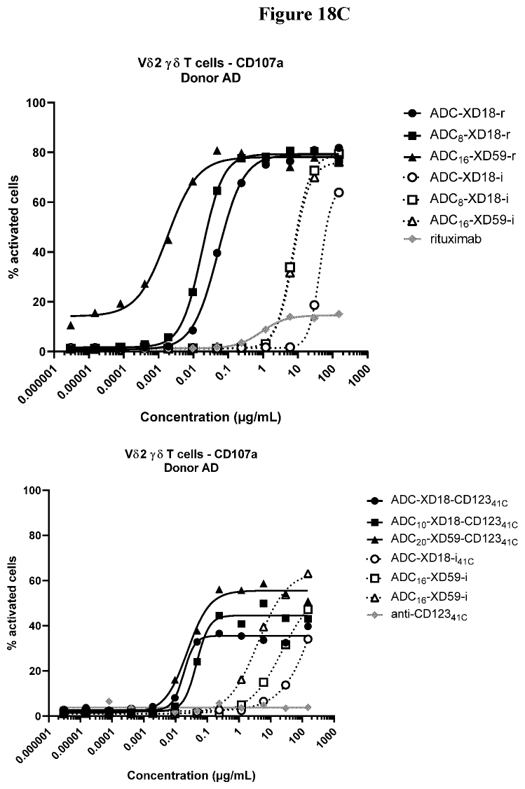

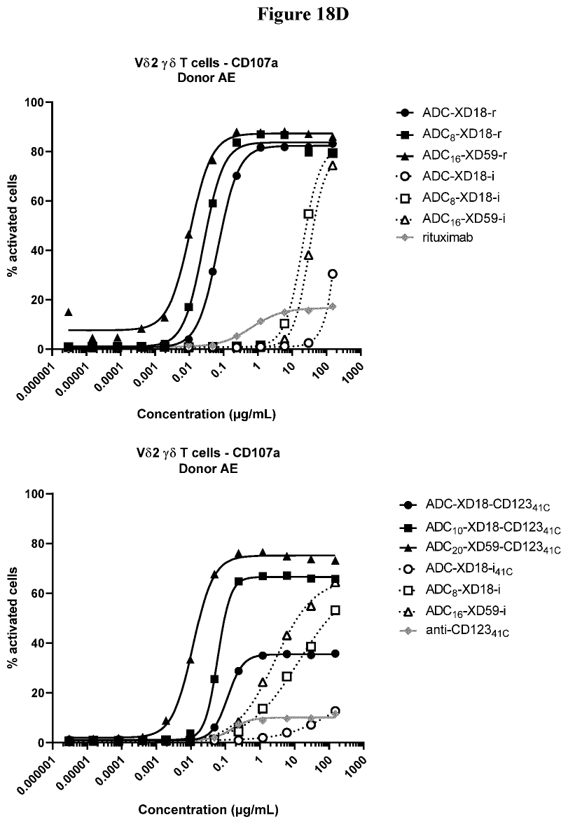

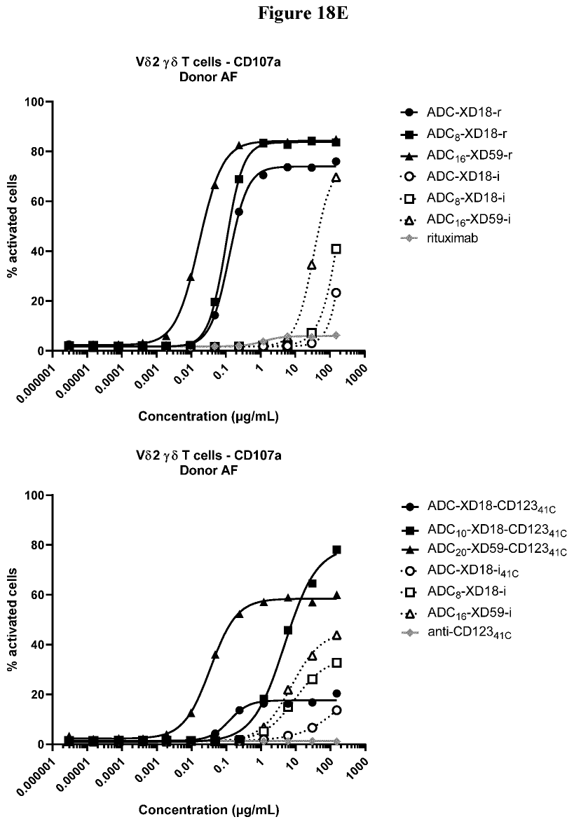

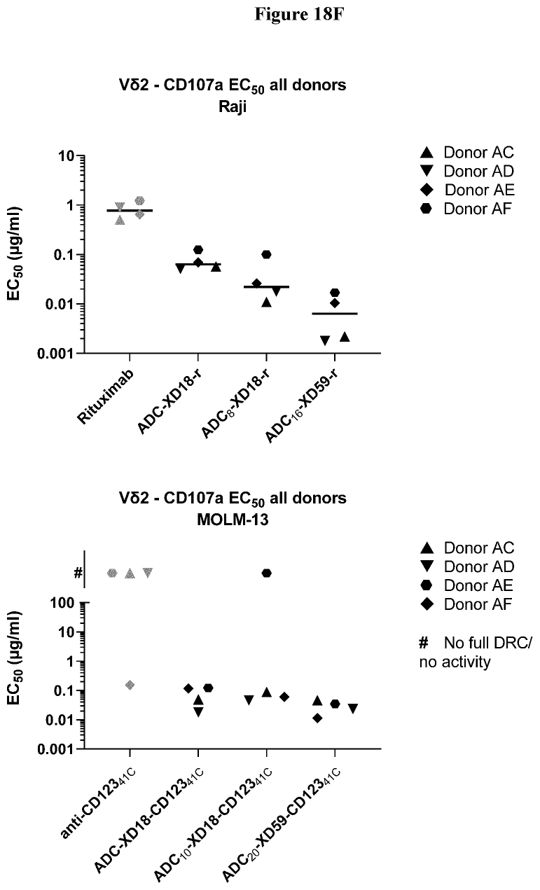

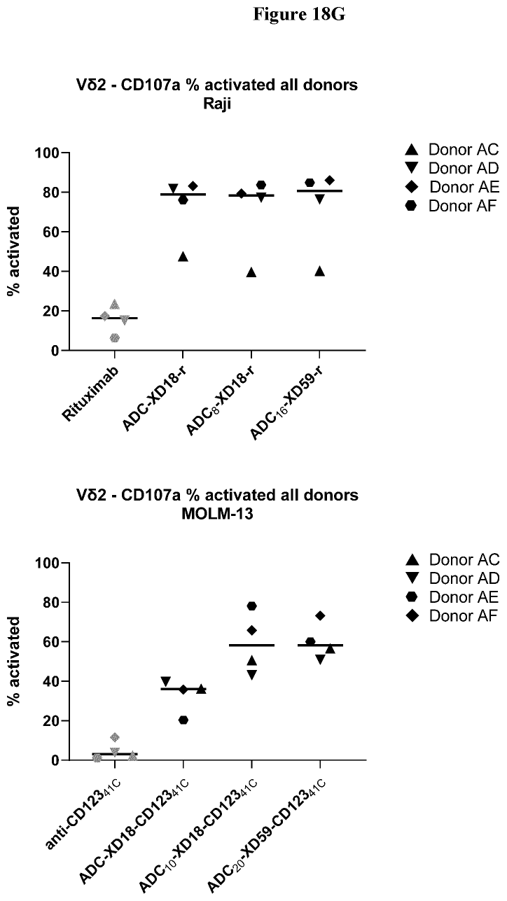

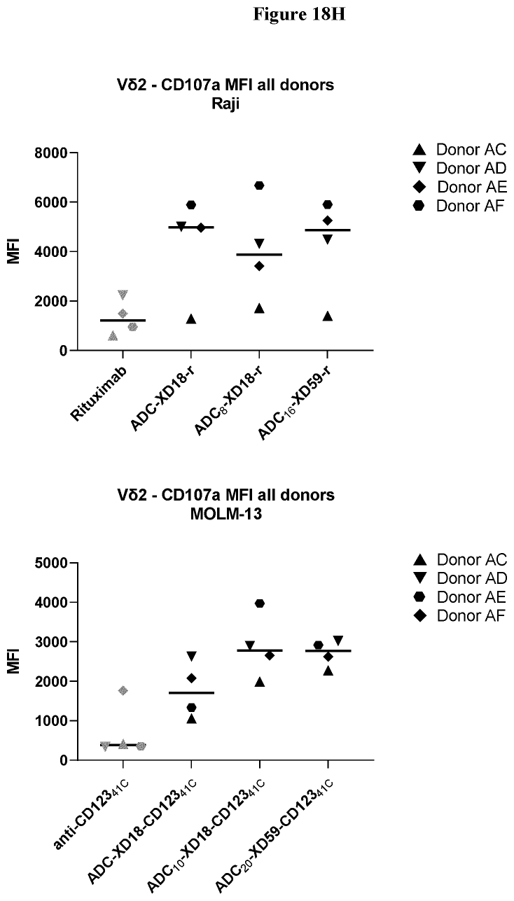

Both MOLM-13 and Raji cells were capable of activating Vδ2 γδ T-cells after pretreatment with HMBPP (FIG. 18A). The generated pAg conjugates and control compounds were tested for their ability to induce selective Vδ2 γδ T-cell activation after overnight incubation with MOLM-13 or Raji cells, followed by a 6 hour coculture with Vδ2 γδ T-cell containing PBMCs. Dose-response curves for Vδ2 γδ T-cell degranulation were generated (FIG. 18B-E) and these results showed that cells pretreated with non-binding isotype controls pAg conjugates activated Vδ2 γδ T-cells with low potency. Therefore, EC₅₀ values could not be calculated reliably. For the rituximab/anti-CD12341c MoAb pAg conjugates, EC₅₀ values, efficacies and MFIs were calculated (FIG. 18 F-H).

When anti-CD12341c MoAb pre-treated MOLM-13 cells were cocultured with PBMCs, no Vδ2 γδ T-cell activation was measured for 3 out of 4 donors. When ADC-XD18-CD123_{41C} pre-treated MOLM-13 cells were cocultured with PBMCs, the percentage of activated Vδ2 γδ T-cells was increased. In addition, the amount of CD107a produced per cell was higher with pAg conjugates versus the naked mAb (FIG. 18H). Increasing the DAR led to a further increase in the percentage of CD107a positive Vδ2 γδ T cells (FIG. 18G, CD123_{41C} ADCs). A higher DAR did not improve the efficacy of the anti-CD20 based ADCs (FIG. 4G), probably because the maximum activity was already reached with a DAR2. The potency of the anti-CD20 ADCs improved with a higher DAR, but also the potency of the non-binding isotype controls. Overall, these results show that anti-CD123 pAg conjugates can activate Vδ2 γδ T cells better than the mAb only and that a higher DAR improves the efficacy of the anti-CD123 pAg conjugates.

1. A conjugate, comprising a targeting moiety covalently linked to an immunomodulating moiety, wherein the immunomodulating moiety is a phosphoantigen moiety.

2. The conjugate according to claim 1 wherein the targeting moiety is a tumor-targeting antibody or antigen binding fragment thereof.

3. The conjugate according to claim 2 wherein the phosphoantigen moiety is a pyrophosphate, pyrophosphonate, bisphosphonate, monophosphate, monophosphonate, or prodrugs thereof, and comprises an allylalcohol group.

4. The conjugate according to claim 1, wherein the targeting moiety is linked to the phosphoantigen moiety via a linking moiety.

5. C The conjugate according to claim 1, represented by formula I
Tm-(L-(pAg)ₓ)_{y} (I)
wherein
Tm represents a targeting-moiety,
L represents a linking moiety,
pAg represents a phosphoantigen moiety,
x represents the number of phosphoantigen moieties per linking moiety, and is an integer ranging from 1-5,
y represents the number of L-(pAg)ₓ per Tm (targeting moiety) and has a value ranging from 1-10.

6. The conjugate according to claim 5, wherein y has a value ranging from 1-8.

7. The conjugate according to claim 1, wherein the linking moiety comprises a cleavable linker.

8. A linker-drug compound comprising one or more phosphoantigen moieties covalently bound to a linking moiety (L), with the general structure reflected in formula (II) 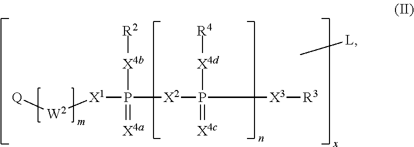
wherein
Q represents a structure reflected in formula IIa or IIb: 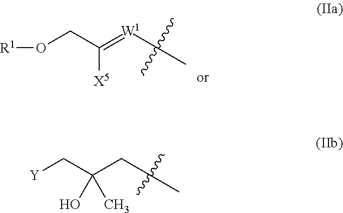
Y is a halogen,
W¹ is N, CH or CF;
W² is CH₂, CHF, CF₂ or O;
X¹ is O, S, NH, CH₂, CHF or CF₂;
X² is O, CH₂, CHF or CF₂;
X³ is absent or O or NH;
each of X^{4a-d} is independently selected from O and S;
X⁵ is CH₃, CH₂F, CHF₂, CF₃, or CCl₃;
x is an integer ranging from 1-5;
m is 1, 2 or 3;
n is 0, 1 or 2;
R¹ is H or a connection to the linking moiety (L) or a prodrug moiety;
R² is H or a connection to the linking moiety (L) or Cat+ or a prodrug moiety;
R³ is H or a connection to the linking moiety (L) or Cat+ or a prodrug moiety;
R⁴ is H or a connection to the linking moiety (L) or Cat+ or a prodrug moiety;
or, when n is O, R³ and R² are connected by a C₁₋₆ (hetero)alkyl group or;
when n is 1 or 2, R³ and R⁴ are connected by a C₁₋₆ (hetero)alkyl group.

9. The linker-drug compound according to claim 8, wherein Q represents a structure reflected in formula IIa.

10. The linker-drug compound according to claim 9, wherein W¹ is CH, X⁵ is CH₃ and R¹ is H or a connection to the linking moiety (L).

11. The linker-drug compound according to claim 9, wherein X³ is O, R³ is a connection to a cleavable linking moiety and R¹ is H.

12. The linker-drug compound according to claim 8, wherein W² is CH₂ and m is 1.

13. The linker-drug compound according to claim 8, wherein X¹ is CH₂.

14. The linker-drug compound according to claim 9, wherein X³ is O, R³ is a connection to a cleavable linking moiety, W¹ is CH, X⁵ is CH₃, R¹ is H, W² is CH₂ and m is 1, and X¹ is CH₂.

15. The linker-drug compound according to claim 8, wherein n is 0 or 1, X^{4a-b} and X^{4c-d} (when present) are O and wherein R² and R⁴ (when present) are H.

16. The linker-drug compound according to claim 8, wherein R² and R³ are prodrug moieties selected from the group consisting of:
a pivaloyloxymethyl (POM) and isopropyloxycarbonyloxymethyl (POC) group,
a substituted or non-substituted (hetero)aryl group, and
a structure according to formula IV or V 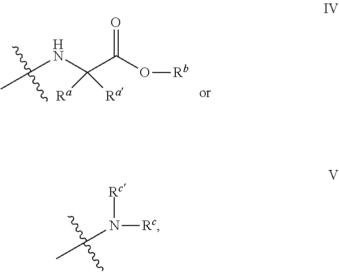
wherein;
R^{a} and R^{a′} are independently selected from H, an optionally substituted amino acid side chain and a non-polar side chain comprising an optionally substituted C₁₋₁₄ alkyl chain,
R^{b} is H, benzyl or a substituted or non-substituted (C₁₋₈)alkyl,
R^{c} and R^{c′} are independently selected from H and an, optionally substituted, C₁-C₆ alkyl, C₃-C₆ cycloalkyl, aryl or heteroaryl.

17. The linker-drug compound according to claim 16, wherein R² and R³ are independently selected from a POM- and POC-group.

18. The linker-drug compound according to claim 16, wherein n is 0, R² is a substituted or non-substituted 5 or 6 membered (hetero)aryl group and R³ is a structure according to formula IV or V, or vice versa.

19. The linker-drug compound according to claim 8, wherein the linking moiety (L) comprises a structure according to formula VI or VII 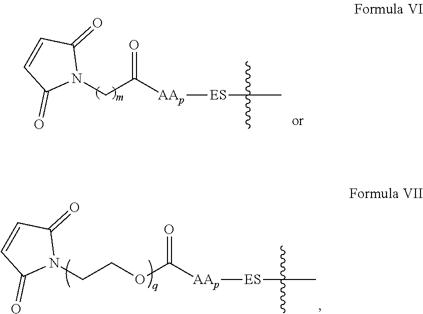
wherein
m is an integer ranging from 1 to 10;
AA is an amino acid; and
p is 0, 1, 2, 3, or 4;
q is an integer ranging from 1 to 12;
ES is either absent or an elongation spacer selected from 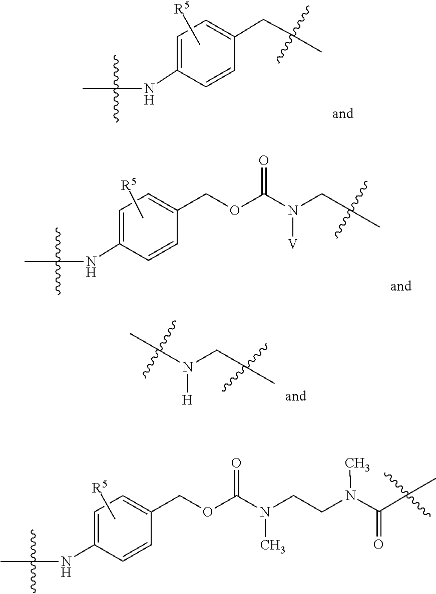
wherein R⁴ is H, halogen, CF₃, C₁₋₄ alkyl, C₂₋₄ alkenyl, C₂₋₄ alkynyl, C₁₋₄ alkoxyl, or C₁₋₄ alkylthio;
wherein V is H, ethyl, —(CH₂CH₂O)ₚ—OMe, CH₂CH₂SO₂Me or CH₂CH₂N(Me)₂;
wherein p is an integer ranging from 1 to 12

20. The linker-drug compound according to claim 8, having the following structure:

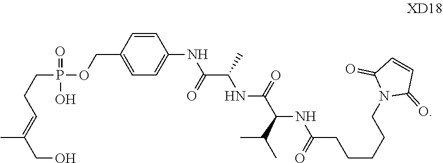 [SMILES: C/C(=C\CCP(=O)(O)OCc1ccc(NC(=O)[C@H](C)NC(=O)[C@@H](NC(=O)CCCCCN2C(=O)C=CC2=O)C(C)C)cc1)CO]

21. (canceled)

22. (canceled)

23. A method for treating a subject with cancer, autoimmune disease or an infection, which comprises administering a correspondingly therapeutically effective amount of the conjugate according to claim 1 to a subject in need thereof.

24. A conjugate, comprising a tumor targeting antibody or antigen binding fragment thereof, conjugated to a linker drug compound having the following structure:

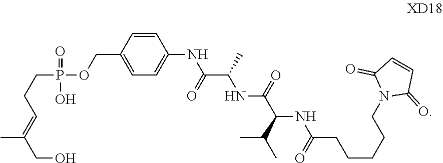 [SMILES: C/C(=C\CCP(=O)(O)OCc1ccc(NC(=O)[C@H](C)NC(=O)[C@@H](NC(=O)CCCCCN2C(=O)C=CC2=O)C(C)C)cc1)CO]

25. A pharmaceutical composition comprising a conjugate according to claim 1, and one or more pharmaceutical excipients.
